(19) United States
(12) Patent Application Publication
(10) Pub. No.: US 2005/0043315 A1
(43) Pub. Date: Feb. 24, 2005

(54) Aminopyrimidine compounds, processes for their preparation and pharmaceutical compositions containing them

(76) Inventors: Hideo Tsutsumi, Osaka (JP); Satoshi Yonishi, Osaka (JP); Atsushi Akahane, Osaka (JP)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 10/498,016

(22) PCT Filed: Dec. 27, 2002
(86) PCT No.: PCT/JP02/13796

(30) Foreign Application Priority Data
Jan. 2, 2002 (AU) PR 9796
Apr. 12, 2002 (AU) PS 1724
Sep. 16, 2002 (AU) 2002-951403

(51) Int. Cl. C07D 43/02; A61K 31/501

(52) U.S. Cl. 514/252.02; 514/252.03; 544/238

(57) ABSTRACT

An aminopyrimidine compound of the following formula (I) wherein Q is (a) or (b) in which R and R′ are each optionally substituted aryl or heterocyclic group,R⁵ is hydrogen, halogen, lower alkyl,optionally substituted hydroxy, optionally substituted amino which may form N-containing heterocyclic group, optionally substituted mercapto, lower alkylsulfinyl or lower alkylsulfonyl, and X is oxygen or sulfur; R¹ is hydrogen, optionally substituted lower alkyl or cyclo(lower) alkyl which may be interrupted by an oxygen atom; R² and R³ are each independently hydrogen, lower alkyl, acyl, aryl or heterocyclic(lower)alkyl, R² and R³ may be combined together with N atom to which they are attached to form N-containing heterocyclic group; or a salt thereof. The aminopyrimidine compound (I) and salt thereof of the present invention are adenosine antagonists and are useful for the prevention and/or treatment of depression, dementia (e.g. Alzheimer's disease, cerebrovascular dementia, dementia accompanying Parkinson's disease, etc.), Parkinson's disease, anxiety, pain, cerebrovascular disease (e.g. stroke, etc.), heart failure and the like.

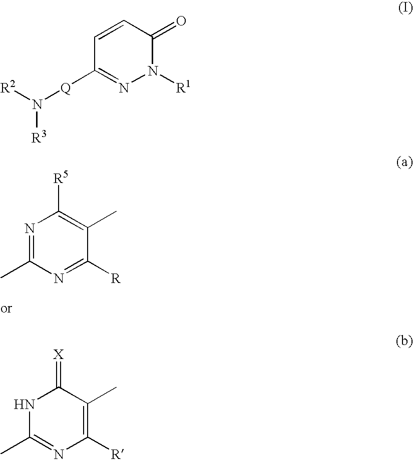

### TECHNICAL FIELD

The present invention relates to a novel aminopyrimidine compound and a salt thereof, which are useful as medicaments.

### BACKGROUND ART

2-Amino-4-aryl-5-(6-oxo-1,6-dihydro-pyridazin-3-yl)-pyrimidine compounds and derivatives thereof are novel, so there has been no knowledge about these compounds. In addition, any aminopyrimidine compounds having both of adenosine A₁ and A₂ₐ inhibitory activities are not known.

### DISCLOSURE OF INVENTION

The present invention relates to a novel aminopyrimidine compound and a pharmaceutically acceptable salt thereof, which are useful as medicaments; processes for the preparation of said aminopyrimidine compound and a salt thereof; a pharmaceutical composition comprising, as an active ingredient, said aminopyrimidine compound or a pharmaceutically acceptable salt thereof; a use of said aminopyrimidine compound or a pharmaceutically acceptable salt thereof as a medicament; and a method for using said aminopyrimidine compound or a pharmaceutically acceptable salt thereof for therapeutic purposes, which comprises administering said aminopyrimidine compound or a pharmaceutically acceptable salt thereof to a human being or an animal.

The aminopyrimidine compound and a salt thereof are adenosine antagonists (especially, A₁ receptor and A₂ (particularly A₂ₐ) receptor dual antagonists) and possess various pharmacological actions such as anticatalepsy action, cognitive enhancing action, analgesic action, locomotor action, antidepressant action, diuretic action, cardioprotective action, cardiotonic action, vasodilating action (e.g. cerebral vasodilating action, etc.), the action of increasing the renal blood flow, renal protective action, improvement action of renal function, enhancing action of lipolysis, inhibition action of anaphylactic bronchoconstriction, acceleration action of the insulin release, the action of increasing the production of erythropoietin, inhibiting action of platelet aggregation, or the like.

They are useful as cognitive enhancer, antianxiety drug, antidementia drug, psychostimulant, analgesic, cardioprotective agent, antidepressant, ameliorants of cerebral circulation, tranquilizer, drug for heart failure, cardiotonic agent, antihypertensive agent, drug for renal failure (renal insufficiency), drug for renal toxicity, renal protective agent, drug for improvement of renal function, diuretic, drug for edema, antiobesity, antiasthmatic, bronchodilator, drug for apnea, drug for gout, drug for hyperuricemia, drug for sudden infant death syndrome (SIDS), ameliorants of immunosuppressive action of adenosine, antidiabetic agent, drug for ulcer, drug for pancreatitis, drug for Meniere's syndrome, drug for anemia; drug for thrombosis, drug for myocardial infarction, drug for obstruction, drug for arteriosclerosis obliterans, drug for thrombophlebitis, drug for cerebral infarction, drug for transient ischemic attack, drug for angina pectoris, or the like;
and useful for the prevention and/or treatment of depression, dementia (e.g. Alzheimer's disease, cerebrovascular dementia, dementia accompanying Parkinson's disease, etc.), Parkinson's disease, anxiety, pain, cerebrovascular disease (e.g. stroke, etc.), heart failure;
hypertension (e.g. essential hypertension, nephrogenous hypertension, etc.);
circulatory insufficiency (acute circulatory insufficiency) cuased by, for example, ischemia/reperfusion injury (e.g. myocardial ischemia/reperfusion injury, cerebral ischemia/reperfusion injury, peripheral ischemia/reperfusion injury, etc.), shock (e.g. endotoxin shock, hemorrhagic shock, etc.), surgical procedure, or the like; post-resuscitation asystole;
bradyarrhythmia;
electro-mechanical dissociation;
hemodynamic collapse;
SIRS (systemic inflammatory response syndrome);
multiple organ failure;
renal failure (renal insufficiency) (e.g. acute renal failure, etc.), renal toxicity [e.g. renal toxicity induced by a drug such as cisplatins, gentamicin, FR-900506 (disclosed in EP-0184162), cyclosporin (e.g. cyclosporin A) or the like;
glycerol, etc.], nephrosis, nephritis, edema (e.g. cardiac edema, nephrotic edema, hepatic edema, idiopathic edema, drug edema, acute angioneurotic edema, hereditary angioneurotic edema, carcinomatous ascites, gestational edema, etc.);
obesity, bronchial asthma, gout, hyperuricemia, sudden infant death syndrome, immunosuppression, diabetes, ulcer such as peptic ulcer (e.g. gastric ulcer, duodenal ulcer, etc.), pancreatitis, Meniere's syndrome, anemia, dialysis-induced hypotension, constipation, ischemic bowel disease, ileus (e.g. mechanical ileus, adynamic ileus, etc.); and
myocardial infarction, thrombosis (e.g. arterial thrombosis, cerebral thrombosis, etc.), obstruction, arteriosclerosis obliterans, thrombophlebitis, cerebral infarction, transient ischemic attack, angina pectoris, or the like.

The novel aminopyrimidine compound of the present invention can be shown by the following formula (I). 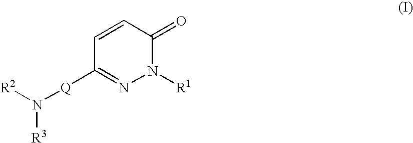
wherein
Q is 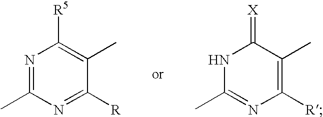
in which
R and R′ are each optionally substituted aryl or heterocyclic group,
R⁵ is hydrogen, halogen, lower alkyl, optionally substituted hydroxy, optionally substituted amino which may form N-containing heterocyclic group, optionally substituted mercapto, lower alkylsulfinyl or lower alkylsulfonyl, and
X is oxygen or sulfur;
R¹ is hydrogen, optionally substituted lower alkyl or cyclo(lower)alkyl which may be interrupted by an oxygen atom;
R² and R³ are each independently hydrogen, lower alkyl, acyl, aryl or heterocyclic (lower) alkyl,
R² and R³ may be combined together with N atom to which they are attached to form N-containing heterocyclic group;
or a salt thereof.

The preferred embodiments of the aminopyrimidine compound of the present invention represented by the general formula (I) are as follows.

(1) The aminopyrimidine compound of the general formula (I)

wherein

Q is 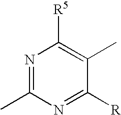
in which R and R⁵ are each as defined above,
R¹ is optionally substituted lower alkyl, and
R² and R³ are defined above.
   (2) The aminopyrimidine compound of (1) above
   wherein
R¹ is lower alkyl or lower alkoxy(lower)alkyl, and
R⁵ is hydrogen.
   (3) The aminopyrimidine compound of (2) above
   wherein
R¹ is lower alkyl, and
R² is hydrogen.
   (4) The aminopyrimidine compound of the general formula (I)
   wherein
R¹ is hydrogen, lower alkyl, hydroxy(lower)alkyl, lower alkoxy(lower)alkyl or phenyl(lower)alkyl,
R² is hydrogen, lower alkyl, lower alkanoyl or optionally substituted benzoyl,
R³ is hydrogen, lower alkyl, phenyl, pyridinyl(lower)alkyl or —CO—R³¹,
   in which R³¹ is lower alkyl, cyclo(lower)alkyl, lower alkoxy(lower)alkyl, optionally substituted lower alkoxy, optionally substituted phenyl or pyridinyl,
R² and R³ may be combined together with N atom to which they are attached to form N-containing heterocyclic group;
R and R′ are each 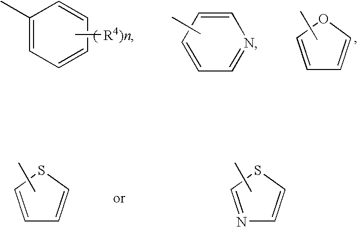
   in which R⁴ is hydrogen, halogen, hydroxy, lower alkyl, optionally substituted lower alkoxy, trihalo (lower) alkyl, lower alkylthio, lower alkylsulfinyl or lower alkylsulfonyl, and n is an integer from 1 to 3,
   provided R⁴ may be different from each other when n is 2 or 3; and
R⁵ is hydrogen, halogen, lower alkyl, lower alkylthio, lower alkanoylthio, arylthio, lower alkylsulfinyl, lower alkylsulfonyl, —O—R⁵¹,
   in which R⁵¹ is hydrogen, optionally substituted lower alkyl, lower alkenyl, lower alkynyl, cyclo(lower)alkyl, aryl or heterocyclic group, or 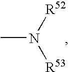
   in which R⁵² is hydrogen or lower alkyl;
   R⁵³ is hydrogen, optionally substituted lower alkyl, lower alkenyl, cyclo(lower)alkyl, amidino, aryl or heterocyclic group,
   R⁵² and R⁵³ may be combined together with N atom to which they are attached to form N-containing heterocyclic group;
      or a salt thereof.
      (5) The aminopyrimidine compound of (4) above
      wherein
R¹ is hydrogen, methyl, ethyl, propyl, isopropyl, hydroxyisopropyl, methoxyisopropyl or benzyl;
R² is hydrogen, methyl,acetyl, benzoyl, toluoyl, methoxybenzoyl, trifluoromethylbenzoyl, fluorobenzoyl or chlorobenzoyl;
R³ is hydrogen, methyl, phenyl, pyridinylmethyl or —CO—R³¹,
   in which R³¹ is methyl, propyl, isopropyl, isobutyl, tert-butyl, cyclopropyl, cyclohexyl, methoxy, methoxymethyl, trichloroethoxy, phenyl, tolyl, methoxyphenyl, trifluoromethylphenyl, fluorophenyl, chlorophenyl or pyridinyl, and
R² and R³ may be combined together with N atom to which they are attached to form morpholino;
R and R′ are each 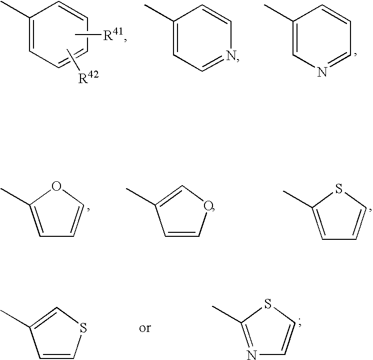
   in which R⁴¹ and R⁴² are each independently hydrogen, fluoro, bromo, chloro, hydroxy, methyl, trifluoromethyl, methoxy, ethoxy, propoxy, isopropoxy, butoxy, isobutoxy, isopentyloxy, hexyloxy, methoxyethoxy, fluoroethoxy, fluoropropoxy, dimethylaminoethoxy, morpholinylethoxy, methylthio, methylsulfinyl or methylsulfonyl; and
R⁵ is hydrogen, fluoro, methyl, methylthio, acetylthio, phenylthio, methylsulfinyl, methylsulfonyl, —O—R⁵¹,
   in which R⁵¹ is hydrogen, methyl, ethyl, propyl, isopropyl, allyl, propynyl, cyclobutyl, cyclohexyl, hydroxyethyl, methoxyethyl, carboxymethyl, aminoethyl, dimethylaminoethyl, fluoroethyl, carbamoylmethyl, methylcarbamoylmethyl, dimethylcarbamoylmethyl, cyclopropylcarbamoylmethyl, methoxycarbonylmethyl, tert-butoxycarbonylmethyl, acetylmethyl, benzoylmethyl, phenyl, benzyl, pyridinylmethyl, pyridinylethyl, tetrahydro-2H-pyranyl or 1,3(2H)-dioxoisoindolinylethyl, or 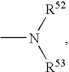
   in which R⁵² is hydrogen or methyl, R⁵³ is hydrogen, methyl, ethyl, propyl, isopropyl, tert-butyl, allyl, cyclopropyl, hydroxyethyl, methoxyethyl, aminoethyl, dimethylaminoethyl, carbamoylmethyl, amidino, phenyl, benzyl, pyridinyl, pyridinylmethyl, furylmethyl or dimethylthiazolyl,
   R⁵² and R⁵³ may be combined together with N atom to which they are attached to form pyrrolidinyl, piperidinyl, morpholino, piperazinyl, methylpiperazinyl, imidazolyl, triazolyl or benzimidazolyl,
      or a salt thereof.

The object compound (I) and a salt thereof of the present invention can be prepared by the following processes. 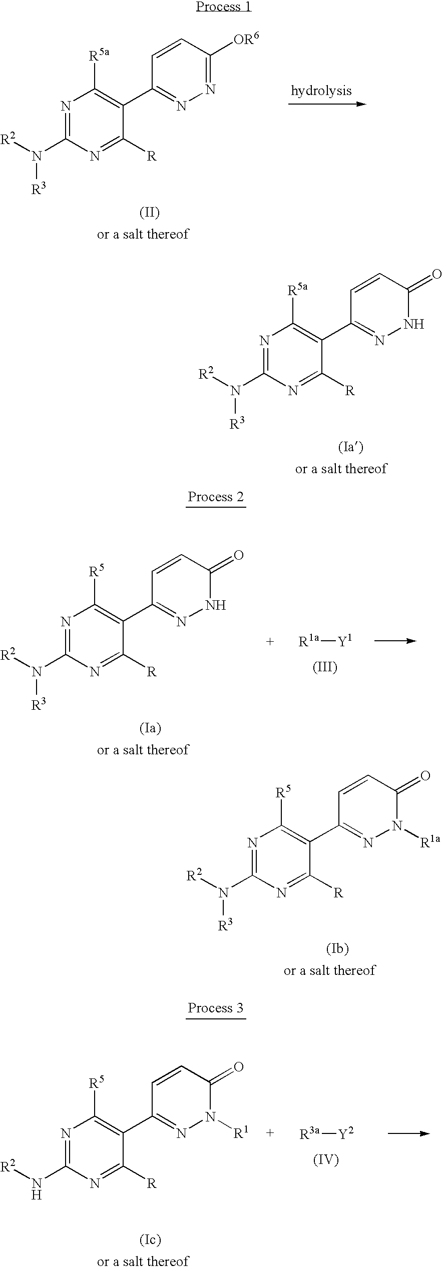 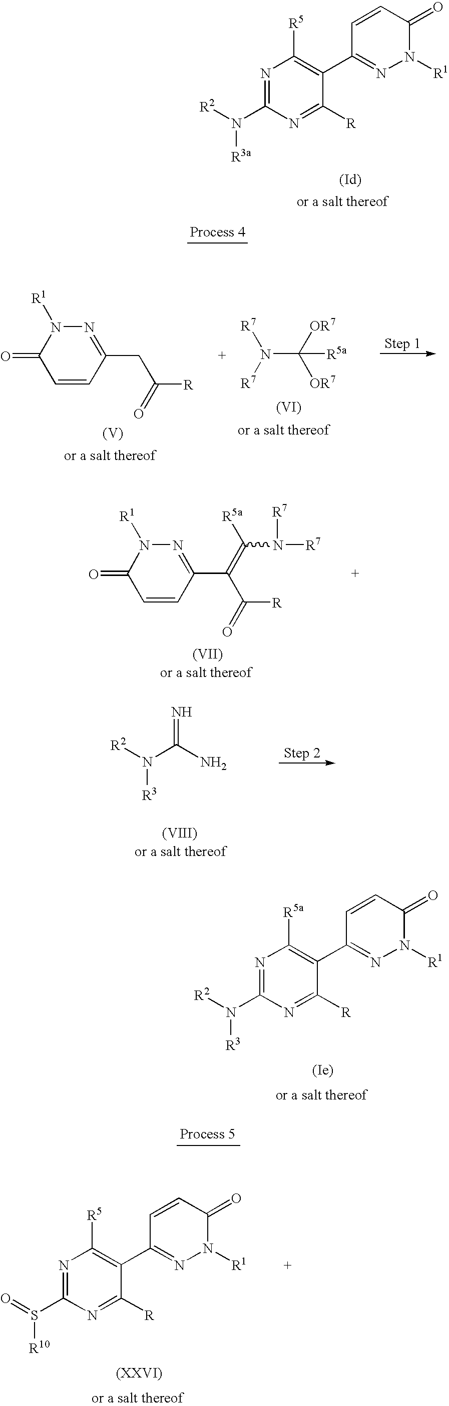 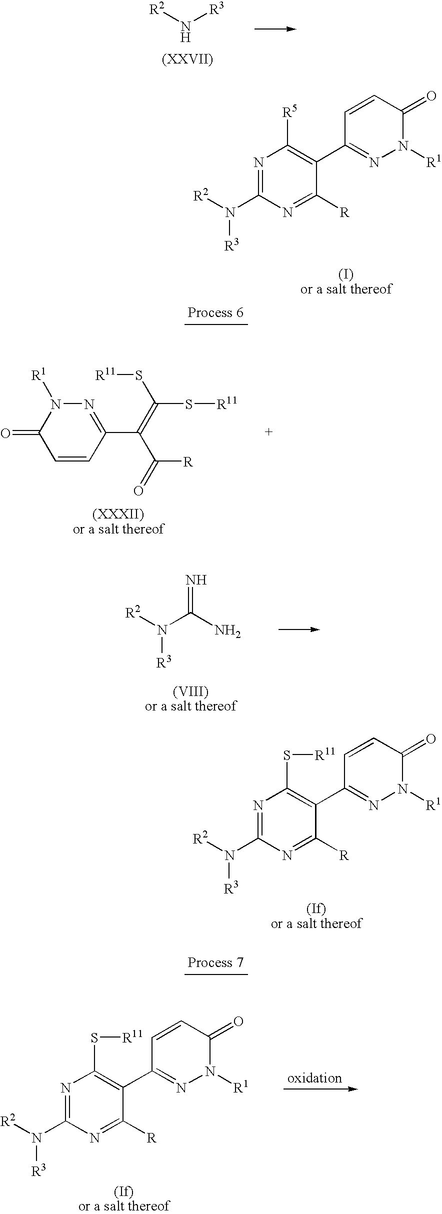 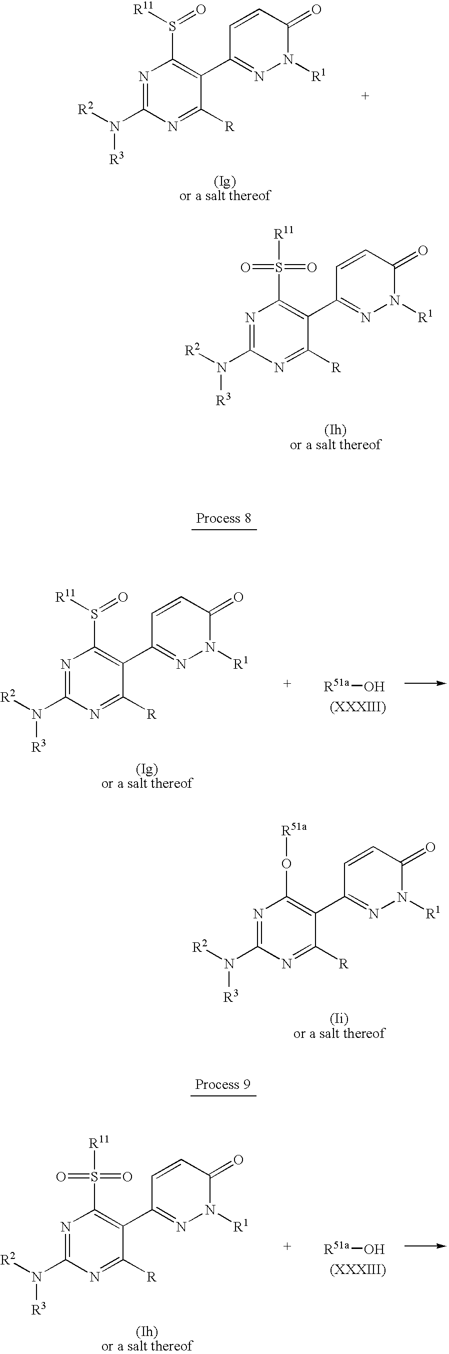 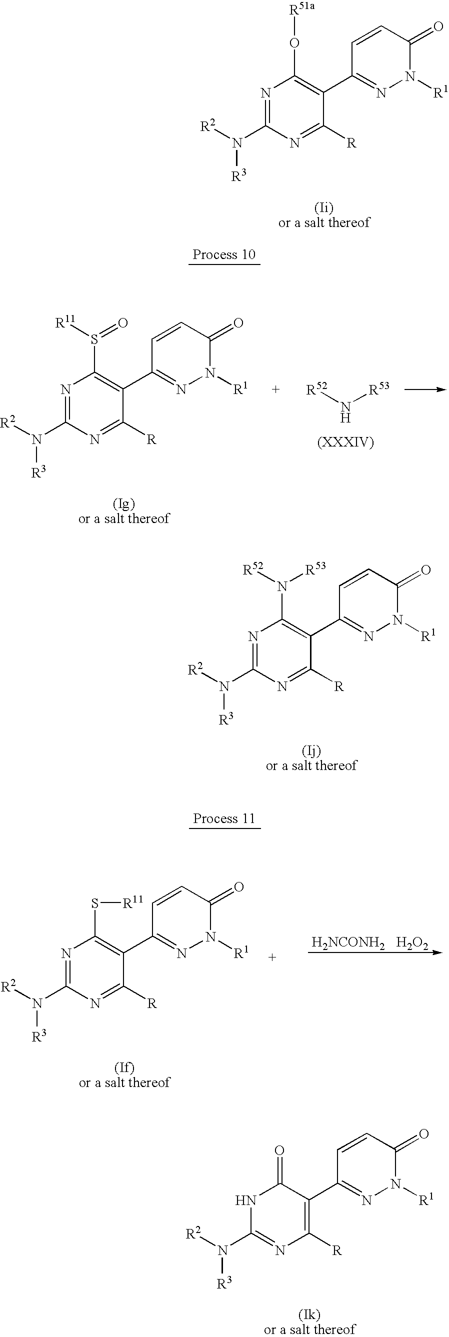 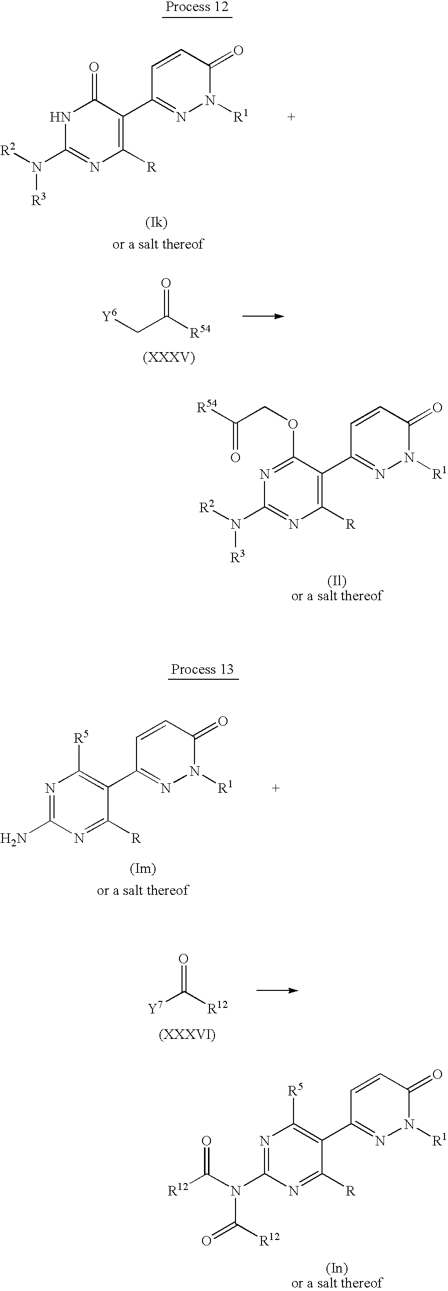 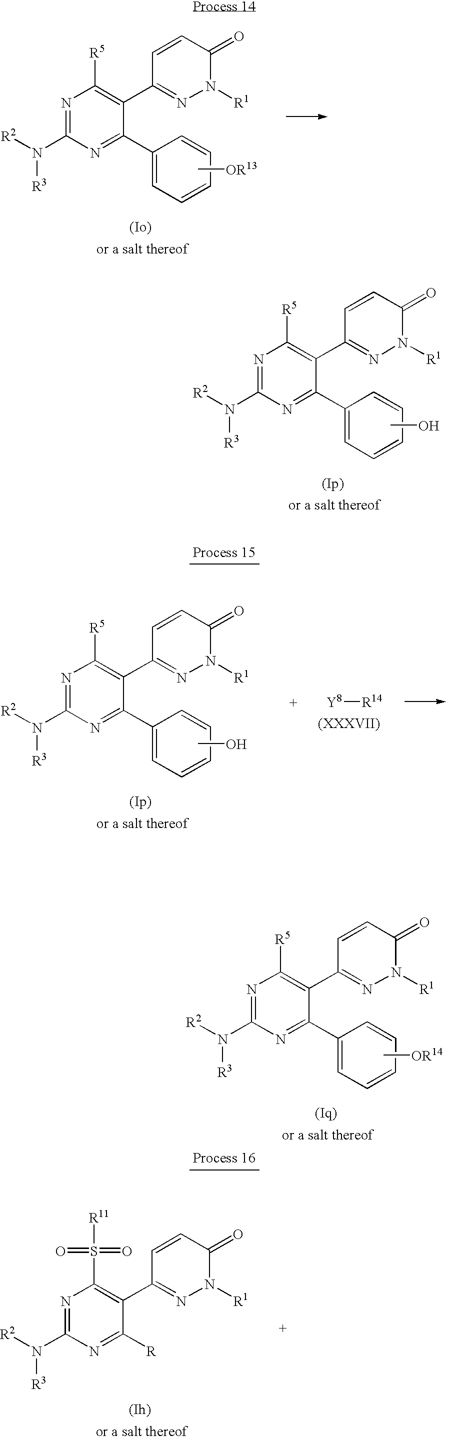 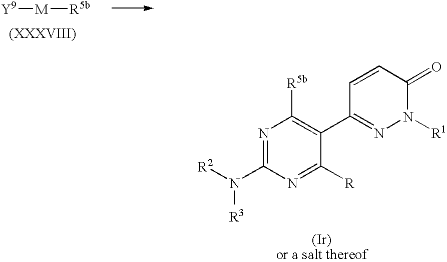
wherein
R is optionally substituted aryl or heterocyclic group;
R¹ is hydrogen, optionally substituted lower alkyl, cyclo(lower)alkyl which may be interrupted by an oxygen atom or aryl(lower)alkyl;
R^{1a} is optionally substituted lower alkyl, cyclo(lower)alkyl which may be interrupted by an oxygen atom or aryl (lower) alkyl;
R² and R³ are each independently hydrogen, lower alkyl, acyl, aryl or heterocyclic (lower) alkyl,
R² and R³ may be combined together with N atom to which they are attached to form N-containing heterocyclic group;
R^{3a} is lower alkyl, acyl, aryl or heterocyclic(lower)alkyl;
R⁵ is hydrogen, halogen, lower alkyl, optionally substituted hydroxy, optionally substituted amino which may form N-containing heterocyclic group, optionally substituted mercapto, lower alkylsulfinyl or lower alkylsulfonyl;
R^{5a} is hydrogen, lower alkyl, optionally substituted hydroxy or optionally substituted amino;
R^{5b} is lower alkyl;
R^{51a} is optionally substituted lower alkyl, lower alkenyl, lower alkynyl, cyclo(lower)alkyl, aryl or heterocyclic group,
R⁵² is hydrogen or lower alkyl;
R⁵³ is hydrogen, optionally substituted lower alkyl, lower alkenyl, cyclo(lower)alkyl, amidino, aryl or heterocyclic group,
R⁵² and R⁵³ may be combined together with N atom to which they are attached to form N-containing heterocyclic group;
R⁵⁴ is lower alkyl, cyclo(lower)alkyl, lower alkoxy or aryl;
R⁶, R⁷, R¹⁰, R¹¹ and R¹³ are each lower alkyl;
R¹² is optionally substituted aryl or lower alkoxy;
R¹⁴ is optionally substituted lower alkyl;
M is metal; and
Y¹, Y², Y⁶, Y⁷, Y⁸ and Y⁹ are each a leaving group.

The starting compound(II) or a salt thereof is novel and can be prepared, for example, by the following reaction schemes. 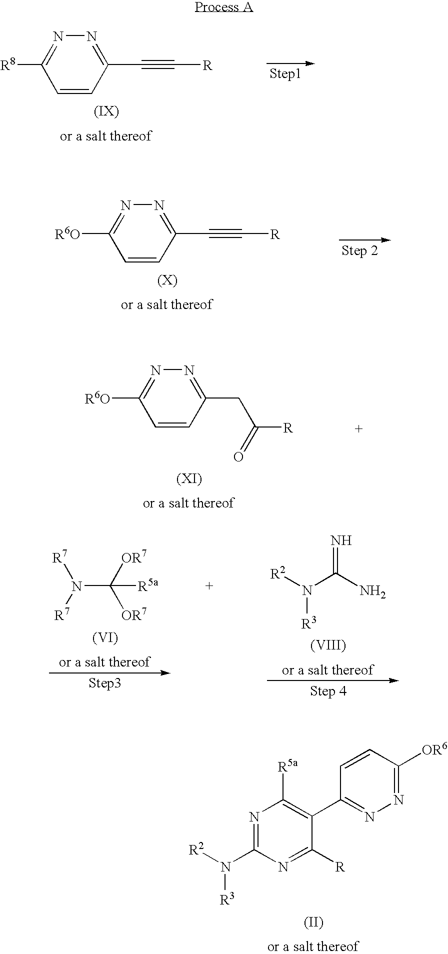
wherein R, R², R³, R^{5a}, R⁶ and R⁷ are as defined above, and
R⁸ is arylsulfonyl which may have one or more suitable substituent(s); 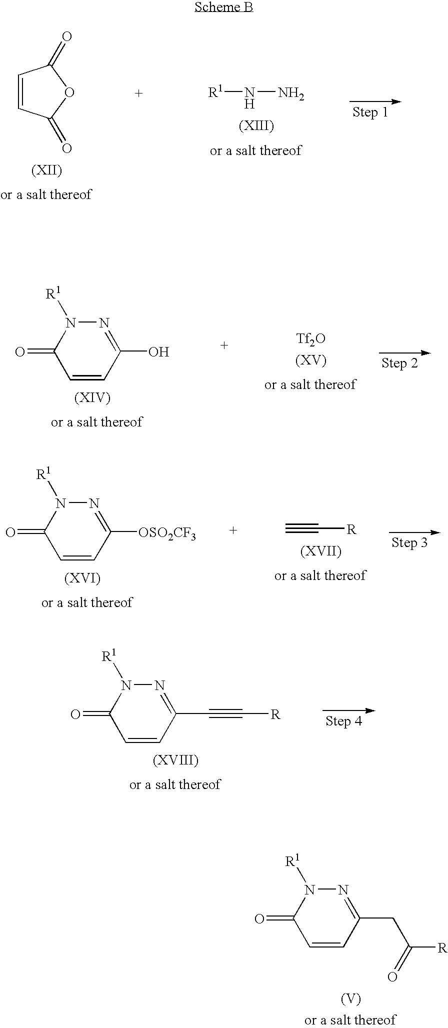
   wherein R and R¹ are as defined above, and
Tf₂O is trifluoromethanesulfonic anhydride. 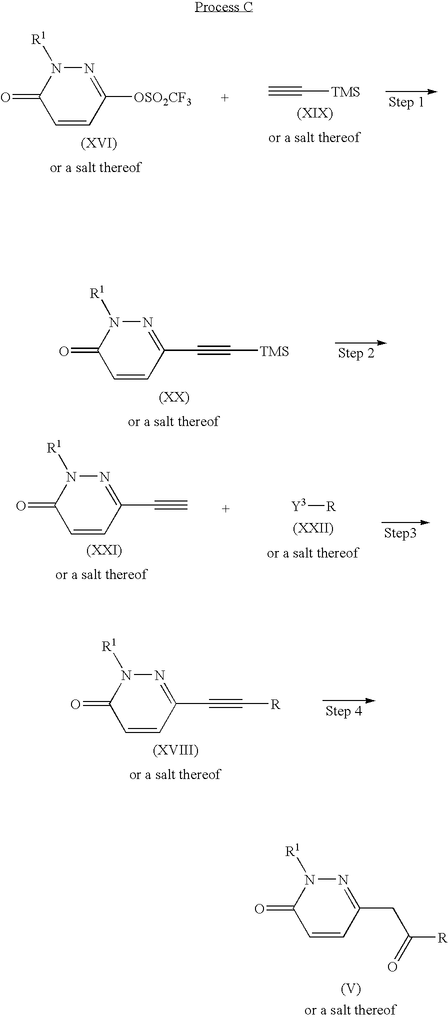
   wherein R and R¹ are as defined above,
Y³ is a leaving group, and TMS is trimethylsilyl. 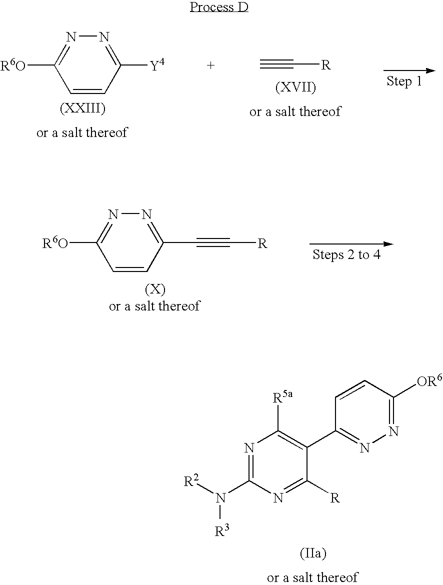
   wherein R, R², R³, R^{5a} and R⁶ are as defined above,
Y⁴ is a leaving group, and
Steps 2 to 4 in Process D are as same as those of Process A. 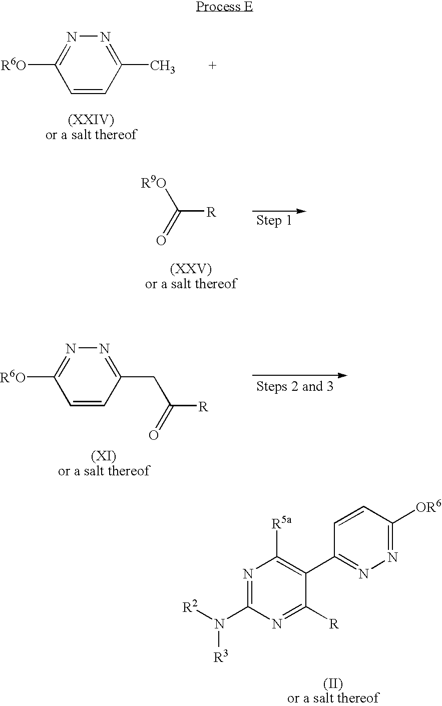
   wherein R, R², R³, R^{5a} and R⁶ are as defined above,
R⁹ is lower alkyl, and
Steps 2 and 3 in Process E are as same as Steps 3 and 4 of Process A respectively. 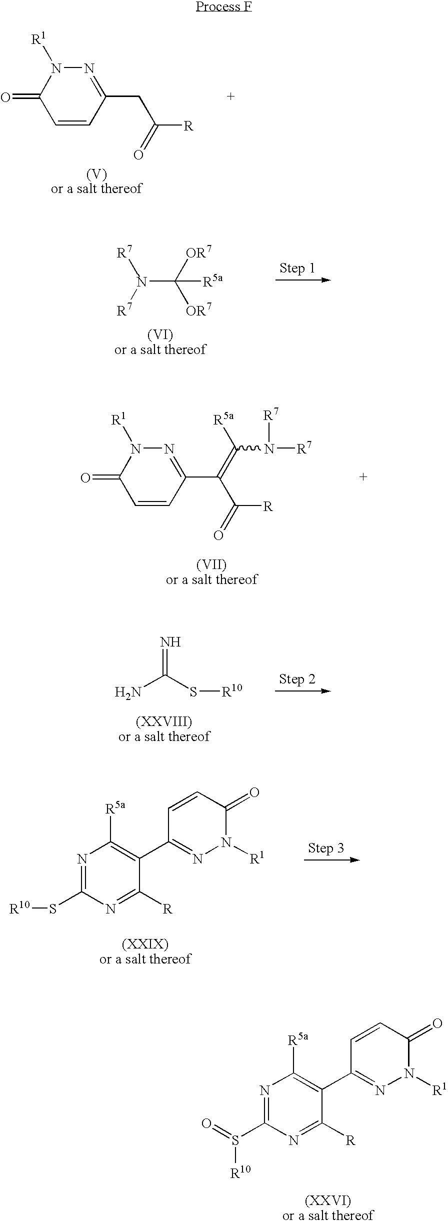
   wherein R, R¹, R^{5a}, R⁷ and R¹⁰ are as defined above, and
Step 1 in Process F is as same as Step 1 of Process 4. 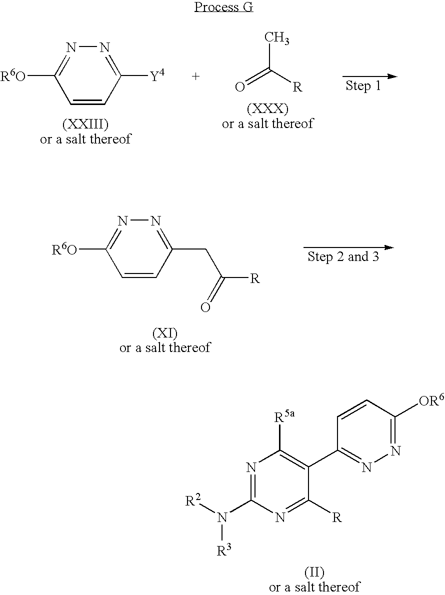
   wherein R, R², R³, R^{5a}, R⁶ and Y⁴ are as defined above, and Steps 2 and 3 in Process G are as same as Steps 3 and 4 of Process A respectively. 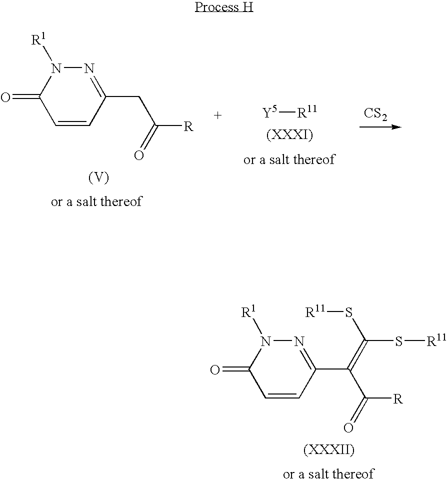
   wherein R, R¹ and R¹¹ are as defined above, and
Y⁵ is a leaving group.

In addition to the processes as mentioned above, the object compound (I) and a salt thereof can be prepared, for example, according to the procedures as illustrated in Examples in the present specification or in a manner similar thereto.

The starting compounds can be prepared, for example, according to the procedures as illustrated in Preparations in the present specification or in a manner similar thereto.

The object compound (I) and a salt thereof can be prepared according to the methods as shown in Preparations or Examples, or in a manner similar thereto.

It is to be noted that the object compound (I) may include the geometrical isomer(s) due to the double bond(s) and/or the stereo isomer(s) due to the asymmetric carbon atom(s). In this regard, one isomer can be converted to another according to a conventional method in this field of the art.

It is also to be noted that the solvating form of the compound (I) (e.g. hydrate, etc.) and any form of the crystal of the compound (I) are included within the scope of the present invention.

Suitable salts of the object compound (I) are conventional pharmaceutically acceptable ones and include a metal salt such as an alkali metal salt (e.g. sodium salt, potassium salt, etc.) and an alkaline earth metal salt (e.g. calcium salt, magnesium salt, etc.), an ammonium salt, an organic base salt (e.g. trimethylamine salt, triethylamine salt, pyridine salt, picoline salt, dicyclohexylamine salt, N,N′-dibenzylethylenediamine salt, etc.), an organic acid salt (e.g. acetate, trifluoroacetate, maleate, tartrate, fumarate, methanesulfonate, benzenesulfonate, formate, toluenesulfonate, etc.), an inorganic acid salt (e.g. hydrochloride, hydrobromide, hydriodide, sulfate, phosphate, etc.), a salt with an amino acid (e.g. arginine, aspartic acid, glutamic acid, etc.), and the like.

Suitable examples and illustrations of the various definitions which the present invention includes within the scope thereof and which appear in the above and following description in the present specification are explained in detail as follows.

The term “lower” is intended to mean 1 to 6 carbon atom(s) unless otherwise indicated.

Suitable “lower alkyl” may include straight or branched ones such as methyl, ethyl, propyl, isopropyl, butyl, isobutyl, tert-butyl, pentyl, hexyl or the like, in which the preferred one may be methyl, ethyl, propyl, isopropyl, isobutyl or tert-butyl.

Suitable “lower alkenyl” may include straight or branched ones such as vinyl, 1-propenyl, allyl, butenyl, pentenyl, hexenyl, or the like, in which the preferred one may be allyl.

Suitable “lower alkynyl” may include straight or branched ones such as ethynyl, propynyl, butynyl, or the like, in which the preferred one may be propynyl.

Suitable “lower alkoxy” may include straight or branched ones such as methoxy, ethoxy, propoxy, isopropoxy, butoxy, isobutoxy, tert-butoxy, pentyloxy, isopentyloxy, hexyloxy or the like.

Suitable “cyclo(lower)alkyl” may be cyclo(C3-C8)alkyl such as cyclopropyl, cyclobutyl, cyclopentyl, cyclohexyl, cycloheptyl, cyclooctyl or the like, in which the preferred one may be cyclopropyl, cyclobutyl or cyclohexyl.

Said “cyclo(lower)alkyl” may be interrupted by an oxygen atom, in which the preferred one may be saturated 3-8-membered heteromonocyclic group containing an oxygen atom such as tetrahydrofuranyl or tetrahydropyranyl.

Suitable “acyl” may include “optionally substituted carbonyl” such as carboxy, optionally substituted lower alkanoyl, optionally substituted lower alkoxycarbonyl, lower cycloalkanoyl, optionally substituted benzoyl, pyridinylcarbonyl or optionally substituted carbamoyl, or the like.

Suitable examples of aforesaid “lower alkanoyl” may include formyl, acetyl, propionyl, isopropionyl, butyryl, isobutyryl, tert-butyryl, valeryl, isovaleryl, pivaloyl, hexanoyl or the like, in which the preferred one may be (C1-C4)alkanoyl and the more preferred one may be acetyl propionyl, isopropionyl, butyryl, isobutyryl, tert-butyryl,.

Suitable substituent of aforesaid “substituted lower alkanoyl” may include lower alkoxy (e.g. methoxy etc.), or the like.

Suitable examples of aforesaid “lower cycloalkanoyl” may be cyclopropanoyl, cyclobutanoyl, cyclopentanoyl, cyclohexanoyl, in which the preferred one is cyclopropanoyl, cyclohexanoyl.

Suitable examples of aforesaid “optionally substituted lower alkoxycarbonyl” may be methoxycarbonyl, ethoxycarbonyl, tert-butoxycarbonyl, trichloroethoxycarbonyl, or the like.

Suitable examples of aforesaid “optionally substituted benzoyl” may include benzoyl, halobenzoyl (e.g. fluorobenzoyl, chlorobenzoyl, etc.), loweralkylbenzoyl (e.g. toluoyl, etc.), lower alkoxybenzoyl (e.g. methoxybenzoyl, etc.), trihalo(lower)alkyl benzoyl (e.g. trifluoromethoxybenzoyl, etc.), or the like.

Suitable examples of aforesaid “optionally substituted carbamoyl” may include carbamoyl or N-substituted carbamoyl such as N-(lower)alkylcarbamoyl, N-cyclo(lower)alkylcarbamoyl, N,N-di(lower) alkylcarbamoyl, or the like, in which the preferred examples of “optionally substituted carbamoyl” may be carbamoyl, methylcarbamoyl, dimethylcarbamoyl, cyclopropylcarbamoyl, or the like.

Suitable “aryl” may include phenyl, naphthyl, indenyl, anthryl, or the like, in which the preferred one may be (C6-C10) aryl, and the more preferred one may be phenyl.

Suitable “heterocyclic group” may be saturated or unsaturated monocyclic or polycyclic heterocyclic groups containing at least one hetero atom selected from among oxygen, sulfur and nitrogen, and may be optionally substituted with lower alkyl such as methyl.

Preferable examples of “heterocyclic group” are described in the following.
3- through 8-membered unsaturated heteromonocyclic groups containing 1 through 4 nitrogen atom(s), such as pyrrolyl, pyrrolinyl, imidazolyl, pyrazolyl, pyridinyl (also referred to as pyridyl), and its N-oxide, pyrimidyl, pyrazinyl, pyridazinyl, triazolyl (e.g. 1H-1,2,4-triazolyl, 1H-1,2,3-triazolyl, 2H-1,2,3-triazolyl, etc.), tetrazolyl (e.g. 1H-tetrazolyl, 2H-tetrazolyl, etc.), dihydrotriazinyl (e.g. 4,5-dihydro-1,2,4-triazinyl, 2,5-dihydro-1,2,4-triazinyl, etc.), etc.;
3- through 8-membered saturated heteromonocyclic groups containing 1 through 4 nitrogen atom(s), such as pyrrolidinyl, imidazolidinyl, piperidyl (e.g. piperidino, etc.), piperazinyl, etc.;
unsaturated condensed heterocyclic groups containing 1 through 5 nitrogen atom(s), such as indolyl, isoindolyl, indolizinyl, benzimidazolyl, quinolyl, isoquinolyl, indazolyl, benzotriazolyl, tetrazolopyridyl, tetrazolopyridazinyl (e.g. tetrazolo[1,5-b]pyridazinyl etc.), dihydrotriazolopyridazinyl, etc.;
3- through 8-membered unsaturated heteromonocyclic groups containing 1 or 2 oxygen atoms and 1 through 3 nitrogen atom(s), such as oxazolyl, isoxazolyl, oxadiazolyl (e.g. 1,2,4-oxadiazolyl, 1,3,4-oxadiazolyl, 1,2,5-oxadiazolyl, etc.), etc.;
3- through 8-membered saturated heteromonocyclic groups containing 1 or 2 oxygen atom(s) and 1 through 3 nitrogen atoms, such as morpholinyl, oxazolidinyl (e.g. 1,3-oxazolidinyl etc.), etc.;
unsaturated condensed heterocyclic groups containing 1 or 2 oxygen atom(s) and 1 through 3 nitrogen atom(s), such as benzoxazolyl, benzoxadiazolyl, etc.;
3- through 8-membered unsaturated heteromonocyclic groups containing 1 or 2 sulfur atom(s) and 1 through 3 nitrogen atom(s), such as 1,3-thiazolyl, 1,2-thiazolyl, thiazolinyl, thiadiazolyl (e.g. 1,2,4-thiadiazolyl, 1,3,4-thiadiazolyl, 1,2,5-thiadiazolyl, 1,2,3-thiadiazolyl), etc.;
3- through 8-membered saturated heteromonocyclic groups containing 1 or 2 sulfur atom(s) and 1 through 3 nitrogen atom(s), such as thiazolidinyl etc.;
3- through 8-membered unsaturated heteromonocyclic groups containing 1 sulfur atom, such as thienyl etc.;
unsaturated condensed heterocyclic groups containing 1 or 2 sulfur atoms and 1 through 3 nitrogen atom(s), such as benzothiazolyl, benzothiadiazolyl, etc.;
3- through 8-membered unsaturated heteromonocyclic groups containing 1 or 2 oxygen atom(s), such as furyl, pyranyl, dioxolyl, etc.;
3- through 8-membered saturated heteromonocyclic groups containing 1 or 2 oxygen atom(s), such as oxolanyl, tetrahydropyranyl (e.g. tetrahydro-2H-pyran-2-yl etc.), dioxolanyl, etc.; and
unsaturated condensed heterocyclic groups containing 1 or 2 oxygen atom(s), such as isobenzofuranyl, chromenyl (e.g. 2H-chromen-3-yl etc.), dihydrochromenyl (e.g. 3,4-dihydro-2H-chromen-4-yl etc.), etc.

The particularly preferred example of said “heterocyclic group” may include pyrrolyl, pyrrolinyl, imidazolyl, pyrazolyl, pyridinyl, pyrimidyl, pyrazinyl, pyridazinyl, triazolyl, furyl, thienyl, thiazolyl, dimethylthiazolyl, morpholinyl, pyrrolidinyl, piperidinyl, piperazinyl, methylpiperazinyl, tetrahydro-2H-pyranyl, benzimidazolyl, 1,3(2H)-dioxoisoindolinyl.

Suitable “N-containing heterocyclic group” may be aforesaid “heterocyclic group”, in which said group contains at least one N atom in its ring members, and may be optionally substituted with lower alkyl such as methyl.

Preferable examples of “N-containing heterocyclic group” may include pyrrolidinyl, piperidinyl, morpholino, piperazinyl, methylpiperazinyl, imidazolyl, triazolyl, benzimidazolyl, etc.

Suitable “halogen” may be fluoro, chloro, bromo and iodo, in which the preferred one may be fluoro or bromo.

Suitable “trihalo(lower)alkyl” may include trifluoromethyl, trichloromethyl and tribromomethyl, in which more preferred one is trifluoromethyl.

Suitable “metal” may include magnesium, zinc, or the like.

Suitable “a leaving group” may include halogen as mentioned above, hydroxy, acyloxy such as alkanoyloxy (e.g. acetoxy, propionyloxy, etc.), sulfonyloxy (e.g. mesyloxy, tosyloxy, etc.), or the like.

Suitable “arylsulfonyl” may include phenylsulfonyl, tolylsulfonyl, naphthylsulfonyl and the like, and said “arylsulfonyl” may have one or more (preferably 1 to 3) suitable substituent(s) such as aforesaid lower alkoxy, aforesaid halogen, or the like.

Suitable examples of the substituent of “optionally substituted hydroxy” may include optionally substituted lower alkyl, lower alkenyl, lower alkynyl, cyclo(lower)alkyl, aryl or heterocyclic group or the like.

Preferable examples of the substituent of “optionally substituted hydroxy” may include hydroxy, methoxy, ethoxy, propoxy, isopropoxy, allyloxy, propynyloxy, cyclobutoxy, cyclohexyloxy, hydroxyethoxy, methoxyethoxy, carboxymethoxy, aminoethoxy, dimethylaminoethoxy, fluoroethoxy, carbamoylmethoxy, methylcarbamoylmethoxy, dimethylcarbamoylmethoxy, cyclopropylcarbamoylmethoxy, methoxycarbonylmethoxy, tert-butoxycarbonylmethoxy, acetylmethoxy, benzoylmethoxy, phenoxy, benzyloxy, pyridinylmethoxy, pyridinylethoxy, tetrahydro-2H-pyranyloxy or 1,3(2H)-dioxoisoindolinylethoxy.

Suitable examples of the substituent of “optionally substituted amino which may form N-containing heterocyclic group” may include optionally substituted lower alkyl, lower alkenyl, cyclo(lower) alkyl, amidino, aryl, heterocyclic group or the like, and they may be combined together with N atom to which they are attached to form N-containing heterocyclic group.

Preferable examples of “optionally substituted amino which may form N-containing heterocyclic group” may include amino, methylamino, ethylamino, propylamino, isopropylamino, tert-butylamino, allylamino, cyclopropylamino, hydroxyethylamino, methoxyethylamino, aminoethylamino, dimethylaminoethylamino, carbamoylmethylamino, amidinoamino, anilino, benzylamino, pyridinylamino, pyridinylmethylamino, furylmethylamino, dimethylthiazolylamino, dimethylamino, benzyl(methyl)amino, pyrrolidinyl, piperidinyl, morpholino, piperazinyl, methylpiperazinyl, imidazolyl, triazolyl or benzimidazolyl.

Suitable examples of the substituent of “optionally substituted lower alkyl” may include halo, trihalo, hydroxy, lower alkoxy, optionally substituted amino, acyl, aryl, heterocyclic group or the like.

Preferable examples of “optionally substituted lower alkyl” may include methyl, ethyl, propyl, isopropyl, isobutyl, tert-butyl, fluoroethyl, trifluoromethyl, hydroxyethyl, hydroxyisopropyl, methoxyethyl, methoxyisopropyl, carboxymethyl, aminoethyl, dimethylaminoethyl, carbambylmethyl, methylcarbamoylmethyl, dimethylcarbamoylmethyl, cyclopropylcarbamoylmethyl, methoxycarbonylmethyl, tert-butoxycarbonylmethyl, acetylmethyl, benzoylmethyl, benzyl, pyridinylmethyl, pyridinylethyl, furylmethyl or 1,3(2H)-dioxoisoindolinylethyl.

Suitable examples of the substituent of “optionally substituted lower alkoxy” may include halo, trihalo, lower alkoxy, optionally substituted amino, or heterocyclic group or the like.

Preferable examples of “optionally substituted lower alkoxy” may include methoxy, ethoxy, propoxy, isopropoxy, butoxy, isobutoxy, tert-butoxy, pentyloxy, isopentyloxy, hexyloxy fluoroethoxy, fluoropropoxy, trichloroethoxy, methoxyethoxy, dimethylaminoethoxy or morpholinylethoxy.

Suitable examples of the substituent of “optionally substituted aryl”, “optionally substituted phenyl” or “optionally substituted benzoyl” may include halo, hydroxy, lower alkyl, optionally substituted lower alkoxy, trihalo(lower)alkyl, lower alkylthio, lower alkylsulfinyl or lower alkylsulfonyl, or the like.

Preferable examples of “optionally substituted aryl” may include phenyl which may be substituted with fluoro, bromo, chloro, hydroxy, methyl, trifluoromethyl, methoxy, ethoxy, propoxy, isopropoxy, butoxy, isobutoxy, isopentyloxy, hexyloxy, methoxyethoxy, fluoroethoxy, fluoropropoxy, dimethylaminoethoxy, morpholinylethoxy, methylthio, methylsulfinyl or methylsulfonyl.

Suitable examples of the substituent of “optionally substituted mercapto” may include lower alkyl, lower alkanoyl, aryl, or the like.

Preferable examples of “optionally substituted mercapto” may include methylthio, acetylthio or phenylthio.

The processes for preparing the object aminopyrimidine compound(I) are explained in detail in the following.

Process 1

The compound (Ia) or a salt thereof can be prepared by subjecting the compound (II) or a salt thereof to hydrolysis.

Suitable salt of the compound (II) can be referred to an acid addition salt as exemplified for the compound (I).

This reaction is carried out in accordance with a conventional method.

The hydrolysis is preferably carried out in the presence of a base or an acid including Lewis acid.

Suitable base includes an inorganic base and an organic base such as an alkali metal (e.g. sodium, potassium, etc.), an alkaline earth metal (e.g. magnesium, calcium, etc.), the hydroxide or carbonate or hydrogencarbonate thereof, trialkylamide (e.g. trimethylamine, triethylamine, N-ethyl-N,N-diisopropylamine, etc.), hydrazine, picoline, 1,5-diazabicyclo[4.3.0]non-5-ene, 1,4-diazabicyclo[2.2.2]-octane, 1,8-diazabicyclo[5.4.0]undec-7-ene, or the like.

Suitable acid includes an organic acid (e.g. formic acid, acetic acid, propionic acid, trichloroacetic acid, trifluoroacetic acid, etc.) and an inorganic acid (e.g. hydrochloric acid, hydrobromic acid, sulfuric acid, hydrogen chloride, hydrogen bromide, etc.).

The elimination using Lewis acid such as BBr₃, BCl₃, BF₃, AlCl₃, TiCl₄ or the like is preferably carried out in the presence of cation trapping agents (e.g. anisole, phenol, etc.).

The reaction is usually carried out in a solvent such as water, an alcohol (e.g. methanol, ethanol, isopropyl alcohol, etc.), tetrahydrofuran, dioxane, toluene, methylene chloride, ethylene dichloride, chloroform, N,N-dimethylformamide, N,N-dimethylacetamide, or any other organic solvents which do not adversely affect the reaction, or a mixture thereof.

A liquid base or acid can be also used as the solvent. The reaction temperature is not critical and the reaction is usually carried out under cooling to heating.

Process 2

The compound (Ib) or a salt thereof can be prepared by reacting the compound (Ia) or a salt thereof with the compound (III) or a salt thereof.

Suitable salt of the compound (Ia) can be referred to an acid addition salt as exemplified for the compound (I).

Suitable salt of the compound (III) can be referred to the ones as exemplified for the compound (I).

The present reaction may be carried out in a solvent such as water, phosphate buffer, acetone, chloroform, acetonitrile, nitrobenzene, methylene chloride, ethylene chloride, formamide, N,N-dimethylformamide, N,N-dimethylacetamide, methanol, ethanol, sec-butanol, amyl alcohol, diethyl ether, dioxane, tetrahydrofuran, dimethyl sulfoxide, or any other organic solvent which does not adversely affect the reaction, preferably in ones having strong polarities. Among the solvents, hydrophilic solvents may be used in a mixture with water. When the compound (III) is in liquid, it can also be used as a solvent. The reaction is preferably conducted in the presence of a base, for example, inorganic base such as alkali metal hydroxide, alkali metal carbonate, alkali metal bicarbonate, alkali metal hydride (e.g. sodium hydride, etc.), organic base such as trialkylamine (e.g. trimethylamine, triethylamine, N-ethyl-N,N-diisopropylamine, etc.), and the like.

The reaction temperature is not critical, and the reaction is usually carried out at ambient temperature, under warming or under heating.

The present reaction is preferably carried out in the presence of alkali metal halide (e.g. sodium iodide, potassium iodide, etc.), alkali metal thiocyanate (e.g. sodium thiocyanate, potassium thiocyanate, etc.), di(lower) alkyl azodicarboxylate (e.g. diethyl azodicarboxylate, diisopropyl azodicarboxylate, etc.) or the like.

When Y¹ is —OH, activation of OH with triphenylphosphine and the like may be necessary.

Process 3

The compound (Id) or a salt thereof can be prepared by reacting the compound (Ic) or a salt thereof with the compound (IV) or a salt thereof.

Suitable salt of the compound (Ic) and (IV) can be referred to the ones as exemplified for the compound (I).

The present reaction may be carried out in a solvent such as water, phosphate buffer, acetone, chloroform, acetonitrile, nitrobenzene, methylene chloride, ethylene chloride, formamide, N,N-dimethylformamide, N,N-dimethylacetamide, methanol, ethanol, sec-butanol, amyl alcohol, diethyl ether, dioxane, tetrahydrofuran, dimethyl sulfoxide, pyridine or any other organic solvent which does not adversely affect the reaction, preferably in ones having strong polarities. Among the solvents, hydrophilic solvents may be used in a mixture with water. When the compound (IV) is in liquid, it can also be used as a solvent.

The reaction is preferably conducted in the presence of abase, for example, inorganic base such as alkali metal hydroxide (e.g. sodium hydroxide, potassium hydroxide, etc.), alkali metal carbonate, alkali metal bicarbonate, alkali metal hydride (e.g. sodium hydride), alkali metal alkoxide (e.g. EtONa, t-BuOK, etc.) organic base such as trialkylamine (e.g. trimethylamine, triethylamine, N-ethyl-N,N-diisopropylamine, etc.), and the like.

The reaction temperature is not critical, and the reaction is usually carried out at ambient temperature, under warming or under heating.

The present reaction is preferably carried out in the presence of alkali metal halide (e.g. sodium iodide, potassium iodide, etc.), alkali metal thiocyanate (e.g. sodium thiocyanate, potassium thiocyanate, etc.), di(lower)alkyl azodicarboxylate (e.g. diethyl azodicarboxylate, diisopropyl azodicarboxylate, etc.) or the like.

When Y² is —OH, activation of OH with triphenylphosphine and the like may be necessary.

Process 4

The compound (Ie) or a salt thereof can be prepared by subjecting the compound (V) or a salt thereof to formation reaction of pyrimidine ring.

Suitable salt of the compound (V) and (VI) can be referred to the ones as exemplified for the compound (I).

Suitable salt of the compound (VIII) can be referred to an acid addition salt as exemplified for the compound (I), in which the preferred one is hydrochloride.

This reaction can be carried out by reacting the compound (V) or a salt thereof with the compound (VI) or a salt thereof (Step 1), and further reacting with the compound (VIII) or a salt thereof (Step 2).

The reactions may be carried out in a conventional solvent such as water, alcohol (e.g. methanol, ethanol, etc.), acetone, dioxane, acetonitrile, chloroform, methylene chloride, ethylene chloride, tetrahydrofuran, ethyl acetate, N,N-dimethylformamide, pyridine or any other organic solvent which does not adversely affect the reaction. These conventional solvents may also be used in a mixture with water. In case of Step 1, the compound VI can also be used as a single solvent. The reaction is preferably conducted in the presence of a base, for example, inorganic base such as alkali metal hydroxide (e.g. sodium hydroxide, potassium hydroxide, etc.), alkali metal carbonate(e.g. sodium carbonate, potassium carbonate, etc.), alkali metal bicarbonate (e.g. sodium hydrogen carbonate, potassium hydrogen carbonate, etc.), alkali metal hydride (e.g. sodium hydride), alkali metal alkoxide (e.g. MeONa, EtONa, t-BuOK, etc.) organic base such as trialkylamine (e.g. trimethylamine, triethylamine, N-ethyl-N,N-diisopropylamine, etc.), and the like.

The reaction temperature is not critical, and the reaction is usually carried out at ambient temperature, under warming or under heating.

Process 5

The compound (I) or a salt thereof can be prepared by reacting the compound (XXVI) or a salt thereof with the compound (XXVII) or a salt thereof.

Suitable salt of the compound (XXVI) and (XXVII) can be referred to the ones as exemplified for the compound (I).

The present reaction may be carried out in a solvent such asdioxane, chloroform, methylene chloride, 1,2-dichloroethane, tetrahydrofuran, pyridine, acetonitrile, N,N-dimethylformamide N,N-dimethylacetamide or any other solvent which does not adversely affect the reaction.

The reaction is preferably conducted in the presence of abase, for example, inorganic base such as alkali metal hydroxide (e.g. sodium hydroxide, potassium hydroxide, etc.), alkali metal carbonate, alkali metal bicarbonate, alkali metal hydride (e.g. sodium hydride), alkali metal alkoxide (e.g. EtONa, t-BuOK, etc.) organic base such as trialkylamine (e.g. trimethylamine, triethylamine, N-ethyl-N,N-diisopropylamine, etc.), and the like.

The reaction temperature is not critical, and the reaction is usually carried out at ambient temperature, under warming or under heating.

The reaction can be carried out by the method disclosed in Example 17 mentioned later or the similar manner thereto.

Process 6

The compound (If) or a salt thereof can be prepared by reacting the compound (XXXII) or a salt thereof with the compound (VIII) or a salt thereof.

Suitable salt of the compound (XXXII) and (VIII) can be referred to the ones as exemplified for the compound (I).

The present reaction may be carried out in a solvent such asdioxane, chloroform, methylene chloride, 1,2-dichloroethane, tetrahydrofuran, pyridine, acetonitrile, N,N-dimethylformamide, N,N-dimethylacetamide or any other solvent which does not adversely affect the reaction.

The reaction temperature is not critical, and the reaction is usually carried out at ambient temperature, under warming or under heating.

The reaction can be carried out by the method disclosed in Example 78 mentioned later or the similar manner thereto.

Process 7

The compounds (Ig) and (Ih) or salts thereof can be prepared by oxidizing the compound (If) or a salt thereof.

The oxidation is carried out in the presence of an oxidizer such as 3-chloroperbenzoic acid.

Suitable salt of the compound (If) can be referred to the ones as exemplified for the compound (I).

The present reaction may be carried out in a solvent such as dioxane, chloroform, methylene chloride, 1,2-dichloroethane, tetrahydrofuran, pyridine, acetonitrile, N,N-dimethylformamide, N,N-dimethylacetamide or any other solvent which does not adversely affect the reaction.

The reaction temperature is not critical, and the reaction is usually carried out at ambient temperature, under warming or under heating.

The reaction can be carried out by the method disclosed in Example 79 mentioned later or the similar manner thereto.

Process 8

The compound (Ii) or a salt thereof can be prepared by reacting the compound (Ig) or a salt thereof with the compound (XXXIII) or a salt thereof.

This reaction can be carried out in the same manner as in the aforementioned Process 5, and therefore the reagents to be used and the reaction conditions (e.g., solvent, reaction temperature, etc.) can be referred to those of Process 5.

The reaction can be carried out by the method disclosed in Example 80, 81, 85 and 89 mentioned later or the similar manner thereto.

Process 9

The compound (Ii) or a salt thereof can be prepared by reacting the compound (Ih) or a salt thereof with the compound (XXXIII) or a salt thereof.

This reaction can be carried out in the same manner as in the aforementioned Process 5, and therefore the reagents to be used and the reaction conditions (e.g., solvent, reaction temperature, etc.) can be referred to those of Process 5.

The reaction can be carried out by the method disclosed in Example 91, 93, 94 and 95 mentioned later or the similar manner thereto.

Process 10

The compound (Ij) or a salt thereof can be prepared by reacting the compound (Ig) or a salt thereof with the compound (XXXIV) or a salt thereof.

This reaction can be carried out in the same manner as in the aforementioned Process 5, and therefore the reagents to be used and the reaction conditions (e.g., solvent, reaction temperature, etc.) can be referred to those of Process 5.

The reaction can be carried out by the method disclosed in Example 105, 114, 115, 118, 119, 120, 128, 130 and 133 mentioned later or the similar manner thereto.

Process 11

The compound (Ik) or a salt thereof can be prepared by reacting the compound (If) or a salt thereof with urea hydrogen peroxide addition compound.

This reaction can be carried out by the method disclosed in Example 140 mentioned later or the similar manner thereto.

Process 12

The compound (Il) or a salt thereof can be prepared by reacting the compound (Ik) or a salt thereof with the compound (XXXV) or a salt thereof.

Suitable salt of the compound (Ik) and (XXXV) can be referred to the ones as exemplified for the compound (I).

The present reaction may be carried out in a solvent such as dioxane, chloroform, methylene chloride, 1,2-dichloroethane, tetrahydrofuran, pyridine, acetonitrile, N,N-dimethylformamide, N,N-dimethylacetamide or any other solvent which does not adversely affect the reaction.

The reaction temperature is not critical, and the reaction is usually carried out at ambient temperature, under warming or under heating.

This reaction can be carried out by the method disclosed in Example 159 mentioned later or the similar manner thereto.

Process 13

The compound (In) or a salt thereof can be prepared by reacting the compound (Im) or a salt thereof with the compound (XXXVI) or a salt thereof.

This reaction can be carried out in the same manner as in the aforementioned Process 3, and therefore the reagents to be used and the reaction conditions (e.g., solvent, reaction temperature, etc.) can be referred to those of Process 3.

The reaction is preferably conducted in the presence of N-ethyl-N,N-diisopropylamine.

The reaction can be carried out by the method disclosed in Example 178 mentioned later or the similar manner thereto.

Process 14

The compound (Ip) or a salt thereof can be prepared by subjecting the compound (Io) or a salt thereof to elimination reaction of alkyl group.

Suitable salts of the compound (Io) can be referred to the ones as exemplified for the compound (I).

This reaction is carried out in accordance with a conventional method such as hydrolysis.

The hydrolysis is preferably carried out in the presence of a base or an acid including Lewis acid.

Suitable base includes an inorganic base and an organic base such as an alkali metal (e.g. sodium, potassium, etc.), an alkaline earth metal (e.g. magnesium, calcium, etc.), hydroxide or carbonate or bicarbonate thereof, trialkylamine (e.g. trimethylamine, triethylamine, N-ethyl-N,N-diisopropylamine, etc.),hydrazine, picoline, 1,5-diazabicyclo[4.3.0]non-5-ene, 1,4-diazabicyclo[2.2.2]octane, 1,8-diazabicyclo[5.4.0]undec-7-ene, or the like.

Suitable acid includes an organic acid (e.g. formic acid, acetic acid, propionic acid, trichloroacetic acid, trifluoroacetic acid, etc.) and an inorganic acid (e.g. hydrochloric acid, hydrobromic acid, sulfuric acid, hydrogen chloride, hydrogen bromide, etc.).

The elimination using Lewis acid (e.g. aluminium chloride, boron tribromide, boron trichloride, titanium trichloride, tin tetrachloride, etc.) or the like is preferably carried out in the presence of cation trapping agents (e.g. anisole, phenol, etc.).

The reaction is usually carried out in a solvent such as water, alcohol (e.g. methanol, ethanol, isopropyl alcohol, etc.), tetrahydrofuran, dioxane, toluene, methylene chloride, ethylene dichloride, chloroform, N,N-dimethylformamide, N,N-dimethylacetamide, or any other organic solvents which do not adversely affect the reaction, or a mixture thereof. A liquid base or acid can be also used as the solvent.

The reaction of this process can be also carried out according to a conventional reduction method employed in this field of the art (e.g. chemical reduction, catalytic reduction, etc.).

The reaction temperature is not critical and the reaction is usually carried out at ambient temperature, under warming or under heating.

Process 15

The compound (Iq) or a salt thereof can be prepared by reacting the compound (Ip) or a salt thereof with the compound (XXXVII) or a salt thereof.

Suitable salt of the compound (Ip) can be referred to an acid addition salt as exemplified for the compound (I).

Suitable salt of the compound (XXXVII) can be referred to the ones as exemplified for the compound (I).

The present reaction may be carried out in a solvent such as water, phosphate buffer, acetone, chloroform, acetonitrile, nitrobenzene, methylene chloride, ethylene chloride, formamide, N,N-dimethylformamide, N,N-dimethylacetamide, methanol, ethanol, sec-butanol, amyl alcohol, diethyl ether, dioxane, tetrahydrofuran, dimethyl sulfoxide, or any other organic solvent which does not adversely affect the reaction, preferably in ones having strong polarities. Among the solvents, hydrophilic solvents may be used in a mixture with water. When the compound (XXXVII) is in liquid, it can also be used as a solvent. The reaction is preferably conducted in the presence of a base, for example, inorganic base such as alkali metal hydroxide, alkali metal carbonate, alkali metal bicarbonate, alkali metal hydride (e.g. sodium hydride, etc.), organic base such as trialkylamine (e.g. trimethylamine, triethylamine, N-ethyl-N,N-diisopropylamine, etc.), and the like.

The reaction temperature is not critical, and the reaction is usually carried out at ambient temperature, under warming or under heating.

The present reaction is preferably carried out in the presence of alkali metal halide (e.g. sodium iodide, potassium iodide, etc.), alkali metal thiocyanate (e.g. sodium thiocyanate, potassium thiocyanate, etc.), di(lower)alkyl azodicarboxylate (e.g. diethyl azodicarboxylate, diisopropyl azodicarboxylate, etc.) or the like.

When Y⁸ is —OH, activation of OH with triphenylphosphine and the like may be necessary.

Process 16

The compound (Ir) or a salt thereof can be prepared by reacting the compound (Ih) or a salt thereof with the compound (XXXVIII) or a salt thereof.

This reaction can be carried out by the method disclosed in Example 253 mentioned later or the similar manner thereto.

Process A

The reactions of steps 1 and 2 can be respectively carried out by the methods disclosed in Preparations 1 and 2 mentioned later or the similar manners thereto.

The reactions of steps 3 and 4 can be carried out in the same manner as in the aforementioned Process 4, and therefore the reagents to be used and the reaction conditions (e.g., solvent, reaction temperature, etc.) can be referred to those of Process 4.

Process B

The reactions of steps 1 to 4 can be respectively carried out by the methods disclosed in Preparations 6 to 9 mentioned later or the similar manners thereto.

Process C

The reactions of steps 1 to 4 can be respectively carried out by the methods disclosed in Preparations 10 to 12 and 9 mentioned later or the similar manners thereto.

Process D

The reaction of Step 1 can be carried out by the method disclosed in Preparation 13 mentioned later or the similar manners thereto.

The reaction of step 2 can be carried out by the method disclosed in Preparation 2 mentioned later or the similar manners thereto.

The reactions of steps 3 and 4 can be carried out in the same manner as in the aforementioned Process 4, and therefore the reagents to be used and the reaction conditions (e.g., solvent, reaction temperature, etc.) can be referred to those of Process 4.

Process E

The reaction of Step 1 can be carried out by the method disclosed in Preparation 15 mentioned later or the similar manners thereto.

The reactions of steps 2 and 3 can be carried out in the same manner as in the aforementioned Process 4, and therefore the reagents to be used and the reaction conditions (e.g., solvent, reaction temperature, etc.) can be referred to those of Process 4.

Process F

The reaction of step 1 can be carried out in the same manner as in the aforementioned Process 4, and therefore the reagents to be used and the reaction conditions (e.g., solvent, reaction temperature, etc.) can be referred to those of Process 4.

The reaction of Step 2 can be carried out by the method disclosed in Preparation 20 mentioned later or the similar manners thereto.

The oxidation reaction of step 3 can be carried out by the method disclosed in Preparation 21 mentioned later or the similar manners thereto.

Process G

The reaction of Step 1 can be carried out by the method disclosed in Preparation 43 mentioned later or the similar manners thereto.

The reactions of steps 2 and 3 can be carried out in the same manner as in the aforementioned Process 4, and therefore the reagents to be used and the reaction conditions (e.g., solvent, reaction temperature, etc.) can be referred to those of Process 4.

Process H

The compound (XXXII) or a salt thereof can be prepared by reacting the compound (V) or a salt thereof with the compound (XXXI) or a salt thereof.

This reaction can be carried out by the method disclosed in Preparation 77 mentioned later or the similar manners thereto.

The object compound (I) of the present invention is an adenosine antagonist and possesses the various pharmacological actions as stated before.

In order to show the usefulness of the compound (I) of the present invention, the pharmacological test result of the representative compound of the present invention is shown in the following.

[I] Test Compound

6-(2-Amino-4-phenyl-5-pyrimidinyl)-3(2H)-pyridazinone (Example 1)
6-(2-Amino-4-phenyl-5-pyrimidinyl)-2-ethyl-3(2H)-pyridazinone (Example 4)
N-[5-(1-Isopropyl-6-oxo-1,6-dihydro-3-pyridazinyl)-4-phenyl-2-pyrimidinyl]acetamide (Example 7)
2-Isopropyl-6-{4-phenyl-2-[(2-pyridinylmethyl)amino]-5-pyrimidinyl}-3(2H)-pyridazinone (Example 18)
6-[2-Amino-4-(2-furyl)-5-pyrimidinyl]-2-isopropyl-3(2H)-pyridazinone (Example 47)
6-(2-Amino-4-fluoro-6-phenyl-5-pyrimidinyl)-2-isopropyl-3(2H)-pyridazinone (Example 136)
6-{2-Amino-4-[4-(2-methoxyethoxy)phenyl]-5-pyrimidinyl}-2-isopropyl-3(2H)-pyridazinone (Example 212)
   [II] Test Method
   Test 1: Adenosine Antagonistic Activity

The adenosine antagonistic activity [Ki(nM)] of the test compound was examined by radioligand binding techniques using 8-cyclopentyl-1,3-dipropylxanthine, [dipropyl-2,3-³H(N)] ([³H]DPCPX, 4.5 nM) for human A₁ receptor and [³H]CGS 21680 (20 nM) for human A₂ₐ receptor.

Test 2: Anticatalepsy Activity in Mouse

The test compound (3.2 mg/kg) was administered orally with ddY mice(n=7). Then, haloperidol (0.32 mg/kg) was injected intraperitoneally 30 min. after the administration of the compound. Thirty min. after the injection, the cataleptic responses of mice were measured. The forelimbs of each mouse were placed on a 3 cm high, 3 mm wide horizontal bar, and the duration of cataleptic posture was measured for up to 30 sec.

[III] Test Result

TABLE 1
Test 1: Adenosine antagonistic activity

| | Adenosine receptor | |
|---|---|---|
| Test compound | binding (Ki:nM) | |
| (Example No.) | A₁ | A₂ₐ |
| 1 | 11.35 | 3.85 |
| 4 | 3.14 | 4.35 |
| 7 | 6.47 | 2.09 |
| 18 | 10.78 | 7.38 |
| 47 | 7.74 | 1.38 |
| 136 | 1.75 | 1.41 |
| 212 | 2.36 | 1.70 |

TABLE 2
Test 2: Anticatalepsy activity in Mouse

| Test compound | Manifestation rate of catalepsy |
|---|---|
| (Example No.) | (number of mouse) |
| 1 | 4/7 |
| 4 | 0/7 |
| 7 | 0/7 |
| 18 | 0/7 |
| 47 | 1/7 |
| 136 | 0/7 |
| 212 | 0/7 |

The aminopyrimidine compound (I) and a salt thereof of this invention are useful as adenosine antagonists (especially, A₁ receptor and A₂ (particularly A₂ₐ) receptor dual antagonists) and for the prevention and/or the treatment of depression, dementia (e.g. Alzheimer's disease, cerebrovascular dementia, dementia accompanying Parkinson's disease, etc.), Parkinson's disease, anxiety, pain, cerebrovascular disease, heart failure, hypertension, circulatory insufficiency, post-resuscitation, asystole, bradyarrhythmia, electromechanical dissociation, hemodynamic collapse, SIRS (systemic inflammatory response syndrome), multiple organ failure, renal failure (renal insufficiency), renal toxicity, nephrosis, nephritis, edema, obesity, bronchial asthma, gout, hyperuricemia, sudden infant death syndrome, immunosuppression, diabetes, ulcer, pancreatitis, Meniere's syndrome, anemia, dialysis-induced hypotension, constipation, ischemic bowel disease, ileus, myocardial infarction, thrombosis, obstruction, arteriosclerosis obliterans, thrombophlebitis, cerebral infarction, transient ischemic attack, angina pectoris, and the like.

The pharmaceutical composition of this invention can be used in the form of a pharmaceutical preparation, for example, in a solid, semisolid or liquid form, which contains the aminopyrimidine compound (I) or a pharmaceutically acceptable salt thereof as an active ingredient in admixture with an organic or inorganic carrier or excipient suitable for rectal, pulmonary (nasal orbuccal inhalation), nasal, ocular, external (topical), oral or parenteral (including subcutaneous, intravenous and intramuscular) administrations or insufflation. The active ingredient may be compounded, for example, with the usual non-toxic, pharmaceutically acceptable carriers for tablets, pellets, troches, capsules, suppositories, creams, ointments, aerosols, powders for insufflation, solutions, emulsions, suspensions, and any other form suitable for use. In addition, auxiliary, stabilizing agents, thickening agents, coloring agents and perfumes may be used where necessary. The aminopyrimidine compound (I) or a pharmaceutically acceptable salt thereof is included in a pharmaceutical composition in an amount sufficient to produce the desired aforesaid pharmaceutical effect upon the process or condition of diseases.

For applying the composition to a human being or an animal, it is preferable to apply it by intravenous, intramuscular, pulmonary or oral administration, or insufflation. While the dosage of therapeutically effective amount of the aminopyrimidine compound (I) varies depending on the age and condition of each individual patient to be treated, in the case of intravenous administration, a daily dose of 0.01-100 mg of the aminopyrimidine compound (I) per kg weight of a human being or an animal, in the case of intramuscular administration, a daily dose of 0.01-100 mg of the aminopyrimidine compound (I) per kg weight of a human being or an animal, and in case of oral administration, a daily dose of 0.01-100 mg of the aminopyrimidine compound (I) per kg weight of a human being or an animal is generally given for the prevention and/or treatment of the aforesaid diseases.

The following Preparations and Examples are given for the purpose of illustrating the present invention in more detail.

Preparation 1

A suspension of 3-(phenylethynyl)-6-(phenylsulfonyl)-pyridazine (138.8 g) in MeOH (1.4 l) was cooled in an ice bath. 28% NaOMe in MeOH (92 g) was added dropwise to the mixture at 10° C. over a period of 10 minutes and the mixture was stirred at 5-10° C. for 1 hour and 15 minutes. After the solvent was removed under reduced pressure, the residue was partitioned between EtOAc (1000 ml) and water (500 ml). After an additional extraction with EtOAc (500 ml), the combined extracts were washed with brine (500 ml), dried over anhydrous MgSO₄, and concentrated to give crude material, which was then purified by silica gel column chromatography (n-Hexane:EtOAc, 2:1 v/v) to afford 3-methoxy-6-(phenylethynyl)pyridazine (68.8 g) as colorless crystals.

mp: 98-99° C.

IR (Nujol): 2214, 1651 cm⁻¹

NMR (DMSO-d₆, δ): 4.10 (3H, s), 7.30 (1H, d, J=9.1 Hz), 7.46-7.53 (3H, m), 7.64-7.69 (2H, m), 7.86 (1H, d, J=9.1 Hz).

ESI/MS: 233 [M+Na]⁺

Preparation 2

To a solution of 3-methoxy-6-(phenylethynyl)pyridazine (24.0 g) in AcOH (360 ml) was added dropwise conc. H₂SO₄ (120 ml) and the mixture was heated to reflux for 4.5 hours. AcOH was removed under reduced pressure and the residue was poured into ice (1 kg). Aqueous NaOH was added to the mixture to neutralize and extracted with EtOAc (×3). The combined extracts were washed with brine, dried over MgSO₄, and concentrated under reduced pressure. The crude material was purified by column chromatography on silica-gel (n-Hexane-EtOAc, 1:1 v/v) to give 2-(6-methoxy-3-pyridazinyl)-1-phenylethanone (24.53 g) as yellow crystals.

mp: 103-104° C.

IR (Nujol): 1678, 1652 cm⁻¹

NMR (CDCl₃, δ): 4.11 (3H, s), 4.61 (2H, s), 6.95 (1H, d, J=9.1 Hz), 7.39-7.59 (4H, m), 8.07-8.12 (2H, m).

ESI/MS: 229 [M+H]⁺

Preparation 3

A mixture of 2-(6-methoxy-3-pyridazinyl)-1-phenylethanone (1.50 g) and N,N-dimethylformamide dimethylacetal (1.57 g) was heated to reflux for 2.5 hours. The mixture was cooled to ambient temperature, washed with n-hexane (3 times) and dried to give an oil (1.91 g). The oil was dissolved in EtOH, and guanidine hydrochloride (1.26 g) and 28% NaOMe/in MeOH (2.60 g) was added to the mixture, which was then heated to reflux for 2.5 hours. The mixture was poured into ice/water and extracted with EtOAc (×2). The combined extracts were washed with water and brine, dried over MgSO₄ and concentrated under reduced pressure. The crude material was purified by silica-gel column chromatograpy (CHCl₃—MeOH, 50:1 v/v) to give 2-amino-5-(6-methoxy-3-pyridazinyl)-4-phenylpyrimidine (1.36 g) as colorless crystals.

IR (Nujol): 3325, 3192, 1653, 1562, 1538 cm⁻¹

NMR (DMSO-d₆, δ): 4.03 (3H, s), 6.99-7.11 (4H, m), 7.25-7.43 (5H, m), 8.49 (1H, s)

ESI/MS: 279 [M+Na]⁺

Elemental Analysis for C₁₅H₁₃N₅O

Calcd.: C,64.51; H,4.69; N,25.08

Found : C,64.45; H,4.74; N,24.93

Preparation 4

5-(6-Methoxy-3-pyridazinyl)-N-methyl-4-phenyl-2-pyrimidinamine was obtained from 2-(6-methoxy-3-pyridazinyl)-1-phenylethanone, N,N-dimethylformamide dimethylacetal and 1-methylguanidine hydrochloride according to a similar manner to that of Preparation 3.

NMR (DMSO-d₆, δ): 2.91 (3H, d, J=4.8 Hz)., 4.03 (3H, s), 7.02 (1H, d, J=9.2 Hz), 7.09 (1H, d, J=9.2 Hz), 7.32-7.52 (6H, m), 8.53 (1H, brd. s)

ESI/MS: 316 [M+Na]⁺

Preparation 5

5-(6-Methoxy-3-pyridazinyl)-N,N-dimethyl-4-phenyl-2-pyrimidinamine was obtained from 2-(6-methoxy-3-pyridazinyl)-1-phenylethanone, N,N-dimethylformamide dimethylacetal and 1,1-dimethylguanidine hydrochloride according to a similar manner to that of Preparation 3.

NMR (DMSO-d₆, δ): 3.24 (6H, s), 4.03 (3H, s), 7.03 (1H, d, J=9.2 Hz), 7.10 (1H, d, J=9.2 Hz), 7.31-7.41 (5H, m), 8.59 (1H, s).

ESI/MS: 330 [M+Na]⁺

Preparation 6

To a solution of maleic anhydride (41.57 g) in glacial acetic acid (310 ml) was added 1-isopropylhydrazine (31.43 g) at ambient temperature. The mixture was heated under reflux for 5 hours and concentrated under reduced pressure to give a solid. The solid was triturated by isopropyl ether, collected by filtration, and recrystalized from a mixture of methanol and isopropyl ether to give 6-hydroxy-2-isopropyl-3(2H)-pyridazinone (60.27 g).

mp: 162-164° C. (methanol-isopropyl ether)

IR (KBr): 1504 cm⁻¹

¹H NMR (CDCl₃, δ): 1.22 (6H, d, J=6.66 Hz), 5.03 (1H, 7-plet, J=6.65 Hz), 6.85 (1H, d, J=9.62 Hz), 7.01 (1H, d, J=9.62 Hz), 10.95 (1H, br.s)

APCI/MS: 155 [M+H]⁺

Elemental Analysis for C₇H₁₀N₂O₂

Calcd.: C,54.54; H,6.54; N,18.17

Found : C,54.72; H,6.61; N,18.13

Preparation 7

To a solution of 6-hydroxy-2-isopropyl-3(2H)-pyridazinone (5.00 g) in pyridine (32 ml) was dropwise added tifluoromethanesulfonic anhydride (5.51 ml) under ice-cooling. The mixture was stirred under ice-cooling for one hour and at ambient temperature for 3 hours. Pyridine was removed under reduced pressure to give a residue. The residue was dissolved a mixture of ethyl acetate and water. An organic layer was washed with brine, dried over magnesium sulfate, and concentrated under reduced pressure to give a residue. The residue was purified by column chromatography on silica gel (n-Hexane-EtOAc, 8:2 v/v) to give 1-isopropyl-6-oxo-1,6-dihydro-3-pyridazinyl trifluoromethane-sulfonate as a solid (8.66 g).

mp: 45-46° C. (n-hexane)

IR (KBr): 1660, 1587 cm⁻¹

¹H NMR (CDCl₃, δ): 1.34 (6H, d, J=6.62 Hz), 5.23 (1H, 7-plet, J=6.61 Hz), 7.04 (1H, d, J=9.83 Hz), 7.16 (1H, d, J=9.83 Hz)

APCI/MS: 287 [M+H]⁺

Elemental Analysis for C₈H₉F₃N₂O₄S

Calcd.: C,33.57; H,3.17; N,9.79

Found : C,33.80; H,2.96; N,9.79

Preparation 8

In the presence of dichlorobis(triphenylphosphine)-palladium(II) (0.42 g ) and copper(I) iodide (0.42 g), triethylamine (3.9 ml) was added dropwise to a mixture of 1-isopropyl-6-oxo-1,6-dihydro-3-pyridazinyl trifluoromethane-sulfonate (5.73 g), 1-ethynyl-4-fluorobenzene (3.65 g) indioxane (60 ml) at 75-80° C. for 0.5 hour. The mixture was stirred at 75-80° C. for 1.5 hours. After cooling, water and chloroform were added to the reaction mixture. The organic layer was washed with brine, dried over magnesium sulfate, and concentrated under reduced pressure to give a residue. The residue was purified by column chromatography on silica gel (n-Hexane-EtOAc, 7:3 v/v) to give 6-[(4-fluorophenyl)ethynyl]-2-isopropyl-3(2H)-pyridazinone as a solid (4.22 g).

mp: 105.5-106.5° C. (n-hexane)

IR (KBr): 2208, 1664, 1587 cm⁻¹

¹H NMR (CDCl₃, δ): 1.40 (6H, d, J=6.64 Hz), 5.33 (1H, 7-plet, J=6.64 Hz), 6.87 (1H, d, J=9.57 Hz), 7.01-7.14 (2H, m), 7.28 (1H, d, J=9.57 Hz), 7.51-7.61 (2H, m)

APCI/MS: 257 [M+H]⁺, 215

Elemental Analysis for C₁₅H₁₃FN₂O

Calcd.: C,70.30; H,5.11; N,10.93

Found : C,70.33; H,5.34; N,11.05

Preparation 9

To a mixture of sulfuric acid (6 ml) and acetic acid (15 ml) was added 6-[(4-fluorophenyl)ethynyl]-2-isopropyl-3(2H)-pyridazinone (3.00 g) and the mixture was heated at 100-105° C. for 40 minutes. The solution was poured into a mixuture of ice (90 g) and sodium carbonate (25.4 g), extracted with ethyl acetate (24 ml×2), dried over magnesium sulfate, and concentrated under reduced pressure to give a residue. The residue was purified by column chromatography on silica gel (n-Hexane-EtOAc, 3:7 v/v) to give 6-[2-(4-fluorophenyl)-2-oxoethyl]-2-isopropyl-3(2H)-pyridazinone as a solid (451 mg).

mp: 67-68° C. (n-hexane)

IR (KBr): 1689, 1660, 1596 cm⁻¹

¹H NMR (CDCl₃, δ): 1.32 (6H, d, J=6. 62 Hz), 4.28 (2H, s), 5.29 (1H, 7-plet, J=6.62 Hz), 6.89 (1H, d, J=9.50 Hz), 7.11-7.23 (3H, m), 8.04-8.13 (2H, m)

APCI/MS: 275 [M+H]+, 233

Elemental Analysis for C₁₅H₁₅FN₂O₂

Calcd.: C,65.68; H,5.51; N,10.21

Found : C,65.72; H,5.65; N,10.21

Preparation 10

In the presence of dichlorobis(triphenylphosphine)-palladium(II) (1.47 g) and copper(I) iodide (1.47 g), triethylamine (14.67 ml) was added dropwise to a mixture of 1-isopropyl-6-oxo-1,6-dihydro-3-pyridazinyl trifluoromethanesulfonate (20.10 g), (trimethylsilyl)acetylene (24.81 ml) in tetrahydrofuran (300 ml) under ice-cooling for 2 hours. The mixture was stirred at ambient temperature for 3 hours. The reaction mixture was poured into a mixture of water and ethyl acetate. An organic layer was washed with brine, dried over magnesium sulfate, and concentrated under reduced pressure to give a residue. The residue was purified by column chromatography on silica gel (n-Hexane-EtOAc, 9:1 v/v) to give 2-isopropyl-6-[(trimethylsilyl)ethynyl]-3(2H)-pyridazinone as a solid (16.10 g).

mp: 61-62.5° C. (n-hexane)

IR (KBr): 2160, 1664, 1587 cm⁻¹

¹H NMR (CDCl₃, δ): 0.27 (9H, s), 1.37 (6H, d, J=6.64 Hz), 5.29 (1H, 7-plet, J=6.64 Hz), 6.81 (1H, d, J=9.54 Hz), 7.21 (1H, d, J=9.54 Hz), 7.51-7.61 (2H, m)

ESI/MS: 491 [2M+Na]⁺, 257 [M+Na]⁺, 235 [M+H]⁺

Elemental Analysis for C₁₂H₁₈N₂OSi

Calcd.: C,61.50; H,7.74; N,11.95

Found : C,61.25; H,7.82; N,12.00

Preparation 11

To a solution of 2-isopropyl-6-[(trimethylsilyl)ethynyl]-3(2H)-pyridazinone and benzyltriethylammonium chloride (0.52 g) in a mixture of tetrahydrofuran (45 ml) and acetonitrile (45 ml) was added dropwise 12 N aqueous sodium hydroxide (60 ml) under ice-cooling. After stirring for 30 minutes, the mixture was acidified with concentrated hydrochloric acid under ice-cooling, extracted with chloroform, dried over magnesium sulfate and concentrated under reduced pressure to give a residue. The residue was purified by column chromatography on silica gel (n-Hexane-EtOAc, 8:2 v/v) to give 6-ethynyl-2-isopropyl-3(2H)-pyridazinone as a solid(10.42 g).

mp: 103-104° C. (acetone-n-hexane)

IR (KBr): 3194, 2108, 1655, 1587 cm⁻¹

¹H NMR (CDCl₃, δ): 1.38 (6H, d, J=6.64 Hz), 3.19 (1H, s), 5.31 (1H, 7-plet, J=6.64 Hz), 6.85 (1H, d, J=9.52 Hz), 7.22 (1H, d, J=9.52 Hz)

ESI/MS: 185 [M+Na]⁺, 163 [M+H]⁺

Elemental Analysis for C₉H₁₀N₂O

Calcd.: C,66.65; H,6.21; N,17.27

Found : C,66.92; H,6.28; N,17.36

Preparation 12

In the presence of dichlorobis(triphenylphosphine)-palladium(II) (0.42 g) and copper(I) iodide (0.42 g), triethylamine (3.9 ml) was added dropwise to a mixture of 6-ethynyl-2-isopropyl-3(2H)-pyridazinone (3.25 g), 1-fluoro-4-iodobenzene(6.67 g) in dioxane (60 ml) at 75-80° C. for 0.5 hour. The mixture was stirred at 75-80° C. for 1.5 hours. After cooling, a mixture of water and ethyl acetate was added to the reaction mixture. An organic layer was washed with brine, dried over magnesium sulfate, and concentrated under reduced pressure to give a residue. The residue was purified by column chromatography on silica gel (n-Hexane-EtOAc, 7:3 v/v) to give 6-[(4-fluorophenyl)ethynyl]-2-isopropyl-3(2H)-pyridazinone as a solid (3.81 g).

mp: 105.5-106.5° C. (n-hexane)

IR (KBr): 2208, 1664, 1587 cm⁻¹

¹H NMR (CDCl₃, δ): 1.40 (6H, d, J=6.64 Hz), 5.33 (1H, 7-plet, J=6.64 Hz), 6.87 (1H, d, J=9.57 Hz), 7.01-7.14 (2H, m), 7.28 (1H, d, J=9.57 Hz), 7.51-7.61 (2H, m)

APCI/MS: 257 [M+H]⁺, 215

Elemental Analysis for C₁₅H₁₃FN₂O

Calcd.: C,70.30; H,5.11; N,10.93

Found : C,70.33; H,5.34; N,11.05

Preparation 13

To a mixture of 3-methoxy-6-iodopyridazine (30.0 g), 2-bromo-1-ethynylbenzene (34.5 g), dichlorobis(triphenylphosphine)palladium (II) (892 mg) and copper (I) iodide (242 mg) in DMF (150 ml) was added triethylamine (23.0 ml) at ambient temperature under N₂ atmosphere and the resultant mixture was allowed to stir at the same temperature for 13 hours. The reaction mixture was poured into water and extracted with ethyl acetate (×2). The combined extracts were washed with brine and water, dried over MgSO₄ and concentrated under reduced pressure. The crude material was purified by column chromatography (SiO₂, n-Hexane-EtOAc, 10:1) to afford 3-[(2-bromophenyl)ethynyl]-6-methoxypyridazine.

mp: 99-100° C.

IR (Nujol): 1699, 1651, 1558, 1540 cm⁻¹

NMR (CDCl₃, δ): 4.18 (3H, s), 6.97 (1H, d, J=9.2 Hz), 7.24-7.37 (2H, m), 7.56-7.67 (3H, m)

ESI/MS: 311, 313 [M+Na]⁺, APCI/MS: 289, 291 [M+H]⁺

Preparation 14

1-(2-Bromophenyl)-2-(6-methoxy-3-pyridazinyl)ethanone was obtained from 3-[(2-bromophenyl)ethynyl]-6-methoxypyridazine according to a similar manner to that of Preparation 2.

IR (Neat): 2956, 2925, 1733, 1699, 1651 cm⁻¹

ESI/MS: 380, 382 [M+Na]⁺

Preparation 15

To a solution of 3-methoxy-6-methylpyridazine (371 mg) and ethyl 2-bromobenzoate (753 mg) in THF (4 ml) was added dropwise 1M lithium bis(trimethylsilyl)amide in THF (5.98 ml) at 5° C. over a period of 20 minutes under N₂ atmosphere and the resulting solution was stirred at the same temperature for 30 minutes. The reaction mixture was poured into ice/water and pH was adjusted to neutral with 1N HCl, and the mixture was extracted with EtOAc (×2). The combined extracts were washed with brine, dried over MgSO₄ and concentrated under reduced pressure to give 935 mg of yellow oil. The crude material was purified by silica-gel column chromatography (n-Hexane-EtOAc, 10:3) to afford 1-(2-bromophenyl)-2-(6-methoxy-3-pyridazinyl)ethanone as yellow oil.

Preparation 16

4-(Bromophenyl)-5-(6-methoxy-3-pyridazinyl)-2-pyrimidinamine was obtained from 1-(2-bromophenyl)-2-(6-methoxy-3-pyridazinyl)ethanone according to a similar manner to that of Preparation 3.

mp: 180-181° C. (EtOH)

IR (Nujol): 1649, 1637, 1576, 1560, 1538 cm⁻¹

NMR (DMSO-d₆, δ): 3.98 (3H, s), 7.02 (1H, d, J=9.1 Hz), 7.08 (1H, d, J=9.1 Hz), 7.16 (2H, brd. s), 7.30-7.45 (3H, m), 7.58 (1H, d, J=7.9 Hz), 8.65 (1H, s)

ESI/MS: 380, 382 [M+Na]⁺

Preparation 17

In the presence of dichlorobis(triphenylphosphine)palladium (II) (0.49 g) and copper(I) iodide (0.133 g), a solution of triethylamine (11.7 ml) in dioxane (10 ml) was added dropwise to a mixture of 1-isopropyl-6-oxo-1,6-dihydro-3-pyridazinyl-trifluoromethanesulfonate (20.00 g) and ethynylbenzene (8.56 g) in dioxane (70 ml) at 75-80° C. for 0.5 hour. The mixture was stirred at 75-80° C. for 1.5 hours. After cooling, water and chloroform were added to the reaction mixture. The organic layer was washed with brine, dried over magnesium sulfate, and concentrated under reduced pressure to give a residue. The residue was purified by column chromatography on silica gel (hexane-ethyl acetate 85:15 v/v) to give 2-isopropyl-6-(phenylethynyl)-3(2H)-pyridazinone as a solid (16.17 g).

mp: 75.5-77° C. (heptane)

IR (KBr): 2218, 1669, 1583 cm⁻¹

¹H NMR (CDCl₃, δ): 1.40 (6H, d, J=6.65 Hz), 5.33 (1H, 7-plet, J=6.65 Hz), 6.87 (1H, d, J=9.57 Hz), 7.26-7.42 (4H, m), 7.52-7.60 (2H, m)

ESI/MS: 499 [2M+Na]⁺, 261 [M+Na]⁺, 239 [M+H]⁺

Elemental Analysis for C₁₅H₁₄N₂O

Calcd.: C,75.61; H,5.92; N,11.76

Found : C,75.79; H,5.88; N,11.74

Preparation 18

To a mixture of sulfuric acid (1 ml) and acetic acid (3 ml) was added 2-isopropyl-6-(phenylethynyl)-3(2H)-pyridazinone (479 mg) and the mixture was heated at 100-105° C. for 2 hours. The solution was poured into water (80 ml), extracted with ethyl acetate (30 ml×3), dried over magnesium sulfate, and concentrated under reduced pressure to give a residue. The residue was purified by column chromatography on silica gel (hexane-ethyl acetate 25:75 v/v) to give 2-isopropyl-6-(2-oxo-2-phenylethyl)-3(2H)-pyridazinone as a solid (451 mg).

mp: 50-53° C. (diisopropyl ether-hexane)

IR (KBr): 1687, 1660, 1595 cm⁻¹

¹H NMR (CDCl₃, δ): 1.32 (6H, d, J=6.66 Hz), 4.32 (2H, s), 5.29 (1H, 7-plet, J=6.66 Hz), 6.88 (1H, d, J=9.50 Hz), 7.18 (1H, d, J=9.50 Hz), 7.45-7.62 (3H, m), 8.01-8.07 (2H, m)

ESI/MS: 535 [2M+Na]⁺, 279 [M+Na]⁺, 257 [M+H]⁺

Elemental Analysis for C₁₅H₁₆N₂O₂

Calcd.: C,70.29; H,6.29; N,10.93

Found : C,69.17; H,6.32; N,10.74

Preparation 19

To a mixture of 2-isopropyl-6-(2-oxo-2-phenylethyl)-3(2H)-pyridazinone (500 mg) and N,N-dimethylformamide dimethyl acetal (0.518 ml) was heated at 100-105° C. for one hour. The mixture was concentrated under reduced pressure to give a residue. The residue was purified by column chromatography on silica gel (chloroform) to give 6-[1-benzoyl-2-(dimethylamino)ethenyl]-2-isopropyl-3(2H)-pyridazinone as a solid (604 mg).

mp: 103-104.5° C. (diisopropyl ether)

IR (KBr): 1647, 1628, 1583, 1554 cm⁻¹

¹H NMR (CDCl₃, δ): 1.32 (6H, d, J=6.64 Hz), 2.89 (6H, s), 5.33 (1H, 7-plet, J=6.64 Hz), 6.75 (1H, d, J=9.43 Hz), 7.11 (1H, d, J=9.43 Hz), 7.26-7.48 (6H, m)

ESI/MS: 645 [2M+Na]⁺, 334 [M+Na]⁺, 312 [M+H]⁺

Elemental Analysis for C₁₈H₂₁N₃O₂.0.1H₂O

Calcd.: C,69.03; H,6.82; N,13.42

Found : C,69.08; H,6.75; N,13.34

Preparation 20

Under ice-cooling, potassium t-butoxide (1.75 g) was added to a suspension of 6-[1-benzoyl-2-(dimethylamino)ethenyl]-2-isopropyl-3(2H)-pyridazinone (1.21 g) and S-methylthiouronium sulfate (2.17 g) in methanol (15 ml). The mixture was stirred for one hour under ice-cooling and at ambient temperature for 30 hours. After removal of methanol under reduced pressure, A mixture of chloroform and water was added to a reaction mixture. An organic layer was dried over magnesium sulfate and concentrated under reduced pressure to give a residue. The residue was purified by column chromatography on silica gel (hexane-ethyl acetate 70:30 v/v) to give 2-isopropyl-6-[2-(methylthio)-4-phenyl-5-pyrimidinyl]-3(2H)-pyridazinone as a solid (943 mg).

mp: 143-144.5° C. (ethanol-diisopropyl ether)

IR (KBr): 1655, 1593 cm⁻¹

H NMR (CDCl₃, δ): 1.33 (6H, d, J=6.62 Hz), 2.66 (3H, s), 5.33 (1H, 7-plet, J=6.62 Hz), 6.71 (1H, d, J=9.70 Hz), 6.75 (1H, d, J=9.70 Hz), 7.34-7.52 (5H, m), 8.73 (1H, s)

ESI/MS: 699 [2M+Na]⁺, 361 [M+Na]⁺, 399 [M+H]⁺

Elemental Analysis for C₁₈H₁₈N₄OS

Calcd.: C,63.88; H,5.36; N,16.56

Found : C,64.00; H,5.24; N,16.57

Preparation 21

A mixture of 2-isopropyl-6-[2-(methylthio)-4-phenyl-5-pyrimidinyl]-3(2H)-pyridazinone (990 mg) and urea-hydrogen peroxide complex (550 mg) in acetic acid (0.99 ml) was stirred at ambient temperature for 40 hours. After addition of chloroform, the mixture was washed with water, aqueous sodium hydrogen carbonate solution and brine, dried over magnesium sulfate and concentrated under reduced pressure to give a residue. The residue was purified by column chromatography on silica gel (methanol-chloroform 3:97 v/v) to give 2-isopropyl-6-[2-(methylsulfinyl)-4-phenyl-5-pyrimidinyl]-3(2H)-pyridazinone as a solid (516 mg).

mp: 178-179° C. (acetone-diisopropyl ether)

IR (KBr): 1666, 1591 cm⁻¹

¹H NMR (CDCl₃, δ): 1.35 (6H, d, J=6.66 Hz), 3.06 (3H, s), 5.35 (1H, 7-plet, J=6.66 Hz), 6.77 (1H, d, J=9.56 Hz), 6.84 (1H, d, J=9.56 Hz), 7.38-7.57 (5H, m), 9.06 (1H, s)

ESI/MS: 731 [2M+Na]⁺, 377 [M+Na]⁺

Elemental Analysis for C₁₈H₁₈N₄O₂S

Calcd.: C,61.00; H,5.12; N,15.81

Found : C,61.12; H,5.16; N,15.56

Preparation 22

6-[(2-Fluorophenyl)ethynyl]-2-isopropyl-3(2H)-pyridazinone was obtained from 6-ethynyl-2-isopropyl-3(2H)-pyridazinone and 1-fluoro-2-iodobenzene according to a similar manner to that of Preparation 12.

mp: 84.5-86° C. (diisopropyl ether-hexane)

IR (KBr): 2224, 1660, 1644, 1583 cm⁻¹

¹H NMR (CDCl₃, δ): 1.41 (6H, d, J=6.62 Hz), 5.34 (1H, 7-plet, J=6.62 Hz), 6.88 (1H, d, J=9.52 Hz), 7.12-7.20 (2H, m), 7.32 (1H, d, J=9.52 Hz), 7.33-7.41 (1H, m), 7.52-7.60 (1H, m)

ESI/MS: 535 [2M+Na]+, 279 [M+Na]⁺, 257 [M+H]⁺

Elemental Analysis for C₁₅H₁₃FN₂O

Calcd.: C,70.30; H,5.11; N,10.93

Found : C,70.38; H,5.14; N,10.95

Preparation 23

6-[2-(2-Fluorophenyl)-2-oxoethyl]-2-isopropyl-3(2H)-pyridazinone was prepared from 6-[(2-fluorophenyl)ethynyl]-2-isopropyl-3(2H)-pyridazinone according to a similar manner to that of Preparation 9.

IR (Neat): 1685, 1664, 1593 cm⁻¹

¹H NMR (CDCl₃, δ): 1.32 (6H, d, J=6.65 Hz), 4.28 (2H, s), 5.29 (1H, 7-plet, J=6.65 Hz), 6.89 (1H, d, J=9.50 Hz), 7.17 (1H, d, J=9.50 Hz), 7.40-7.49 (1H, m), 7.55-7.62 (1H, m), 7.89-7.95 (1H, m), 8.02-8.04 (1H, m)

ESI/MS: 571 [2M+Na]⁺, 297 [M+Na]⁺, 275 [M+H]⁺

Preparation 24

6-[2-(Dimethylamino)-1-(2-fluorobenzoyl)ethenyl]-2-isopropyl-3(2H)-pyridazinone was prepared from 6-[2-(2-fluorophenyl)-2-oxoethyl]-2-isopropyl-3(2H)-pyridazinone and N-(dimethoxymethyl)-N,N-dimethylamine according to a similar manner to that of Example 13(1).

mp: 79.5-81.5° C. (chloroform-hexane)

IR (KBr): 1668, 1591 cm⁻¹

¹H NMR (CDCl₃, δ): 1.28 (6H, d, J=6.62 Hz), 2.89 (6H, br.s), 5.29 (1H, 7-plet, J=6.62 Hz), 6.77 (1H, d, J=9.46 Hz), 6.95-7.39 (6H, m)

ESI/MS: 681 [2M+Na]⁺, 352 [M+Na]⁺, 330 [M+H]⁺

Elemental Analysis for C₁₈H₂₀FN₃O₂

Calcd.: C,65.64; H,6.12; N,12.76

Found : C,65.49; H,6.36; N,12.80

Preparation 25

6-[(3-Fluorophenyl)ethynyl]-2-isopropyl-3(2H)-pyridazinone was prepared from 6-ethynyl-2-isopropyl-3(2H)-pyridazinone and 1-fluoro-3-iodobenzene according to a similar manner to that of Preparation 12.

mp: 95.5-96.5° C. (acetone-hexane)

IR (KBr): 2220, 1660, 1606, 1585 cm⁻¹

¹H NMR (CDCl₃, δ): 1.41 (6H, d, J=6.62 Hz), 5.34 (1H, 7-plet, J=6.62 Hz), 6.88 (1H, d, J=9.52 Hz), 7.12-7.20 (2H, m), 7.32 (1H, d, J=9.52 Hz), 7.33-7.41 (1H, m), 7.52-7.60 (1H, m)

ESI/MS: 535 [2M+Na]⁺, 279 [M+Na]⁺, 257 [M+H]⁺

Elemental Analysis for C₁₅H₁₃FN₂O

Calcd.: C,70.30; H,5.11; N,10.93

Found : C,70.22; H,5.16; N,10.94

Preparation 26

6-[2-(3-Fluorophenyl)-2-oxoethyl]-2-isopropyl-3(2H)-pyridazinone was prepared from 6-[(3-fluorophenyl)ethynyl]-2-isopropyl-3(2H)-pyridazinone according to a similar manner to that of Preparation 9.

mp: 80-81° C. (diisopropyl ether-hexane)

IR (KBr): 1680, 1658, 1591 cm⁻¹

¹H NMR (CDCl₃, δ): 1.32 (6H, d, J=6.60 Hz), 4.29 (2H, s), 5.29 (1H, 7-plet, J=6.60 Hz), 6.89 (1H, d, J=9.48 Hz), 7.18 (1H, d, J=9.48 Hz), 7.26-7.33 (1H, m), 7.43-7.53 (1H, m), 7.70-7.77 (1H, m), 7.80-7.86 (1H, m)

ESI/MS: 274 [2M+Na]⁺, 297 [M+Na]⁺, 275 [M+H]⁺

Elemental Analysis for C₁₅H₁₅FN₂O₂

Calcd.: C,65.68; H,5.51; N,10.21

Found : C,65.73; H,5.61; N,10.24

Preparation 27

6-[2-(Dimethylamino)-1-(3-fluorobenzoyl)ethenyl]-2-isopropyl-3(2H)-pyridazinone was prepared from 6-[2-(3-fluorophenyl)-2-oxoethyl]-2-isopropyl-3(2H)-pyridazinone and N-(dimethoxymethyl)-N,N-dimethylamine according to a similar manner to that of Example 13(1).

IR (Neat): 1651, 1574, 1558 cm⁻¹

¹H NMR (CDCl₃, δ): 1.32 (6H, d, J=6.63 Hz), 2.89 (6H, br.s), 5.33 (1H, 7-plet, J=6.63 Hz), 6.75 (1H, d, J=9.46 Hz), 7.07-7.36 (5H, m), 7.45 (1H, s)

ESI/MS: 681 [2M+Na]⁺, 352 [M+Na]⁺, 330 [M+H]⁺

Preparation 28

6-[(2-Chlorophenyl)ethynyl]-2-isopropyl-3(2H)-pyridazinone was prepared from 6-ethynyl-2-isopropyl-3(2H)-pyridazinone and 1-chloro-2-iodobenzene according to a similar manner to that of Preparation 12.

mp: 95.5-96° C. (acetone-hexane)

IR (KBr): 1660, 1585 cm⁻¹

¹H NMR (CDCl₃, δ): 1.41 (6H, d, J=6.63 Hz), 5.34 (1H, 7-plet, J=6.63 Hz), 6.88 (1H, d, J=9.56 Hz), 7.24-7.37 (3H, m), 7.43-7.47 (1H, m), 7.58-7.64 (1H, m)

ESI/MS: 569 and 567 [2M+Na]⁺, 297 and 295 [M+Na]⁺

Elemental Analysis for C₁₅H₁₃ClN₂O

Calcd.: C,66.06; H,4.80; N,10.27

Found : C,66.17; H,4.80; N,10.26

Preparation 29

6-[2-(2-Chlorophenyl)-2-oxoethyl]-2-isopropyl-3(2H)-pyridazinone was prepared from 6-[(2-chlorophenyl)ethynyl]-2-isopropyl-3(2H)-pyridazinone according to a similar manner to that of Preparation 9.

mp: 81.5-82° C. (diisopropyl ether-hexane)

IR (KBr): 1685, 1657, 1589 cm⁻¹

¹H NMR (CDCl₃, δ): 1.27 (6H, d, J=6.66 Hz), 4.03 (2H, s), 5.26 (1H, 7-plet, J=6.66 Hz), 6.89 (1H, d, J=9.48 Hz), 7.19 (1H, d, J=9.48 Hz), 7.29-7.46 (3H, m), 7.55-7.60 (1H, m)

ESI/MS: 605 and 603 [2M+Na]⁺, 315 and 313 [M+Na]⁺, 293 and 291 [M+H]⁺

Elemental Analysis for C₁₅H₁₅ClN₂O₂

Calcd.: C,61.94; H,5.20; N,9.63

Found : C,61.54; H,5.35; N,9.54

Preparation 30

6-[2-(Dimethylamino)-1-(2-chlorobenzoyl)ethenyl]-2-isopropyl-3(2H)-pyridazinone was prepared from 6-[2-(2-chlorophenyl)-2-oxoethyl]-2-isopropyl-3 (2H)-pyridazinone and N-(dimethoxymethyl)-N,N-dimethylamine according to a similar manner to that of Example 13(1).

IR (Neat): 1655, 1576 cm⁻¹

¹H NMR (CDCl₃, δ): 1.32 (6H, d, J=6.64 Hz), 2.89 (6H, br.s), 5.31 (1H, 7-plet, J=6.64 Hz), 6.76 (1H, d, J=9.44 Hz), 7.21-7.36 (6H, m)

ESI/MS: 681 [2M+Na]⁺, 352 [M+Na]⁺, 330 [M+H]⁺

Preparation 31

6-[(3-Chlorophenyl)ethynyl]-2-isopropyl-3(2H)-pyridazinone was prepared from 1-isopropyl-6-oxo-1,6-dihydro-3-pyridazinyl trifluoromethanesulfonate and 1-chloro-3-ethynylbenzene according to a similar manner to that of

Preparation 8.

mp: 94-95° C. (heptane)

IR (KBr): 1664, 1589 cm⁻¹

¹H NMR (CDCl₃, δ): 1.40 (6H, d, J=6.65 Hz), 5.33 (1H, 7-plet, J=6.65 Hz), 6.88 (1H, d, J=9.54 Hz), 7.25-7.48 (4H, m), 7.55-7.58 (1H, m)

ESI/MS: 569 and 567 [2M+Na]⁺, 297 and 295 [M+Na]⁺, 275 and 273 [M+H]⁺

Elemental Analysis for C₁₅H₁₃ClN₂O

Calcd.: C,66.06; H,4.80; N,10.27

Found : C,66.10; H,4.83; N,10.27

Preparation 32

6-[2-(3-Chlorophenyl)-2-oxoethyl]-2-isopropyl-3(2H)-pyridazinone was prepared from 6-[(3-chlorophenyl)ethynyl]-2-isopropyl-3(2H)-pyridazinone according to a similar manner to that of Preparation 9.

mp: 85-86° C. (diisopropyl ether-hexane)

IR (KBr): 1676, 1658, 1591 cm⁻¹

¹H NMR (CDCl₃, δ): 1.32 (6H, d, J=6.65 Hz), 4.28 (2H, s), 5.29 (1H, 7-plet, J=6.65 Hz), 6.89 (1H, d, J=9.50 Hz), 7.17 (1H, d, J=9.50 Hz), 7.40-7.49 (1H, m), 7.55-7.62 (1H, m), 7.89-7.95 (1H, m), 8.02-8.04 (1H, m)

ESI/MS: 605 and 603 [2M+Na]⁺, 315 and 313 [M+Na]⁺, 293 and 291 [M+H]⁺

Elemental Analysis for C₁₅H₁₅ClN₂O₂

Calcd.: C,61.97; H,5.20; N,9.63

Found : C,62.10; H,5.25; N,9.68

Preparation 33

6-[2-(Dimethylamino)-1-(3-chlorobenzoyl)ethenyl]-2-isopropyl-3(2H)-pyridazinone was prepared from 6-[2-(3-chlorophenyl)-2-oxoethyl]-2-isopropyl-3(2H)-pyridazinone and N-(dimethoxymethyl)-N,N-dimethylamine according to a similar manner to that of Example 13(1).

IR (Neat): 1657, 1579, 1556 cm⁻¹

¹H NMR (CDCl₃, δ): 1.32 (6H, d, J=6.66 Hz), 2.90 (6H, br.s), 5.34 (1H, 7-plet, J=6.66 Hz), 6.76 (1H, d, J=9.40 Hz), 7.08 (1H, d, J=9.40 Hz), 7.21-7.38 (4H, m), 7.44 (1H, s)

ESI/MS: 348 and 346 [M+H]⁺

Elemental Analysis for C₁₈H₂₀ClN₃O₂.0.4H₂O

Calcd.: C,61.24; H,5.94; N,11.90

Found : C,61.20; H,6.06; N,11.78

Preparation 34

2-Isopropyl-6-{[2-(trifluoromethyl)phenyl]ethynyl}-3(2H)-pyridazinone was prepared from 6-ethynyl-2-isopropyl-3(2H)-pyridazinone and 1-iodo-2-(trifluoromethyl)benzene according to a similar manner to that of Preparation 12.

mp: 88.5-90° C. (hexane)

IR (KBr): 1664, 1591 cm⁻¹

¹H NMR (CDCl_{3, δ):} 1.41 (6H, d, J=6.60 Hz), 5.34 (1H, 7-plet, J=6.60 Hz), 6.89 (1H, d, J=9.54 Hz), 7.30 (1H, d, J=9.54 Hz), 7.45-7.60 (2H, m), 7.69-7.76 (2H, m)

ESI/MS: 635 [2M+Na]⁺, 329 [M+Na]⁺, 307 [M+H]⁺

Elemental Analysis for C₁₆H₁₃F₃N₂O

Calcd.: C,62.74; H,4.28; N,9.15

Found : C,62.82; H,4.36; N,9.14

Preparation 35

2-Isopropyl-6-{2-oxo-2-[2-(trifluoromethyl)phenyl]ethyl}-3(2H)-pyridazinone was prepared from 2-isopropyl-6-{[2-(trifluoromethyl)phenyl]ethynyl}-3(2H)-pyridazinone according to a similar manner to that of Preparation 9.

mp: 91-92.5° C. (diisopropyl ether-hexane)

IR (KBr): 1709, 1654, 1586 cm⁻¹

¹H NMR (CDCl₃, δ): 1.29 (6H, d, J=6.68 Hz), 4.18 (2H, s), 5.28 (1H, 7-plet, J=6.68 Hz), 6.89 (1H, d, J=9.50 Hz), 7.17 (1H, d, J=9.50 Hz), 7.50-7.79 (4H, m)

ESI/MS: 671 [2M+Na]⁺, 347 [M+Na]⁺, 325 [M+H]⁺

Elemental Analysis for C₁₆H₁₅F₃N₂O₂

Calcd.: C,59.26; H,4.66; N,8.64

Found : C,59.39; H,4.69; N,8.63

Preparation 36

6-{2-(Dimethylamino)-1-[2-(trifluoromethyl)benzoyl]-ethenyl}-2-isopropyl-3(2H)-pyridazinone was prepared from 2-isopropyl-6-{2-oxo-2-[2-(trifluoromethyl)phenyl]-ethyl}-3(2H)-pyridazinone and N-(dimethoxymethyl)-N,N-dimethylamine according to a similar manner to that of Example 13(1).

IR (KBr): 1654, 1587 cm⁻¹

¹H NMR (CDCl₃, δ): 1.33 (6H, d, J=6.65 Hz), 2.84 (6H, br.s), 5.33 (1H, 7-plet, J=6.65 Hz), 6.79 (1H, d, J=8.96 Hz), 7.21-7.70 (6H, m)

ESI/MS: 781 [2M+Na]⁺, 402 [M+Na]⁺, 380 [M+H]⁺

Preparation 37

2-Isopropyl-6-{[3-(trifluoromethyl)phenyl]ethynyl}-3(2H)-pyridazinone was prepared from 6-ethynyl-2-isopropyl-3(2H)-pyridazinone and 1-iodo-3-(trifluoromethyl)benzene according to a similar manner to that of Preparation 12.

mp: 102.5-104° C. (hexane)

IR (KBr): 1662, 1589 cm⁻¹

¹H NMR (CDCl₃, δ): 1.41 (6H, d, J=6.60 Hz), 5.34 (1H, 7-plet, J=6.60 Hz), 6.89 (1H, d, J=9.54 Hz), 7.30 (1H, d, J=9.54 Hz), 7.47-7.56 (1H, m), 7.65 (1H, d, J=7.86 Hz), 7.74 (1H, d, J=7.86 Hz), 7.84 (1H, s)

ESI/MS: 635 [2M+Na]⁺, 329 [M+Na]⁺

Elemental Analysis for C₁₆H₁₃F₃N₂O

Calcd.: C,62.74; H,4.28; N,9.15

Found : C,62.72; H,4.29; N,9.19

Preparation 38

2-Isopropyl-6-{2-oxo-2-[3-(trifluoromethyl)phenyl]ethyl}-3(2H)-pyridazinone was prepared from 2-isopropyl-6-{[3-(trifluoromethyl)phenyl]ethynyl}-3(2H)-pyridazinone according to a similar manner to that of Preparation 9.

IR (Neat): 1695, 1657, 1591 cm⁻¹

¹H NMR (CDCl₃, δ): 1.31 (6H, d, J=6.62 Hz), 4.34 (2H, s), 5.28 (1H, 7-plet, J=6.62 Hz), 6.90 (1H, d, J=9.50 Hz), 7.18 (1H, d, J=9.50 Hz), 7.66 (1H, t, J=7.80 Hz), 7.87 (1H, d, J=7.90 Hz), 8.23 (1H, d, J=7.90 Hz), 8.30 (1H, s)

ESI/MS: 671 [2M+Na]⁺, 347 [M+Na]⁺, 325 [M+H]⁺

Elemental Analysis for C₁₆H₁₅F₃N₂O₂

Calcd.: C,59.26; H,4.66; N,8.64

Found : C,58.99; H,4.75; N,8.57

Preparation 39

6-{3-(Dimethylamino)-1-[2-(trifluoromethyl)benzoyl]-ethenyl}-2-isopropyl-3(2H)-pyridazinone was prepared from 2-isopropyl-6-{2-oxo-2-[3-(trifluoromethyl)phenyl]ethyl}-3(2H)-pyridazinone and N-(dimethoxymethyl)-N,N-dimethylamine according to a similar manner to that of Example 13(1).

IR (Neat): 1651, 1558 cm⁻¹

¹H NMR (CDCl₃, δ): 1.33 (6H, d, J=6.66 Hz), 2.92 (6H, br.s), 5.32 (1H, J=6.66 Hz), 6.76 (1H, d, J=9.40 Hz), 7.07 (1H, d, J=9.40 Hz), 7.41-7.68 (5H, m)

ESI/MS: 781 [2M+Na]⁺, 402 [M+Na]⁺, 380 [M+H]⁺

Preparation 40

2-Isopropyl-6-{[4-(trifluoromethyl)phenyl]ethynyl}-3(2H)-pyridazinone was prepared from 6-ethynyl-2-isopropyl-3(2H)-pyridazinone and 1-iodo-4-(trifluoromethyl)benzene according to a similar manner to that of Preparation 12.

mp: 48-50° C. (hexane)

IR (KBr): 1662, 1585 cm⁻¹

¹H NMR (CDCl₃, δ): 1.41 (6H, d, J=6.60 Hz), 5.34 (1H, 7-plet, J=6.60 Hz), 6.89 (1H, d, J=9.54 Hz), 7.30 (1H, d, J=9.54 Hz), 7.64 (1H, d, J=8.88 Hz), 7.68 (1H, d, J=8.88 Hz)

ESI/MS: 635 [2M+Na]⁺, 329 [M+Na]⁺, 307 [M+H]⁺

Elemental Analysis for C₁₆H₁₃F₃N₂O

Calcd.: C,62.74; H,4.28; N,9.15

Found : C,62.91; H,4.47; N,9.06

Preparation 41

2-Isopropyl-6-{2-oxo-2-[4-(trifluoromethyl)phenyl]ethyl}-3(2H)-pyridazinone was prepared from 2-isopropyl-6-{[4-(trifluoromethyl)phenyl]ethynyl}-3(2H)-pyridazinone according to a similar manner to that of Preparation 9.

mp: 99-101° C. (hexane)

IR (KBr): 1686, 1660, 1595 cm⁻¹

¹H NMR (CDCl₃, δ): 1.31 (6H, d, J=6.62 Hz), 4.34 (2H, s), 5.28 (1H, 7-plet, J=6.62 Hz), 6.90 (1H, d, J=9.50 Hz), 7.18 (1H, d, J=9.50 Hz), 7.77 (2H, d, J=8.15 Hz), 8.15 (2H, d, J=8.15 Hz)

ESI/MS: 671 [2M+Na]⁺, 347 [M+Na]⁺, 325 [M+H]⁺

Elemental Analysis for C₁₆H₁₅F₃N₂O₂

Calcd.: C,59.26; H,4.66; N,8.64

Found : C,59.45; H,4.66; N,8.70

Preparation 42

6-{4-(Dimethylamino)-1-[2-(trifluoromethyl)benzoyl]-ethenyl}-2-isopropyl-3(2H)-pyridazinone was prepared from 2-isopropyl-6-{2-oxo-2-[4-(trifluoromethyl)phenyl]ethyl}-3(2H)-pyridazinone and N-(dimethoxymethyl)-N,N-dimethylamine according to a similar manner to that of Example 13(1).

IR (Neat): 1651, 1558 cm⁻¹

¹H NMR (CDCl₃, δ): 1.29 (6H, d, J=6.64 Hz), 2.91 (6H, br.s), 5.32 (1H, 7-plet, J=6.64 Hz), 6.79 (1H, d, J=9.48 Hz), 7.12 (1H, d, J=9.48 Hz), 7.41 (1H, s), 7.51-7.63 (4H, m)

ESI/MS: 781 [2M+Na]⁺, 402 [M+Na]⁺, 380 [M+H]⁺

Preparation 43

4′-Methoxyacetophenone (1.5 g) was dissolved in tetrahydrofuran (25 ml). To the solution was added sodium tert-butoxide (575 mg) at 25° C. To the solution was added tris(dibenzylideneacetone)-dipalladium(0) (229 mg) and racemic-2,2′-bis(diphenyl phosphino)-1,1′-binaphthyl (311 mg), followed by 3-methoxy-6-chloropyridazine (720 mg). The mixture was heated at 75° C. and stirred for 3 hours. The reaction mixture was portioned to dichloromethane and 0.1N-hydrochloric acid. The organic layer was separated. The aqeous layer was extracted with dichlorometane. The combined organic layer was dried over magnesium sulfate. The solution was concentrated under reduced pressure to give a residue. The residue was purified by column chromatography on silica gel (ethyl acetate:hexane=1:1) to give 1-(4-methoxyphenyl)-2-(6-methoxy-3-pyridazinyl)ethanone (671 mg).

IR (KBr): 3490, 1668, 1469, 1263 cm⁻¹

NMR (DMSO-d₆, δ): 3.86 (3H, s), 4.00 (3H, s), 4.63 (2H, s), 7.06 (2H, d, J=9.8 Hz), 7.20 (1H, d, J=9.2 Hz), 7.56 (1H, d, J=9.2 Hz), 8.05 (1H, d, J=9.8 Hz)

ESI/MS: 259 [M+H]⁺, 281 [M+Na]⁺

Preparation 44

1-(3-Fluorophenyl)-2-(6-methoxy-3-pyridazinyl)ethanone was prepared from 3-methoxy-6-chloropyridazine and 3′-fuoroacetophenone according to a similar manner to that of Preparation 43.

IR (KBr): 3488, 1670, 1469, 1260 cm⁻¹

NMR (DMSO-d₆, δ): 3.99 (3H, s), 4.72 (2H, s), 7.19-7.94 (6H, m)

ESI/MS: 247 [M+H]⁺, 269 [M+Na]⁺

Preparation 45

1-(3-Fluoro-4-methoxyphenyl)-2-(6-methoxy-3-pyridazinyl)-ethanone was prepared from 3-methoxy-6-chloropyridazine and 3′-fluoro-4′-methoxyacetophenone according to a similar manner to that of Preparation 43.

IR (KBr): 3480, 1685, 1444, 1230 cm⁻¹

NMR (DMSO-d₆, δ): 3.95 (3H, s), 4.00 (3H, s), 4.65 (2H, s), 7.20 (1H, d, J=9.0 Hz), 7.29-7.38 (1H, m), 7.67 (1H, d, J=9.0 Hz), 7.83-7.98 (2H, m)

ESI/MS: 277 [M+H]⁺, 299 [M+Na]⁺

Preparation 46

1-(4-Chlorophenyl)-2-(6-methoxy-3-pyridazinyl)ethanone was prepared from 3-methoxy-6-chloropyridazine and 4′-chloroacetophenone according to a similar manner to that of Preparation 43.

IR (KBr): 3485, 1675, 1470, 1265 cm⁻¹

NMR (DMSO-d₆, δ): 4.00 (3H, s), 4.70 (2H, s), 7.19-8.10 (6H, m)

ESI/MS: 263 and 265 [M+H]⁺, 285 and 287 [M+Na]⁺

Preparation 47

2-(6-Methoxy-3-pyridazinyl)-1-(3-pyridinyl)ethanone was prepared from 3-methoxy-6-chloropyridazine and 3-acetylpyridine according to a similar manner to that of

Preparation 43.

IR (KBr): 3485, 1675, 1470, 1265 cm⁻¹

NMR (DMSO-d₆, δ): 4.00 (3H, s), 4.76 (2H, s), 7.15-9.24 (6H, m)

ESI/MS: 230 [M+H]⁺, 252 [M+Na]⁺

Preparation 48

2-(6-Methoxy-3-pyridazinyl)-1-(4-pyridinyl)ethanone was prepared from 3-methoxy-6-chloropyridazine and 4-acetylpyridine according to a similar manner to that of

Preparation 43.

IR (KBr): 3490, 1665, 1465, 1245 cm⁻¹

NMR (DMSO-d₆, δ): 4.01 (3H, s), 4.75 (2H, s), 7.14-7.39 (2H, m), 7.58-7.93 (2H, m), 8.67-8.86 (2H, m)

ESI/MS: 230 [M+H]⁺

Preparation 49

2-(6-Methoxy-3-pyridazinyl)-1-(1,3-thiazol-2-yl)ethanone was prepared from 3-methoxy-6-chloropyridazine and 2-acetylthiazole according to a similar manner to that of

Preparation 43.

IR (KBr): 3485, 1675, 1470, 1265 cm⁻¹

NMR (DMSO-d₆, δ): 3.93 (3H, s), 4.76 (2H, s), 7.20-8.50 (4H, m)

ESI/MS: 236 [M+H]⁺, 258 [M+Na]⁺

Preparation 50

1-(2-Furyl)-2-(6-methoxy-3-pyridazinyl)ethanone was prepared from 3-methoxy-6-chloropyridazine and 2-acetylfuran according to a similar manner to that of Preparation 43.

IR (KBr): 3490, 1675, 1460, 1265 cm⁻¹

NMR (CDCl₃, δ): 4.11 (3H, s), 4.45 (2H, s), 6.55-6.58 (1H, m), 6.96 (1H, d, J=7.0 Hz), 7.26 (1H, s), 7.37-7.39 (1H, m), 7.44 (1H, d, J=7.0 Hz), 7.63 (1H, s)

ESI/MS: 219 [M+H]⁺, 241 [M+Na]⁺

Preparation 51

2-(6-Methoxy-3-pyridazinyl)-1-(4-methylphenyl)ethanone was prepared from 3-methoxy-6-chloropyridazine and 4′-methylacetophenone according to a similar manner to that of Preparation 43.

IR (KBr): 3490, 1675, 1460, 1265 cm⁻¹

NMR (CDCl₃, δ): 1.59 (3H, s), 4.11 (3H, s), 4.58 (2H, s), 6.95 (1H, d, J=9.0 Hz), 7.27 (2H, d, J=6.6 Hz), 7.42 (1H, d, J=9.0 Hz), 8.00 (2H, d, J=6.6 Hz)

ESI/MS: 243 [M+H]⁺, 265 [M+Na]⁺

Preparation 52

1-(3,4-Difluorophenyl)-2-(6-methoxy-3-pyridazinyl)-ethanone was prepared from 3-methoxy-6-chloropyridazine and 3′,4′-difluoroacetophenone according to a similar manner to that of Preparation 43.

IR (KBr): 3485, 1685, 1600, 1465, 1260 cm⁻¹

NMR (DMSO-d₆, δ): 4.03 (3H, s), 4.55 (2H, s), 6.91 (1H, d, J=9.6 Hz), 7.18 (1H, d, J=9.6 Hz), 7.63-7.97 (3H, m)

ESI/MS: 265 [M+H]⁺, 287 [M+Na]⁺

Preparation 53

1-(3,4-Dimethoxyphenyl)-2-(6-methoxy-3-pyridazinyl)-ethanone was prepared from 3-methoxy-6-chloropyridazine and 3′,4′-dimethoxyacetophenone according to a similar manner to that of Preparation 43.

IR (KBr): 3480, 1680, 1595, 1455, 1260 cm⁻¹

NMR (DMSO-d₆, δ): 3.93 (3H, s), 3.95 (3H, s), 4.11 (3H, s), 4.56 (2H, s), 6.89-6.97 (2H, m), 7.43 (1H, d, J=9.0 Hz), 7.59 (1H, d, J=2.0 Hz), 7.82 (1H, dd, J=8.2, 2 Hz)

ESI/MS: 289 [M+H]⁺, 311 [M+Na]⁺

Preparation 54

1-(3,4-Dichlorophenyl)-2-(6-methoxy-3-pyridazinyl)-ethanone was prepared from 3-methoxy-6-chloropyridazine and 3′,4′-dichloroacetophenone according to a similar manner to that of Preparation 43.

IR (KBr): 3480, 1680, 1605, 1460, 1260 cm⁻¹

NMR (CDCl₃, δ): 4.12 (3H, s), 4.56 (2H, s), 6.91-7.00 (2H, m), 7.22-8.16 (3H, m)

ESI/MS: 297, 299 and 301 [M+H]⁺, 319, 321 and 323 [M+Na]⁺

Preparation 55

2-(6-Methoxy-3-pyridazinyl)-1-(2-methylphenyl)ethanone was prepared from 3-methoxy-6-chloropyridazine and 2′-methylacetophenone according to a similar manner to that of Preparation 43.

IR (KBr): 3475, 1690, 1600, 1465, 1260 cm⁻¹

NMR (CDCl₃, δ): 2.52 (3H, s), 4.12 (3H, s), 4.56 (2H, s), 6.97 (1H, d, J=9.0 Hz), 7.10-7.42 (4H, m), 7.95 (2H, d, J=6.6 Hz)

ESI/MS: 243 [M+H]⁺, 265 [M+Na]⁺

Preparation 56

1-(2-Methoxyphenyl)-2-(6-methoxy-3-pyridazinyl)ethanone was prepared from 3-methoxy-6-chloropyridazine and 2′-methoxyacetophenone according to a similar manner to that of Preparation 43.

IR (KBr): 3375, 1690, 1600, 1460, 1260 cm⁻¹

NMR (CDCl₃, δ): 3.91 (3H, s), 4.11 (3H, s), 4.64 (2H, s), 6.95 (1H, d, J=9.0 Hz), 7.02 (2H, m), 7.39 (1H, d, J=9.0 Hz), 7.46-7.94 (1H, m), 7.76-7.81 (1H, m)

ESI/MS: 259 [M+H]⁺, 281 [M+Na]⁺

Preparation 57

2-(6-Methoxy-3-pyridazinyl)-1-(2-thienyl)ethanone was prepared from 3-methoxy-6-chloropyridazine and 2-acetylthiophene according to a similar manner to that of

Preparation 43.

IR (KBr): 3385, 1685, 1585, 1460, 1260 cm⁻¹

NMR (CDCl₃, δ): 4.11 (3H, s), 4.53 (2H, s), 6.90 (1H, d, J=9.0 Hz)7.13-7.17 (1H, m), 7.68 (1H, d, J=9.0 Hz), 7.69 (1H, d, J=5.0 Hz), 7.97 (1H, d, J=4.0 Hz)

ESI/MS: 235 [M+H]⁺, 257 [M+Na]⁺

Preparation 58

2-(6-Methoxy-3-pyridazinyl)-1-(3-methylphenyl)ethanone was prepared from 3-methoxy-6-chloropyridazine and 3′-methylacetophenone according to a similar manner to that of Preparation 43.

IR (KBr): 3475, 1690, 1600, 1465, 1260 cm⁻¹

NMR (CDCl₃, δ): 2.42 (3H, s), 4.12 (3H, s), 4.61 (2H, s), 6.96 (1H, d, J=9.0 Hz), 7.10-7.45 (4H, m), 7.90 (2H, d, J=6.6 Hz)

ESI/MS: 243 [M+H]⁺, 265 [M+Na]⁺

Preparation 59

1-(3-Methoxyphenyl)-2-(6-methoxy-3-pyridazinyl)ethanone was prepared from 3-methoxy-6-chloropyridazine and 3′-methoxyacetophenone according to a similar manner to that of Preparation 43.

IR (KBr): 3470, 1688, 1605, 1470, 1260 cm⁻¹

NMR (CDCl₃, δ): 3.89 (3H, s), 4.21 (3H, s), 4.61 (2H, s), 6.96 (1H, d, J=9.0 Hz), 7.02 (2H, m), 7.42 (1H, d, J=9.0 Hz), 7.46-7.94 (1H, m), 7.76-7.81 (1H, m)

ESI/MS: 259 [M+H]⁺, 281 [M+Na]⁺

Preparation 60

1-(4-Methoxyphenyl)-2-(6-methoxy-3-pyridazinyl)ethanone (516 mg) was dissolved in N,N-dimethyl formamide dimethylacetal (10 ml). The solution was heated at 90° C. for 2 hours. The solution was cooled to ambient temperature. Evaporation of solvent in vacuo gave oily black residue. The residue was dissolved in ethanol (10 ml). To the solution was added guanidine hydrochloride (384 mg) and 28% sodium methylate in methanol solution (0.77 ml). The reaction mixture was heated at 80-90° C., and stirred for 2 hours. The mixture was cooled to ambient temperature, and portioned to ethyl acetate and water. The organic layer was separated and washed with brine. The combined aqueous layer was extracted with ethyl acetate. The combined organic layer was dried over magnesium sulfate. The solution was concentrated under reduced pressure to give oily residue. The above residue was purified by column chromatography on silica gel (chloroform:methanol=20:1) to give 4-(4-methoxyphenyl)-5-(6-methoxy-3-pyridazinyl)-2-pyrimidinamine, which was crystallized from ethanol-water (1:2 30 ml) (509 mg).

IR (KBr): 3376, 1610, 1533, 1463, 1251 cm⁻¹

NMR (DMSO-d₆, δ): 3.75 (3H, s), 4.03 (3H, s), 6.90 (1H, d, J=10 Hz), 6.99 (2H, brs), 7.02-7.13 (4H, m), 7.25 (1H, d), 8.43 (1H, s)

ESI/MS: 310 [M+H]⁺, 332 [M+Na]⁺

Preparation 61

4-(3-Fluorophenyl)-5-(6-methoxy-3-pyridazinyl)-2-pyrimidinamine was prepared from 1-(3-fuorophenyl)-2-(6-methoxy-3-pyridazinyl)ethanone according to a similar manner to that of Preparation 60.

IR (KBr): 3322, 1654, 1575, 1444, 1297 cm⁻¹

NMR (DMSO-d₆, δ): 4.02 (3H, s), 6.99-7.45 (6H, m), 8.51 (1H, s)

ESI/MS: 298 [M+H]⁺, 320 [M+Na]⁺

Preparation 62

4-(3-Fluoro-4-methoxyphenyl)-5-(6-methoxy-3-pyridazinyl)-2-pyrimidinamine was prepared from 1-(3-fuoro-4-methoxyphenyl)-2-(6-methoxy-3-pyridazinyl)ethanone according to a similar manner to that of Preparation 60.

IR (KBr): 3330, 1660, 1575, 1440, 1280 cm⁻¹

NMR (DMSO-d₆, δ): 3.83 (3H, s), 4.04 (3H, s), 6.94 (1H, dd, J=1.16, 8.6 Hz), 7.05-7.26 (6H, m), 8.45 (1H, s)

ESI/MS: 328 [M+H]⁺, 350 [M+Na]⁺

Preparation 63

4-(4-Chlorophenyl)-5-(6-methoxy-3-pyridazinyl)-2-pyrimidinamine was prepared from 1-(4-chlorophenyl)-2-(6-methoxy-3-pyridazinyl)ethanone according to a similar manner to that of Preparation 60.

IR (KBr): 3380, 1620, 1533, 1463, 1260 cm⁻¹

NMR (DMSO-d₆, δ): 4.03 (3H, s), 7.06-7.44 (6H, m), 8.49 (1H, s)

ESI/MS: 314 and 316 [M+H]⁺, 336 and 338 [M+Na]⁺

Preparation 64

5-(6-Methoxy-3-pyridazinyl)-4-(3-pyridinyl)-2-pyrimidinamine was prepared from 2-(6-methoxy-3-pyridazinyl)-1-(3-pyridinyl)ethanone according to a similar manner to that of Preparation 60.

IR (KBr): 3530, 1680, 1575, 1450, 1260 cm⁻¹

NMR (DMSO-d₆, δ): 4.02 (3H, s), 7.11 (1H, d, J=9.2 Hz), 7.16 (2H, brs), 7.32 (1H, d, J=9.2 Hz), 7.36-7.40 (1H, m), 7.64-7.70 (1H, m), 8.44-8.45 (1H, m), 8.54 (1H, s), 8.53-8.57 (1H, m)

ESI/MS: 281 [M+H]⁺, 303 [M+Na]⁺

Preparation 65

5-(6-Methoxy-3-pyridazinyl)-4-(4-pyridinyl)-2-pyrimidinamine was prepared from 2-(6-methoxy-3-pyridazinyl)-1-(4-pyridinyl)ethanone according to a similar manner to that of Preparation 60.

IR (KBr): 3535, 1680, 1580, 1460, 1250 cm⁻¹

NMR (DMSO-d₆, δ): 4.01 (3H, s), 7.12 (1H, d, J=9.2 Hz), 7.21-7.25 (4H, m), 7.32 (1H, d, J=9.2 Hz), 8.55 (1H, s), 8.53-8.57 (2H, m)

ESI/MS: 281 [M+H]⁺, 303 [M+Na]⁺

Preparation 66

5-(6-Methoxy-3-pyridazinyl)-4-(1,3-thiazol-2-yl)-2-pyrimidinamine was prepared from 2-(6-methoxy-3-pyridazinyl)-1-(1,3-thiazol-2-yl)ethanone according to a similar manner to that of Preparation 60.

IR (KBr): 3540, 1620, 1580, 1455, 1255 cm⁻¹

NMR (DMSO-d₆, δ): 4.06 (3H, s), 7.13 (1H, d, J=9.2 Hz), 7.21 (2H, s), 7.60 (1H, d, J=9.2 Hz), 7.77 (1H, d, J=1.1 Hz), 7.90 (1H, d, J=1.1 Hz), 8.43 (1H, s)

ESI/MS: 287 [M+H]⁺, 309 [M+Na]⁺

Preparation 67

4-(2-Furyl)-5-(6-methoxy-3-pyridazinyl)-2-pyrimidinamine was prepared from 1-(2-furyl)-2-(6-methoxy-3-pyridazinyl)-ethanone according to a similar manner to that of Preparation 60.

IR (KBr): 3199, 1662, 1570, 1463, 1295 cm⁻¹

NMR (DMSO-d₆, δ): 4.05 (3H, s), 6.55-6.58 (1H, m), 6.77 (1H, d, J=3.6 Hz), 7.01 (2H, s), 7.21 (1H, d, J=9.2 Hz), 7.49 (1H, d, J=9.2 Hz), 7.66-7.67 (1H, m), 8.33 (1H, s)

ESI/MS: 270 [M+H]⁺, 292 [M+Na]⁺

Preparation 68

5-(6-Methoxy-3-pyridazinyl)-4-(4-methylphenyl)-2-pyrimidinamine was prepared from 2-(6-methoxy-3-pyridazinyl)-1-(4-methylphenyl)ethanone according to a similar manner to that of Preparation 60.

IR (KBr): 3180, 1625, 1567, 1460, 1298 cm⁻¹

NMR (DMSO-d₆, δ): 2.29 (3H, s), 4.03 (sH, s), 7.00-7.21 (8H, m), 8.45 (1H, s)

ESI/MS: 316 [M+Na]⁺

Preparation 69

4-(3,4-Difluorophenyl)-5-(6-methoxy-3-pyridazinyl)-2-pyrimidinamine was prepared from 1-(3,4-difluorophenyl)-2-(6-methoxy-3-pyridazinyl)ethanone according to a similar manner to that of Preparation 60.

IR (KBr): 3178, 1654, 1575, 1465, 1282 cm⁻¹

NMR (DMSO-d₆, δ): 4.03 (3H, s), 7.06-7.47 (7H, m), 8.51 (1H, s)

ESI/MS: 316 [M+H]⁺, 388 [M+Na]⁺

Preparation 70

4-(3,4-Dimethoxyphenyl)-5-(6-methoxy-3-pyridazinyl)-2-pyrimidinamine was prepared from 1-(3,4-dimethoxyphenyl)-2-(6-methoxy-3-pyridazinyl)ethanone according to a similar manner to that of Preparation 60.

IR (KBr): 3174, 1644, 1587, 1465, 1265 cm⁻¹

NMR (DMSO-d₆, δ): 3.59 (3H, s), 3.74 (3H, s), 4.03 (3H, s), 6.74-7.14 (7H, m), 8.43 (1H, s)

ESI/MS: 340 [M+H]⁺, 362 [M+Na]⁺

Preparation 71

4-(3,4-Dichlorophenyl)-5-(6-methoxy-3-pyridazinyl)-2-pyrimidinamine was prepared from 1-(3,4-dichlorophenyl)-2-(6-methoxy-3-pyridazinyl)ethanone according to a similar manner to that of Preparation 60.

IR (KBr): 3318, 1629, 1583, 1461, 1294 cm⁻¹

NMR (DMSO-d₆, δ): 4.03 (3H, s), 7.06-7.44 (6H, m), 7.09 (1H, d, J=9.0 Hz), 7.14 (1H, d, J=8.2 Hz), 7.18 (2H, s), 7.33 (1H, d, J=9.0 Hz), 7.56 (1H, d, J=8.2 Hz), 7.65 (1H, s), 8.52 (1H, s)

ESI/MS: 348, 350 and 352 [M+H]⁺, 370, 372 and 374 [M+Na]⁺

Preparation 72

5-(6-Methoxy-3-pyridazinyl)-4-(2-methylphenyl)-2-pyrimidinamine was prepared from 2-(6-methoxy-3-pyridazinyl)-1-(2-methylphenyl)ethanone according to a similar manner to that of Preparation 60.

IR (KBr): 3120, 1630, 1580, 1465, 1250 cm⁻¹

NMR (DMSO-d₆, δ): 1.98 (3H, s), 3.99 (3H, s), 6.85-7.31 (6H, m), 7.06 (2H, s), 8.60 (1H, s)

ESI/MS: 294 [M+H]⁺, 316 [M+Na]⁺

Preparation 73

4-(2-Methoxyphenyl)-5-(6-methoxy-3-pyridazinyl)-2-pyrimidinamine was prepared from 1-(2-methoxyphenyl)-2-(6-methoxy-3-pyridazinyl)ethanone according to a similar manner to that of Preparation 60.

IR (KBr): 3127, 1634, 1580, 1455, 1267 cm⁻¹

NMR (DMSO-d₆, δ): 3.28 (3H, s), 3.98 (3H, s), 6.88 (1H, d, J=8.6 Hz), 7.01 (2H, brs), 7.02-7.05 (3H, m), 7.34-7.42 (2H, m)

ESI/MS: 310 [M+H]⁺, 332 [M+Na]⁺

Preparation 74

5-(6-Methoxy-3-pyridazinyl)-4-(2-thienyl)-2-pyrimidinamine was prepared from 2-(6-methoxy-3-pyridazinyl)-1-(2-thienyl)ethanone according to a similar manner to that of Preparation 60.

IR (KBr): 3124, 1637, 1575, 1458, 1285 cm⁻¹

NMR (DMSO-d₆, δ): 4.05 (3H, s), 6.55-6.58 (1H, m), 6.77 (1H, d, J=3.6 Hz), 7.01 (2H, s), 7.21 (1H, d, J=9.2 Hz), 7.49 (1H, d, J=9.2 Hz), 7.66-7.67 (1H, m), 8.33 (1H, s)

ESI/MS: 286 [M+H]⁺, 308 [M+Na]⁺

Preparation 75

5-(6-Methoxy-3-pyridazinyl)-4-(3-methylphenyl)-2-pyrimidinamine was prepared from 2-(6-methoxy-3-pyridazinyl)-1-(3-methylphenyl)ethanone according to a similar manner to that of Preparation 60.

IR (KBr): 3135, 1625, 1585, 1466, 1260 cm⁻¹

NMR (DMSO-d₆, δ): 2.26 (3H, s), 4.03 (3H, s), 6.91-7.37 (8H, m), 8.48 (1H, s)

ESI/MS: 294 [M+H]⁺, 316 [M+Na]⁺

Preparation 76

4-(3-Methoxyphenyl)-5-(6-methoxy-3-pyridazinyl)-2-pyrimidinamine was prepared from 1-(3-methoxyphenyl)-2-(6-methoxy-3-pyridazinyl)ethanone according to a similar manner to that of Preparation 60.

IR (KBr): 3140, 1627, 1568, 1465, 1240 cm⁻¹

NMR (DMSO-d₆, δ): 2.29 (3H, s), 4.03 (sH, s), 7.00-7.21 (8H, m), 8.45 (1H, s)

ESI/MS: 310 [M+H]⁺, 332 [M+Na]⁺

Preparation 77

Carbon disulfide (0.248 ml) was dropwise added to a solution of 2-isopropyl-6-(2-oxo-2-phenyl-ethyl)-3 (2H)-pyridazinone (1.00 g) and sodium hydroxide (343 mg) in a mixture of water (1.15 ml) and dimethyl sulfoxide (5 ml) at 5° C. After 30 minutes, iodomethane (0.607 ml) was dropwise added to the mixture at 5° C. and the mixture was stirred at the same temperature for 2 hours. The mixture was poured into water (25 ml) and stirred for 1 hour to give a solid. The solid was dissolved in chloroform, dried over magnesium sulfate and concentrated under reduced pressure to give a residue. The residue was purified by column chromatography on silica gel (n-hexane-ethyl acetate 60:40 v/v) to give 6-[1-benzoyl-2,2-bis(methylthio)vinyl]-2-isopropyl-3(2H)-pyridazinone as a solid (1.23 g).

mp: 103-104.5° C. (chloroform-n-hexane)

IR (KBr): 1670, 1581 cm⁻¹

ESI/MS: 743[2M+Na]⁺, 383[M+Na]⁺, 361[M+H]⁺

¹H NMR (CDCl₃, δ): 1.17 (6H, d, J=6.66 Hz), 2.17 (3H, s), 2.42 (3H, s), 5.22 (1H, 7-plet, J=6.66 Hz), 6.84 (1H, d, J=9.82 Hz), 7.41-7.57 (3H, m), 7.69 (1H, d, J=9.82 Hz), 7.89-7.94 (2H, m)

Elemental Analysis for C₁₈H₂₀N₂O₂S₂

Calcd.: C,59.97; H,5.59; N,7.77

Found : C,60.08; H,5.58; N,7.78

Preparation 78

6-[1-Benzoyl-2,2-bis(methylthio)vinyl]-2-methyl-3(2H)-pyridazinone was prepared from 2-methyl-6-(2-oxo-2-phenylethyl)-3(2H)-pyridazinone, carbon disulfide and methyl iodide according to a similar manner to that of Preparation 77.

mp: 143-146° C. (chloroform-hexane)

IR (KBr): 1662, 1581 cm⁻¹

ESI/MS: 687[2M+Na]⁺, 355[M+Na]⁺, 333[M+H]⁺

¹H NMR (CDCl₃, δ): 2.14 (3H, s), 2.41 (3H, s), 3.71 (3H, s), 6.87 (1H, d, J=9.70 Hz), 7.43-7.58 (3H, m), 7.65 (1H, d, J=9.70 Hz), 7.90-7.96 (2H, m)

Elemental Analysis for C₁₆H₁₆N₂O₂S₂.0.3H₂O

Calcd.: C,56.88; H,4.95; N,8.29

Found : C,56.83; H,4.76; N,8.36

Preparation 79

6-[1-(4-fluorobenzoyl)-2,2-bis(methylthio)vinyl]-2-isopropyl-3(2H)-pyridazinone was prepared from 6-[2-(4-fluorophenyl)-2-oxoethyl]-2-isopropyl-3(2H)-pyridazinone, carbon disulfide and methyl iodide according to a similar manner to that of Preparation 77.

mp: 92-94° C. (diisopropyl ether)

IR (KBr): 1678-1660, 1597, 1583 cm⁻¹

ESI/MS: 779[2M+Na]⁺, 401[M+Na]⁺

¹H NMR (CDCl₃, δ): 1.17 (6H, d, J=6.60 Hz), 2.18 (3H, s), 2.42 (3H, s), 5.23 (1H, 7-plet, J=6.60 Hz), 6.85 (1H, d, J=9.64 Hz), 7.11-7.18 (2H, m), 7.69 (1H, d, J=9.64 Hz), 7.92-7.98 (2H, m)

Preparation 80

1-(4-Bromophenyl)-2-(6-methoxy-3-pyridazinyl)ethanone was prepared according to a similar manner to that of Preparation 15.

¹H NMR (DMSO-d₆, δ): 4.00 (3H, s), 4.69 (2H, s), 7.21 (1H, d, J=9.0 Hz), 7.58 (1H, d, J=9.0 Hz), 7.78 (2H, d, J=8.6 Hz), 7.99 (2H, d, J=8.6 Hz)

ESI/MS: 307, 309[M+H]⁺, 329, 331[M+Na]⁺

IR (KBr): 1639, 1585, 1465, 1012 cm⁻¹

Preparation 81

1-(3-Bromophenyl)-2-(6-methoxy-3-pyridazinyl)ethanone was prepared according to a similar manner to that of Preparation 15

¹H NMR (DMSO-d₆, δ): 4.00 (3H, s), 4.69 (2H, s), 7.21 (1H, d, J=9.0 Hz), 7.58 (1H, d, J=9.0 Hz), 7.78 (2H, d, J=8.6 Hz), 7.99 (2H, d, J=8.6 Hz),

ESI/MS: 307, 309[M+H]⁺, 329, 331[M+Na]⁺

IR (KBr): 1641, 1558, 1467, 1010 cm⁻¹

Preparation 82

1-(2,6-Difluorophenyl)-2-(6-methoxy-3-pyridazinyl)ethanone was prepared according to a similar manner to that of Preparation 15.

¹H NMR (DMSO-d₆, δ): 4.00 (3H, s), 4.55 (2H, s), 7.14-7.34 (3H, m), 7.43-7.72 (2H, m)

ESI/MS: 265[M+H]⁺, 287[M+Na]⁺

IR (neat): 1648, 1558, 1484 cm⁻¹

Preparation 83

1-(3,5-Difluorophenyl)-2-(6-methoxy-3-pyridazinyl)ethanone was prepared according to a similar manner to that of Preparation 15.

¹H NMR (DMSO-d₆, δ): 4.00 (3H, s), 4.72 (2H, s), 7.22 (9.2H, d), 7.31-7.79 (4H, m)

ESI/MS: 265[M+H]⁺, 287[M+Na]⁺

IR (KBr): 1621, 1594, 1473, 1120 cm⁻¹

Preparation 84

1-(2,6-Dichlorophenyl)-2-(6-methoxy-3-pyridazinyl)ethanone was prepared according to a similar manner to that of Preparation 15.

¹H NMR (DMSO-d₆, δ): 4.02 (3H, s), 4.52 (2H, s), 7.21-7.64 (5H, m)

ESI/MS: 297, 299[M+H]⁺, 319, 321[M+Na]⁺

IR (KBr): 1621, 1594, 1473, 1018 cm⁻¹

Preparation 85

1-(2,6-Dimethylphenyl)-2-(6-methoxy-3-pyridazinyl)ethanone was prepared according to a similar manner to that of Preparation 15.

¹H NMR (DMSO-d₆, δ): 2.24 (3H, s), 2.28 (3H, s), 4.02 (2H, s), 7.07-7.30 (4H, m), 7.52 (1H, m)

ESI/MS: 257[M+H]⁺, 279[M+Na]⁺

IR (KBr): 1643, 1540, 1311, 1012 cm⁻¹

Preparation 86

1-(3-Furyl)-2-(6-methoxy-3-pyridazinyl)ethanone was prepared according to a similar manner to that of Preparation 15.

¹H NMR (CDCl₃, δ): 4.12 (3H, s), 4.36 (2H, s), 6.805 (1H, d, J=2 Hz), 6.88 (1H, d, J=9.4 Hz), 7.41-7.46 (2H, m), 8.26 (1H, s),

ESI/MS: 219.3[M+H]⁺, 241.1[M+Na]⁺

IR (KBr): 1678, 1598, 1471, 1153 cm⁻¹

Preparation 87

2-(6-Methoxy-3-pyridazinyl)-1-(3-thienyl)ethanone was prepared according to a similar manner to that of Preparation 15.

¹H NMR (CDCl₃, δ): 4.11 (3H, s), 4.42 (2H, s), 6.73 (1H, d, J=2 Hz), 6.96 (1H, d, J=9.6 Hz), 7.41-7.46 (2H, m), 8.40 (1H, s)

ESI/MS: 235.1[M+H]⁺, 257.2[M+Na]⁺

IR (KBr): 1668, 1602, 1473, 1014 cm⁻¹

Preparation 88

1-(2,6-Dimethoxyphenyl)-2-(6-methoxy-3-pyridazinyl) ethanone was prepared according to a similar manner to that of Preparation 43.

¹H NMR (DMSO-d₆, δ): 3.75 (6H, s), 4.05 (3H, s), 4.28 (2H, s), 6.71 (2H, d, J=8.4 Hz), 7.18 (1H, d, J=9.2 Hz), 7.33 (1H, d, J=8.4 Hz), 7.42 (1H, d, J=9.2 Hz)

ESI/MS: 289[M+H]⁺, 311[M+Na]⁺

IR (KBr): 1716, 1592, 1473, 1110 cm⁻¹

Preparation 89

4-(4-Bromophenyl)-5-(6-methoxy-3-pyridazinyl)-2-pyrimidinamine was prepared according to a similar manner to that of Preparation 60.

¹H NMR (DMSO-d₆, δ): 4.02 (3H, s), 7.10 (2H, s), 7.07-7.26 (2H, m), 7.23 (2H, d, J=8.6 Hz), 7.54 (2H, d, J=8.6 Hz), 8.69 (1H, s)

ESI/MS: 358, 360[M+H]⁺, 380, 382[M+Na]⁺

IR (KBr): 3326, 1629, 1583, 1463 cm⁻¹

Preparation 90

4-(3-Bromophenyl)-5-(6-methoxy-3-pyridazinyl)-2-pyrimidinamine was prepared according to a similar manner to that of Preparation 60.

¹H NMR (DMSO-d₆, δ): 4.03 (3H, s), 7.10 (1H, d, J=9.0 Hz), 7.15 (2H, s), 7.25 (1H, d, J=9.0 Hz), 7.21-7.61 (2H, m), 7.56-7.60 (2H, m), 8.51 (1H, s),

ESI/MS: 358, 360[M+H]⁺, 380[M+Na]⁺

IR (KBr): 3318, 1627, 1529, 1461 cm⁻¹

Preparation 91

4-(2,6-Difluorophenyl)-5-(6-methoxy-3-pyridazinyl)-2-pyrimidinamine was prepared according to a similar manner to that of Preparation 60.

¹H NMR (DMSO-d₆, δ): 3.96 (3H, s), 7.13 (1H, d, J=9.2 Hz), 7.06-7.14 (2H, m), 7.22 (2H, s), 7.46 (1H, d, J=9.2 Hz), 7.42-7.60 (1H, m), 8.64 (1H, s)

ESI/MS: 316[M+H]⁺, 338[M+Na]⁺

Preparation 92

4-(3,5-Difluorophenyl)-5-(6-methoxy-3-pyridazinyl)-2-pyrimidinamine was prepared according to a similar manner to that of Preparation 60.

¹H NMR (DMSO-d₆, δ): 4.03 (3H, s), 6.93-6.98 (2H, m), 7.14 (1H, d, J=9.2 Hz), 7.18 (2H, s), 7.32 (1H, d, J=9.2 Hz), 7.24-7.37 (1H, m), 8.54 (1H, s)

ESI/MS: 316[M+H]⁺, 338[M+Na]⁺

IR (KBr): 3478, 1627, 1581, 1459 cm⁻¹

Preparation 93

4-(2,6-Dichlorophenyl)-5-(6-methoxy-3-pyridazinyl)-2-pyrimidinamine was prepared according to a similar manner to that of Preparation 60.

¹H NMR (DMSO-d₆, δ): 3.96 (3H, s), 7.12 (1H, d, J=9.4 Hz), 7.22 (2H, s), 7.33 (1H, d, J=9.4 Hz), 7.30-7.52 (3H, m), 8.69 (1H, s)

ESI/MS: 348, 350[M+H]⁺, 370, 372[M+Na]⁺

IR (KBr): 3309, 1621, 1581, 1459 cm⁻¹

Preparation 94

4-(2,6-Dimethylphenyl)-5-(6-methoxy-3-pyridazinyl)-2-pyrimidinamine was prepared according to a similar manner to that of Preparation 60.

¹H NMR (DMSO-d₆, δ): 1.93 (6H, s), 3.97 (3H, s), 6.84-7.20 (5H, m), 8.64 (1H, s)

ESI/MS: 308[M+H]⁺, 330[M+Na]⁺

IR (KBr): 3309, 1621, 1581,. 1459 cm⁻¹

Preparation 95

4-(3-Furyl)-5-(6-methoxy-3-pyridazinyl)-2-pyrimidinamine was prepared according to a similar manner to that of Preparation 60.

¹H NMR (DMSO-d₆, δ): 4.07 (3H, s), 6.32 (1H, s), 6.94 (2H, s), 7.21 (1H, d, J=9.2 Hz), 7.52 (1H, d, J=9.2 Hz), 7.55 (1H, s), 7.64 (1H, s), 8.31 (1H, s)

ESI/MS: 270[M+H]⁺, 292[M+Na]⁺

IR (KBr): 3168, 1652, 1581, 1461 cm⁻¹

Preparation 96

5-(6-Methoxy-3-pyridazinyl)-4-(3-thienyl)-2-pyrimidinamine was prepared according to a similar manner to that of Preparation 60.

¹H NMR (DMSO-d₆, δ): 4.05 (3H, s), 6.94-6.97 (3H, m), 7.13 (1H, d, J=9.2 Hz), 7.28 (1H, d, J=9.2 Hz), 7.46-7.52 (2H, m), 8.39 (1H, s)

APCI/MS: 286[M+H]⁺

IR (KBr): 3191, 1656, 1581, 1461 cm⁻¹

Preparation 97

4-(2,6-Dimethoxyphenyl)-5-(6-methoxy-3--pyridazinyl)-2-pyrimidinamine was prepared according to a similar manner to that of Preparation 60.

¹H NMR (DMSO-d₆, δ): 3.55 (6H, s), 3.97 (3H, s), 6.64 (2H, d, J=8.6 Hz), 6.91 (2H, s), 6.96-7.02 (2H, m), 7.31 (1H, t, J=5.6 Hz), 8.52 (1H, s)

ESI/MS: 340[M+H]⁺, 340[M+Na]⁺

IR (KBr): 3309, 1621, 1581, 1459 cm⁻¹

### EXAMPLE 1

A mixture of 2-amino-5-(6-methoxy-3-pyridazinyl)-4-phenylpyrimidine (5.41 g), conc. HCl (0.1 ml) and 4N HCl/dioxane (48.5 ml) in dioxane (54 ml) was heated to 110° C. for 1.5 hours. The reaction mixture was poured into ice/water and pH was adjusted to circa 7-8 with aqueous sodium hydroxide solution. Precipitates were collected by filtration, washed with water and dried to give a crude material, which was recrystallized from 90% aqueous EtOH to give 6-(2-amino-4-phenyl-5-pyrimidinyl)-3(2H)-pyridazinone (4.34 g) as colorless crystals.

mp: >280° C.

IR (Nujol): 3269, 1676, 1651 cm⁻¹

NMR (DMSO-d₆, δ): 6.67 (1H, d, J=9.8 Hz), 6.87 (1H, d, J=9.8 Hz), 7.04 (2H, brd. s), 7.37-7.44 (5H, m), 8.39 (1H, s), 13.07 (1H, s),

ESI/MS: 288 [M+Na]⁺

Elemental Analysis for C₁₄H₁₁N₅O

Calcd.: C,63.39; H,4.18; N,26.40

Found : C,63.55; H,4.24; N,26.32

### EXAMPLE 2

A suspension of 6-(2-amino-4-phenyl-5-pyrimidinyl)-3(2H)-pyridazinone (503 mg) in DMF (10 ml) was cooled in an ice/water bath and 60% NaH (83.9 mg) was added to the mixture. After the mixture was stirred for 15 mimutes, isopropyl iodide was added and the mixture was stirred at ambient temperature overnight. The mixture was poured into ice/water and resultant precipitates were filtered, washed with water, and dried to give 430 mg of powder, which was then purified by silica-gel column chromatography (CHCl₃—MeOH, 50:1) to give 6-(2-amino-4-phenyl-5-pyrimidinyl)-2-isopropyl-3(2H)-pyridazinone (354 mg) as colorless crystals.

mp: 216-217° C. (90% aqueous EtOH)

IR (Nujol): 3356, 3313, 3170, 1671, 1651 cm⁻¹

NMR (DMSO-d₆, δ): 1.04 (6H, d, J=6.6 Hz), 4.97-5.10 (1H, m), 6.80 (1H, d, J=9.6 Hz), 7.06 (2H, brd. s), 7.18 (1H, d, J=9.6 Hz), 7.31-7.41 (5H, m), 8.46 (1H, s)

ESI/MS: 330 [M+Na]⁺

Elemental Analysis for C₁₇H₁₇N₅O

Calcd.: C,66.43; H,5.58; N,22.79

Found : C,66.17; H,5.58; N,22.69

### EXAMPLE 3

6-(2-Amino-4-phenyl-5-pyrimidinyl)-2-methyl-3(2H)-pyridazinone was obtained according to a similar manner to that of Example 2.

mp: 254-255° C. (90% aqueous EtOH)

IR (Nujol): 3313, 3168, 1670, 1649 cm⁻¹

NMR (DMSO-d₆, δ): 3.65 (3H, s), 6.71 (1H, d, J=9.6 Hz), 6.83 (1H, d, J=9.6 Hz), 7.08 (2H, brd. s), 7.38-7.46 (5H, m), 8.42 (1H, s)

ESI/MS: 302 [M+Na]⁺

Elemental Analysis for C₁₅H₁₃N₅O

Calcd.: C,64.51; H,4.69; N,25.08

Found : C,64.32; H,4.75; N,24.91

### EXAMPLE 4

6-(2-Amino-4-phenyl-5-pyrimidinyl)-2-ethyl-3(2H)-pyridazinone was obtained according to a similar manner to that of Example 2.

mp: 255-256° C. (90% aqueous EtOH)

IR (Nujol): 1649 cm⁻¹

NMR (DMSO-d₆, δ): 1.13 (3H, t, J=7.2 Hz), 4.01 (2H, q, J=7.2 Hz), 6.75 (1H, d, J=9.6 Hz), 6.99 (1H, d, J=9.6 Hz), 7.07 (2H, brd. s), 7.35-7.43 (5H, m), 8.44 (1H, s)

ESI/MS: 316 [M+Na]⁺

Elemental Analysis for C₁₆H₁₅N₅O

Calcd.: C,65.52; H,5.15; N,23.88

Found : C,65.48; H,5.21; N,23.78

### EXAMPLE 5

6-(2-Amino-4-phenyl-5-pyrimidinyl)-2-propyl-3(2H)-pyridazinone was obtained according to a similar manner to that of Example 2.

mp: 188-189° C. (90% aqueous EtOH)

IR (Nujol): 1651 cm⁻¹

NMR (DMSO-d₆, δ): 0.80 (3H, t, J=7.4 Hz), 1.48-1.66 (2H, m), 3.96 (2H, t, J=7.2 Hz), 6.75 (1H, d, J=9.6 Hz), 6.99 (1H, d, J=9.6 Hz), 7.07 (2H, brd. s), 7.34-7.43 (5H, m), 8.42 (1H, s)

ESI/MS: 330 [M+Na]⁺

Elemental Analysis for C₁₇H₁₇N₅O

Calcd.: C,66.43; H,5.58; N,22.79

Found : C,66.25; H,5.61; N,22.72

### EXAMPLE 6

6-(2-Amino-4-phenyl-5-pyrimidinyl)-2-benzyl-3(2H)-pyridazinone was obtained according to a similar manner to that of Example 2.

mp: 229-230° C. (90% aqueous EtOH)

IR (Nujol): 3353, 3324, 3191, 1670, 1651 cm⁻¹

NMR (DMSO-d₆, δ): 5.19 (2H, s), 6.80 (1H, d, J=9.6 Hz), 7.01 (1H, d, J=9.6 Hz), 7.08 (2H, brd. s), 7.17-7.46 (10H, m), 8.38 (1H, s)

ESI/MS: 378 [M+Na]⁺

Elemental Analysis for C₂₁H₁₇N₅O

Calcd.: C,70.97; H,4.82; N,19.71

Found : C,70.85; H,4.92; N,19.68

### EXAMPLE 7

A solution of 6-(2-amino-4-phenyl-5-pyrimidinyl)-2-isopropyl-3(2H)-pyridazinone (177 mg) in pyridine (1.8 ml) was cooled in an ice/water bath. Acetyl chloride (49.7 mg) was added to the solution and the mixture was stirred at ambient temperature for 4 hours. Pyridine was removed under reduced pressure and the residue was partitioned between water and CHCl₃. After an additional extraction with CHCl₃, the combined extracts were washed with saturated sodium hydrogencarbonate solution and brine, dried over MgSO₄, and concentrated under reduced pressure. The crude material was purified by silica-gel column chromatography (CHCl₃—MeOH, 50:1) to give N-[5-(1-isopropyl-6-oxo-1,6-dihydro-3-pyridazinyl)-4-phenyl-2-pyrimidinyl]acetamide (107 mg) as colorless crystals.

mp: 209-210° C. (90% aqueous EtOH)

IR (Nujol): 3157, 3128, 1670, 1652 cm⁻¹

NMR (DMSO-d₆, δ) 1.04 (6H, d, J=6.6 Hz), 2.27 (3H, s), 5.02-5.08 (1H, m), 6.90 (1H, d, J=9.6 Hz), 7.34 (1H, d, J=9.6 Hz), 7.44 (5H, s), 8.85 (1H, s), 10.79 (1H, s),

ESI/MS: 349 [M+Na]⁺

Elemental Analysis for C₁₉H₁₉N₅O₂

Calcd.: C,65.32; H,5.48; N,20.04

Found : C,65.27; H,5.53; N,19.86

### EXAMPLE 8

6-[2-(Methylamino)-4-phenyl-5-pyrimidinyl]-3(2H)-pyridazinone was obtained from 5-(6-methoxy-3-pyridazinyl)-N-methyl-4-phenyl-2-pyrimidinamine according to a similar manner to that of Example 1.

mp: >255° C. (90% aqueous EtOH)

IR (Nujol): 3311, 1670, 1651 cm⁻¹

NMR (DMSO-d₆, δ): 2.89 (3H, d, J=4.7 Hz), 6.67 (1H, d, J=9.7 Hz), 6.87 (1H, d, J=9.7 Hz), 7.42-7.51 (6H, m), 8.43 (1H, brd. s), 13.07 (1H, brd. s)

ESI/MS: 302 [M+Na]⁺

Elemental Analysis for C₁₅H₁₃N₅O

Calcd.: C,64.51; H,4.69; N,25.08

Found : C,64.39; H,4.72; N,24.92

### EXAMPLE 9

6-[2-(Dimethylamino)-4-phenyl-5-pyrimidinyl]-3(2H)-pyridazinone was obtained from 5-(6-methoxy-3-pyridazinyl)-N,N-dimethyl-4-phenyl-2-pyrimidinamine according to a similar manner to that of Example 1.

mp: 245-246° C. (90% aqueous EtOH)

IR (Nujol): 3213, 1674, 1650 cm⁻¹

NMR (DMSO-d₆, δ): 3.22 (6H, s), 6.69 (1H, d, J=9.8 Hz), 6.88 (1H, d, J=9.8 Hz), 7.38-7.50 (5H, m), 8.49 (1H, s), 13.09 (1H, brd. s)

ESI/MS: 316 [M+Na]⁺

Elemental Analysis for C₁₆H₁₅N₅O

Calcd.: C,65.52; H,5.15; N,23.88

Found : C,65.44; H,5.12; N,23.85

### EXAMPLE 10

2-Isopropyl-6-[2-(methylamino)-4-phenyl-5-pyrimidinyl]-3(2H)-pyridazinone was obtained from 6-[2-(methylamino)-4-phenyl-5-pyrimidinyl]-3(2H)-pyridazinone according to a similar manner to that of Example 2.

mp: 160-161° C. (EtOAc)

IR (Nujol): 1651 cm⁻¹

NMR (DMSO-d₆, δ): 1.03 (6H, d, J=6.5 Hz), 2.89 (3H, d, J=4.8 Hz), 5.02-5.05 (1H, m), 6.81 (1H, d, J=9.6 Hz), 7.19 (1H, d, J=9.6 Hz), 7.39 (5H, brd. s), 7.54 (1H, brd. s), 8.50 (1H, brd. s)

ESI/MS: 322 [M+H]⁺, 344 [M+Na]⁺

Elemental Analysis for C₁₈H₁₉N₅O

Calcd.: C,67.27; H,5.96; N,21.79

Found : C,67.26; H,6.01; N,21.75

### EXAMPLE 11

6-[2-(Dimethylamino)-4-phenyl-5-pyrimidinyl]-2-isopropyl-3(2H)-pyridazinone was obtained from 6-[2-(dimethylamino)-4-phenyl-5-pyrimidinyl]-3(2H)-pyridazinone according to a similar manner to that of Example 2.

mp: 155-156° C. (EtOAc)

IR (Nujol): 1650, 1590 cm⁻¹

NMR (DMSO-d₆, δ): 1.03 (6H, d, J=6.6 Hz), 3.22 (6H, s), 5.02-5.05 (1H, m), 6.83 (1H, d, J=9.6 Hz), 7.20 (1H, m, J=9.6 Hz), 7.40 (5H, s), 8.56 (1H, s)

ESI/MS: 336 [M+H]⁺, 358 [M+Na]⁺

Elemental Analysis for C₁₉H₂₁N₅O

Calcd.: C,68.04; H,6.31; N,20.88

Found : C,68.01; H,6.34; N,20.87

### EXAMPLE 12

6-(2-Amino-4-phenyl-5-pyrimidinyl)-2-isopropyl-3(2H)-pyridazinone was obtained from 2-isopropyl-6-(2-oxo-2-phenylethyl)-(2H)-pyridazinone according to a similar manner to that of Preparation 3.

mp: 216-217° C. (90% aqueous EtOH).

### EXAMPLE 13

(1)

To a mixture of 6-[2-(4-fluorophenyl)-2-oxoethyl]-2-isopropyl-3(2H)-pyridazinone (1.65 g) and N,N-dimethylformamide dimethyl acetal (1.6 ml) was heated at 100-105° C. for 30 minutes. The mixture was concentrated under reduced pressure to give a residue. The residue was purified by column chromatography on silica gel (MeOH-EtOAc, 1:99 v/v) to give a stereomixture of 6-[2-(dimethylamino)-1-(4-fluorobenzoyl)-ethenyl]-2-isopropyl-3(2H)-pyridazinone as a solid (1.57 g).

mp: 161-162.5° C. (chloroform-n-hexane)

IR (KBr): 1647, 1626, 1581, 1552 cm⁻¹

¹H NMR (CDCl₃, δ): major product 1.32 (6H, d, J=6.67 Hz), 2.90 (6H, s), 5.33 (1H, 7-plet, J=6.67 Hz), 6.76 (1H, d, J=9.48 Hz), 6.95-7.11 (3H, m), 7.43-7.52 (3H, m);
minor product 1.24 (6H, d, J=6.60 Hz), 2.99 (6H, s)

ESI/MS: 681 [2M+Na]⁺, 352 [M+Na]⁺, 330[M+H]⁺

Elemental Analysis for C₁₈H₂₀FN₃O₂

Calcd.: C,65.64; H,6.12; N,12.76

Found : C,65.35; H,6.38; N,12.58

(2)

To a solution of potassium t-butoxide (80.8 mg) in methanol (0.6 ml), guanidine hydrochloride (68.8 mg) was added under ice-cooling. After 15 minutes, a stereomixture of 6-[2-(dimethylamino)-1-(4-fluorobenzoyl)ethenyl]-2-isopropyl-3(2H)-pyridazinone (198 mg) was added and heated under reflux for 4 hours. Water was added to a reaction mixture. After stirring, an aqueous layer was removed by decantation to give a residue. The residue was dissolved in chloroform, dried over magnesium sulfate, concentrated under reduced pressure and was recrystallized from a mixture of chloroform and hexane to give 6-[2-amino-4-(4-fluorophenyl)-5-pyrimidinyl]-2-isopropyl-3(2H)-pyridazinone as a solid (138 mg).

mp: 238-239° C. (ethanol-isopropyl ether)

IR (KBr): 3413, 3182, 1647, 1577, 1492 cm⁻¹

¹H NMR (CDCl₃, δ): 1.31 (6H, d, J=6.62 Hz), 5.33 (1H, 7-plet, J=6.62 Hz), 5.37 (2H, br.s), 6.70 (1H, d, J=9.56 Hz), 6.74 (1H, d, J=9.56 Hz), 7.05-7.10 (2H, m), 7.40-7.45 (2H, m), 8.51 (1H, s)

ESI/MS: 673 [2M+Na]⁺, 348 [M+Na]⁺, 326 [M+H]⁺

Elemental Analysis for C₁₇H₁₆FN₅O

Calcd.: C,62.76; H,4.96; N,21.53

Found : C,62.76; H,4.90; N,21.54

### EXAMPLE 14

6-[2-Amino-4-(2-bromophenyl)-5-pyrimidinyl]-3(2H)-pyridazinone was obtained according to a similar manner to that of Example 1.

NMR (DMSO-d₆, δ): 6.68 (1H, d, J=9.8 Hz), 6.95 (1H, d, J=9.8 Hz), 7.14 (2H, brd. s), 7.32-7.47 (3H, m), 7.63 (1H, d, J=7.9 Hz), 8.50 (1H, s), 13.0 (1H, brd. s)

ESI/MS: 366, 368 [M+Na]⁺

### EXAMPLE 15

6-[2-Amino-4-(2-bromophenyl)-5-pyrimidinyl]-2-isopropyl-3(2H)-pyridazinone was obtained according to a similar manner to that of Example 2.

mp: 203-205° C. (EtOH)

IR (Nujol): 3396, 3319, 3197, 1649, 1630, 1579, 1560 cm⁻¹

NMR (DMSO-d₆, δ): 0.86 (6H, d, J=6.4 Hz), 4.87-5.00 (1H, m), 6.85 (1H, d, J=9.7 Hz), 7.15 (2H, brd. s), 7.29-7.58 (4H, m), 7.60 (1H, d, J=7.4 Hz), 8.57 (1H, s)

ESI/MS: 408, 410 [M+Na]⁺

### EXAMPLE 16

A suspension of 6-[2-amino-4-(2-bromophenyl)-5-pyrimidinyl]-2-isopropyl-3(2H)-pyridazinone (138 mg), 10% palladium on carbon (19.7 mg) and sodium acetate (58.6 mg) in methanol(14 ml) was hydrogenated at 20° C. under hydrogen atmosphere for 8 hours. Insoluble material was filtered off and the solvent was removed under reduced pressure. The residue was partitioned between sodium bicarbonate solution and dichloromethane. After additional extraction with dichloromethane (×2), the combined extracts were washed with water, dried over MgSO₄ and concentrated under reduced pressure to afford colorless crystals of 6-(2-amino-4-phenyl-5-pyrimidinyl)-2-isopropyl-3(2H)-pyridazinone.

### EXAMPLE 17

A solution of 2-isopropyl-6-[2-(methylsulfinyl)-4-phenyl-5-pyrimidinyl]-3(2H)-pyridazinone (177 mg) and morpholine (0.0653 ml) in dioxane (0.35 ml) was heated at 100-105° C. for 25 hours. Water (3.5 ml) was added to the reaction mixture to give a solid. The solid was collected by filltration, dissolved in chloroform, dried over magnesium sulfate and purified by column chromatography on silica gel (methanol-chloroform 1:99 v/v) to give 2-isopropyl-6-[2-morpholino-4-phenyl-5-pyrimidinyl]-3(2H)-pyridazinone as a solid (125 mg).

mp: 200.5-202° C. (ethanol-diisopropyl ether)

IR (KBr): 1666, 1591, 1574, 1514 cm⁻¹

¹H NMR (CDCl₃, δ): 1.31 (6H, d, J=6.64 Hz), 3.77-3.83 (4H, m), 3.91-3.97 (4H, m), 5.31 (1H, 7-plet, J=6.64 Hz), 6.66 (1H, d, J=9.54 Hz), 6.71 (1H, d, J=9.54 Hz), 7.33-7.48 (5H, m), 8.56 (1H, s)

ESI/MS: 777 [2M+Na]⁺, 400 [M+Na]⁺, 378 [M+H]⁺

Elemental Analysis for C₂₁H₂₃N₅O₂

Calcd.: C,66.83; H,6.14; N,18.55

Found : C,66.84; H,6.33; N,18.48

### EXAMPLE 18

2-Isopropyl-6-{4-phenyl-2-[(2-pyridinylmethyl)amino]-5-pyrimidinyl}-3(2H)-pyridazinone was prepared from2-isopropyl-6-[2-(methylsulfinyl)-4-phenyl-5-pyrimidinyl]-3(2H)-pyridazinone and 2-pyridinylmethylamine according to a similar manner to that of Example 17.

mp: 143-144.5° C. (ethanol-diisopropyl ether)

IR (KBr): 3275, 1660, 1587, 1576 cm⁻¹

¹H NMR (CDCl₃, δ): 1.31 (6H, d, J=6.62 Hz), 4.86 (2H, d, J=5.46 Hz), 5.31 (1H, 7-plet, J=6.62 Hz), 6.45-6.49 (1H, m), 6.66 (1H, d, J=9.60 Hz), 6.70 (1H, d, J=9.60 Hz), 7.17-7.24 (1H, m), 7.33-7.68 (6H, m), 7.63-7.73 (1H, m), 8.55-8.61 (2H, m)

ESI/MS: 421 [M+Na]⁺, 399 [M+H]⁺

Elemental Analysis for C₂₃H₂₂N₆O.0.2H₂O

Calcd.: C,68.71; H,5.62; N,20.90

Found : C,68.65; H,5.65; N,20.88

### EXAMPLE 19

6-(2-Anilino-4-phenyl-5-pyrimidinyl)-2-isopropyl-3(2H)-pyridazinone was prepared from 2-isopropyl-6-[2-(methylsulfinyl)-4-phenyl-5-pyrimidinyl]-3(2H)-pyridazinone and aniline according to a similar manner to that of Example 17.

mp: 200-202° C. (ethanol-diisopropyl ether)

IR (KBr): 1662, 1587, 1568 cm⁻¹

¹H NMR (CDCl₃, δ): 1.33 (6H, d, J=6.62 Hz), 5.32 (1H, 7-plet, J=6.62 Hz), 6.69 (1H, d, J=9.55 Hz), 6.74 (1H, d, J=9.55 Hz), 7.08 (1H, t, J=7.42 Hz), 7.32-7.52 (9H, m), 7.70 (1H, d, J=7.68 Hz), 8.66 (1H, s)

ESI/MS: 789 [2M+Na]⁺, 406 [M+Na]⁺, 384 [M+H]⁺

Elemental Analysis for C₂₃H₂₁N₅O

Calcd.: C,72.04; H,5.52; N,18.26

Found : C,71.88; H,5.58; N,18.17

### EXAMPLE 20

6-[2-Amino-4-(2-fluorophenyl)-5-pyrimidinyl]-2-isopropyl-3(2H)-pyridazinone was prepared from 6-[2-(dimethylamino)-1-(2-fluorobenzoyl) ethenyl]-2-isopropyl-3(2H)-pyridazinone and guanidine hydrochloride according to a similar manner to that of Example 13(2).

mp: 239-240.5° C. (ethanol)

IR (KBr): 3392, 3325, 3203, 1649, 1633, 1585 cm⁻¹

¹H NMR (CDCl₃, δ): 1.11 (6H, d, J=6.60 Hz), 5.20 (1H, 7-plet, J=6.60 Hz), 5.45 (2H, br.s), 6.79 (1H, d, J=9.56 Hz), 6.94-7.04 (2H, m), 7.20-7.55 (3H, m), 8.51 (1H, s)

ESI/MS: 673 [2M+Na]⁺, 348 [M+Na]⁺, 326 [M+H]⁺

Elemental Analysis for C₁₇H₁₆FN₅O

Calcd.: C,62.76; H,4.96; N,21.53

Found : C,62.74; H,5.07; N,21.39

### EXAMPLE 21

6-[2-Amino-4-(3-fluorophenyl)-5-pyrimidinyl]-2-isopropyl-3(2H)-pyridazinone was prepared from 6-[2-(dimethylamino)-1-(3-fluorobenzoyl) ethenyl]-2-isopropyl-3(2H)-pyridazinone and guanidine hydrochloride according to a similar manner to that of Example 13(2).

mp: 220-221.5° C. (ethanol)

IR (KBr): 3425, 3305, 3194, 1660, 1637, 1581 cm⁻¹

¹H NMR (CDCl₃, δ): 1.29 (6H, d, J=6.60 Hz), 5.30 (1H, 7-plet, J=6.60 Hz), 5.39 (2H, br.s), 6.73 (1H, d, J=9.56 Hz), 6.78 (1H, d, J=9.56 Hz), 7.09-7.33 (4H, m), 8.53 (1H, s)

ESI/MS: 673 [2M+Na]⁺, 348 [M+Na]⁺, 326 [M+H]⁺

Elemental Analysis for C₁₇H₁₆FN₅O

Calcd.: C,62.76; H,4.96; N,21.53

Found : C,62.77; H,5.02; N,21.55

### EXAMPLE 22

6-[2-Amino-4-(2-chlorophenyl)-5-pyrimidinyl]-2-isopropyl-3(2H)-pyridazinone was prepared from 6-[2-(dimethylamino)-1-(2-chlorobenzoyl) ethenyl]-2-isopropyl-3(2H)-pyridazinone and guanidine hydrochloride according to a similar manner to that of Example 13(2).

mp: 227-228.5° C. (ethanol)

IR (KBr): 3398, 3327, 3201, 1649, 1631 cm⁻¹

¹H NMR (DMSO-d₆, δ): 0.85 (6H, d, J=6.58 Hz), 5.30 (1H, 7-plet, J=6.58 Hz), 6.86 (1H, d, J=9.58 Hz), 7.15 (2H, br.s), 7.33-(4H, m), 7.48 (1H, d, J=9.58 Hz), 8.57 (1H, s)

ESI/MS: 707 and 705 [2M+Na]⁺, 366 and 364 [M+Na]⁺, 344 and 342 [M+H]⁺(mobile phase MeOH-H₂O)

Elemental Analysis for C₁₇H₁₆ClN₅O

Calcd.: C,59.74; H,4.72; N,20.49

Found : C,59.92; H,4.79; N,20.45

### EXAMPLE 23

6-[2-Amino-4-(3-chlorophenyl)-5-pyrimidinyl]-2-isopropyl-3(2H)-pyridazinone was prepared from 6-[2-(dimethylamino)-1-(3-chlorobenzoyl) ethenyl]-2-isopropyl-3(2H)-pyridazinone and guanidine hydrochloride according to a similar manner to that of Example 13(2).

mp: 236-237° C. (ethanol)

IR (KBr): 3471, 3298, 3180, 1664, 1622, 1585 cm⁻¹

¹H NMR (CDCl₃, δ): 1.28 (6H, d, J=6.62 Hz), 5.30 (1H, 7-plet, J=6.62 Hz), 5.37 (2H, br.s), 6.74 (1H, d, J=9.54 Hz), 6.79 (1H, d, J=9.54 Hz), 7.17-7.42 (3H, m), 7.51-7.53 (1H, m), 8.53 (1H, s)

ESI/MS: 707 and 705 [2M+Na]⁺, 366 and 364 [M+Na]⁺, 344 and 342 [M+H]⁺

Elemental Analysis for C₁₇H₁₆ClN₅O

Calcd.: C,59.74; H,4.72; N,20.49

Found : C,59.58; H,4.76; N,20.41

### EXAMPLE 24

6-{2-Amino-4-[2-(trifluoromethyl)phenyl]-5-pyrimidinyl}-2-isopropyl-3(2H)-pyridazinone was prepared from 6-{2-(dimethylamino)-1-[2-(trifluoromethyl)benzoyl]-ethenyl}-2-isopropyl-3(2H)-pyridazinone and guanidine hydrochloride according to a similar manner to that of Example 13(2).

mp: 160-161° C. (ethanol-hexane)

IR (KBr): 3411, 3329, 3207, 1649, 1630, 1583 cm⁻¹

¹H NMR (CDCl₃, δ): 1.07 (6H, d, J=6.62 Hz), 5.18 (1H, 7-plet, J=6.62 Hz), 5.35 (2H, br.s), 6.73 (1H, d, J=9.56 Hz), 6.92 (1H, d, J=9.56 Hz), 7.24-7.29 (1H, m), 7.47-7.61 (2H, m), 7.71-7.77 (1H, m), 8.57 (1H, s)

ESI/MS: 773 [2M+Na]⁺, 398 [M+Na]⁺

Elemental Analysis for C₁₈H₁₆F₃N₅O

Calcd.: C,57.60; H,4.30; N,18.66

Found : C,57.86; H,4.29; N,18.72

### EXAMPLE 25

6-{2-Amino-4-[3-(trifluoromethyl)phenyl]-5-pyrimidinyl}-2-isopropyl-3(2H)-pyridazinone was prepared from 6-{2-(dimethylamino)-1-[3-(trifluoromethyl)benzoyl]-ethenyl}-2-isopropyl-3(2H)-pyridazinone and guanidine hydrochloride according to a similar manner to that of Example 13(2).

mp: 163.5-165° C. (ethanol-hexane)

IR (KBr): 3454, 3305, 3178, 1662, 1630, 1585 cm⁻¹

¹H NMR (CDCl₃, δ): 1.23 (6H, d, J=6.62 Hz), 5.28 (1H, 7-plet, J=6.62 Hz), 5.41 (2H, br.s), 6.75 (1H, d, J=9.56 Hz), 6.82 (1H, d, J=9.56 Hz), 7.44-7.57 (2H, m), 7.65-7.69 (1H, m), 7.78 (1H, br.s), 8.53 (1H, s)

ESI/MS: 773 [2M+Na]⁺, 398 [M+Na]⁺, 376 [M+H]⁺

Elemental Analysis for C₁₈H₁₆F₃N₅O

Calcd.: C,57.60; H,4.30; N,18.66

Found : C,57.67; H,4.36; N,18.52

### EXAMPLE 26

6-{2-Amino-4-[4-(trifluoromethyl)phenyl]-5-pyrimidinyl}-2-isopropyl-3(2H)-pyridazinone was prepared from 6-{2-(dimethylamino)-1-[4-(trifluoromethyl)benzoyl3-ethenyl}-2-isopropyl-3(2H)-pyridazinone and guanidine hydrochloride according to a similar manner to that of Example 13(2).

mp: 209-210.5° C. (ethanol-hexane)

IR (KBr): 3338, 3311, 3180, 1643, 1583 cm⁻¹

¹H NMR (CDCl₃, δ): 1.20 (6H, d, J=6.62 Hz), 5.27 (1H, 7-plet, J=6.62 Hz), 5.42 (2H, br.s), 6.76 (1H, d, J=9.54 Hz), 6.84 (1H, d, J=9.54 Hz), 7.53 (2H, d, J=8.26 Hz), 7.65 (2H, d, J=8.26 Hz), 8.54 (1H, s)

ESI/MS: 773 [2M+Na]⁺, 398 [M+Na]⁺, 376 [M+H]⁺

Elemental Analysis. for C₁₈H₁₆F₃N₅O

Calcd.: C,57.60; H,4.30; N,18.66

Found : C,57.81; H,4.29; N,18.79

### EXAMPLE 27

4-(4-Methoxyphenyl)-5-(6-methoxy-3-pyridazinyl)-2-pyrimidinamine (309 mg) was dissolved in c-hydrochloric acid (5 ml) and dioxane (20 ml). The solution was heated at 55° C., and stirred overnight. Evaporation of solvent in vacuo gave a residue. To the residue was added water (10 ml). The pH of the supension was adjusted to 7-8 with 1N-sodium hydroxide solution. The crystals were collected by filtration, washed with water, dried in vacuo, to give 6-[2-amino-4-(4-methoxyphenyl)-5-pyrimidinyl]-3(2H)-pyridazinone (285 mg).

mp: 142° C. (water)

IR (KBr): 3409, 1639, 1585, 1251 cm⁻¹

NMR (DMSO-d₆, δ): 3.78 (3H, s), 6.95 (1H, d, J=9.8 Hz), 6.89 (1H, d, J=9.8 Hz), 6.97 (1H, d, J=9.0 Hz), 6.99 (1H, s), 7.36 (1H, d, J=9.0 Hz), 8.33 (1H, s), 13.1 (1H, s)

ESI/MS: 296 [M+H]⁺

### EXAMPLE 28

6-[2-Amino-4-(3-fluorophenyl)-5-pyrimidinyl]-3(2H)-pyridazinone was prepared from 4-(3-fluorophenyl)-5-(6-methoxy-3-pyridazinyl)-2-pyrimidinamine according to a similar manner to that of Example 27.

mp: 249° C. (water)

IR (KBr): 3156, 1677, 1577, 1440, 1263 cm⁻¹

NMR (DMSO-d₆, δ): 6.73 (1H, d, J=9.8 Hz), 6.98 (1H, d, J=9.8 Hz), 7.12-7.45 (7H, m), 8.42 (1H, s), 13.1 (1H, s)

ESI/MS: 284 [M+H]⁺

### EXAMPLE 29

6-[2-Amino-4-(3-fluoro-4-methoxyphenyl)-5-pyrimidinyl]-3(2H)-pyridazinone was prepared from 4-(3-fluoro-4-methoxyphenyl)-5-(6-methoxy-3-pyridazinyl)-2-pyrimidinamine according to a similar manner to that of Example 27.

mp: 250° C. (water)

IR (KBr): 3164, 1654, 1550, 1417, 1238 cm⁻¹

NMR (DMSO-d₆, δ): 3.85 (3H, s), 6.73 (1H, d, J=9.8 Hz), 6.98 (1H, d, J=9. 8 Hz), 7.06 (2H, s), 7.14-7.36 (4H, m), 8.36 (1H, s), 13.1 (1H, s)

ESI/MS: 314 [M+H]⁺

### EXAMPLE 30

6-[2-Amino-4-(4-chlorophenyl)-5-pyrimidinyl]-3(2H)-pyridazinone was prepared from 4-(4-chlorophenyl)-5-(6-methoxy-3-pyridazinyl)-2-pyrimidinamine according to a similar manner to that of Example 27.

IR (KBr): 3317, 1630, 1566, 1481, 1228 cm⁻¹

NMR (DMSO-d₆, δ): 6.74 (1H, d, J=9.8 Hz), 6.97 (1H, d, J=9.8 Hz), 7.11 (1H, s), 7.32-7.51 (4H, m), 8.40 (1H, s), 13.1 (1H, s)

ESI/MS: 300 and 302 [M+H]⁺

### EXAMPLE 31

6-[2-Amino-4-(3-pyridinyl)-5-pyrimidinyl]-3(2H)-pyridazinone was prepared from 5-(6-methoxy-3-pyridazinyl)-4-(3-pyridinyl)-2-pyrimidinamine according to a similar manner to that of Example 27.

mp: 256° C. (water)

IR (KBr): 3170, 1646, 1550, 1409, 1220 cm⁻¹

NMR (DMSO-d₆, δ): 6.76 (1H, d, J=9.8 Hz), 7.09 (1H, d, J=9.8 Hz), 7.16 (2H, s), 7.40-7.48 (1H, m), 7.73-7.79 (1H, m), 8.44 (1H, s), 8.57-8.61 (2H, m), 13.1 (1H, brs)

ESI/MS: 265 [M−H]⁻

### EXAMPLE 32

6-[2-Amino-4-(4-pyridinyl)-5-pyrimidinyl]-3(2H)-pyridazinone was prepared from 5-(6-methoxy-3-pyridazinyl)-4-(4-pyridinyl)-2-pyrimidinamine according to a similar manner to that of Example 27.

IR (KBr): 3295, 1658, 1585, 1411, 1236 cm⁻¹

NMR (DMSO-d₆, δ): 6.77 (1H, d, J=9.8 Hz), 7.11 (1H, d, J=9.8 Hz), 7.21 (2H, s), 7.33-7.36 (2H, m), 8.47 (1H, s), 8.60-8.63 (2H, m), 13.0 (1H, s)

ESI/MS: 265 [M−H]⁻

### EXAMPLE 33

6-[2-Amino-4-(1,3-thiazol-2-yl)-5-pyrimidinyl]-3(2H)-pyridazinone was prepared from 5-(6-methoxy-3-pyridazinyl)-4-(1,3-thiazol-2-yl)-2-pyrimidinamine according to a similar manner to that of Example 27.

IR (KBr): 3324, 1633, 1531, 1442, 1228 cm⁻¹

NMR (DMSO-d₆, δ): 6.75 (1H, d, J=9.8 Hz), 7.22 (2H, s), 7.34 (1H, d, J=9.8 Hz), 7.89-8.04 (2H, m), 8.38 (1H, s), 13.3 (1H, s)

ESI/MS: 273 [M+H]⁺, 295 [M+Na]⁺

### EXAMPLE 34

6-[2-Amino-4-(2-furyl)-5-pyrimidinyl]-3(2H)-pyridazinone was prepared from 4-(2-furyl)-5-(6-methoxy-3-pyridazinyl)-2-pyrimidinamine according to a similar manner to that of Example 27.

mp: 262-268° C. (water)

IR (KBr): 3342, 1629, 1573, 1471, 1224 cm⁻¹

NMR (DMSO-d₆, δ): 6.61-6.64 (1H, m), 6.82-6.93 (2H, m), 7.01 (2H, s), 7.26 (1H, d, J=9.8 Hz), 7.78 (1H, s), 8.28 (1H, s), 13.1 (1H, s)

ESI/MS: 278 [M+Na]⁺

### EXAMPLE 35

6-[2-Amino-4-(4-methylphenyl)-5-pyrimidinyl]-3(2H)-pyridazinone was prepared from 5-(6-methoxy-3-pyridazinyl)-4-(4-methylphenyl)-2-pyrimidinamine according to a similar manner to that of Example 27.

mp: 249° C. (water)

IR (KBr): 3154, 1641, 1583, 1481, 1226 cm⁻¹

NMR (DMSO-d₆, δ): 2.32 (3H, s), 6.68 (1H, d, J=9.8 Hz), 6.88 (1H, d, J=9.8 Hz), 7.03 (2H, s), 7.20 (2H, d, J=8.0 Hz), 7.30 (2H, d, J=8.0 Hz), 8.37 (1H, s), 13.1 (1H, s)

ESI/MS: 280 [M+H]⁺, 302 [M+Na]⁺

### EXAMPLE 36

6-[2-Amino-4-(3,4-difluorophenyl)-5-pyrimidinyl]-3(2H)-pyridazinone was prepared from 4-(3,4-difluorophenyl)-5-(6-methoxy-3-pyridazinyl)-2-pyrimidinamine according to a similar manner to that of Example 27.

mp: 307° C. (water)

IR (KBr): 3334, 1681, 1589, 1482, 1232 cm⁻¹

NMR (DMSO-d₆, δ): 6.75 (1H, d, J=9.8 Hz), 7.03 (1H, d, J=9.8 Hz), 7.14-7.19 (2H, m), 7.40-7.94 (3H, m), 8.43 (1H, s), 13.1 (1H, s)

ESI/MS: 301 [M+H]⁺, 323 [M+Na]⁺

### EXAMPLE 37

6-[2-Amino-4-(3,4-dimethoxyphenyl)-5-pyrimidinyl]-3(2H)-pyridazinone was prepared from 4-(3,4-dimethoxyphenyl)-5-(6-methoxy-3-pyridazinyl)-2-pyrimidinamine according to a similar manner to that of Example 27.

mp: 225° C. (water)

IR (KBr): 3174, 1644, 1587, 1425, 1265 cm⁻¹

NMR (DMSO-d₆, δ): 3.68 (3H, s), 3.77 (3H, s), 6.70 (1H, d, J=9.8 Hz), 6.86-7.10 (6H, m), 8.34 (1H, s), 13.1 (1H, s)

ESI/MS: 326 [M+H]⁺, 348 [M+Na]⁺

### EXAMPLE 38

6-[2-Amino-4-(3,4-dichlorophenyl)-5-pyrimidinyl]-3(2H)-pyridazinone was prepared from 4-(3,4-dichlorophenyl)-5-(6-methoxy-3-pyridazinyl)-2-pyrimidinamine according to a similar manner to that of Example 27.

mp: 300° C. (water)

IR (KBr): 3321, 1681, 1583, 1475, 1226 cm⁻¹

NMR (DMSO-d₆, δ): 6.78 (1H, d, J=9.8 Hz), 7.11 (1H, d, J=9.8 Hz), 7.17 (2H, brs), 7.26 (1H, d, J=8.4 Hz), 7.69 (1H, d, J=8.4 Hz), 7.71 (1H, d, J=2.0 Hz), 8.43 (1H, s), 13.1 (1H, s)

ESI/MS: 356, 358 and 360 [M+Na]⁺

### EXAMPLE 39

6-[2-Amino-4-(2-methylphenyl)-5-pyrimidinyl]-3(2H)-pyridazinone was prepared from 5-(6-methoxy-3-pyridazinyl)-4-(2-methylphenyl)-2-pyrimidinamine according to a similar manner to that of Example 27.

mp: 313° C. (water)

IR (KBr): 3378, 1643, 1581, 1490, 1234 cm⁻¹

NMR (DMSO-d₆, δ): 2.06 (3H, s), 6.57 (1H, d, J=9.8 Hz), 6.72 (1H, d, J=9.8 Hz), 7.08-7.32 (4H, m), 8.44 (1H, s), 13.04 (1H, s)

ESI/MS: 302 [M+Na]⁺

### EXAMPLE 40

6-[2-Amino-4-(2-methoxyphenyl)-5-pyrimidinyl]-3(2H)-pyridazinone was prepared from 4-(2-methoxyphenyl)-5-(6-methoxy-3-pyridazinyl)-2-pyrimidinamine according to a similar manner to that of Example 27.

mp: 273° C. (water)

IR (KBr): 3365, 1646, 1575, 1490, 1228 cm⁻¹

NMR (DMSO-d₆, δ): 3.57 (3H, s), 6.66 (1H, d, J=9.8 Hz), 6.91 (1H, d, J=9.8 Hz), 7.00 (2H, s), 6.98-7.07 (2H, m), 7.36-7.45 (2H, m), 8.35 (1H, s), 12.09 (1H, s)

ESI/MS: 318 [M+Na]⁺

### EXAMPLE 41

6-[2-Amino-4-(2-thienyl)-5-pyrimidinyl]-3(2H)-pyridazinone was prepared from 5-(6-methoxy-3-pyridazinyl)-4-(2-thienyl)-2-pyrimidinamine according to a similar manner to that of Example 27.

mp: 265° C. (water)

IR (KBr): 3330, 1654, 1523, 1430, 1213 cm⁻¹

NMR (DMSO-d₆, δ): 6.87 (1H, d, J=9.8 Hz), 7.00-7.10 (2H, m), 7.01 (2H, s), 7.26 (1H, d, J=9.8 Hz), 7.73 (1H, s), 8.28 (1H, s), 13.1 (1H, s)

ESI/MS: 294 [M+Na]⁺

### EXAMPLE 42

6-[2-Amino-4-(3-methylphenyl)-5-pyrimidinyl]-3(2H)-pyridazinone was prepared from 5-(6-methoxy-3-pyridazinyl)-4-(3-methylphenyl)-2-pyrimidinamine according to a similar manner to that of Example 27.

mp : 216° C. (water)

IR (KBr): 3403, 1641, 1575, 1442, 1207 cm⁻¹

NMR (DMSO-d₆, δ): 2.30 (3H, s), 6.58 (1H, d, J=9.8 Hz), 6.86 (1H, d, J=9.8 Hz), 7.05 (2H,s), 7. 09-7.40 (4H, m), 8.38 (1H,s), 13.08 (1H, s)

ESI/MS: 302 [M+Na]⁺

### EXAMPLE 43

6-[2-Amino-4-(3-methoxyphenyl)-5-pyrimidinyl)-3(2H)-pyridazinone was prepared from 4-(3-methoxyphenyl)-5-(6-methoxy-3-pyridazinyl)-2-pyrimidinamine according to a similar manner to that of Example 27.

mp: 134° C. (water)

IR (KBr): 3336, 1671, 1575, 1491, 1216 cm⁻¹

NMR (DMSO-d₆, δ): 3.72 (3H, s), 6.68 (1H, d, J=9.8 Hz), 6.88 (1H, d, J=9.8 Hz), 7.02 (2H, s), 6.87-7.07 (2H, m), 7.26-7.43 (2H, m), 8.38 (1H, s), 13.1 (1H, s)

ESI/MS: 318 [M+Na]⁺

### EXAMPLE 44

6-[2-Amino-4-(4-methoxyphenyl)-5-pyrimidinyl]-3(2H)-pyridazinone (59.0 mg) was dissolved in dimethylformamide(5 ml). To the solution was added potassium t-butoxide (29.2 mg) and isopropyl iodide (44.2 mg). The reaction mixture was stirred at 25° C. for 2 hours. The reaction mixture was portioned to ethyl acetate and water. The organic layer was separated and washed with brine. The combined aqueous layer was extracted with ethyl acetate. The combined organic layer was passed to diatomaceous earth column. The organic solution was concentrated under reduced pressure to give a residue. The above residue was purified by column chromatography on silica gel (chloroform (100%) and chloroform-methanol (95:5)). The fraction containing product was concentrated under reduced pressure to give crystal residue. The residue was recrystallized from ethanol-water(4:1) to give 6-[2-amino-4-(4-methoxyphenyl)-5-pyrimidinyl]-2-isopropyl-3(2H)-pyridazinone (44.0 mg).

mp: 216.5° C. (ethanol-water)

IR (KBr): 3477, 1650, 1555, 1481, 1411 cm⁻¹

NMR (DMSO-d₆, δ): 1.14 (6H, d, J=6.6 Hz), 3.76 (3H, s), 5.02-5.15 (1H, m), 6.78 (1H, d, J=9.6 Hz), 6.94 (2H, d, J=6.8 Hz), 6.98 (2H, s), 7.08 (1H, d, J=9.6 Hz), 7.32 (2H, d, J=6.8 Hz), 8.38 (1H, s)

ESI/MS: 338 [M+H]⁺, 360 [M+Na]⁺

### EXAMPLE 45

6-[2-Amino-4-(4-chlorophenyl)-5-pyrimidinyl]-2-isopropyl-3(2H)-pyridazinone was prepared from 6-[2-amino-4-(4-chlorophenyl)-5-pyrimidinyl]-3(2H)-pyridazinone and isopropyl iodide according to a similar manner to that of Example 44.

mp: 195° C. (ethanol-water)

IR (KBr): 3477, 1658, 1550, 1488, 1411 cm⁻¹

NMR (DMSO-d₆, δ): 1.02 (6H, d, J=6.6 Hz), 4.96-5.10 (1H, m), 6.85 (1H, d, J=9.6 Hz), 7.12 (2H, s), 7.28 (1H, d, J=9.6 Hz), 7.35 (2H, d, J=6.6 Hz), 7.47 (2H, d, J=6.6 Hz), 8.47 (1H, s)

ESI/MS: 342 and 344 [M+H]⁺, 364 and 366 [M+Na]⁺

### EXAMPLE 46

6-[2-Amino-4-(3-fluoro-4-methoxyphenyl)-5-pyrimidinyl]-2-isopropyl-3(2H)-pyridazinone was prepared from 6-[2-amino-4-(3-fluoro-4-methoxyphenyl)-5-pyrimidinyl]-3(2H)-pyridazinone and isopropyl iodide according to a similar manner to that of Example 44.

mp: 174° C. (ethanol-water)

IR (KBr): 3477, 1650, 1555, 1481, 1411 cm⁻¹

NMR (DMSO-d₆, δ): 1.11 (6H, d, J=6.6 Hz), 3.84 (3H, s), 5.01-5.15 (1H, m), 6.78 (1H, d, J=9.6 Hz), 6.94 (2H, d, J=6.8 Hz), 6.98 (2H, s), 7.08 (1H, d, J=9.6 Hz), 7.32 (2H, d, J-=6.8 Hz), 8.38 (1H, s)

ESI/MS: 356 [M+H]⁺, 378 [M+Na]⁺

### EXAMPLE 47

6-[2-Amino-4-(2-furyl)-5-pyrimidinyl]-2-isopropyl-3(2H)-pyridazinone was prepared from 6-[2-amino-4-(2-furyl)-5-pyrimidinyl]-3(2H)-pyridazinone and isopropyl iodide according to a similar manner to that of Example 44.

mp: 215° C. (ethanol-water)

IR (KBr): 3318, 1668, 1646, 1590, 1533 cm⁻¹

NMR (DMSO-d₆, δ): 1.30 (6H, d, J=6.8 Hz), 5.09-5.20 (1H, m), 6.60-6.62 (1H, m), 6.87-6.92 (2H, m), 7.03 (2H, s), 7.32 (1H, d, J=9.6 Hz), 7.60 (1H, d, J=1.2 Hz), 8.33 (1H, s)

ESI/MS: 298 [M+H]⁺, 320 [M+Na]⁺

### EXAMPLE 48

6-[2-Amino-4-(4-methylphenyl)-5-pyrimidinyl]-2-isopropyl-3(2H)-pyridazinone was prepared from 6-[2-amino-4-(4-methylphenyl)-5-pyrimidinyl]-3(2H)-pyridazinone and isopropyl iodide according to a similar manner to that of Example 44.

mp: 224° C. (ethanol-water)

IR (KBr): 3467, 1633, 1567, 1482, 1411 cm⁻¹

NMR (DMSO-d₆, δ): 1.10 (6H, d, J=6.6 Hz), 2.30 (3H, s), 5.03-5.09 (1H, m), 6.80 (1H, d, J=9.6 Hz), 7.04 (2H, s), 7.09 (1H, d, J=9.6 Hz), 7.16-7.27 (4H, m), 8.41 (1H, s)

ESI/MS: 322 [M+H]⁺, 344 [M+Na]⁺

### EXAMPLE 49

6-[2-Amino-4-(3,4-difluorophenyl)-5-pyrimidinyl]-2-isopropyl-3(2H)-pyridazinone was prepared from 6-[2-amino-4-(3,4-difluorophenyl)-5-pyrimidinyl]-3(2H)-pyridazinone and isopropyl iodide according to a similar manner to that of Example 44.

mp: 208° C. (ethanol-water)

IR (KBr): 3405, 1643, 1581, 1515, 1490, 1282 cm⁻¹

NMR (DMSO-d₆, δ): 1.02 (6H, d, J=6.6 Hz), 4.97-5.10 (1H, m), 6.87 (1H, d, J=9.6 Hz), 7.06 (2H, s), 7.34 (1H, d, J=9.6 Hz), 7.39-7.53 (3H, m), 8.49 (1H, s)

ESI/MS: 344 [M+H]⁺, 366 [M+Na]⁺

### EXAMPLE 50

6-[2-Amino-4-(3,4-dichlorophenyl)-5-pyrimidinyl]-2-isopropyl-3(2H)-pyridazinone was prepared from 6-[2-amino-4-(3,4-dichlorophenyl)-5-pyrimidinyl]-3(2H)-pyridazinone and isopropyl iodide according to a similar manner to that of Example 44.

mp: 176° C. (ethanol-water)

IR (KBr): 3342, 1645, 1581, 1483, 1409 cm⁻¹

NMR (DMSO-d₆, δ): 0.98 (6H, d, J=6.6 Hz), 4.96-5.09 (1H, m), 6.90 (1H, d, J=9.6 Hz), 7.19 (2H, s), 7.26 (1H, dd, J=2,8.2 Hz), 7.36 (1H, d, J=9.6 Hz), 7.61-7.67 (2H, m), 8.51 (1H, s)

ESI/MS: 376, 378 and 380 [M+H]⁺, 398, 400 and 402 [M+Na]⁺

### EXAMPLE 51

6-[2-Amino-4-(3,4-dimethoxyphenyl)-5-pyrimidinyl]-2-isopropyl-3(2H)-pyridazinone was prepared from 6-[2-amino-4-(3,4-dimethoxyphenyl)-5-pyrimidinyl]-3(2H)-pyridazinone and isopropyl iodide according to a similar manner to that of Example 44.

mp: 167° C. (ethanol-water)

IR (KBr): 3390, 1659, 1639, 1575, 1465, 1265 cm⁻¹

NMR (DMSO-d₆, δ): 1.17 (6H, d, J=6.6 Hz), 3.63 (3H, s), 3.76 (3H, s), 5.07-5.17 (1H, m), 6.78 (1H, d, J=9.6 Hz), 6.88-7.04 (6H, m), 8.38 (1H, s)

ESI/MS: 368 [M+H]⁺, 390 [M+Na]⁺

### EXAMPLE 52

6-[2-Amino-4-(3-pyridinyl)-5-pyrimidinyl]-2-isopropyl-3(2H)-pyridazinone was prepared from 6-[2-amino-4-(3-pyridinyl)-5-pyrimidinyl]-3(2H)-pyridazinone and isopropyl iodide according to a similar manner to that of Example 44.

mp: 204° C. (ethanol-water)

IR (KBr): 3343, 1654, 1589, 1550, 1290 cm⁻¹

NMR (DMSO-d₆, δ): 0.94 (6H, d, J=6.6 Hz), 4.92-5.06 (1H, m), 6.89 (1H, d, J=9.6 Hz), 7.17 (2H, s), 7.39-7.46 (2H, m), 7.71-7.78 (1H, m), 8.50-8.58 (3H, m)

ESI/MS: 309 [M+H]⁺, 331 [M+Na]⁺

### EXAMPLE 53

6-[2-Amino-4-(4-pyridinyl)-5-pyrimidinyl]-2-isopropyl-3(2H)-pyridazinone was prepared from 6-[2-amino-4-(4-pyridinyl)-5-pyrimidinyl]-3(2H)-pyridazinone and isopropyl iodide according to a similar manner to that of Example 44.

mp: 219° C. (ethanol-water)

IR (KBr): 3343, 1662, 1644, 1585, 1482, 1216 cm⁻¹

NMR (DMSO-d₆, δ): 0.88 (6H, d, J=6.6 Hz), 4.91-5.04 (1H, m), 6.90 (1H, d, J=9.6 Hz), 7.21 (2H, s), 7.25-7.31 (2H, m), 7.47 (1H, d, J=9.6 Hz), 8.56-8.61 (3H, m)

ESI/MS: 309 [M+H]⁺, 331 [M+Na]⁺

### EXAMPLE 54

6-[2-Amino-4-(1,3-thiazol-2-yl)-5-pyrimidinyl]-2-isopropyl-3(2H)-pyridazinone was prepared from 6-[2-amino-4-(1,3-thiazol-2-yl)-5-pyrimidinyl]-3(2H)-pyridazinone and isopropyl iodide according to a similar manner to that of Example 44.

IR (KBr): 3324, 1689, 1633, 1589, 1531, 1228 cm⁻¹

NMR (DMSO-d₆, δ): 1.23 (6H, d, J=6.6 Hz), 5.09-5.19 (1H, m), 6.80 (1H, d, J=9.6 Hz), 7.24 (2H, s), 7.38 (1H, d, J=9.6 Hz), 7.89 (1H, d, J=3.2 Hz), 7.93 (1H, d, J=3.2 Hz), 8.43 (1H, s)

ESI/MS: 315 [M+H]⁺, 337 [M+Na]⁺

### EXAMPLE 55

6-(2-Amino-4-(2-thienyl)-5-pyrimidinyl]-2-isopropyl-3(2H)-pyridazinone was prepared from 6-[2-amino-4-(2-thienyl)-5-pyrimidinyl]-3(2H)-pyridazinone and isopropyl iodide according to a similar manner to that of Example 44.

mp: 213° C. (ethanol-water)

IR (KBr): 3363, 1666, 1646, 1590, 1535, 1482, 1211 cm⁻¹

NMR (DMSO-d₆, δ): 1.24 (6H, d, J=6.6 Hz), 5.11-5.24 (1H, m), 6.92 (1H, d, J=9.6 Hz), 7.04 (2H, s), 6.96-7.08 (2H, m), 7.32 (1H, d, J=9.6 Hz), 7.71 (1H, d, J=5.0 Hz), 8.29 (1H, s)

ESI/MS: 314 [M+H]⁺, 336 [M+Na]⁺

### EXAMPLE 56

6-[2-Amino-4-(2-methylphenyl)-5-pyrimidinyl]-2-isopropyl-3(2H)-pyridazinone was prepared from 6-[2-amino-4-(2-methylphenyl)-5-pyrimidinyl]-3(2H)-pyridazinone and isopropyl iodide according to a similar manner to that of Example 44.

mp: 203° C. (ethanol-water)

IR (KBr): 3293, 1666, 1627, 1594, 1538, 1486, 1205 cm⁻¹

NMR (DMSO-d₆, δ): 0.96 (6H, d, J=6.8 Hz), 2.03 (3H, s), 4.90-5.04 (1H, m), 6.76 (1H, d, J=9.6 Hz), 7.07 (2H,s), 7.10-7.29 (5H, m), 8.51 (1H, s)

ESI/MS: 322 [M+H]⁺, 344 [M+Na]⁺

### EXAMPLE 57

6-[2-Amino-4-(3-methylphenyl)-5-pyrimidinyl]-2-isopropyl-3(2H)-pyridazinone was prepared from 6-[2-amino-4-(3-methylphenyl)-5-pyrimidinyl]-3(2H)-pyridazinone and isopropyl iodide according to a similar manner to that of Example 44.

mp: 198° C. (ethanol-water)

IR (KBr): 3475, 1664, 1624, 1585, 1531, 1482, 1209 cm⁻¹

NMR (DMSO-d₆, δ): 1.06 (6H, d, J=6.6 Hz), 2.28 (3H, s), 4.98-5.12 (1H, m), 6.79 (1H, d, J=9.6 Hz), 7.07 (2H, s), 7.14 (1H, d, J=9.6 Hz), 7.21-7.37 (4H, m), 8.50 (1H, s)

ESI/MS: 322 [M+H]⁺, 344 [M+Na]⁺

### EXAMPLE 58

6-[2-Amino-4-(2-methoxyphenyl)-5-pyrimidinyl]-2-isopropyl-3(2H)-pyridazinone was prepared from 6-[2-amino-4-(2-methoxyphenyl)-5-pyrimidinyl]-3(2H)-pyridazinone and isopropyl iodide according to a similar manner to that of Example 44.

mp: 170° C. (ethanol-water)

IR (KBr): 3330, 1651, 1587, 1535, 1486, 1254 cm⁻¹

NMR (DMSO-d₆, δ): 0.95 (6H, d, J=6.6 Hz), 3.40 (3H, s), 4.90-5.03 (1H, m), 6.79 (1H, d, J=9.6 Hz),.6.98 (2H,s), 6.89-7.05 (2H, m), 7.22 (1H, d, J=9.6 Hz), 7.31-7.43 (2H, m), 8.39 (1H, s)

ESI/MS: 338 [M+H]⁺, 360 [M+Na]⁺

### EXAMPLE 59

6-[2-Amino-4-(3-methoxyphenyl)-5-pyrimidinyl]-2-isopropyl-3(2H)-pyridazinone was prepared from 6-[2-amino-4-(3-methoxyphenyl)-5-pyrimidinyl]-3(2H)-pyridazinone and isopropyl iodide according to a similar manner to that of Example 44.

mp: 211° C. (ethanol-water)

IR (KBr): 3467, 1658, 1623, 1583, 1488, 1288 cm⁻¹

NMR (DMSO-d₆, δ): 1.07 (6H, d, J=6.6 Hz), 3.68 (3H, s), 4.99-512 (1H, m), 6.80 (1H, d, J=9.6 Hz), 6.87-6.98 (3H, m), 7.09 (2H, s), 7.15 (1H, d, J=9.6 Hz), 7.25-7.39 (1H, m), 8.50 (1H, s)

ESI/MS: 338 [M+H]⁺, 360 [M+Na]⁺

### EXAMPLE 60

6-[2-Amino-4-(4-methoxyphenyl)-5-pyrimidinyl]-2-methyl-3(2H)-pyridazinone was prepared from 6-[2-amino-4-(4-methoxyphenyl)-5-pyrimidinyl]-3(2H)-pyridazinone and methyl iodide according to a similar manner to that of Example 44.

mp: 259° C. (ethanol-water)

IR (KBr): 3335, 1685, 1580, 1485, 1411, 1270 cm⁻¹

NMR (DMSO-d₆, δ): 3.68 (3H, s), 3.78 (3H, s), 6.73 (1H, d, J=9.6 Hz), 6.83 (1H, d, J=9.6 Hz), 6.95 (2H, d, J=6.8 Hz), 6.99 (2H, s), 7.38 (2H, d, J=6.8 Hz), 8.35 (1H, s)

ESI/MS: 310 [M+H]⁺, 332 [M+Na]⁺

### EXAMPLE 61

6-[2-Amino-4-(4-chlorophenyl)-5-pyrimidinyl]-2-methyl-3(2H)-pyridazinone was prepared from 6-[2-amino-4-(4-chlorophenyl)-5-pyrimidinyl]-3(2H)-pyridazinone and methyl iodide according to a similar manner to that of Example 44.

mp: 264° C. (ethanol-water)

IR (KBr): 3330, 1685, 1572, 1480, 1411, 1230 cm⁻¹

NMR (DMSO-d₆, δ): 3.65 (3H, s), 6.76 (1H, d, J=9.6 Hz), 6.91 (1H, d, J=9.6 Hz), 7.14 (2H, s), 7.34-7.51 (4H, m), 8.43 (1H, s)

ESI/MS: 336 and 338 [M+H]⁺

### EXAMPLE 62

6-[2-Amino-4-(3-fluorophenyl)-5-pyrimidinyl]-2-methyl-3(2H)-pyridazinone was prepared from 6-[2-amino-4-(3-fluorophenyl)-5-pyrimidinyl]-3(2H)-pyridazinone and methyl iodide according to a similar manner to that of Example 44.

mp: 212.5° C. (ethanol-water)

IR (KBr): 3330, 1666, 1580, 1485, 1411 cm⁻¹

NMR (DMSO-d₆, δ): 3.64 (3H, s), 6.76 (1H, d, J=9.6 Hz), 6.93 (1H, d, J=9.6 Hz), 7.15 (2H, s), 7.13-7.45 (4H, m), 8.45 (1H, s)

ESI/MS: 298 [M+H]⁺, 320 [M+Na]⁺

### EXAMPLE 63

6-(2-Amino-4-(3-fluoro-4-methoxyphenyl)-5-pyrimidinyl]-2-methyl-3(2H)-pyridazinone was prepared from 6-[2-amino-4-(3-fluoro-4-methoxyphenyl)-5-pyrimidinyl]-3(2H)-pyridazinone and methyl iodide according to a similar manner to that of Example 44.

mp: 245° C. (ethanol-water)

IR (KBr): 3326, 1652, 1585, 1481, 1411 cm⁻¹

NMR (DMSO-d₆, δ): 3.67 (3H, s), 3.86 (3H, s), 6.77 (1H, d, J=9.6 Hz), 7.07 (2H, s), 7.12-7.38 (3H, m), 8.38 (1H, s)

ESI/MS: 328 [M+H]⁺, 350 [M+Na]⁺

### EXAMPLE 64

6-[2-Amino-4-(2-furyl)-5-pyrimidinyl]-2-methyl-3(2H)-pyridazinone was prepared from 6-[2-amino-4-(2-furyl)-5-pyrimidinyl]-3(2H)-pyridazinone and methyl iodide according to a similar manner to that of Example 44.

mp: 234° C. (ethanol-water)

IR (KBr): 3326, 1658, 1571, 1411 cm⁻¹

NMR (DMSO-d₆, δ): 3.69 (3H, s), 6.61-6.64 (1H, m), 6.89 (1H, d, J=9.6 Hz), 6.92-6.96(1H, m), 7.03 (2H, s), 7.27 (1H, d, J=9.6 Hz), 7.77-7.79 (1H, m), 8.30 (1H, s)

ESI/MS: 270 [M+H]⁺, 292 [M+Na]⁺

### EXAMPLE 65

6-[2-Amino-4-(4-methylphenyl)-5-pyrimidinyl]-2-methyl-3(2H)-pyridazinone was prepared from 6-[2-amino-4-(4-methylphenyl)-5-pyrimidinyl]-3(2H)-pyridazinone and methyl iodide according to a similar manner to that of Example 44.

mp: 230° C. (ethanol-water)

IR (KBr): 3326, 1652, 1585, 1481, 1411 cm⁻¹

NMR (DMSO-d₆, δ): 2.37 (3H, s), 3.67 (3H, s), 6.71 (1H, d, J=9.6 Hz), 6.81 (1H, d, J=9.6 Hz), 7.05 (2H, s), 7.20 (2H, d, J=8.2 Hz), 7.31 (2H, d, J=8.2 Hz), 8.38 (1H, s)

ESI/MS: 294 [M+H]⁺, 316 [M+Na]⁺

### EXAMPLE 66

6-[2-Amino-4-(3,4-difluorophenyl)-5-pyrimidinyl]-2-methyl-3(2H)-pyridazinone was prepared from 6-[2-amino-4-(3,4-difluorophenyl)-5-pyrimidinyl]-3(2H)-pyridazinone and methyl iodide according to a similar manner to that of Example 44.

mp: 231° C. (ethanol-water)

IR (KBr): 3322, 1641, 1581, 1515, 1488, 1280 cm⁻¹

NMR (DMSO-d₆, δ): 3.64 (3H, s), 6.78 (1H, d, J=9.6 Hz), 6.98 (1H, d, J=9.6 Hz), 7.15-7.19 (3H, m), 7.43-7.57 (2H, m), 8.45 (1H, s)

ESI/MS: 316 [M+H]⁺, 338 [M+Na]⁺

### EXAMPLE 67

6-[2-Amino-4-(3,4-dimethoxyphenyl)-5-pyrimidinyl]-2-methyl-3(2H)-pyridazinone was prepared from 6-[2-amino-4-(3,4-dimethoxyphenyl)-5-pyrimidinyl]-3(2H)-pyridazinone and methyl iodide according to a similar manner to that of Example 44.

mp: 232° C. (ethanol-water)

IR (KBr): 3386, 1629, 1575, 1481, 1263 cm⁻¹

NMR (DMSO-d₆, δ): 3.69 (3H, s), 3.77 (3H, s), 6.74 (1H, d, J=9.6 Hz), 6.83 (1H, d, J=9.6 Hz), 6.87-7.06 (5H, m), 8.36 (1H, s)

ESI/MS: 340 [M+H]⁺, 362 [M+Na]⁺

### EXAMPLE 68

6-[2-Amino-4-(3,4-dichlorophenyl)-5-pyrimidinyl]-2-methyl-3(2H)-pyridazinone was prepared from 6-[2-amino-4-(3,4-dichlorophenyl)-5-pyrimidinyl]-3(2H)-pyridazinone and methyl iodide according to a similar manner to that of Example 44.

mp: 238° C. (ethanol-water)

IR (KBr): 3324, 1641, 1583, 1482, 1407 cm⁻¹

NMR (DMSO-d₆, δ): 3.64 (3H, s), 6.81 (1H, d, J=9.6 Hz), 7.05 (1H, d, J=9.6 Hz), 7.20 (2H, s), 7.23-7.29 (1H, m), 7.62-7.76 (2H, m), 8.46 (1H, s)

ESI/MS: 370, 372 and 374 [M+Na]⁺

### EXAMPLE 69

6-[2-Amino-4-(2-thienyl)-5-pyrimidinyl]-2-methyl-3(2H)-pyridazinone was prepared from 6-[2-amino-4-(2-thienyl)-5-pyrimidinyl]-3(2H)-pyridazinone and methyl iodide according to a similar manner to that of Example 44.

mp: 270° C. (ethanol-water)

IR (KBr): 3365, 1654, 1589, 1535, 1485, 1430 cm⁻¹

NMR (DMSO-d₆, δ): 3.71 (3H, s), 6.92 (1H, d, J=9.6 Hz), 7.03 (4H, m), 7.29 (1H, d, J=9.6 Hz), 7.72-7.75 (1H, m), 8.27 (1H, s)

ESI/MS: 286 [M+H]⁺, 308 [M+Na]⁺

### EXAMPLE 70

6-[2-Amino-4-(2-methylphenyl)-5-pyrimidinyl]-2-methyl-3(2H)-pyridazinone was prepared from 6-[2-amino-4-(2-methylphenyl)-5-pyrimidinyl]-3(2H)-pyridazinone and methyl iodide according to a similar manner to that of Example 44.

mp: 229° C. (ethanol-water)

IR (KBr): 3307, 1670, 1617, 1592, 1538, 1477, 1267 cm⁻¹

NMR (DMSO-d₆, δ): 2.06 (3H, s), 3.56 (3H, s), 6.45 (1H, d, J=9.6 Hz), 6.78 (1H, d, J=9.6 Hz), 7.09 (2H, s), 7.12-7.41 (4H, m), 8.49 (1H, s)

ESI/MS: 294 [M+H]⁺, 316 [M+Na]⁺

### EXAMPLE 71

6-[2-Amino-4-(3-methylphenyl)-5-pyrimidinyl]-2-methyl-3(2H)-pyridazinone was prepared from 6-[2-amino-4-(3-methylphenyl)-5-pyrimidinyl]-3(2H)-pyridazinone and methyl iodide according to a similar manner to that of Example 44.

mp: 214° C. (ethanol-water)

IR (KBr): 3413, 1639, 1579, 1531, 1488, 1402 cm⁻¹

NMR (DMSO-d₆, δ): 2.31 (3H, s), 3.67 (3H, s), 6.70 (1H, d, J=9.6 Hz), 6.81 (1H, d, J=9.6 Hz), 7.08 (1H, s), 7.24-7.41 (1H, m), 8.41 (1H, s)

ESI/MS: 294 [M+H]⁺, 316 [M+Na]⁺

### EXAMPLE 72

6-[2-Amino-4-(2-methoxyphenyl)-5-pyrimidinyl]-2-methyl-3(2H)-pyridazinone was prepared from 6-[2-amino-4-(2-methoxyphenyl)-5-pyrimidinyl]-3(2H)-pyridazinone and methyl iodide according to a similar manner to that of Example 44.

mp: 225° C. (ethanol-water)

IR (KBr): 3340, 1652, 1589, 1535, 1488, 1255 cm⁻¹

NMR (DMSO-d₆, δ): 3.44 (3H, s), 3.53 (3H, s), 6.71 (1H, d, J=9.6 Hz), 6.92 (1H, d, J=9.6 Hz), 6.93-7.08 (2H, m), 7.34-7.45 (2H, m), 8.39 (1H, s)

ESI/MS: 310 [M+H]⁺, 332 [M+Na]⁺

### EXAMPLE 73

6-[2-Amino-4-(3-methoxyphenyl)-5-pyrimidinyl]-2-methyl-3(2H)-pyridazinone was prepared from 6-[2-amino-4-(3-methoxyphenyl)-5-pyrimidinyl]-3(2H)-pyridazinone and methyl iodide according to a similar manner to that of Example 44.

mp: 195° C. (ethanol-water)

IR (KBr): 3365, 1656, 1624, 1569, 1477, 1268 cm⁻¹

NMR (DMSO-d₆, δ): 3.66 (3H, s), 3.72 (3H, s), 6.72 (1H, d, J=9.6 Hz), 6.83 (1H, d, J=9.6 Hz), 6.87-7.03 (2H, m), 7.10 (1H, s), 7.26-7.40 (2H, m), 8.41 (1H, s)

ESI/MS: 310 [M+H]⁺, 332 [M+Na]⁺

### EXAMPLE 74

6-[2-Amino-4-(2-bromophenyl)-5-pyrimidinyl]-2-methyl-3(2H)-pyridazinone was obtained according to a similar manner to that of Example 2.

mp: >250° C. (95% EtOH)

NMR (DMSO-d₆, δ): 3.44 (3H, s), 6.77 (1H, d, J=9.6 Hz), 7.10 (1H, d, J=9.6 Hz), 7.18 (2H, brd. s), 7.32-7.47 (3H, m), 7.62 (1H, d, J=7.9 Hz), 8.56 (1H, s)

ESI/MS: 380 and 382 [M+Na]⁺

### EXAMPLE 75

6-(2-Amino-4-phenyl-5-pyrimidinyl)-2-methyl-3(2H)-pyridazinone was obtained according to a similar manner to that of Example 16.

ESI/MS: 302 [M+Na]⁺

### EXAMPLE 76

6-[2-Amino-4-(2-bromophenyl)-5-pyrimidinyl]-2-(heptadeutero-isopropyl)-3(2H)-pyridazinone was obtained according to a similar manner to that of Example 2.

mp: 203-204° C. (95% EtOH)

NMR (CDCl₃, δ): 5.39 (2H, brd. s), 6.73 (1H, d, J=9.6 Hz), 6.93 (1H, d, J=9.6 Hz), 7.22-7.41 (3H, m), 7.55 (1H, d, J=8.0 Hz), 8.56 (1H, s)

ESI/MS: 415 and 417 [M+Na]⁺

### EXAMPLE 77

6-(2-Amino-4-phenyl-5-pyrimidinyl)-2-(heptadeuteroisopropyl)-3(2H)-pyridazinone was obtained according to a similar manner to that of Example 16.

mp: 214-215° C. (95% EtOH)

NMR (DMSO-d₆, δ): 6.80 (1H, d, J=9.6 Hz), 7.18 (1H, d, J=9.6 Hz), 7.30-7.41 (5H, m), 8.46 (1H, s)

ESI/MS: 373 [M+Na]⁺

### EXAMPLE 78

A solution of 6-[1-benzoyl-2,2-bis(methylthio)vinyl]-2-isopropyl-3(2H)-pyridazinone (4.00 g) and guanidine carbonate (1.00 g) in N,N-dimethylacetamide (4 ml) was stirred at 130-135° C. for 2 hours. After addition of guanidine carbonate (1.00 g), the mixture was stirred at the same temperature for 8 hours and poured into water (80 ml) to give a precipitate. The precipitate was collected by filtration, dissolved in chloroform, dried over magnesium sulfate and concentrated under reduced pressure to give a residue. The residue was dissolved in acetone under reflux and diisopropyl ether was added to the solution to give 6-[2-amino-4-(methylthio)-6-phenyl-5-pyrimidinyl]-2-isopropyl-3(2H)-pyridazinone as a solid (1.26 g).

mp: 221-222° C. (chloroform-diisopropyl ether)

IR (KBr): 3500, 3286, 3151, 1658, 1620, 1587, 1539 cm⁻¹

ESI/MS: 729[2M+Na]⁺, 376[M+Na]⁺, 354[M+H]⁺

¹H NMR (CDCl₃, δ): 1.26 (6H, d, J=6.66 Hz), 2.50 (3H, s), 5.20 (2H, br.s), 5.29 (1H, 7-plet, J=6.66 Hz), 6.70 (1H, d, J=9.50 Hz), 6.82 (1H, d, J=9.50 Hz), 7.24-7.35 (5H, m)

¹H NMR (DMSO-d₆, δ): 1.09 (6H, d, J=6.62 Hz), 2.47 (3H, s), 5.08 (1H, 7-plet, J=6.62 Hz), 6.78 (1H, d, J=9.52 Hz), 7.03 (2H, br.s), 7.15 (1H, d, J=9.52 Hz), 7.23-7.35 (5H, m)

Elemental Analysis for C₁₈H₁₉N₅OS

Calcd.: C,61.17; H,5.42; N,19.81

Found: C,61.27; H,5.53; N,19.71

### EXAMPLE 79

Under ice-cooling, 3-chloroperbenzoic acid (70% purity) (0.70 g) was added to a solution of 6-[2-amino-4-(methylthio)-6-phenyl-5-pyrimidinyl]-2-isopropyl-3(2H)-pyridazinone (1.01 g) in dichloromethane (10 ml). After stirring at ambient temperature for 5 hours, the mixture was washed with saturated aqueous sodium thiosulfate, saturated aqueous sodium hydrogen carbonate and brine, successively, dried over magnesium sulfate and concentrated under reduced pressure to give a residue. The residue was purified by column chromatography on silica gel. With an elution of a mixture of n-hexane and ethyl acetate (30:70 v/v) was given 6-[2-amino-4-(methylsulfonyl)-6-phenyl-5-pyrimidinyl]-2-isopropyl-3(2H)-pyridazinone as a solid (29 mg). Next, with an elution of a mixture of methanol and ethyl acetate (2:98 v/v) was given 6-[2-amino-4-(methylsulfinyl)-6-phenyl-5-pyrimidinyl]-2-isopropyl-3(2H)-pyridazinone as a solid (1.00 g).

6-[2-amino-4-(methylsulfinyl)-6-phenyl-5-pyrimidinyl]-2-isopropyl-3(2H)-pyridazinone

mp: 225-227° C. (dimethyl sulfoxide-acetone)

IR (KBr): 3408, 3302, 3207, 1658, 1630, 1591, 1560, 1512 cm⁻¹

ESI/MS: 761[2M+Na]⁺, 392[M+Na]⁺

ESI/MS-Neg: 368[M−H]⁻

¹H NMR (CDCl₃, δ): 1.36 (3H, d, J=6.60 Hz), 1.39 (3H, d, J=6.60 Hz), 2.99 (3H, s), 5.39 (1H, 7-plet, J=6.60 Hz), 5.83 (2H, br.s), 6.59 (1H, d, J=9.56 Hz), 6.64 (1H, d, j=9.56 Hz), 7.31-7.48 (5H, m)

Elemental Analysis for C₁₈H₁₉N₅O₂S.0.2H₂O

Calcd.: C,57.96; H,5.24; N,18.77

Found: C,58.18; H,5.17; N,18.72

6-[2-amino-4-(methylsulfonyl)-6-phenyl-5-pyrimidinyl]-2-isopropyl-3(2H)-pyridazinone

mp: 210-211° C. (ethanol)

IR (KBr): 3384, 3203, 1653, 1631, 1587, 1564, 1512 cm⁻¹

ESI/MS-Neg: 384[M−H]⁻

¹H NMR (CDCl₃, δ): 1.21 (6H, d, J=6.62 Hz), 3.28 (3H, s), 5.27 (1H, 7-plet, J=6.62 Hz), 5.54 (2H, br.s), 6.74 (1H, d, J=9.52 Hz), 6.96 (1H, d, J=9.52 Hz), 7.28-7.38 (5H, m)

Elemental Analysis for C₁₈H₁₉N₅O₃S.0.2H₂O

Calcd.: C,55.57; H,5.03; N,18.00

Found: C,55.73; H,5.05; N,17.71

### EXAMPLE 80

A mixture of 6-[2-amino-4-(methylsulfinyl)-6-phenyl-5-pyrimidinyl]-2-isopropyl-3(2H)-pyridazinone (150 mg) and sodium methoxide (35 mg) in methanol (0.6 ml) was heated under reflux for 5 hours. After concentration of the mixture, a residue was purified by column chromatography on silica gel (n-hexane-ethyl acetate 20:80 v/v) to give 6-(2-amino-4-methoxy-6-phenyl-5-pyrimidinyl)-2-isopropyl-3(2H)-pyridazinone as a solid (107 mg).

mp: 230-233° C. (ethanol)

IR (KBr): 3519, 3394, 1660, 1606, 1581, 1543 cm⁻¹

ESI/MS: 697[2M+Na]⁺, 360[M+Na]⁺, 338[M+H]⁺

¹H NMR (CDCl₃, δ): 0.92 (6H, d, J=6.62 Hz), 3.95 (3H, s), 5.11 (1H, 7-plet, J=6.62 Hz), 5.22 (2H, br.s), 6.84 (1H, d, J=9.52 Hz), 7.21-7.30 (6H, m)

Elemental Analysis for C₁₈H₁₉N₅O₂

Calcd.: C,64.08; H,5.68; N,20.76

Found: C,64.24; H,5.64; N,20.75

### EXAMPLE 81

Sodium hydride (60% in oil suspension) (19.5 mg) was added in ethanol (0.6 ml) under ice-cooling. After stirring at the ambient temperature for 30 minutes, 6-[2-amino-4-(methylsulfinyl)-6-phenyl-5-pyrimidinyl]-2-isopropyl-3(2H)-pyridazinone (150 mg) was added to the mixture. The mixture was heated at 100-105° C. for 5 hours in a sealed tube. The mixture was concentrated under reduced pressure and purified by column chromatography on silica gel (n-hexane-ethyl acetate 50 : 50 v/v) to give 6-(2-amino-4-ethoxy-6-phenyl-5-pyrimidinyl)-2-isopropyl-3(2H)-pyridazinone as a solid (99 mg).

mp: 187.5-188.5° C. (ethanol-diisopropyl ether)

IR (KBr): 3521, 3388, 1658, 1608, 1579, 1545 cm⁻¹

ESI/MS: 725[2M+Na]⁺, 374[M+Na]⁺, 352[M+H]⁺

¹H NMR (CDCl₃, δ): 0.94 (6H, d, J=6.62 Hz), 1.35 (3H, t, J=7.06 Hz), 4.41 (2H, q, J=7.06 Hz), 5.05-5.19 (3H, m), 6.82 (1H, d, J=9.50 Hz), 7.21 (1H, d, J=9.50 Hz), 7.22-7.30 (5H, m)

Elemental Analysis for C₁₉H₂₁N₅O₂.0.1H₂O

Calcd.: C,64.61; H,6.05; N,19.83

Found: C,64.60; H,5.93; N,19.81

### EXAMPLE 82

6-(2-Amino-4-isopropoxy-6-phenyl-5-pyrimidinyl)-2-isopropyl-3(2H)-pyridazinone was prepared from 6-[2-amino-4-(methylsulfinyl)-6-phenyl-5-pyrimidinyl]-2-isopropyl-3(2H)-pyridazinone and 2-propanol according to a similar manner to that of Example 81.

mp: 176.5-177.5° C. (diisopropyl ether)

IR (KBr): 3352, 3307, 3184, 1664, 1647, 1635, 1572, 1545 cm⁻¹

ESI/MS: 753[2M+Na]⁺, 388[M+Na]⁺, 366[M+H]⁺

¹H NMR (CDCl₃, δ): 0.95 (6H, d, J=6.72 Hz), 1.32 (6H, d, J=6.25 Hz), 5.07-5.19 (3H, m), 5.40 (1H, 7-plet, J=6.25 Hz), 6.81 (1H, d, J=9.52 Hz), 7.17 (1H, d, J=9.52 Hz), 9.26 (5H, s)

### EXAMPLE 83

6-[2-Amino-4-(cyclobutyloxy)-6-phenyl-5-pyrimidinyl]-2-isopropyl-3(2H)-pyridazinone was prepared from 6-[2-amino-4-(methylsulfinyl)-6-phenyl-5-pyrimidinyl]-2-isopropyl-3(2H)-pyridazinone and cyclobutanol according to a similar manner to that of Example 81.

mp: 161-162.5° C. (acetone-n-hexane)

IR (KBr): 3381, 3325, 3155, 1653, 1589, 1572, 1547 cm⁻¹

ESI/MS: 400[M+Na]⁺, 378[M+H]⁺

¹H NMR (CDCl₃, δ): 0.94 (6H, d, J=6.60 Hz), 1.62-1.85 (2H, m), 2.02-2.18 (2H, m), 2.39-2.50 (2H, m), 5.05-5.33 (4H, m), 6.82 (1H, d, J=9.52 Hz), 7.19-7.30 (6H, m)

Elemental Analysis for C₂₁H₂₃N₅O₂

Calcd.: C,66.83; H,6.14; N,18.55

Found: C,66.98; H,6.17; N,18.60

### EXAMPLE 84

6-[4-(Allyloxy)-2-amino-6-phenyl-5-pyrimidinyl]-2-isopropyl-3(2H)-pyridazinone was prepared from 6-[2-amino-4-(methylsulfinyl)-6-phenyl-5-pyrimidinyl]-2-isopropyl-3(2H)-pyridazinone and 2-propen-1-ol according to a similar manner to that of Example 81.

mp: 130.5-132.5° C. (diisopropyl ether)

IR (KBr): 3489, 3311, 3192, 1647, 1626, 1576, 1545 cm⁻¹

ESI/MS: 749[2M+Na]⁺, 386[M+Na]⁺, 364[M+H]⁺

¹H NMR (CDCl₃, δ): 0.94 (6H, d, J=6.56 Hz), 4.85-4.90 (2H, m), 5.06-5.36 (5H, m), 5.91-6.10 (1H, m), 6.83 (1H, d, J=9.52 Hz), 7.23 (1H, d, J=9.52 Hz), 7.27 (5H, s)

### EXAMPLE 85

Sodium hydride (60% in oil suspension) (19.5 mg) was added in 2-propyn-1-ol (0.6 ml) under ice-cooling. After stirring at the ambient temperature for 30 minutes, 6-[2-amino-4-(methylsulfinyl)-6-phenyl-5-pyrimidinyl)-2-isopropyl-3(2H)-pyridazinone (150 mg) was added to the mixture. The mixture was heated at 100-105° C. for 5 hours. The mixture was concentrated under reduced pressure and purified by column chromatography on silica gel (n-hexane-ethyl acetate 50:50 v/v) to give 6-[2-amino-4-phenyl-6-(2-propynyloxy)-5-pyrimidinyl]-2-isopropyl-3(2H)-pyridazinone as a solid (101 mg).

mp: 145-146.6° C. (acetone-diisopropyl ether)

IR (KBr): 3410, 3294, 3176, 2121, 1657, 1637, 1591, 1574, 1545 cm⁻¹

ESI/MS: 745[2M+Na]⁺, 384[M+Na]⁺, 362[M+H]⁺

¹H NMR (CDCl₃, δ): 0.93 (6H, d, J=6.60 Hz), 2.49 (1H, t, J=2.35 Hz), 5.00 (2H, d, J=2.35 Hz), 5.09 (1H, 7-plet, J=6.60 Hz), 5.20 (2H, br.s), 6.84 (1H, d, J=9.52 Hz), 7.24 (1H, d, J=9.52 Hz), 7.27 (5H, s)

Elemental Analysis for C₂₀H₁₉N₅O₂.0.1H₂O

Calcd.: C,66.14; H,5.33; N,19.28

Found: C,66.19; H,5.25; N,19.19

### EXAMPLE 86

6-[2-Amino-4-(2-hydroxyethoxy)-6-phenyl-5-pyrimidinyl]-2-isopropyl-3(2H)-pyridazinone was prepared from 6-[2-amino-4-(methylsulfinyl)-6-phenyl-5-pyrimidinyl]-2-isopropyl-3(2H)-pyridazinone and ethylene glycol according to a similar manner to that of Example 85.

mp: 182-183° C. (ethanol)

IR (KBr): 3352, 3178, 1646, 1579, 1546 cm⁻¹

ESI/MS: 757[2M+Na]⁺, 390[M+Na]⁺, 368[M+H]⁺

¹H NMR (DMSO-d₆, δ): 0.76 (6H, d, J=6.60 Hz), 3.63-3.72 (2H, m), 4.36 (2H, t, J=5.04 Hz), 4.81 (1H, t, J=5.22 Hz), 4.87 (1H, 7-plet, J=6.60 Hz), 6.85 (1H, d, J=9.54 Hz), 6.95 (2H, br.s), 7.16-7.34 (5H, m), 7.55 (1H, d, J=9.54 Hz)

Elemental Analysis for C₁₉H₂₁N₅O₃

Calcd.: C,62.11; H,5.76; N,19.06

Found: C,62.19; H,5.78; N,18.98

### EXAMPLE 87

6-[2-Amino-4-(2-methoxyethoxy)-6-phenyl-5-pyrimidinyl]-2-isopropyl-3(2H)-pyridazinone was prepared from 6-[2-amino-4-(methylsulfinyl)-6-phenyl-5-pyrimidinyl]-2-isopropyl-3(2H)-pyridazinone and 2-methoxyethanol according to a similar manner to that of Example 85.

mp: 128-130° C. (ethanol)

IR (KBr): 3475, 3325, 3215, 1647, 1630, 1576, 1547 cm⁻¹

ESI/MS: 785[2M+Na]⁺, 404[M+Na]⁺, 382[M+H]⁺

¹H NMR (CDCl₃, δ): 0.93 (6H, d, J=6.60 Hz), 3.37 (3H, s), 3.65-3.71 (2H, m), 4.48-4.54 (2H, m), 5.02-5.19 (3H, m), 6.82 (1H, d, J=9.52 Hz), 7.24-7.31 (6H, m)

### EXAMPLE 88

6-{2-Amino-4-[2-(dimethylamino)ethoxy]-6-phenyl-5-pyrimidinyl}-2-isopropyl-3(2H)-pyridazinone was prepared from 6-[2-amino-4-(methylsulfinyl)-6-phenyl-5-pyrimidinyl]-2-isopropyl-3(2H)-pyridazinone and 2-(dimethylamino)ethanol according to a similar manner to that of Example 85.

mp: 130-131.5° C. (acetone-diisopropyl ether)

IR (KBr): 3365, 3172, 1664, 1649, 1589, 1570, 1550 cm⁻¹

ESI/MS: 811[2M+Na]⁺, 417[M+Na]⁺, 395[M+H]⁺

¹H NMR (CDCl₃, δ): 0.93 (6H, d, J=6.62 Hz), 2.27 (6H, s), 2.66 (2H, t, J=5.95 Hz), 4.47 (2H, t, J=5.95 Hz), 5.11 (1H, 7-plet, J=6.62 Hz), 5.18 (2H, br.s), 6.82 (1H, d, J=9.52 Hz), 7.24-7.30 (6H, m)

### EXAMPLE 89

Sodium hydride (60% in oil suspension) (19.5 mg) was added to a solution of methyl hydroxyacetate (40.7 ml) in N,N-dimethylacetamide (0.45 ml) under ice-cooling. After stirring for 30 minutes, 6-[2-amino-4-(methylsulfinyl)-6-phenyl-5-pyrimidinyl]-2-isopropyl-3(2H)-pyridazinone (150 mg) was added to the mixtutre and the mixture was heated at 100-105° C. for 7 hours. The mixture was concentrated under reduced pressure and purified by column chromatography on silica gel (n-hexane-ethyl acetate 40:60 v/v) to give methyl {[2-amino-5-(1-isopropyl-6-oxo-1,6-dihydro-3-pyridazinyl)-6-phenyl-4-pyrimidinyl]oxy}acetate as an amorphous (120 mg).

mp: 150-152.5° C.

IR (KBr): 3419, 3386, 3309, 3197, 1761, 1657, 1635, 1591, 1545 cm⁻¹

ESI/MS: 813[M+Na]⁺, 418[M+Na]⁺, 396[M+H]⁺

¹H NMR (CDCl₃, δ): 0.89 (6H, d, J=6.60 Hz), 3.80 (3H, s), 4.94 (2H, s), 5.10 (1H, 7-plet, J=6.60 Hz), 5.24 (2H, br.s), 6.86 (1H, d, J=9.52 Hz), 7.27 (5H, s), 7.43 (1H, d, J=9.52 Hz)

Elemental Analysis for C₂₀H₂₁N₅O₄.0.3H₂O

Calcd.: C,59.93; H,5.43; N,17.47

Found: C,60.13; H,5.39; N,17.26

### EXAMPLE 90

2-(2-{[2-Amino-5-(1-isopropyl-6-oxo-1,6-dihydro-3-pyridazinyl)-6-phenyl-4-pyrimidinyl]oxy}-ethyl)-1H-isoindole-1,3(2H)-dione was prepared from 6-[2-amino-4-(methylsulfinyl)-6-phenyl-5-pyrimidinyl]-2-isopropyl-3(2H)-pyridazinone and 2-(2-hydroxyethyl)-1H-isoindole-1,3 (2H)-dione according to a similar manner to that of Example 89.

mp: 231.5-232.5° C. (acetone-diisopropyl ether)

IR (KBr): 3313, 3195, 1774, 1716, 1653, 1622, 1577, 1545 cm⁻¹

ESI/MS: 1015[2M+N]⁺, 519[M+N]⁺, 497[M+H]⁺

¹H NMR (CDCl₃, δ): 0.83 (6H, d, J=6.62 Hz), 4.07 (2H, t, J=5.35 Hz), 4.66 (2H, t, J=5.35 Hz), 4.93-5.10 (3H, m), 6.71 (1H, d, J=9.50 Hz), 7.18-7.27 (6H, m), 7.69-7.84 (4H, m)

Elemental Analysis for C₂₇H₂₄N₆O₄.0.1H₂O

Calcd.: C,65.08; H,4.89; N,16.86

Found: C,64.98; H,4.91; N,16.78

### EXAMPLE 91

Sodium hydride (60% in oil suspension) (19.5 mg) was added in 1-propanol (0.6 ml) under ice-cooling. After stirring at the ambient temperature for 30 minutes, 6-[2-amino-4-(methylsulfonyl)-6-phenyl-5-pyrimidinyl]-2-isopropyl-3(2H)-pyridazinone (156.5 mg) was added to the mixture. The mixture was heated at 100-105° C. for 7 hours in a sealed tube. The mixture was concentrated under reduced pressure and purified by column chromatography on silica gel (n-hexane-ethyl acetate 20:80 v/v) to give 6-(2-amino-4-phenyl-6-propoxy-5-pyrimidinyl)-2-isopropyl-3(2H)-pyridazinone as a solid (136 mg).

mp: 127.5-129° C. (ethanol-diisopropyl ether)

IR (KBr): 3354, 3311, 1662, 1589, 1570, 1547 cm⁻¹

ESI/MS: 388[M+Na]⁺, 366[M+H]⁺

¹H NMR (CDCl₃, δ): 0.92-1.00 (9H, m), 1.65-1.83 (2H, m), 4.30 (2H, t, J=6.55 Hz), 5.05-5.19 (3H, m), 6.82 (1H, d, J=9.52 Hz), 7.21 (1H, d, J=9.52 Hz), 7.26 (5H, s)

### EXAMPLE 92

6-[2-Amino-4-(2-fluoroethoxy)-6-phenyl-5-pyrimidinyl]-2-isopropyl-3(2H)-pyridazinone was prepared from 6-[2-amino-4-(methylsulfonyl)-6-phenyl-5-pyrimidinyl]-2-isopropyl-3(2H)-pyridazinone and 2-fluoroethanol according to a similar manner to that of Example 91.

mp: 129-130.5° C. (ethanol-diisopropyl ether)

IR (KBr): 3491, 3327, 3207, 1649, 1635, 1576, 1545 cm⁻¹

ESI/MS: 761[2M+Na]⁺, 392[M+Na]⁺, 370[M+H]⁺

¹H NMR (CDCl₃, δ): 0.93 (6H, d, J=6.60 Hz), 4.54-4.58 (2H, m), 4.66-4.72 (1H, m), 4.79-4.84 (1H, m), 5.06-5.20 (3H, m), 6.83 (1H, d, J=9.52 Hz), 7.21-7.28 (6H, m)

### EXAMPLE 93

Sodium hydride (60% in oil suspension) (18.7 mg) was added to a solution of benzyl alcohol (52.4 ml) in N,N-dimethylacetamide (0.45 ml) under ice-cooling. After stirring for 30 minutes, 6-[2-amino-4-(methylsulfonyl)-6-phenyl-5-pyrimidinyl]-2-isopropyl-3(2H)-pyridazinone (150 mg) was added to the mixtutre and the mixture was heated at 100-105° C. for 7 hours. Water (4.5 ml) was added to the mixture to give a solid. The solid was collected by filtration, dissolved in chloroform (5 ml), dried over magnesium sulfate and concentrated under reduced pressure to give a residue. The residue was purified by column chromatography on silica gel (n-hexane-ethyl acetate 40:60 v/v) to give 6-[2-amino-4-(benzyloxy)-6-phenyl-5-pyrimidinyl]-2-isopropyl-3(2H)-pyridazinone as a solid (124 mg).

mp: 162-163° C. (acetone)

IR (KBr): 3357, 3309, 3168, 1651, 1589, 1570, 1545, 1490 cm⁻¹

ESI/MS: 849[2M+Na]⁺, 436[M+Na]⁺, 414[M+H]⁺

¹H NMR (CDCl₃, δ): 0.93 (6H, d, J=6.61 Hz), 5.1 (1H, 7-plet, J=6.61 Hz), 5.17 (2H, s), 5.43 (2H, s), 6.79 (1H, d, J=9.52 Hz), 7.18 (1H, d, J=9.52 Hz), 7.23-7.39 (10H, m)

Elemental Analysis for C₂₄H₂₃N₅O₂.0.1H₂O

Calcd.: C,69.41; H,5.63; N,16.86

Found: C,69.26; H,5.59; N,16.91

### EXAMPLE 94

Sodium hydride (60% in oil suspension) (18.7 mg) was added to a solution of 2-pyridinylmethanol (48.8 ml) in N,N-dimethylacetamide (0.45 ml) under ice-cooling. After stirring for 30 minutes, 6-[2-amino-4-(methylsulfonyl)-6-phenyl-5-pyrimidinyl]-2-isopropyl-3(2H)-pyridazinone (150 mg) was added to the mixtutre and the mixture was heated at 100-105° C. for 7 hours. Water (4.5 ml) was added to the mixture to give a solid. The solid was collected by filtration, dissolved in chloroform (5 ml), dried over magnesium sulfate and concentrated under reduced pressure to give a residue. The residue was purified by column chromatography on silica gel (n-hexane-ethyl acetate 50:50 v/v) to give 6-[2-amino-4-phenyl-6-(2-pyridinylmethoxy)-5-pyrimidinyl]-2-isopropyl-3(2H)-pyridazinone as a solid (51 mg).

mp: 177-178° C. (acetone-diisopropyl ether)

IR (KBr): 3491, 3141, 1658, 1631, 1591, 1554 cm⁻¹

ESI/MS: 851[2M+Na]⁺, 437[M+Na]⁺, 415[M+H]⁺

¹H NMR (DMSO-d₆, δ): 0.81 (6H, d, J=6.62 Hz), 4.93 (1H, 7-plet, J=6.62 Hz), 5.49 (2H, s), 6.86 (1H, d, J=9.52 Hz), 7.02 (2H, s), 7.21-7.42 (7H, m), 7.54 (1H, d, J=9.52 Hz), 7.77-7.86 (1H, m), 8.54-8.57 (1H, m)

### EXAMPLE 95

Sodium hydride (60% in oil suspension) (18.7 mg) was added to a solution of 2-(2-pyridinyl)ethanol (57 ml) in N,N-dimethylacetamide (0.45 ml) under ice-cooling. After stirring for 30 minutes, 6-[2-amino-4-(methylsulfonyl)-6-phenyl-5-pyrimidinyl]-2-isopropyl-3(2H)-pyridazinone (150 mg) was added to the mixture and the mixture was heated at 100-105° C. for 7 hours. The mixture was concentrated under reduced pressure and purified by column chromatography on silica gel (methanol-ethyl acetate 1:99 v/v) to give 6-{2-Amino-4-phenyl-6-[2-(2-pyridinyl)ethoxy]-5-pyrimidinyl}-2-isopropyl-3(2H)-pyridazinone was prepared as a syrup (71 mg).

IR (KBr): 3471-3327, 3201, 1651, 1589, 1572, 1545 cm⁻¹

ESI/MS: 879[2M+Na]⁺, 451[M+Na]⁺, 429[M+H]⁺

¹H NMR (CDCl₃, δ): 0.88 (6H, d, J=6.62 Hz), 3.20 (2H, t, J=6.47 Hz), 4.77 (2H, t, J=6.47 Hz), 5.02-5.13 (3H, m), 6.67 (1H, d, J=9.52 Hz), 6.91 (1H, d, J=9.52 Hz), 7.11-7.32 (7H, m), 7.53-7.62 (1H, m), 8.54-8.57 (1H, m)

### EXAMPLE 96

6-[2-Amino-4-(cyclohexyloxy)-6-phenyl-5-pyrimidinyl]-2-isopropyl-3(2H)-pyridazinone was prepared from 6-[2-amino-4-(methylsulfonyl)-6-phenyl-5-pyrimidinyl]-2-isopropyl-3(2H)-pyridazinone and cyclohexanol according to a similar manner to that of Example 94.

mp: 173-175° C. (diisopropyl ether)

IR (KBr): 3419-3309, 3182, 1653, 1589, 1570, 1545 cm⁻¹

ESI/MS: 833[2M+Na]⁺, 428[M+Na]⁺, 406[M+H]⁺

¹H NMR (CDCl₃, δ): 0.84-1.61 (8H, m), 0.94 (6H, d, J=6.62 Hz), 1.87-1.95 (2H, m), 5.05-5.22 (4H, m), 6.82 (1H, d, J=9.52 Hz), 7.21 (1H, d, J=9.52 Hz), 7.23-7.31 (5H, m)

### EXAMPLE 97

6-[2-Amino-4-phenyl-6-(tetrahydro-2H-pyran-4-yloxy)-5-pyrimidinyl]-2-isopropyl-3(2H)-pyridazinone was prepared from 6-[2-amino-4-(methylsulfonyl)-6-phenyl-5-pyrimidinyl]-2-isopropyl-3(2H)-pyridazinone and tetrahydro-2H-pyran-4-ol according to a similar manner to that of Example 94.

mp: 207-209° C. (ethanol-diisopropyl ether)

IR (KBr): 3373, 3329, 3211, 1670, 1649, 1589, 1572, 1539 cm⁻¹

ESI/MS: 837[2M+Na]⁺, 430[M+Na]⁺, 408[M+H]⁺

¹H NMR (CDCl₃, δ): 0.97 (6H, d, J=6.60 Hz), 1.65-1.85 (2H, m), 1.99-2.09 (2H, m), 3.54-3.67 (2H, m), 3.79-3.91 (2H, m), 5.07-5.21 (3H, m), 5.29-5.40 (1H, m), 6.82 (1H, d, J=9.52 Hz), 7.18 (1H, d, J=9.52 Hz), 7.25-7.27 (5H, m)

Elemental Analysis for C₂₂H₂₅N₅O₃

Calcd.: C,64.85; H,6.18; N,17.19

Found: C,65.05; H,6.20; N,17.20

### EXAMPLE 98

6-(2-Amino-4-phenoxy-6-phenyl-5-pyrimidinyl)-2-isopropyl-3(2H)-pyridazinone was prepared from 6-[2-amino-4-(methylsulfonyl)-6-phenyl-5-pyrimidinyl]-2-isopropyl-3(2H)-pyridazinone and phenol according to a similar manner to that of Example 94.

mp: 218-221° C. (methanol)

IR (KBr): 3394, 3305, 3190, 1666, 1631, 1593, 1572, 1539 cm⁻¹

ESI/MS: 821[2M+Na]⁺, 422[M+Na]⁺, 400[M+H]⁺

¹H NMR (DMSO-d₆, δ): 0.83 (6H, d, J=6.60 Hz), 4.94 (1H, 7-plet, J=6.60 Hz), 6.89 (1H, d, J=9.54 Hz), 6.99 (2H, br. s), 7.20-7.48 (10H, m), 7.68 (1H, d, J=9.54 Hz)

Elemental Analysis for C₂₃H₂₁N₅O₂.3H₂O

Calcd.: C,68.23; H,5.38; N,17.30

Found: C,68.09; H,5.22; N,17.24

### EXAMPLE 99

Potassium tert-butoxide (30 mg) was added to a mixture of methyl {[2-amino-5-(1-isopropyl-6-oxo-1,6-dihydro-3-pyridazinyl)-6-phenyl-4-pyrimidinyl]oxy}acetate (150 mg) in formamide (1.5 ml) and the mixture was heated at 100-105° C. for 3 hours. Water (9 ml) was added to give 2-{[2-amino-5-(1-isopropyl-6-oxo-1,6-dihydro-3-pyridazinyl)-6-phenyl-4-pyrimidinyl]oxy}acetamide as a solid (101 mg).

mp: 257-261° C. (formamide-water)

IR (KBr): 3475, 3411, 3205, 1678, 1637, 1593, 1545 cm⁻¹

ESI/MS: 783[2M+Na]⁺, 403[M+Na]⁺, 381[M+H]⁺

¹H NMR (DMSO-d₆, δ): 0.75 (6H, d, J=6.61 Hz), 4.77 (2H, s), 4.90 (1H, 7-plet, J=6.61 Hz), 6.89 (1H, d, J=9.56 Hz), 6.96 (2H, s), 7.04-7.39 (7H, m), 7.69 (1H, d, J=9.56 Hz)

Elemental Analysis for C₁₉H₂₀N₆O₃

Calcd.: C,59.99; H,5.30; N,22.09

Found: C,59.95; H,5.27; N,22.06

### EXAMPLE 100

A solution of methyl {[2-amino-5-(1-isopropyl-6-oxo-1,6-dihydro-3-pyridazinyl)-6-phenyl-4-pyrimidinyl)oxy}acetate (600 mg) in a mixture of 1N aqueous sodium hydroxide (4.5 ml) and tetrahydrofuran (4.5 ml) was heated at 60-65° C. for 3 hours. Under ice-cooling, 1N hydrochloric acid (4.5 ml) was added to the mixture. The mixture was extracted with ethyl acetate, dried over magnesium sulfate and concentrated under reduced pressure to give a residue. The residue was suspended with acetone to give {[2-amino-5-(1-isopropyl-6-oxo-1,6-dihydro-3-pyridazinyl)-6-phenyl-4-pyrimidinyl]oxy}acetic acid as a solid (406 mg).

mp: 215-217° C. (acetone)

IR (KBr): 1653, 1591, 1576 cm⁻¹

ESI/MS: 404[M+Na]⁺, 382[M+H]⁺

¹H NMR (DMSO-d₆, δ): 0.76 (6H, d, J=6.60 Hz), 4.83-4.98 (3H, m), 6.90 (1H, d, J=9.53 Hz), 7.00 (2H, s), 7.19-7.37 (5H, m), 7.54 (1H, d, J=9.53 Hz), 13.2 (1H, br.s)

### EXAMPLE 101

A mixture of {[2-amino-5-(1-isopropyl-6-oxo-1,6-dihydro-3-pyridazinyl)-6-phenyl-4-pyrimidinyl]oxy}acetic acid (150 mg), methylamine hydrochloride (31.9 mg), 1-hydroxybenzotriazole (63.8 mg), triethylamine (65.8 μl) and 1-[3-(dimethylamino)propyl]-3-ethylcarbodiimide hydrochloride (90.5 mg) in dimethylformamide (0.9 ml) was stirred at ambient temperature for 70 hours. The mixture was concentrated under reduced pressure to give a residue. The residue was added to a mixture of water and ethyl acetate. The organic layer was dried over magnesium sulfate, and concentrated under reduced pressure and purified by column chromatography on silica gel (methanol-ethyl acetate 2:98 v/v) to give 2-{[2-amino-5-(1-isopropyl-6-oxo-1,6-dihydro-3-pyridazinyl)-6-phenyl-4-pyrimidinyl]oxy}-N-methylacetamide as a solid (142 mg).

mp: 204-205.5° C. (ethanol-diisopropyl ether)

IR (KBr): 3440, 3307, 3168, 1680, 1658, 1595, 1539 cm⁻¹

ESI/MS-Neg: 393[M−H]⁻

¹H NMR (CDCl₃, δ): 1.00 (6H, d, J=6.62 Hz), 2.87 (3H, d, J=4.94 Hz), 4.89 (2H, s), 5.15-5.31 (3H, m), 6.10 (1H, br.s), 6.81 (1H, d, J=9.50 Hz), 7.04 (1H, d, J=9.50 Hz), 7.26-7.35 (5H, m)

Elemental Analysis for C₂₀H₂₂N₆O₃

Calcd.: C,60.90; H,5.62; N,21.31

Found: C,60.82; H,5.64; N,21.22

### EXAMPLE 102

2-{[2-Amino-5-(1-isopropyl-6-oxo-1,6-dihydro-3-pyridazinyl)-6-phenyl-4-pyrimidinyl]oxy}-N,N-dimethylacetamide was prepared from {[2-amino-5-(1-isopropyl-6-oxo-1,6-dihydro-3-pyridazinyl)-6-phenyl-4-pyrimidinyl]oxy}acetic acid and N,N-dimethylamine hydrochloride according to a similar manner to that of Example 101.

mp: 133-135.5° C. (acetone-hexane)

IR (KBr): 3462, 3309, 3192, 1657, 1591, 5174, 1545 cm⁻¹

ESI/MS: 839[2M+Na]⁺, 431[M+Na]⁺, 409[M+H]⁺

¹H NMR (CDCl₃, δ): 0.87 (6H, d, J=6.62 Hz), 3.01 (3H, s, J=3.01 Hz), 3.04 (3H, s, J=3.04 Hz), 5.00-5.14 (5H, m), 6.86 (1H, d, J=9.52 Hz), 7.21-7.30 (5H, m), 7.67 (1H, d, J=9.52 Hz)

### EXAMPLE 103

2-{[2-Amino-5-(1-isopropyl-6-oxo-1,6-dihydro-3-pyridazinyl)-6-phenyl-4-pyrimidinyl]oxy}-N-cyclopropylacetamide was prepared from {[2-amino-5-(1-isopropyl-6-oxo-1,6-dihydro-3-pyridazinyl)-6-phenyl-4-pyrimidinyl]oxy}acetic acid and cyclopropylamine according to a similar manner to that of Example 101.

mp: 87-92° C. (amorphous)

IR (KBr): 3316, 1651, 1589, 1576, 1541 cm⁻¹

ESI/MS: 863[2M+Na]⁺, 443[M+Na]⁺, 421[M+H]⁺

¹H NMR (CDCl₃, δ): 0.41-0.50 (2H, m), 0.75-0.90 (2H, m), 1.06 (6H, d, J=6.60 Hz), 2.64-2.75 (1H, m), 4.84 (2H, s), 5.21 (1H, 7-plet, J=6.60 Hz), 5.29 (2H, s), 6.15 (1H, br.s), 6.82 (1H, d, J=9.52 Hz), 7.09 (1H, d, J=9.52 Hz), 7.26-7.34 (5H, m)

### EXAMPLE 104

A solution of 2-(2-{[2-amino-5-(1-isopropyl-6-oxo-1,6-dihydro-3-pyridazinyl)-6-phenyl-4-pyrimidinyl]oxy}ethyl)-1H-isoindole-1,3 (2H)-dione (225 mg) and hydrazine hydrate (0.5 ml) in ethanol (4.5 ml) was heated under reflux for 3 hours. The precipitate was filtered off and the mother liquid was concentrated under reduced pressure to give a residue. The residue was purified by column chromatography on silica gel (Chromatorex NH) (methanol-ethyl acetate 1: 99 v/v) to give 6-[2-amino-4-(2-aminoethoxy)-6-phenyl-5-pyrimidinyl]-2-isopropyl-3(2H)-pyridazinone as a solid (108 mg).

mp: 213-216° C. (acetone)

IR (KBr): 3431, 3352, 3248, 1666, 1647, 1564 cm⁻¹

ESI/MS: 755[2M+Na]⁺, 389[M+Na]⁺, 367[M+H]⁺

¹H NMR (CDCl₃, δ): 1.41 (6H, d, J=6.62 Hz), 3.60-3.72 (2H, m), 3.78-3.87 (2H, m), 5.05 (2H, br.s), 5.35 (1H, 7-plet, J=6.62 Hz), 6.47 (1H, d, J=9.60 Hz), 6.50 (1H, d, J=9.60 Hz), 7.26-7.42 (7H, m)

Elemental Analysis for C₁₉H₂₂N₆O₂.0.4H₂O

Calcd.: C,61.08; H,6.15; N,22.49

Found: C,61.21; H,6.23; N,22.32

### EXAMPLE 105

In a sealed tube, a mixture of 6-[2-amino-4-(methylsulfinyl)-6-phenyl-5-pyrimidinyl]-2-isopropyl-3(2H)-pyridazinone (200 mg) in 2M ammonia solution in methanol (2 ml) was heated at 60-65° C. for 90 hours. After cooling at ambient temperature, the precipitate was removed by filtration and the mother liquid was concentrated under reduced pressure to give a residue. The residue was purified by column chromatography on silica gel (methanol-ethyl acetate 3:97 v/v) to give 6-(2,4-diamino-6-phenyl-5-pyrimidinyl)-2-isopropyl-3(2H)-pyridazinone as a solid (20 mg).

mp: 258-260° C. (ethanol suspension)

IR (KBr): 3417, 3363, 1645, 1610, 1576, 1552 cm⁻¹

ESI/MS: 345[M+Na]⁺, 323[M+H]⁺

¹H NMR (DMSO-d₆, δ): 1.01 (6H, d, J=6.60 Hz), 5.05 (1H, 7-plet, J=6.60 Hz), 6.27 (2H, br.s), 6.34 (2H, br.s), 6.68 (1H, d, J=9.50 Hz), 6.93 (1H, d, J=9.50 Hz), 7.18-7.30 (5H, m)

### EXAMPLE 106

6-[2-Amino-4-(methylamino)-6-phenyl-5-pyrimidinyl]-2-isopropyl-3(2H)-pyridazinone was prepared from 6-[2-amino-4-(methylsulfinyl)-6-phenyl-5-pyrimidinyl]-2-isopropyl-3(2H)-pyridazinone and 40% methylamine solution in methanol according to a similar manner to that of Example 105.

mp: 268-270° C. (methanol)

IR (KBr): 3357, 3165, 1657, 1587, 1564 cm⁻¹

ESI/MS: 695[2M+Na]⁺, 359[M+Na]⁺, 337[M+H]⁺

¹H NMR (DMSO-d₆, δ): 1.09 (6H, d, J=6.61 Hz), 2.80 (3H, d, J=4.60 Hz), 5.04 (1H, 7-plet, J=6.61 Hz), 6.34 (2H, br.s), 6.47 (1H, q, J=4.60 Hz), 6.71 (1H, d, J=9.50 Hz), 6.99 (1H, d, J=9.50 Hz), 7.16-7.28 (5H, m)

Elemental Analysis for C₁₈H₂₀N₆O

Calcd.: C,64.27; H,5.99; N,24.98

Found: C,64.21; H,6.00; N,24.85

### EXAMPLE 107

6-[2-Amino-4-(dimethylamino)-6-phenyl-5-pyrimidinyl]-2-isopropyl-3(2H)-pyridazinone was prepared from 6-[2-amino-4-(methylsulfinyl)-6-phenyl-5-pyrimidinyl]-2-isopropyl-3(2H)-pyridazinone and 2M dimethylamine solution in tetrahydrofuran according to a similar manner to that of Example 105.

mp: 293-296° C. (tetrahydrofuran)

IR (KBr): 3494, 3275, 3136, 1658, 1622, 1587, 1558 cm⁻¹

ESI/MS: 723[2M+Na]⁺, 373[M+Na]⁺, 351[M+H]⁺

¹H NMR (DMSO-d₆, δ): 1.01 (6H, d, J=6.60 Hz), 2.78 (6H, s), 5.03 (1H, 7-plet, J=6.60 Hz), 6.39 (2H, br.s), 6.70 (1H, d, J=9.50 Hz), 7.05-7.26 (6H, m)

Elemental Analysis for C₁₉H₂₂N₆O

Calcd.: C,65.12; H,6.33; N,23.98

Found: C,65.07; H,6.39; N,23.93

### EXAMPLE 108

6-[2-Amino-4-(ethylamino)-6-phenyl-5-pyrimidinyl]-2-isopropyl-3(2H)-pyridazinone was prepared from 6-(2-amino-4-(methylsulfinyl)-6-phenyl-5-pyrimidinyl]-2-isopropyl-3(2H)-pyridazinone and 2M ethylamine solution in tetrahydrofuran according to a similar manner to that of Example 105.

mp: 208-209° C. (ethanol-diisopropyl ether)

IR (KBr): 3354, 3305, 3180, 1657, 1635, 1587, 1560 cm⁻¹

ESI/MS: 373[M+Na]⁺, 351[M+H]⁺

¹H NMR (CDCl₃, δ): 1.25 (3H, t, J=5.49 Hz), 1.43 (6H, d, J=6.62 Hz), 3.43-3.57 (2H, m), 4.98 (2H, br.s), 5.38 (1H, 7-plet, J=6.62 Hz), 6.46 (1H, d, J=9.91 Hz), 6.48 (1H, d, J=9.91 Hz), 6.83 (1H, br.s), 7.26-7.41 (5H, m)

Elemental Analysis for C₁₉H₂₂N₆O

Calcd.: C,65.12; H,6.33; N,23.98

Found: C,64.94; H,6.44; N,23.73

### EXAMPLE 109

6-[2-Amino-4-phenyl-6-(propylamino)-5-pyrimidinyl]-2-isopropyl-3(2H)-pyridazinone was prepared from 6-[2-amino-4-(methylsulfinyl)-6-phenyl-5-pyrimidinyl]-2-isopropyl-3(2H)-pyridazinone and propylamine according to a similar manner to that of Example 105.

mp: 209-212° C. (propylamine)

IR (KBr): 3344, 3305, 3134, 1655, 1624, 1587, 1566 cm⁻¹

ESI/MS: 751[2M+Na]⁺, 387[M+Na]⁺, 365[M+H]⁺

¹H NMR (DMSO-d₆, δ): 0.88 (3H, t, J=7.35 Hz), 1.16 (6H, d, J=6.60 Hz), 1.42-1.61 (2H, m), 3.24-3.34 (2H, m), 5.08 (1H,7-plet, J=6.60 Hz), 6.32 (2H, br.s), 6.55 (1H, t, J=5.49 Hz), 6.66 (1H, d, J=9.52 Hz), 6.84 (1H, d, J=9.52 Hz), 7.18-7.30 (5H, m)

Elemental Analysis for C₂₀H₂₄N₆O.0.1H₂O

Calcd.: C,65.59; H,6.66; N,22.95

Found: C,65.57; H,6.96; N,22.97

### EXAMPLE 110

6-[2-Amino-4-(isopropylamino)-6-phenyl-5-pyrimidinyl]-2-isopropyl-3(2H)-pyridazinone was prepared from 6-[2-amino-4-(methylsulfinyl)-6-phenyl-5-pyrimidinyl]-2-isopropyl-3(2H)-pyridazinone and isopropylamine according to a similar manner to that of Example 105.

mp: 234-236° C. (methanol)

IR (KBr): 3340, 3280, 3144, 1657, 16226, 1587, 1562 cm⁻¹

ESI/MS: 751[2M+Na]⁺, 387[M+Na]⁺, 365[M+H]⁺

¹H NMR (DMSO-d₆, δ): 1.14 (6H, d, J=6.50 Hz), 1.22 (6H, d, J=6.60 Hz), 4.26-4.36 (1H, m), 5.12 (1H, 7-plet, J=6.60 Hz), 6.35 (2H, br.s), 6.41 (1H, d, J=7.78 Hz), 6.60 (1H, d, J=9.56 Hz), 6.72 (1H, d, J=9.56 Hz), 7.19-7.32 (5H, m)

Elemental Analysis for C₂₀H₂₄N₆O

Calcd.: C,65.91; H,6.64; N,23.06

Found: C,65.83; H,6.90; N,22.97

### EXAMPLE 111

6-[4-(Allylamino)-2-amino-6-phenyl-5-pyrimidinyl]-2-isopropyl-3(2H)-pyridazinone was prepared from 6-[2-amino-4-(methylsulfinyl)-6-phenyl-5-pyrimidinyl]-2-isopropyl-3(2H)-pyridazinone and allylamine according to a similar manner to that of Example 105.

mp: 191.5-192.5° C. (ethanol-diisopropyl ether)

IR (KBr): 3345, 3180, 1668, 1649, 1595, 1570, 1547 cm⁻¹

ESI/MS: 385[M+Na]⁺, 363[M+H]⁺

¹H NMR (CDCl₃, δ): 1.39 (6H, d, J=6.68 Hz), 4.09-4.16 (2H, m), 5.01 (2H, br.s), 5.15-5.40 (3H, m), 5.85-6.10 (1H, m), 6.48 (2H, s), 6.95 (1H, br.s), 7.26-7.41 (5H, m)

Elemental Analysis for C₂₀H₂₂N₆O

Calcd.: C,66.28; H,6.12; N,23.19

Found: C,66.11; H,6.26; N,23.13

### EXAMPLE 112

6-[2-Amino-4-(cyclopropylamino)-6-phenyl-5-pyrimidinyl]-2-isopropyl-3(2H)-pyridazinone was prepared from 6-[2-amino-4-(methylsulfinyl)-6-phenyl-5-pyrimidinyl]-2-isopropyl-3(2H)-pyridazinone and cyclopropylamine according to a similar manner to that of Example 105.

mp: 237-239° C. (methanol)

IR (KBr): 3325, 3155, 1655, 1624, 1587, 1562 cm⁻¹

ESI/MS: 747[2M+Na]⁺, 385[M+Na]⁺, 363[M+H]⁺

¹H NMR (DMSO-d₆, δ): 0.42-0.51 (2H, m), 0.63-0.73 (2H, m), 1.12 (6H, d, J=6.60 Hz), 2.78-2.91 (1H, m), 5.06 (1H, 7-plet, J=6.60 Hz), 6.39 (2H, br.s), 6.60-6.66 (2H, m), 6.82 (1H, d, J=9.52 Hz), 7.16-7.30 (5H, m)

Elemental Analysis for C₂₀H₂₂N₆O

Calcd.: C,66.28; H,6.12; N,23.19

Found: C,66.39; H,6.25; N,23.13

### EXAMPLE 113

6-[2-Amino-4-(tert-butylamino)-6-phenyl-5-pyrimidinyl]-2-isopropyl-3(2H)-pyridazinone was prepared from 6-[2-amino-4-(methylsulfinyl)-6-phenyl-5-pyrimidinyl]-2-isopropyl-3(2H)-pyridazinone and tert-butylamine according to a similar manner to that of Example 105.

mp: 202-204° C. (ethanol-diisopropyl ether)

IR (KBr): 3502, 3284, 3120, 1668, 1630, 1593, 1566 cm⁻¹

ESI/MS: 401[M+Na]⁺, 379[M+H]⁺

¹H NMR (CDCl₃, δ): 1.42 (6H, d, J=6.62 Hz), 1.49 (9H, s), 4.88 (2H, br.s), 5.36 (1H, 7-plet, J=6.62 Hz), 6.46 (2H, s), 6.58 (1H, br.s), 7.26-7.37 (5H, m)

Elemental Analysis for C₂₁H₂₆N₆O

Calcd.: C,66.64; H,6.92; N,22.20

Found: C,66.88; H,7.10; N,22.21

### EXAMPLE 114

A mixture of 6-[2-amino-4-(methylsulfinyl)-6-phenyl-5-pyrimidinyl]-2-isopropyl-3(2H)-pyridazinone (150 mg) in 2-aminoethanol (2 ml) was heated at 100-105° C. for 10 hours. The mixture was concentrated under reduced pressure to give a residue. The residue was crystallized from ethanol to give 6-{2-amino-4-[(2-hydroxyethyl)amino]-6-phenyl-5-pyrimidinyl}-2-isopropyl-3(2H)-pyridazinone as a solid (96 mg).

mp: 221.5-223.5° C. (ethanol)

IR (KBr): 3431, 3352, 3248, 1666, 1647, 1564 cm⁻¹

ESI/MS: 389[M+Na]⁺, 367[M+H]⁺

¹H NMR (CDCl₃, δ): 1.69 (6H, d, J=6.60 Hz), 3.35-3.51 (4H, m), 4.73 (1H, br.s), 5.08 (1H, 7-plet, J=6.60 Hz), 6.38 (2H, br.s), 6.64 (1H, d, J=9.54 Hz), 6.79 (1H, t, J=5.28 Hz), 6.82 (1H, d, J=9.54 Hz), 7.20-7.32 (5H, m)

Elemental Analysis for C₁₉H₂₂N₆O₂.0.5H₂O

Calcd.: C,60.79; H,6.17; N,22.39

Found: C,60.60; H,6.31; N,22.21

### EXAMPLE 115

In a sealed tube, a mixture of 6-[2-amino-4-(methylsulfinyl)-6-phenyl-5-pyrimidinyl]-2-isopropyl-3(2H)-pyridazinone (150 mg) in2-methoxyethylamine (2 ml) was heated at 100-105° C. for 10 hours. The mixture was concentrated under reduced pressure to give a residue. The residue was crystallized from a mixture of ethanol and diisopropyl ether to give 6-{2-amino-4-[(2-methoxyethyl)amino]-6-phenyl-5-pyrimidinyl}-2-isopropyl-3(2H)-pyridazinone as a solid (88 mg).

mp: 168-170° C. (ethanol-diisopropyl ether)

IR (KBr): 3357, 3172, 1662, 1639, 1589, 1558 cm⁻¹

ESI/MS: 783[2M+Na]⁺, 403[M+Na]⁺, 381[M+H]⁺

¹H NMR (CDCl₃, δ): 1.43 (6H, d, J=6.64 Hz), 3.36 (3H, s), 3.56 (2H, t, J=4.78 Hz), 3.65-3.74 (2H, m), 4.98 (2H, br.s), 5.36 (1H, 7-plet, J=6.64 Hz), 6.45 (1H, d, J=9.93 Hz), 6.49 (1H, d, J=9.63 Hz), 7.12 (1H, t, J=4.98 Hz), 7.26-7.40 (5H, m)

Elemental Analysis for C₂₀H₂₄N₆O₂

Calcd.: C,63.14; H,6.36; N,22.09

Found: C,63.28; H,6.56; N,21.87

### EXAMPLE 116

6-{2-Amino-4-[(2-aminoethyl)amino]-6-phenyl-5-pyrimidinyl}-2-isopropyl-3(2H)-pyridazinone was prepared from 6-[2-amino-4-(methylsulfinyl)-6-phenyl-5-pyrimidinyl]-2-isopropyl-3(2H)-pyridazinone and ethylenediamine according to a similar manner to that of Example 114.

mp: 194-197° C. (diisopropyl ether)

IR (KBr): 3345, 3172, 1653, 1585, 1558 cm⁻¹

ESI/MS: 388[M+Na]⁺, 366[M+H]⁺

¹H NMR (CDCl₃, δ): 1.43 (6H, d, J=6.61 Hz), 1.4-1.7 (2H, m), 2.94 (2H, t, J=6.03 Hz), 3.50-3.65 (2H, m), 4.97 (2H, br.s), 5.36 (1H,7-plet, J=6.61 Hz), 6.49 (2H, s), 7.07 (1H, t, J=5.08 Hz), 7.26-7.39 (5H, m)

### EXAMPLE 117

6-(2-Amino-4-{[2-(dimethylamino)ethyl]amino}-6-phenyl-5-pyrimidinyl)-2-isopropyl-3(2H)-pyridazinone was prepared from 6-[2-amino-4-(methylsulfinyl)-6-phenyl-5-pyrimidinyl]-2-isopropyl-3(2H)-pyridazinone and N,N-dimethylethylenediamine according to a similar manner to that of Example 115.

mp: 164-165.5° C. (ethanol-diisopropyl ether)

IR (KBr): 3375, 3321, 3211, 1666, 1641, 1593, 1547 cm⁻¹

ESI/MS: 809[2M+Na]⁺, 416[M+Na]⁺, 394[M+H]⁺

¹H NMR (CDCl₃, δ): 1.44 (6H, d, J=6.63 Hz), 2.20 (6H, s), 2.49 (2H, t, J=5.99 Hz), 3.49-3.59 (2H, m), 5.05 (2H, br.s), 5.33 (1H,7-plet, J=6.63 Hz), 6.48 (2H, s), 6.99 (1H, t, J=4.34 Hz), 7.25-7.38 (5H, m)

Elemental Analysis for C₂₁H₂₇N₇O

Calcd.: C,64.10; H,6.92; N,24.92

Found: C,64.13; H,7.02; N,24.91

### EXAMPLE 118

A mixture of 6-[2-amino-4-(methylsulfinyl)-6-phenyl-5-pyrimidinyl]-2-isopropyl-3(2H)-pyridazinone (150 mg), 2-aminoacetamide hydrochloride (89.8 mg) and N-ethyl-N,N-diisopropylamine (0.141 ml) in N,N-dimethylacetamide (0.3 ml) was heated at 100-105° C. for 10 hours. Water (3 ml) was added to give a solid. The solid was crystallized from a mixture of ethanol and diisopropyl ether to give 2-{(2-amino-5-(1-isopropyl-6-oxo-1,6-dihydro-3-pyridazinyl)-6-phenyl-4-pyrimidinyl]amino}acetamide as a solid (77 mg).

mp: 235-237.5° C. (ethanol-diisopropyl ether)

IR (KBr): 3338, 3207, 16535, 1585, 1558 cm⁻¹

ESI/MS: 781[2M+Na]⁺, 402[M+Na]⁺, 380[M+H]⁺

¹H NMR (DMSO-d₆, δ): 1.17 (6H, d, J=6.61 Hz), 3.90 (2H, d, J=5.00 Hz), 5.06 (1H, 7-plet, J=6.61 Hz), 6.41 (2H, br.s), 6.68 (1H, d, J=9.54 Hz), 6.84 (1H, t, J=5.00 Hz), 6.96 (1H, d, J=9.54 Hz), 7.13 (1H, br.s), 7.20-7.32 (5H, m), 7.38 (1H, br.s)

Elemental Analysis for C₁₉H₂₁N₇O₂.0.3H₂O

Calcd.: C,59.30; H,5.66; N,25.48

Found: C,59.54; H,5.60; N,25.23

### EXAMPLE 119

A mixture of 6-[2-amino-4-(methylsulfinyl)-6-phenyl-5-pyrimidinyl]-2-isopropyl-3(2H)-pyridazinone (150 mg) and benzylamine (88.7 μl) in N,N-dimethylacetamide (0.3 ml) was heated at 100-105° C. for 10 hours. Water (3 ml) was added to give a solid. The solid was crystallized from a mixture of ethanol and diisopropyl ether to give 6-[2-amino-4-(benzylamino)-6-phenyl-5-pyrimidinyl]-2-isopropyl-3(2H)-pyridazinone as a solid (135 mg).

mp: 163-164.5° C. (ethanol-diisopropyl ether)

IR (KBr): 3388, 3307, 3257, 3195, 1651, 1628, 1583, 1562 cm⁻¹

ESI/MS: 847[2M+Na]⁺, 835[M+Na]⁺, 813[M+H]⁺

¹H NMR (CDCl₃, δ): 1.11 (6H, d, J=6.64 Hz), 4.64 (2H, d, J=5.02 Hz), 5.03 (2H, br.s), 5.21 (1H, 7-plet, J=6.64 Hz), 6.45 (2H, s), 7.16 (1H, t, J=4.87 Hz), 7.13-7.42 (10H, m)

### EXAMPLE 120

A mixture of 6-[2-amino-4-(methylsulfinyl)-6-phenyl-5-pyrimidinyl]-2-isopropyl-3(2H)-pyridazinone (150 mg) and benzylamine (105 μl) in N,N-dimethylacetamide (0.3 ml) was heated at 100-105° C. for 10 hours. Water (3 ml) was added and the mixture was stirred. An aqueous layer was removed by decantation. A residue was dissolved in chloroform, dried over magnesium sulfate, concentrated under reduced pressure and purified by column chromatography on silica gel (n-hexane-ethyl acetate 40:60 v/v) to give 6-{2-amino-4-[benzyl(methyl)amino]-6-phenyl-5-pyrimidinyl}-2-isopropyl-3(2H)-pyridazinone as a solid (126 mg).

mp: 167-168° C. (ethanol-diisopropyl ether)

IR (KBr): 3496, 3273, 3118, 1657, 1622, 1589, 1554, 1531 cm⁻¹

ESI/MS: 449[M+Na]⁺, 427[M+H]⁺

¹H NMR (CDCl₃, δ): 1.15 (6H, d, J=6.60 Hz), 2.67 (3H, s), 4.74 (2H, s), 4.92 (2H, br.s), 5.20 (1H, 7-plet, J=6.60 Hz), 6.53 (1H, d, J=9.50 Hz), 6.76 (1H, d, J=9.50 Hz), 7.13-7.35 (10H, m)

Elemental Analysis for C₂₅H₂₆N₆O

Calcd.: C,70.40; H,6.14; N,19.70

Found: C,70.18; H,6.19; N,19.64

### EXAMPLE 121

6-{2-Amino-4-phenyl-6-[(2-pyridinylmethyl)amino]-5-pyrimidinyl}-2-isopropyl-3(2H)-pyridazinone was prepared from 6-[2-amino-4-(methylsulfinyl)-6-phenyl-5-pyrimidinyl]-2-isopropyl-3(2H)-pyridazinone and 2-pyridinylmethylamine according to a similar manner to that of Example 120.

mp: 196-198° C. (ethanol)

IR (KBr): 3475, 3367, 3327, 3165, 1657, 1653, 1626, 1587, 1550, 1518 cm⁻¹

ESI/MS: 436[M+Na]⁺, 414[M+H]⁺

¹H NMR (CDCl₃, δ): 1.40 (6H, d, J=6.60 Hz), 4.80 (2H, d, J=4.72 Hz), 5.06 (2H, br.s), 5.33 (1H, 7-plet, J=6.60 Hz), 6.51 (2H, s), 7.15-7.40 (7H, m), 7.62-7.70 (2H, m), 8.49-8.53 (1H, m)

Elemental Analysis for C₂₃H₂₃N₇O.0.5H₂O

Calcd.: C,65.39; H,5.73; N,23.21

Found: C,65.47; H,5.74; N,23.24

### EXAMPLE 122

6-{2-Amino-4-phenyl-6-[(3-pyridinylmethyl)amino]-5-pyrimidinyl}-2-isopropyl-3(2H)-pyridazinone was prepared from 6-[2-amino-4-(methylsulfinyl)-6-phenyl-5-pyrimidinyl]-2-isopropyl-3(2H)-pyridazinone and 3-pyridinylmethylamine according to a similar manner to that of Example 120.

mp: 215-216.5° C. (ethanol-diisopropyl ether)

IR (KBr): 3498, 3344, 3296, 3140, 1657, 1630, 1585, 1554 cm⁻¹

ESI/MS: 436[M+Na]⁺, 414[M+H]⁺

¹H NMR (CDCl₃, δ): 1.19 (6H, d, J=6.61 Hz), 4.68 (2H, d, J=5.40 Hz), 5.02 (2H, br.s), 5.26 (1H, 7-plet, J=6.61 Hz), 6.47 (2H, s), 7.25-7.43 (7H, m), 7.66-7.73 (1H, m), 8.55 (1H, dd, J=1.54, 4.77 Hz), 8.64 (1H, d, J=2.04 Hz)

Elemental Analysis for C₂₃H₂₃N₇O

Calcd.: C,66.81; H,5.61; N,23.71

Found: C,66.54; H,5.65; N,23.47

### EXAMPLE 123

6-{2-Amino-4-phenyl-6-[(4-pyridinylmethyl)amino]-5-pyrimidinyl}-2-isopropyl-3(2H)-pyridazinone was prepared from 6-[2-amino-4-(methylsulfinyl)-6-phenyl-5-pyrimidinyl]-2-isopropyl-3(2H)-pyridazinone and 4-pyridinylmethylamine according to a similar manner to that of Example 120.

mp: 221.5-222.5° C. (ethanol-diisopropyl ether)

IR (KBr): 3321, 3195, 1658, 1633, 1587, 1554 cm⁻¹

ESI/MS: 436[M+Na]⁺, 414[M+H]⁺

¹H NMR (CDCl₃, δ): 1.26 (6H, d, J=6.62 Hz), 4.71 (2H, d, J=5.70 Hz), 4.98 (2H, br.s), 5.30 (1H, 7-plet, J=6.62 Hz), 6.49 (2H, s), 7.26-7.43 (8H, m), 8.56-8.60 (2H, m)

Elemental Analysis for C₂₃H₂₃N₇O

Calcd.: C,66.81; H,5.61; N,23.71

Found: C,66.76; H,5.76; N,23.68

### EXAMPLE 124

6-{2-Amino-4-[(2-furylmethyl)amino]-6-phenyl-5-pyrimidinyl}-2-isopropyl-3(2H)-pyridazinone was prepared from 6-[2-amino-4-(methylsulfinyl)-6-phenyl-5-pyrimidinyl]-2-isopropyl-3(2H)-pyridazinone and 2-furylmethylamine according to a similar manner to that of Example 120.

mp: 213-214° C. (ethanol)

IR (KBr): 3361, 3319, 3199, 1668, 1643, 1591, 1547 cm⁻¹

ESI/MS: 425[M+Na]⁺, 403[M+H]⁺

¹H NMR (CDCl₃, δ): 1.10 (6H, d, J=6.60 Hz), 4.54 (2H, d, J=5.51 Hz), 5.05 (1H, 7-plet, J=6.60 Hz), 6.31-6.41 (2H, m), 6.45 (2H, br.s), 6.67 (1H, d, J=9.53 Hz), 6.87 (1H, d, J=9.53 Hz), 7.00 (1H, t, J=5.51 Hz), 7.22-7.31 (5H, m), 7.55-7.57 (1H, m)

Elemental Analysis for C₂₂H₂₂N₆O₂

Calcd.: C,65.66; H,5.51; N,20.88

Found: C,65.43; H,5.59; N,20.74

### EXAMPLE 125

6-[2-Amino-4-phenyl-6-(1-pyrrolidinyl)-5-pyrimidinyl]-2-isopropyl-3(2H)-pyridazinone was prepared from 6-[2-amino-4-(methylsulfinyl)-6-phenyl-5-pyrimidinyl]-2-isopropyl-3(2H)-pyridazinone and pyrrolidine according to a similar manner to that of Example 115.

mp: >300° C. (ethanol)

IR (KBr): 3500, 3276, 3143, 2966, 1666, 1622, 1589, 1552, 1531 cm⁻¹

ESI/MS: 775[2M+Na]⁺, 399[M+Na]⁺, 377[M+H]⁺

¹H NMR (CDCl₃, δ): 1.10 (6H, d, J=6.60 Hz), 1.70-1.77 (4H, m), 3.12-3.18 (4H, m), 5.07 (1H, 7-plet, J=6.60 Hz), 6.28 (2H, br.s), 6.63 (1H, d, J=9.48 Hz), 7.06-7.25 (6H, m)

Elemental Analysis for C₂₁H₂₄N₆O

Calcd.: C,67.00; H,6.43; N,22.32

Found: C,66.75; H,6.52; N,22.19

### EXAMPLE 126

6-[2-Amino-4-phenyl-6-(1-piperidinyl)-5-pyrimidinyl]-2-isopropyl-3(2H)-pyridazinone was prepared from 6-[2-amino-4-(methylsulfinyl)-6-phenyl-5-pyrimidinyl]-2-isopropyl-3(2H)-pyridazinone and piperidine according to a similar manner to that of Example 115.

mp: 221-223° C. (ethanol-diisopropyl ether)

IR (KBr): 3498, 3286, 3157, 1658, 1621, 1585, 1550 cm⁻¹

ESI/MS: 413[M+Na]⁺, 391[M+H]⁺

¹H NMR (CDCl₃, δ): 1.02 (6H, d, J=6.60 Hz), 1.46-1.53 (6H, m), 3.21-3.27 (4H, m), 4.98 (2H, br.s), 5.16 (1H, 7-plet, J=6.60 Hz), 6.79 (1H, d, J=9.50 Hz), 7.09 (1H, d, J=9.50 Hz), 7.11-7.27 (5H, m)

Elemental Analysis for C₂₂H₂₆N₆O

Calcd.: C,67.67; H,6.71; N,21.52

Found: C,67.54; H,6.82; N,21.43

### EXAMPLE 127

6-[2-Amino-4-(4-morpholinyl)-6-phenyl-5-pyrimidinyl]-2-isopropyl-3(2H)-pyridazinone was prepared from 6-[2-amino-4-(methylsulfinyl)-6-phenyl-5-pyrimidinyl]-2-isopropyl-3(2H)-pyridazinone and morpholine according to a similar manner to that of Example 115.

mp: 251-253° C. (ethanol)

IR (KBr): 3498, 3276, 3149, 1660, 1621, 1589, 1554, 1539 cm⁻¹

ESI/MS: 807[2M+Na]⁺, 415[M+Na]⁺, 393[M+H]⁺

¹H NMR (CDCl₃, δ): 1.02 (6H, d, J=6.60 Hz), 3.27 (4H, t, J=4.69 Hz), 3.63 (4H, t, J=4.69 Hz), 5.03 (2H, br.s), 5.16 (1H, 7-plet, J=6.60 Hz), 6.80 (1H, d, J=9.46 Hz), 7.09 (1H, d, J=9.46 Hz), 7.12-7.28 (5H, m)

Elemental Analysis for C₂₁H₂₄N₆O₂

Calcd.: C,64.27; H,6.16; N,21.41

Found: C,64.03; H,6.27; N,21.21

### EXAMPLE 128

A mixture of 6-[2-amino-4-(methylsulfinyl)-6-phenyl-5-pyrimidinyl]-2-isopropyl-3(2H)-pyridazinone (150 mg) and piperazine (1.748 g) in N,N-dimethylacetamide (0.3 ml) was heated at 100-105° C. for 10 hours. Water (3 ml) was added to give a solid. The solid was colleted by filtration and purified by column chromatography on silica gel (methanol-ethyl acetate 10:90 v/v) to give 6-[2-amino-4-phenyl-6-(1-piperazinyl)-5-pyrimidinyl]-2-isopropyl-3(2H)-pyridazinone as a solid (80 mg).

mp: 254-256° C. (ethanol-diisopropyl ether)

IR (KBr): 3496, 3285, 3155, 1657, 1620, 1585, 1552, 1527 cm⁻¹

ESI/MS: 805[2M+Na]⁺, 414[M+Na]⁺, 392[M+H]⁺

¹H NMR (DMSO-d₆, δ): 0.92 (6H, d, J=6.60 Hz), 2.57-2.63 (4H, m), 3.07-3.12 (4H, m), 4.97 (1H, 7-plet, J=6.60 Hz), 6.54 (2H, br.s), 6.80 (1H, d, J=9.52 Hz), 7.06-7.14 (2H, m), 7.22-7.28 (3H, m), 7.30 (1H, d, J=9.52 Hz)

Elemental Analysis for C₂₁H₂₅N₇O.0.1H₂O

Calcd.: C,64.14; H,6.46; N,24.93

Found: C,64.07; H,6.56; N,24.66

### EXAMPLE 129

6-[2-Amino-4-(4-methyl-1-piperazinyl)-6-phenyl-5-pyrimidinyl]-2-isopropyl-3(2H)-pyridazinone was prepared from 6-[2-amino-4-(methylsulfinyl)-6-phenyl-5-pyrimidinyl]-2-isopropyl-3(2H)-pyridazinone and 1-methylpiperazine according to a similar manner to that of Example 114.

mp: 213-215° C. (ethanol-diisopropyl ether)

IR (KBr): 3502, 3288, 3163, 1658, 1622, 1587, 1552, 1539 cm⁻¹

ESI/MS: 833[2M+Na]⁺, 428[M+Na]⁺, 406[M+H]⁺

¹H NMR (DMSO-d₆, δ): 0.92 (6H, d, J=6.60 Hz), 2.13 (3H, s), 2.21-2.26 (4H, m), 3.14-3.18 (4H, m), 4.98 (1H, 7-plet, J=6.60 Hz), 6.58 (2H, br.s), 6.81 (1H, d, J=9.52 Hz), 7.06-7.14 (2H, m), 7.22-7.32 (4H, m)

Elemental Analysis for C₂₂H₂₇N₇O

Calcd.: C,65.16; H,6.71; N,24.18

Found: C,65.03; H,6.81; N,24.10

### EXAMPLE 130

Sodium hydride (60% in oil suspension) (19.5 mg) was added to a solution of imidazole (35.9 mg) in N,N-dimethylacetamide (0.45 ml) under ice-cooling. After stirring for 30 minutes, 6-[2-amino-4-(methylsulfinyl)-6-phenyl-5-pyrimidinyl]-2-isopropyl-3(2H)-pyridazinone (150 mg) was added to the mixtutre and the mixture was heated at 100-105° C. for 15 hours. Water (3 ml) was added to give a solid. The solid was collected by filtration and purified by column chromatography on silica gel (methanol-ethyl acetate 3:97 v/v) to give 6-[2-amino-4-(1H-imidazol-1-yl)-6-phenyl-5-pyrimidinyl]-2-isopropyl-3(2H)-pyridazinone as a solid (44 mg).

mp: 255-258° C. (ethanol)

IR (KBr): 3440, 3136, 1637, 1589, 1574, 1529 cm⁻¹

ESI/MS: 396[M+Na]⁺, 374[M+H]⁺

¹H NMR (DMSO-d₆, δ): 0.95 (6H, d, J=6.61 Hz), 4.99 (1H, 7-plet, J=6.61 Hz), 6.74 (1H, d, J=9.50 Hz), 6.97-7.03 (2H, m), 7.13 (1H, s), 7.23-7.46 (7H, m), 7.76 (1H, s)

### EXAMPLE 131

6-[2-Amino-4-phenyl-6-(1H-1,2,4-triazol-1-yl)-5-pyrimidinyl]-2-isopropyl-3(2H)-pyridazinone was prepared from 6-[2-amino-4-(methylsulfinyl)-6-phenyl-5-pyrimidinyl]-2-isopropyl-3(2H)-pyridazinone and 1H-1,2,4-triazole according to a similar manner to that of Example 130.

mp: 266-268° C. (ethanol)

IR (KBr): 3309, 3165, 1666, 1647, 1595, 1531 cm⁻¹

ESI/MS: 771[2M+Na]⁺, 397[M+Na]⁺, 375[M+H]⁺

¹H NMR (DMSO-d₆, δ): 0.88 (6H, d, J=6.62 Hz), 4.97 (1H, 7-plet, J=6.62 Hz), 6.69 (1H, d, J=9.52 Hz), 7.07 (1H, d, J=9.52 Hz), 7.24-7.38 (5H, m), 7.51 (2H, br.d), 8.10 (1H, s), 9.13 (1H, s)

Elemental Analysis for C₁₉H₁₈N₈O.0.1H₂O

Calcd.: C,60.66; H,4.88; N,29.79

Found: C,60.65; H,4.89; N,29.59

### EXAMPLE 132

6-[2-Amino-4-(1H-benzimidazol-1-yl)-6-phenyl-5-pyrimidinyl]-2-isopropyl-3(2H)-pyridazinone was prepared from 6-[2-amino-4-(methylsulfinyl)-6-phenyl-5-pyrimidinyl]-2-isopropyl-3(2H)-pyridazinone and 1H-benzimidazole according to a similar manner to that of Example 130.

mp: 242-244° C. (ethanol)

IR (KBr): 3464, 3298, 3167, 3103,1660, 1591, 1571 cm⁻¹

ESI/MS: 869[2M+Na]⁺, 446[M+Na]⁺, 424[M+H]⁺

¹H NMR (CDCl₃, δ): 0.92 (6H, d, J=6.62 Hz), 5.08 (1H, 7-plet, J=6.62 Hz), 5.53 (2H, br.s), 6.55 (1H, d, J=9.54 Hz), 6.60 (1H, d, J=9.54 Hz), 7.19-7.47 (7H, m), 7.45-7.55 (1H, m), 7.78-7.80 (1H, m), 7.99 (1H, s)

Elemental Analysis for C₂₄H₂₁N₇O

Calcd.: C,68.07; H,5.00; N,23.15

Found: C,67.77; H,5.01; N,23.07

### EXAMPLE 133

A mixture of 6-[2-amino-4-(methylsulfinyl)-6-phenyl-5-pyrimidinyl]-2-isopropyl-3(2H)-pyridazinone (185 mg) and benzylamine (91.3 ml) in N,N-dimethylacetamide (0.37 ml) was heated at 100-105° C. for 10 hours. Water (3 ml) was added to give a solid. The solid was dissolved in chloroform, dried over magnesium sulfate, concentrated under reduced pressure and purified by column chromatography on silica gel (ethyl acetate) to give 6-(2-amino-4-anilino-6-phenyl-5-pyrimidinyl)-2-isopropyl-3(2H)-pyridazinone as a solid (73 mg).

mp: 240-242° C. (ethanol)

IR (KBr): 3375, 3305, 3190, 1668, 1633, 1593, 1570, 1495 cm⁻¹

ESI/MS: 819[2M+Na]⁺, 421[M+Na]⁺, 399[M+H]⁺

¹H NMR (CDCl₃, δ): 1.51 (6H, d, J=6.61 Hz), 5.10 (1H, br.s), 5.43 (1H, 7-plet, J=6.61 Hz), 6.51 (2H, s), 7.10-7.59 (10H, m), 8.94 (1H, br.s)

Elemental Analysis for C₂₃H₂₂N₆O.0.1H₂O

Calcd.: C,69.02; H,5.59; N,21.00

Found: C,69.05; H,5.69; N,20.75

### EXAMPLE 134

6-{2-Amino-4-[(4,5-dimethyl-1,3-thiazol-2-yl)amino]-6-phenyl-5-pyrimidinyl}-2-isopropyl-3(2H)-pyridazinone was prepared from 6-[2-amino-4-(methylsulfinyl)-6-phenyl-5-pyrimidinyl]-2-isopropyl-3(2H)-pyridazinone, 4,5-dimethyl-1,3-thiazol-2-amine hydrochloride and N-ethyl-N,N-diisopropylamine according to a similar manner to that of Example 133.

mp: 256-257.5° C. (ethanol)

IR (KBr): 3415, 3321, 3155, 1651, 1626, 1576, 1541 cm⁻¹

ESI/MS: 456[M+Na]⁺, 434[M+H]⁺

¹H NMR (DMSO-d₆, δ): 1.07 (6H, d, J=6.60 Hz), 2.11 (3H, s), 2.23 (3H, s), 5.05 (1H, 7-plet, J=6.60 Hz), 6.70-6.76 (3H, m), 7.05-7.33 (6H, m), 12.7 (1H, br.s)

Elemental Analysis for C₂₂H₂₃N₇OS

Calcd.: C,60.95; H,5.35; N,22.62

Found: C,60.89; H,5.40; N,22.49

### EXAMPLE 135

6-[2-Amino-4-phenyl-6-(2-pyridinylamino)-5-pyrimidinyl]-2-isopropyl-3(2H)-pyridazinone was prepared from 6-[2-amino-4-(methylsulfinyl)-6-phenyl-5-pyrimidinyl]-2-isopropyl-3(2H)-pyridazinone and 2-pyridinylamine according to a similar manner to that of Example 133.

mp: 163-164° C. (acetone-hexane)

IR (KBr): 3491, 3300, 3180, 1668, 1630, 1591, 1545 cm⁻¹

ESI/MS: 821[2M+Na]⁺, 422[M+Na]⁺, 400[M+H]⁺

¹H NMR (CDCl₃, δ): 1.55 (6H, d, J=6.60 Hz), 5.14 (2H, br.s), 5.41 (1H, 7-plet, J=6.60 Hz), 6.54 (2H, s), 6.92-7.00 (1H, m), 7.33-7.45 (5H, m), 7.64-7.74 (1H, m), 8.24-8.26 (1H, m), 8.46 (1H, d, J=8.48 Hz), 9.45 (1H, br.s)

Elemental Analysis for C₂₂H₂₁N₇O.0.2H₂O

Calcd.: C,65.56; H,5.35; N,24.33

Found: C,65.66; H,5.30; N,24.43

### EXAMPLE 136

A mixture of 6-[2-amino-4-(methylsulfinyl)-6-phenyl-5-pyrimidinyl]-2-isopropyl-3(2H)-pyridazinone (200 mg) and potassium fluoride (95 mg) in dimethylsulfoxide (1 ml) was heated at 100-105° C. for 15 hours. Water (20 ml) was added to give a solid. The solid was colleted by filtration and purified by column chromatography on silica gel (n-hexane-ethyl acetate 50:50 v/v) to give 6-(2-amino-4-fluoro-6-phenyl-5-pyrimidinyl)-2-isopropyl-3(2H)-pyridazinone as a solid (35 mg).

mp: 231-232.5° C. (ethanol)

IR (KBr): 3396, 3325, 3197, 1647, 1631, 1612, 1581, 1504 cm⁻¹

ESI/MS: 673[2M+Na]⁺, 348[M+Na]⁺, 326[M+H]⁺

¹H NMR (CDCl₃, δ): 1.03 (6H, d, J=6.63 Hz), 5.18 (1H, 7-plet, J=6.63 Hz), 5.46 (2H, br.s), 6.84 (1H, d, J=9.52 Hz), 7.09 (1H, dd, J=1.90,9.52 Hz), 7.28-7.37 (5H, m)

Elemental Analysis for C₁₇H₁₆FN₅O.0.2H₂O

Calcd.: C,62.07; H,5.02; N,21.29

Found: C,62.24; H,4.92; N,21.10

### EXAMPLE 137

A solution of 6-[2-amino-4-(methylsulfonyl)-6-phenyl-5-pyrimidinyl]-2-isopropyl-3(2H)-pyridazinone (193 mg) and guanidine carbonate (92 mg) in N,N-dimethylacetamide (0.38 ml) was heated at 100-105° C. for 15 hours. Water (3 ml) was added to give a solid. The solid was collected by filtration and dried over phosphorous pentoxide to give N-[2-amino-5-(1-isopropyl-6-oxo-1,6-dihydro-3-pyridazinyl)-6-phenyl-4-pyrimidinyl]guanidine as a solid (136 mg).

mp: 272-274.5° C. (N,N-dimethylacetamide-water)

IR (KBr): 3348, 1622, 1564, 1510 cm⁻¹

ESI/MS: 751[2M+Na]⁺, 365[M+H]⁺

¹H NMR (DMSO-d₆, δ): 0.69 (6H, d, J=6.62 Hz), 4.85 (1H, 7-plet, J=6.62 Hz), 6.35 (2H, br.s), 6.72 (1H, d, J=9.50 Hz), 7.00-7.29 (9H, m), 7.55 (1H, d, J=9.50 Hz)

Elemental Analysis for C₁₈H₂₀N₈O.0.5H₂O

Calcd.: C,57.90; H,5.67; N,30.01

Found: C,58.16; H,5.49; N,29.82

### EXAMPLE 138

6-[2-Amino-4-phenyl-6-(phenylthio)-5-pyrimidinyl]-2-isopropyl-3(2H)-pyridazinone was prepared from 6-[2-amino-4-(methylsulfonyl)-6-phenyl-5-pyrimidinyl]-2-isopropyl-3(2H)-pyridazinone and benzenethiol according to a similar manner to that of Example 95.

mp: 214-215° C. (ethanol)

IR (KBr): 3444, 3313, 3199, 1670, 1620, 1593, 1539, 1520 cm⁻¹

ESI/MS: 853[2M+Na]⁺, 438[M+Na]⁺, 416[M+H]⁺

¹H NMR (CDCl₃, δ): 1.32 (6H, d, J=6.63 Hz), 5.02 (2H, br.s), 5.33 (1H, 7-plet, J=6.63 Hz), 6.72 (1H, d, J=9.50 Hz), 6.86 (1H, d, J=9.50 Hz), 7.29-7.56 (10H, m)

Elemental Analysis for C₂₃H₂₁N₅OS

Calcd.: C,66.48; H,5.09; N,16.85

Found: C,66.31; H,5.12; N,16.83

### EXAMPLE 139

To a solution of 6-[2-amino-4-(methylthio)-6-phenyl-5-pyrimidinyl]-2-isopropyl-3(2H)-pyridazinone (5.00 g) in methanol (100 ml) was added a 28% solution of sodium methoxide in methanol (13.6 ml) and the mixture was refluxed for 36 hours. After cooling at ambient temperature, a precipitate was collected by filtration and dried under reduced pressure to give 6-(2-amino-4-methoxy-6-phenyl-5-pyrimidinyl)-2-isopropyl-3(2H)-pyridazinone as a solid (3.76 g).

mp: 230-233° C. (ethanol)

IR (KBr): 3519, 3394, 1660, 1606, 1581, 1543 cm⁻¹

ESI/MS: 697[2M+Na]⁺, 360[M+Na]⁺, 338[M+H]⁺

ESI/MS-Neg: 336[M−H]⁻

¹H NMR (CDCl₃, δ): 0.92 (6H, d, J=6.62 Hz), 3.95 (3H, s), 5.11 (1H, 7-plet, J=6.62 Hz), 5.22 (2H, br.s), 6.84 (1H, d, J=9.52 Hz), 7.21-7.30 (6H, m)

Elemental Analysis for C₁₈H₁₉N₅O₂

Calcd.: C,64.08; H,5.68; N,20.76

Found: C,64.24; H,5.64; N,20.75

### EXAMPLE 140

A mixture of 6-[2-amino-4-(methylthio)-6-phenyl-5-pyrimidinyl]-2-isopropyl-3(2H)-pyridazinone (535 mg) and urea hydrogen peroxide addition compound (143 mg) in acetic acid (0.8 ml) was heated at 70-75° C. for 1 hour. Urea hydrogen peroxide addition compound (143 mg) was added and the mixture was stirred at the same temperature. After 1 hour, urea hydrogen, peroxide addition compound (143 mg) was added and the mixture was heated at 70-75° C. for 3 hours. Water (12 ml) was added to the mixture. A precipitate was collected by filtration, washed with chloroform and dried under reduced pressure to give 6-(2-amino-6-oxo-4-phenyl-1,6-dihydro-5-pyrimidinyl)-2-isopropyl-3(2H)-pyridazinone as a solid (298 mg).

mp: 291-295° C. (acetic acid-water)

IR (KBr): 3363, 3126, 1645, 1576, 1500 cm⁻¹

ESI/MS: 669[2M+Na]⁺, 346[M+Na]⁺, 324[M+H]⁺

¹H NMR (DMSO-d₆, δ): 0.72 (6H, d, J=6.62 Hz), 4.87 (1H, 7-plet, J=6.62 Hz), 6.82 (1H, d, J=9.55 Hz), 6.91 (2H, br.s), 7.17-7.30 (4H, m), 7.47-7.50 (2H, m), 11.33 (1H, br.s)

Elemental Analysis for C₁₇H₁₇N₅O₂.0.8H₂O

Calcd.: C,60.45; H,5.55; N,20.73

Found: C,60.35; H,5.46; N,20.38

### EXAMPLE 141

A mixture of 6-[2-amino-4-(methylsulfinyl)-6-phenyl-5-pyrimidinyl]-2-isopropyl-3(2H)-pyridazinone (300 mg) and potassium thioacetate (111 mg) in N,N-dimethylacetamide (0.6 ml) was heated at 100-105° C. for one hour. The mixture was concentrated under reduced pressure and purified by column chromatography on silica gel. With an elution of a mixture of n-hexane and ethyl acetate (40:60 v/v) was given 5-[2-amino-5-(1-isopropyl-6-oxo-1,6-dihydro-3-pyridazinyl)-6-phenyl-4-pyrimidinyl]ethanethioate as a solid (8 mg). Next, with an elution of a mixture of n-hexane and ethyl acetate (20:80 v/v) was given 6-(2-amino-4-phenyl-6-thioxo-1,6-dihydro-5-pyrimidinyl)-2-isopropyl-3(2H)-pyridazinone as a solid (73 mg).

S-[2-amino-5-(1-isopropyl-6-oxo-1,6-dihydro-3-pyridazinyl)-6-phenyl-4-pyrimidinyl]ethanethioate

mp: 240° C. (ethanol-diisopropyl ether)

ESI/MS: 404[M+Na]⁺, 382[M+H]⁺

¹H NMR (CDCl₃, δ): 0.86 (6H, d, J=6.62 Hz), 2.28 (3H, s), 5.08 (1H, 7-plet, J=6.62 Hz), 6.93 (1H, d, J=9.46 Hz), 7.17-7.41 (6H, m), 7.60 (1H, d, J=9.46 Hz), 9.08 (1H, br.s)

6-(2-amino-4-phenyl-6-thioxo-1,6-dihydro-5-pyrimidinyl)-2-isopropyl-3(2H)-pyridazinone

mp: 295-299° C. (ethanol suspension)

IR (KBr): 1647, 1558 cm⁻¹

ESI/MS: 701[2M+Na]⁺, 362[M+Na]⁺, 340[M+H]⁺

¹H NMR (DMSO-d₆, δ): 0.74 (6H, d, J=6.60 Hz), 4.88 (1H, 7-plet, J=6.60 Hz), 6.80 (1H, d, J=9.50 Hz), 7.04-7.30 (7H, m), 7.62 (1H, d, J=9.50 Hz), 12.60 (1H, br.s)

### EXAMPLE 142

6-[2-Amino-4-(methylthio)-6-phenyl-5-pyrimidinyl]-2-methyl-3(2H)-pyridazinone was prepared from 6-[1-benzoyl-2,2-bis(methylthio)vinyl]-2-methyl-3(2H)-pyridazinone and guanidine carbonate according to a similar manner to that of Example 78.

mp: 233-235° C. (chloroform-diisopropyl ether)

IR (KBr): 3406, 3315, 3217, 1647, 1624, 1581, 1537 cm⁻¹

ESI/MS: 673[2M+Na]⁺, 348[M+Na]⁺, 326[M+H]⁺

¹H NMR (CDCl₃, δ): 2.49 (3H, s), 3.80 (3H, s), 5.23 (2H, br.s), 6.71 (1H, d, J=9.52 Hz), 6.77 (1H, d, J=9.52 Hz), 7.26-7.41 (5H, m)

¹H NMR (DMSO-d₆, δ): 2.45 (3H, s), 3.60 (3H, s), 6.79 (1H, d, J=9.52 Hz), 7.03 (2H, br.s), 7.09 (1H, d, J=9.52 Hz), 7.34 (5H, s)

### EXAMPLE 143

6-[2-Amino-4-(methylsulfinyl)-6-phenyl-5-pyrimidinyl]-2-methyl-3(2H)-pyridazinone and 6-[2-amino-4-(methylsulfonyl)-6-phenyl-5-pyrimidinyl]-2-methyl-3(2H)-pyridazinone was prepared from 6-[2-amino-4-(methylthio)-6-phenyl-5-pyrimidinyl]-2-methyl-3(2H)-pyridazinone and 3-chloroperbenzoic acid according to a similar manner to that of Example 79.

6-[2-amino-4-(methylsulfinyl)-6-phenyl-5-pyrimidinyl]-2-methyl-3(2H)-pyridazinone

mp: >250° C. (ethanol suspension)

IR (KBr): 3498, 3294, 1668, 1618, 1593, 1554, 1516 cm⁻¹

ESI/MS-Neg: 340[M−H]⁻

¹H NMR (CDCl₃, δ): 3.04 (3H, s), 3.84 (3H, s), 6.06 (2H, br.s), 6.56 (1H, d, J=9.52 Hz), 6.65 (1H, d, J=9.52 Hz), 7.34-7.52 (5H, m)

¹H NMR (DMSO-d₆, δ): 2.90 (3H, s), 3.67 (3H, s), 6.72 (1H, d, J=9,56 Hz), 6.92 (1H, d, J=9.56 Hz), 7.37-7.46 (5H, m), 7.62 (2H, br.s) 6-[2-amino-4-(methylsulfonyl)-6-phenyl-5-pyrimidinyl]-2-methyl-3(2H)-pyridazinone

mp: 240-243° C. (ethanol suspension)

IR (KBr): 3340, 3305, 3190, 1643, 1566 cm⁻¹

ESI/MS: 737[2M+Na]⁺, 380[M+Na]⁺, 358[M+H]⁺

¹H NMR (CDCl₃, δ): 3.30 (3H, s), 3.77 (3H, s), 5.60 (2H, br.s), 6.75 (1H, d, J=9.54 Hz), 6.89 (1H, d, J=9.54 Hz), 7.33-7.44 (5H, m)

### EXAMPLE 144

6-(2-Amino-4-methoxy-6-phenyl-5-pyrimidinyl)-2-methyl-3(2H)-pyridazinone was prepared from 6-[2-amino-4-(methylsulfinyl)-6-phenyl-5-pyrimidinyl]-2-methyl-3(2H)-pyridazinone and sodium methoxide according to a similar manner to that of Example 80.

mp: 218-220° C. (ethanol-diisopropyl ether)

IR (KBr): 3421, 1649, 1577 cm⁻¹

ESI/MS: 332[M+Na]⁺, 310[M+H]⁺

¹H NMR (CDCl₃, δ): 3.61 (3H, s), 3.93 (3H, s), 5.20 (2H, br.s), 6.81 (1H, d, J=9.52 Hz), 7.07 (1H, d, J=9.52 Hz), 7.26-7.35 (5H, m)

### EXAMPLE 145

6-[2-Amino-4-(benzylamino)-6-phenyl-5-pyrimidinyl]-2-methyl-3(2H)-pyridazinone was prepared from 6-[2-amino-4-(methylsulfinyl)-6-phenyl-5-pyrimidinyl]-2-methyl-3(2H)-pyridazinone and benzylamine according to a similar manner to that of Example 120.

mp: >250° C. (ethanol suspension)

IR (KBr): 3489, 3346, 3290, 1658, 1633, 1585, 1560 cm⁻¹

ESI/MS: 407[M+Na]⁺, 385[M+H]⁺

¹H NMR (DMSO-d₆, δ): 3.63 (3H, s), 4.58 (2H, d, J=6.01 Hz), 6.34 (2H, br.s), 6.66 (1H, d, J=9.52 Hz), 6.79 (1H, d, J=9.52 Hz), 7.18-7.34(1H, m)

### EXAMPLE 146

6-{2-amino-4-phenyl-6-[(2-pyridinylmethyl)amino]-5-pyrimidinyl}-2-methyl-3(2H)-pyridazinone was prepared from 6-[2-amino-4-(methylsulfinyl)-6-phenyl-5-pyrimidinyl]-2-methyl-3(2H)-pyridazinone and 2-pyridinylmethylamine according to a similar manner to that of Example 120.

mp: 248-250° C. (ethanol suspension)

IR (KBr): 3473, 3278, 1664, 1631, 1589, 1576, 1554 cm⁻¹

ESI/MS: 408[M+Na]⁺, 407[M+H]⁺

¹H NMR (DMSO-d₆, δ) 3.71 (3H, s), 4.67 (2H, d, J=5.31 Hz), 6.41 (2H, br.s), 6.64 (1H, d, J=9.58 Hz), 6.75 (1H, d, J=9.58 Hz), 7.24-7.85 (7H, m), 7.73-7.85 (2H, m), 8.52-8.55 (1H, m)

### EXAMPLE 147

6-(2-Amino-4-anilino-6-phenyl-5-pyrimidinyl)-2-methyl-3(2H)-pyridazinone was prepared from 6-[2-amino-4-(methylsulfinyl)-6-phenyl-5-pyrimidinyl]-2-methyl-3(2H)-pyridazinone and aniline according to a similar manner to that of Example 133.

mp: 252-254° C. (ethanol)

IR (KBr): 3384, 3307, 3149, 1662, 1649, 1580, 1549 cm⁻¹

ESI/MS: 393[M+Na]⁺, 371[M+H]⁺

¹H NMR (CDCl₃, δ): 3.92 (3H, s), 5.08 (2H, br.s), 6.54 (2H, s), 7.08-7.63 (10H, m), 9.23 (1H, br.s)

### EXAMPLE 148

A mixture of 6-[2-amino-4-(4-fluorophenyl)-6-(methylthio)-5-pyrimidinyl]-2-isopropyl-3(2H)-pyridazinone and 6-{2-amino-4-(methylthio)-6-[4-(methylthio)phenyl]-5-pyrimidinyl}-2-isopropyl-3(2H)-pyridazinone was prepared from 6-[l-(4-fluorobenzoyl)-2,2-bis(methylthio)vinyl]-2-isopropyl-3(2H)-pyridazinone and guanidine carbonate according to a similar manner to that of Example 78.

6-[2-amino-4-(4-fluorophenyl)-6-(methylthio)-5-pyrimidinyl]-2-isopropyl-3(2H)-pyridazinone

mp: 232-234° C. (ethanol)

IR (KBr): 3516, 3313, 1658, 1587, 1547 cm⁻¹

ESI/MS: 394[M+Na]⁺, 372[M+H]⁺

¹H NMR (CDCl₃, δ): 1.27 (6H, d, J=6.64 Hz), 2.49 (3H, s), 5.16 (2H, br.s), 5.31 (1H, 7-plet, J=6.64 Hz), 6.74 (1H, d, J=9.50 Hz), 6.83 (1H, d, J=9.50 Hz), 6.94-7.03 (2H, m), 7.26-7.37 (2H, m)

Elemental Analysis for C₁₈H₁₈FN₅O₂S

Calcd.: C,58.21; H,4.88; N,18.86

Found: C,58.45; H,4.95; N,18.65

6-{2-amino-4-(methylthio)-6-[4-(methylthio)phenyl]-5-pyrimidinyl}-2-isopropyl-3(2H)-pyridazinone

ESI/MS: 821[2M+Na]⁺, 422[M+Na]⁺, 400[M+H]⁺

¹H NMR (DMSO-d₆, δ): 1.30 (6H, d, J=6.60 Hz), 2.46 (3H, s), 2.48 (3H, s)

### EXAMPLE 149

A mixture of 6-[2-amino-4-(4-fluorophenyl)-6-methoxy-5-pyrimidinyl]-2-isopropyl-3(2H)-pyridazinone and 6-{2-amino-4-methoxy-6-[4-(methylthio)phenyl]-5-pyrimidinyl}-2-isopropyl-3(2H)-pyridazinone was prepared from a mixture of 6-[2-amino-4-(4-fluorophenyl)-6-(methylthio)-5-pyrimidinyl]-2-isopropyl-3(2H)-pyridazinone and 6-{2-amino-4-(methylthio)-6-[4-(methylthio)phenyl]-5-pyrimidinyl}-2-isopropyl-3(2H)-pyridazinone and a solution of 28% sodium methoxide in methanol according to a similar manner to that of Example 139.

6-[2-amino-4-(4-fluorophenyl)-6-methoxy-5-pyrimidinyl]-2-isopropyl-3(2H)-pyridazinone

mp: 207-209° C. (acetone)

IR (KBr): 3427, 3323, 3217, 1645, 1624, 1581 cm⁻¹

ESI/MS: 733[2M+Na]⁺, 378[M+Na]⁺, 356[M+H]⁺

¹H NMR (CDCl₃, δ): 0.96 (6H, d, J=6.60 Hz), 3.95 (3H, s), 5.07-5.21 (3H, m), 6.85 (1H, d, J=9.52 Hz), 6.92-7.03 (2H, m), 7.21-7.31 (3H, m)

6-{2-amino-4-methoxy-6-[4-(methylthio)phenyl]-5-pyrimidinyl}-2-isopropyl-3(2H)-pyridazinone

ESI/MS: 789[2M+Na]⁺, 406[M+Na]⁺, 384[M+H]⁺

¹H NMR (CDCl₃, δ): 2.44 (3H, s), 3.94 (3H, s)

### EXAMPLE 150

6-[2-Amino-4-(4-fluorophenyl)-6-methoxy-5-pyrimidinyl]-2-isopropyl-3(2H)-pyridazinone and 6-{2-amino-4-methoxy-6-[4-(methylsulfonyl)phenyl]-5-pyrimidinyl}-2-isopropyl-3(2H)-pyridazinone were prepared from a mixture of 6-[2-amino-4-(4-fluorophenyl)-6-methoxy-5-pyrimidinyl]-2-isopropyl-3(2H)-pyridazinone and 6-{2-amino-4-methoxy-6-[4-(methylthio)phenyl]-5-pyrimidinyl}-2-isopropyl-3(2H)-pyridazinone and 3-chloroperbenzoic acid according to a similar manner to that of Example 79.

6-[2-amino-4-(4-fluorophenyl)-6-methoxy-5-pyrimidinyl]-2-isopropyl-3(2H)-pyridazinone

mp: 207-209° C. (acetone)

IR (KBr): 3427, 3323, 3217, 1645, 1624, 1581 cm⁻¹

ESI/MS: 733[2M+Na]⁺, 378[M+Na]⁺, 356[M+H]⁺

¹H NMR (CDCl₃, δ): 0.96 (6H, d, J=6.60 Hz), 3.95 (3H, s), 5.07-5.21 (3H, m), 6.85 (1H, d, J=9.52 Hz), 6.92-7.03 (2H, m), 7.21-7.31 (3H, m) 6-{2-amino-4-methoxy-6-[4-(methylsulfonyl)phenyl]-5-pyrimidinyl}-2-isopropyl-3(2H)-pyridazinone

mp: 148-151° C. (acetone)

IR (KBr): 1660, 1633, 1587, 1566, 1547 cm⁻¹

ESI/MS: 853[2M+Na]⁺, 438[M+Na]⁺, 416[M+H]⁺

¹H NMR (CDCl₃, δ): 0.84 (6H, d, J=6.62 Hz), 2.98 (3H, s), 3.98 (3H, s), 5.10 (1H, 7-plet, J=6.62 Hz), 5.20 (2H, br.s), 6.89 (1H, d, J=9.54 Hz), 7.35 (1H, d, J=9.54 Hz), 7.47 (2H, d), 7.88 (2H, d)

¹H NMR (DMSO-d₆, δ): 0.69 (6H, d, J=6.60 Hz), 3.15 (3H, s), 3.90 (3H, s), 4.90 (1H, 7-plet, J=6.60 Hz), 6.91 (1H, d, J=9.54 Hz), 7.12 (2H, br.s), 7.45 (2H, d, J=8.32 Hz), 7.57 (1H, d, J=9.54 Hz), 7.87 (2H, d, J=8.32 Hz)

### EXAMPLE 151

Under ice-cooling, 3-perbenzoic acid (70% purity) (1.33 g) was added to a mixture of 6-[2-amino-4-(4-fluorophenyl)-6-methoxy-5-pyrimidinyl]-2-isopropyl-3(2H)-pyridazinone and 6-{2-amino-4-methoxy-6-[4-(methylsulfonyl)phenyl]-5-pyrimidinyl}-2-isopropyl-3(2H)-pyridazinone (1.47 g, molar ratio 6:4) in dichloromethane (15 ml). After stirring at the same temperature for 2 hours, the mixture was washed with saturated aqueous sodium thiosulfate, saturated aqueous sodium hydrogen carbonate and brine, successively, dried over magnesium sulfate and concentrated under reduced pressure to give a residue. The residue was purified by column chromatography on silica gel (n-hexane-ethyl acetate 10:90, ethyl acetate and methanol-ethyl acetate=4:96, 8:92, 10:90 v/v, in turn). Successively, 6-[2-amino-4-(4-fluorophenyl)-6-(methylthio)-5-pyrimidinyl]-2-isopropyl-3(2H)-pyridazinone (101 mg), 6-[2-amino-4-[4-(methylsulfonyl)-phenyl]-6-(methylthio)-5-pyrimidinyl]-2-isopropyl-3(2H)-pyridazinone (41 mg), 6-[2-amino-4-(4-fluorophenyl)-6-(methylsulfinyl)-5-pyrimidinyl]-2-isopropyl-3(2H)-pyridazinone (510 mg) and 6-{2-amino-4-(methylsulfinyl)-6-[4-(methylsulfinyl)phenyl]-5-pyrimidinyl}-2-isopropyl-3(2H)-pyridazinone (54 mg) were isolated as a solid, respectively.

6-[2-amino-4-(4-fluorophenyl)-6-(methylthio)-5-pyrimidinyl]-2-isopropyl-3(2H)-pyridazinone

mp: 232-234° C. (ethanol)

IR (KBr): 3516, 3313, 1658, 1587, 1547 cm⁻¹

ESI/MS: 394[M+Na]⁺, 372[M+H]⁺

¹H NMR (CDCl₃, δ): 1.27 (6H, d, J=6.64 Hz), 2.49 (3H, s), 5.16 (2H, br.s), 5.31 (1H, 7-plet, J=6.64 Hz), 6.74 (1H, d, J=9.50 Hz), 6.83 (1H, d, J=9.50 Hz), 6.94-7.03 (2H, m), 7.26-7.37 (2H, m)

6-[2-amino-4-[4-(methylsulfonyl)phenyl]-6-(methylthio)-5-pyrimidinyl]-2-isopropyl-3(2H)-pyridazinone

mp: 241-243° C. (ethanol suspension)

IR (KBr): 3438, 3330, 3224, 1653, 1630, 1583, 1537 cm⁻¹

ESI/MS: 885[2M+Na]⁺, 454[M+Na]⁺, 432[M+H]⁺

¹H NMR (CDCl₃, δ): 1.16 (6H, d, J=6.60 Hz), 2.52 (3H, s), 3.01 (3H, s), 5.17-5.35 (3H, m), 6.80 (1H, d, J=9.52 Hz), 6.99 (1H, d, J=9.52 Hz), 7.52 (2H, d, J=8.34 Hz), 7.89 (2H, d, J=8.34 Hz)

6-[2-amino-4-(4-fluorophenyl)-6-(methylsulfinyl)-5-pyrimidinyl]-2-isopropyl-3(2H)-pyridazinone

mp: 238-240° C. (chloroform-acetone)

IR (KBr): 3506, 3290, 3180, 1667, 1625, 1597, 1552 cm⁻¹

ESI/MS: 797[2M+Na]⁺, 410[M+Na]⁺, 388[M+H]⁺

¹H NMR (CDCl₃, δ): 1.37 (3H, d, J=6.64 Hz), 1.40 (3H, d, J=6.64 Hz), 2.98 (3H, s), 5.40 (1H, 7-plet, J=6.64 Hz), 5.75 (2H, br.s), 6.61 (1H, d, J=9.54 Hz), 6.70 (1H, d, J=9.54 Hz), 7.02-7.13 (2H, m), 7.35-7.44 (2H, m)

¹H NMR (DMSO-d₆, δ): 1.19 (6H, d, J=6.60 Hz), 2.85 (3H, s), 5.13 (2H, 7-plet), 6.77 (1H, d, J=9.53 Hz), 7.09 (1H, d, J=9.53 Hz), 7.18-7.41 (4H, m), 7.63 (2H, br.s)

Elemental Analysis for C₁₈H₁₈FN₅O₂

Calcd.: C,58.21; H,4.88; N,18.86

Found: C,58.45; H,4.95; N,18.65

6-{2-amino-4-(methylsulfinyl)-6-[4-(methylsulfinyl)phenyl]-5-pyrimidinyl}-2-isopropyl-3(2H)-pyridazinone

ESI/MS: 454[M+Na]⁺

¹H NMR (CDCl₃, δ): 1.33 (3H, d, J=6.61 Hz), 1.38 (3H, d, J=6.61 Hz), 2.73 (3H, s), 2.98 (3H, s), 5.77 (2H, br.s), 5.38 (1H, 7-plet, J=6.61 Hz), 6.65 (1H, d, J=9.56 Hz), 6.70 (1H, d, J=9.56 Hz), 7.53-7.70 (4H, m)

¹H NMR (DMSO-d₆, δ): 1.10 (6H, d, J=6.63 Hz), 2.73 (3H, s), 2.86 (3H, s), 5.10 (1H, 7-plet, J=6.63 Hz), 6.78 (1H, d, J=9.50 Hz), 7.17 (1H, d, J=9.50 Hz), 7.48 (2H, d, J=8.34 Hz), 7.63-7.71 (4H, m)

### EXAMPLE 152

Successively, 6-[2-amino-4-(4-fluorophenyl)-6-(methylthio)-5-pyrimidinyl]-2-isopropyl-3(2H)-pyridazinone (410 mg), 6-[2-amino-4-[4-(methylsulfonyl)phenyl]-6-(methylthio)-5-pyrimidinyl]-2-isopropyl-3(2H)-pyridazinone (257 mg), 6-[2-amino-4-(4-fluorophenyl)-6-(methylsulfinyl)-5-pyrimidinyl]-2-isopropyl-3(2H)-pyridazinone (128 mg), 6-[2-amino-4-[4-(methylsulfinyl)phenyl]-6-(methylthio)-5-pyrimidinyl]-2-isopropyl-3(2H)-pyridazinone (405 mg) and 6-{2-amino-4-(methylsulfinyl)-6-[4-(methylsulfonyl)phenyl]-5-pyrimidinyl}-2-isopropyl-3(2H)-pyridazinone (248 mg) were isolated as a solid, respectively, from a mixture of 6-[2-amino-4-(4-fluorophenyl)-6-methoxy-5-pyrimidinyl]-2-isopropyl-3(2H)-pyridazinone and 6-{2-amino-4-methoxy-6-[4-(methylsulfonyl)phenyl]-5-pyrimidinyl}-2-isopropyl-3(2H)-pyridazinone and 3-chloroperbenzoic acid (70% purity) according to a similar manner to that of Example 151.

6-[2-amino-4-(4-fluorophenyl)-6-(methylthio)-5-pyrimidinyl]-2-isopropyl-3(2H)-pyridazinone

mp: 232-234° C. (ethanol)

IR (KBr): 3516, 3313, 1658, 1587, 1547 cm⁻¹

ESI/MS: 394[M+Na]⁺, 372[M+H]⁺

¹H NMR (CDCl₃, δ): 1.27 (6H, d, J=6.64 Hz), 2.49 (3H, s), 5.16 (2H, br.s), 5.31 (1H, 7-plet, J=6.64 Hz), 6.74 (1H, d, J=9.50 Hz), 6.83 (1H, d, J=9.50 Hz), 6.94-7.03 (2H, m), 7.26-7.37 (2H, m) 6-[2-amino-4-[4-(methylsulfonyl)phenyl]-6-(methylthio)-5-pyrimidinyl]-2-isopropyl-3(2H)-pyridazinone

mp: 241-243° C. (ethanol suspension)

IR (KBr): 3438, 3330, 3224, 1653, 1630, 1583, 1537 cm⁻¹

ESI/MS: 885[2M+Na]⁺, 454[M+Na]⁺, 432[M+H]⁺

¹H NMR (CDCl₃, δ): 1.16 (6H, d, J=6.60 Hz), 2.52 (3H, s), 3.01 (3H, s), 5.17-5.35 (3H, m), 6.80 (1H, d, J=9.52 Hz), 6.99 (1H, d, J=9.52 Hz), 7.52 (2H, d, J=8.34 Hz), 7.89 (2H, d, J=8.34 Hz) 6-[2-amino-4-(4-fluorophenyl)-6-(methylsulfinyl)-5-pyrimidinyl]-2-isopropyl-3(2H)-pyridazinone

mp: 238-240° C. (chloroform-acetone)

IR (KBr): 3506, 3290, 3180, 1667, 1625, 1597, 1552 cm⁻¹

ESI/MS: 797[2M+Na]⁺, 410[M+Na]⁺, 388[M+H]⁺

¹H NMR (CDCl₃, δ): 1.37 (3H, d, J=6.64 Hz), 1.40 (3H, d, J=6.64 Hz), 2.98 (3H, s), 5.40 (1H, 7-plet, J=6.64 Hz), 5.75 (2H, br.s), 6.61 (1H, d, J=9.54 Hz), 6.70 (1H, d, J=9.54 Hz), 7.02-7.13 (2H, m), 7.35-7.44 (2H, m)

¹H NMR (DMSO-d₆, δ): 1.19 (6H, d, J=6.60 Hz), 2.85 (3H, s), 5.13 (2H, 7-plet), 6.77 (1H, d, J=9.53 Hz), 7.09 (1H, d, J=9.53 Hz), 7.18-7.41 (4H, m), 7.63 (2H, br.s)

Elemental Analysis for C₁₈H₁₈FN₅O₂S

Calcd.: C,58.21; H,4.88; N,18.86

Found: C,58.45; H,4.95; N,18.65

6-[2-amino-4-[4-(methylsulfinyl)phenyl]-6-(methylthio)-5-pyrimidinyl]-2-isopropyl-3(2H)-pyridazinone

mp: 221-223° C. (chloroform-acetone)

IR (KBr): 3390, 3302, 3203, 1658, 1583, 1539 cm⁻¹

ESI/MS: 438[M+Na]⁺, 416[M+H]⁺

¹H NMR (CDCl₃, δ): 1.20 (6H, d, J=6.62 Hz), 2.51 (3H, s), 2.68 (3H, s), 5.23-5.38 (4H, m), 6.76 (1H, d, J=9.52 Hz), 6.93 (1H, d, J=9.52 Hz), 7.46-7.61 (4H, m)

6-{2-amino-4-(methylsulfinyl)-6-[4-(methylsulfonyl)phenyl]-5-pyrimidinyl}-2-isopropyl-3(2H)-pyridazinone

mp: 241-243° C. (ethanol suspension)

IR (KBr): 3438, 3330, 3224, 1653, 1630, 1583, 1537 cm⁻¹

ESI/MS: 885[2M+Na]⁺, 454[M+Na]⁺, 432[M+H]⁺

¹H NMR (CDCl₃, δ): 1.16 (6H, d, J=6.60 Hz), 2.52 (3H, s), 3.01 (3H, s), 5.17-5.35 (3H, m), 6.80 (1H, d, J-9.52 Hz), 6.99 (1H, d, J=9.52 Hz), 7.52 (2H, d, J=8.34 Hz), 7.89 (2H, d, J=8.34 Hz)

### EXAMPLE 153

6-{2-Amino-4-methoxy-6-[4-(methylsulfinyl)phenyl]-5-pyrimidinyl}-2-isopropyl-3(2H)-pyridazinone was prepared from 6-[2-amino-4-[4-(methylsulfinyl)-phenyl]-6-(methylthio)-5-pyrimidinyl]-2-isopropyl-3(2H)-pyridazinone and sodium methoxide according to a similar manner to that of Example 139.

mp: 127-130° C. (chloroform-hexane)

IR (KBr): 3319, 3190, 1660, 1587, 1545 cm⁻¹

ESI/MS: 821[2M+Na]⁺, 422[M+Na]⁺

¹H NMR (CDCl₃, δ): 0.87 (6H, d, J=6.62 Hz), 2.66 (3H, s), 3.95 (3H, s), 5.10 (1H, 7-plet, J=6.62 Hz), 5.22 (2H, br.s), 6.87 (1H, d, J=9.52 Hz), 7.32 (1H, d, J=9.52 Hz), 7.40-7.46 (2H, m), 7.54-7.60 (2H, m)

### EXAMPLE 154

6-{2-Amino-4-methoxy-6-[4-(methylsulfonyl)phenyl]-5-pyrimidinyl}-2-isopropyl-3(2H)-pyridazinone was prepared from 6-[2-amino-4-[4-(methylsulfonyl)-phenyl]-6-(methylthio)-5-pyrimidinyl]-2-isopropyl-3(2H)-pyridazinone and sodium methoxide according to a similar manner to that of Example 139.

mp: 148-151° C. (acetone)

IR (KBr): 1660, 1633, 1587, 1566, 1547 cm⁻¹

ESI/MS: 853[2M+Na]⁺, 438[M+Na]⁺, 416[M+H]⁺

¹H NMR (CDCl₃, δ): 0.84 (6H, d, J=6.62 Hz), 2.98 (3H, s), 3.98 (3H, s), 5.10 (1H, 7-plet, J=6.62 Hz), 5.20 (2H, br.s), 6.89 (1H, d, J=9.54 Hz), 7.35 (1H, d, J=9.54 Hz), 7.47 (2H, d), 7.88 (2H, d)

¹H NMR (DMSO-d₆, δ): 0.69 (6H, d, J=6.60 Hz), 3.15 (3H, s), 3.90 (3H, s), 4.90 (1H, 7-plet, J=6.60 Hz), 6.91 (1H, d, J=9.54 Hz), 7.12 (2H, br.s), 7.45 (2H, d, J=8.32 Hz), 7.57 (1H, d, J=9.54 Hz), 7.87 (2H, d, J=8.32 Hz)

### EXAMPLE 155

6-{2-Amino-4-methoxy-6-[4-(methylsulfonyl)phenyl]-5-pyrimidinyl}-2-isopropyl-3(2H)-pyridazinone was prepared from 6-{2-amino-4-(methylsulfinyl)-6-[4-(methylsulfonyl)phenyl]-5-pyrimidinyl}-2-isopropyl-3(2H)-pyridazinone and sodium methoxide according to a similar manner to that of Example 80.

mp: 148-151° C. (acetone)

IR (KBr): 1660, 1633, 1587, 1566, 1547 cm⁻¹

ESI/MS: 853[2M+Na]⁺, 438[M+Na]⁺, 416[M+H]⁺

¹H NMR (CDCl₃, δ): 0.84 (6H, d, J=6.62 Hz), 2.98 (3H, s), 3.98 (3H, s), 5.10 (1H, 7-plet, J=6.62 Hz), 5.20 (2H, br.s), 6.89 (1H, d, J=9.54 Hz), 7.35 (1H, d, J=9.54 Hz), 7.47 (2H, d), 7.88 (2H, d)

¹H NMR (DMSO-d₆, δ): 0.69 (6H, d, J=6.60 Hz), 3.15 (3H, s), 3.90 (3H, s), 4.90 (1H, 7-plet, J=6.60 Hz), 6.91 (1H, d, J=9.54 Hz), 7.12 (2H, br.s), 7.45 (2H, d, J=8.32 Hz), 7.57 (1H, d, J=9.54 Hz), 7.87 (2H, d, J=8.32 Hz)

### EXAMPLE 156

6-[2-Amino-4-(benzylamino)-6-(4-fluorophenyl)-5-pyrimidinyl]-2-isopropyl-3(2H)-pyridazinone was prepared from 6-[2-amino-4-(4-fluorophenyl)-6-(methylsulfinyl)-5-pyrimidinyl]-2-isopropyl-3(2H)-pyridazinone and benzylamine according to a similar manner to that of Example 120.

mp: 191-192° C. (ethanol)

IR (KBr): 3494, 3352, 3302, 1660, 1631, 1585, 1566, 1552 cm⁻¹

ESI/MS: 453[M+Na]⁺, 431[M+H]⁺

¹H NMR (CDCl₃, δ): 1.12 (6H, d, J=6.62 Hz), 4.63 (2H, d, J=5.02 Hz), 5.00 (2H, br.s), 5.22 (1H, 7-plet, J=6.62 Hz), 6.46 (1H, d, J=9.56 Hz), 6.99 (1H, d, J=9.56 Hz), 6.97-7.44 (10H, m)

### EXAMPLE 157

6-{2-Amino-4-(4-fluorophenyl)-6-[(2-pyridinylmethyl)amino]-5-pyrimidinyl}-2-isopropyl-3(2H)-pyridazinone was prepared from 6-[2-amino-4-(4-fluorophenyl)-6-(methylsulfinyl)-5-pyrimidinyl]-2-isopropyl-3(2H)-pyridazinone and 2-pyridinylmethylamine according to a similar manner to that of Example 120.

mp: 210-212° C. (ethanol)

IR (KBr): 3375, 3303, 1660, 1589, 1552 cm⁻¹

ESI/MS: 454[M+Na]⁺, 432[M+H]⁺

¹H NMR (CDCl₃, δ): 1.40 (6H, d, J=6.63 Hz), 4.79 (2H, d, J=4.64 Hz), 4.98 (2H, br.s), 5.34 (1H, 7-plet, J=6.63 Hz), 6.51 (1H, d, J=9.56 Hz), 6.57 (1H, d, J=9.56 Hz), 6.96-7.06 (2H, m), 7.21-7.43 (4H, m), 7.63-7.69 (2H, m), 8.50-8.53 (1H, m)

### EXAMPLE 158

6-[2-Amino-4-anilino-6-(4-fluorophenyl)-5-pyrimidinyl]-2-isopropyl-3(2H)-pyridazinone was prepared from 6-[2-amino-4-(4-fluorophenyl)-6-(methylsulfinyl)-5-pyrimidinyl]-2-isopropyl-3(2H)-pyridazinone and aniline (according to a similar manner to that of Example 133.

mp: >250° C. (ethanol suspension)

IR (KBr): 3450, 3305, 3192, 1657, 1628, 1601, 1581, 1552 cm⁻¹

ESI/MS: 417[M+H]⁺

¹H NMR (CDCl₃, δ): 1.51 (6H, d, J=6.63 Hz), 5.07 (2H, br.s), 5.43 (1H, 7-plet, J=6.63 Hz), 6.52 (1H, d, J=9.62 Hz), 6.59 (1H, d, J=9.62 Hz), 7.00-7.59 (9H, m), 8.91 (1H, s)

### EXAMPLE 159

A mixture of 6-(2-amino-6-oxo-4-phenyl-1,6-dihydro-5-pyrimidinyl)-2-isopropyl-3(2H)-pyridazinone (324 mg) and sodium hydride (60% in oil suspension) (42 mg) in N,N-dimethylacetamide (1 ml) was heated at 50-55° C. for 30 minutes. tert-Butylbromoacetate (163 ml) was added to the mixture and the mixture was stirred at the same temperature for 3 hours. Water (5 ml) was added, stirred at ambient temperature and removed by decantation to give syrup. The syrup was purified by column chromatography on silica gel. With an elution of a mixture of n-hexane and ethyl acetate (50:50 v/v) was given tert-butyl {[2-amino-5-(1-isopropyl-6-oxo-1,6-dihydro-3-pyridazinyl)-6-phenyl-4-pyrimidinyl]oxy}acetate as a solid (50 mg). With an elution of ethyl acetate was given tert-butyl [2-amino-5-(1-isopropyl-6-oxo-1,6-dihydro-3-pyridazinyl)-6-oxo-4-phenyl-1(6H)-pyrimidinyl]acetate as a solid (265 mg).

tert-butyl {[2-amino-5-(1-isopropyl-6-oxo-1,6-dihydro-3-pyridazinyl)-6-phenyl-4-pyrimidinyl]-oxy}acetate

mp: 160-162° C. (acetone)

IR (KBr): 3431, 3309, 3195, 1745, 1658, 1635, 1591, 1545 cm⁻¹

ESI/MS: 460[M+Na]⁺, 438[M+H]⁺

¹H NMR (CDCl₃, δ): 0.88 (6H, d, J=6.62 Hz), 1.50 (9H, s), 4.80 (2H, s), 5.10 (1H, 7-plet, J=6.62 Hz), 5.12 (2H, br.s), 6.87 (1H, d, J=9.52 Hz), 7.23-7.29 (5H, m), 7.45 (1H, d, J=9.52 Hz)

Elemental Analysis for C₂₃H₂₇N₅O

Calcd.: C,63.14; H,6.22; N,16.01

Found: C,63.09; H,6.29; N,15.90

tert-butyl [2-amino-5-(1-isopropyl-6-oxo-1,6-dihydro-3-pyridazinyl)-6-oxo-4-phenyl-1(6H)-pyrimidinyl]acetate

mp: 138-141° C. (acetone suspension)

IR (KBr): 3342, 3219, 1755, 1705, 1645, 1583, 1535 cm⁻¹

ESI/MS: 897[2M+Na]⁺, 460[M+Na]⁺, 438[M+H]⁺

¹H NMR (CDCl₃, δ): 0.84 (6H, d, J=6.60 Hz), 1.52 (9H, s), 4.75 (2H, s), 5.06 (1H, 7-plet, J=6.60 Hz), 5.38 (2H, br.s), 6.86 (1H, d, J=9.52 Hz), 7.25-7.28 (5H, m), 7.46 (1H, d, J=9.52 Hz)

### EXAMPLE 160

6-[2-amino-4-(2-oxopropoxy)-6-phenyl-5-pyrimidinyl]-2-isopropyl-3(2H)-pyridazinone was prepared from 6-(2-amino-6-oxo-4-phenyl-1,6-dihydro-5-pyrimidinyl)-2-isopropyl-3(2H)-pyridazinone and 1-chloroacetone according to a similar manner to that of Example 159.

ESI/MS: 781[2M+Na]⁺, 402[M+Na]⁺, 380[M+H]⁺

¹H NMR (CDCl₃, δ): 0.92 (6H, d, J=6.62 Hz), 2.21 (3H, s), 4.94 (2H, s), 5.11 (1H, 7-plet, J=6.62 Hz), 5.19 (2H, br.s), 6.86 (1H, d, J=9.50 Hz), 7.26-7.29 (5H, m), 7.40 (1H, d, J=9.50 Hz)

### EXAMPLE 161

6-[2-Amino-4-(2-oxo-2-phenylethoxy)-6-phenyl-5-pyrimidinyl]-2-isopropyl-3(2H)-pyridazinone was prepared from 6-(2-amino-6-oxo-4-phenyl-1,6-dihydro-5-pyrimidinyl)-2-isopropyl-3(2H)-pyridazinone and 2-bromo-1-phenylethanone according to a similar manner to that of Example 159.

mp: 182-183° C. (diisopropyl ether-hexane)

IR (KBr): 3375, 3168, 1709, 1660, 1587 cm⁻¹

ESI/MS: 905[2M+Na]⁺, 464[M+Na]⁺, 442[M+H]⁺

¹H NMR (CDCl₃, δ): 0.90 (6H, d, J=6.60 Hz), 5.05 (2H, br.s), 5.10 (1H, 7-plet, J=6.60 Hz), 5.69 (2H, s), 6.87 (1H, d, J=9.52 Hz), 7.26-7.28 (5H, m), 7.48-7.70 (4H, m), 7.95-8.01 (2H, m)

### EXAMPLE 162

6-(2-Amino-4-phenyl-5-pyrimidinyl)-2-isopropyl-3(2H)-pyridazinone (62.4 mg) was dissolved in pyridine (5 ml). To the solution was added isoamyloyl chloride (32 mg) at 25° C. The solution was stirred for 2 hour at 25° C. The reaction mixture was added to a mixture of water and ethyl acetate. The organic layer was separated and washes with brine, dried over magnesium sulfate. Evaporation of solvent gave an oily residue. The residue was dissolved in chloroform and submitted to silica gel (31.4 ml) column. The column was eluted with chloroform-methanol (9:1). Evaporation of solvent of the fractions containing product gave crystals. Recrystallization from 80%-aqeous ethanol gave pure crystals of N-[5-(1-isopropyl-6-oxo-1,6-dihydro-3-pyridazinyl)-4-phenyl-2-pyrimidinyl]-3-methylbutanamide (33.4 mg)

¹H NMR (DMSO-d₆, δ): 0.94 (6H, d, J=6.6 Hz), 1.04 (6H, d, J=6.6 Hz), 2.03-2.16 (1H, m), 2.42 (2H, d, J=7 Hz), 5.05 (1H, m), 6.90 (1H, d, J=9.6 Hz), 7.34 (1H, d, J=9.6 Hz), 7.43 (5H, s), 8.85 (1H, s), 10.8 (1H, s)

ESI/MS: 392[M+H]⁺, 414[M+Na]⁺

IR (KBr): 3255, 2958, 1722, 1660, 1589, 1484, 1446 cm⁻¹

mp: 161° C. (aq-EtOH)

### EXAMPLE 163

N-[5-(1-isopropyl-6-oxo-1,6-dihydro-3-pyridazinyl)-4-phenyl-2-pyrimidinyl]-2-methylpropanamide was prepared according to a similar manner to that of Example 162.

¹H NMR (DMSO-d₆, δ): 1.04 (6H, d, J=6.6 Hz), 1.10 (6H, d, J=6.8 Hz), 2.84-2.98 (1H, m), 5.05 (1H, m), 6.90 (1H, d, J=9.6 Hz), 7.34 (1H, d, J=9.6 Hz), 7.43 (5H, s), 8.85 (1H, s), 10.8 (1H, s)

ESI/MS: 378[M+H]⁺, 400[M+Na]⁺

IR (KBr): 3255, 2958, 1730, 1660, 1587, 1484, 1446 cm⁻¹

mp: 145-147° C. (aq-EtOH)

### EXAMPLE 164

N-[5-(1-isopropyl-6-oxo-1,6-dihydro-3-pyridazinyl)-4-phenyl-2-pyrimidinyl]butanamide was prepared according to a similar manner to that of Example 162.

¹H NMR (DMSO-d₆, δ): 0.92 (3H, d, J=7.4 Hz), 1.04 (6H, d, J=6.6 Hz), 1.56-1.67 (2H, m), 2.53 (2H, t, J=7.4 Hz), 5.05 (1H, m), 6.90 (1H, d, J=9.6 Hz), 7.34 (1H, d, J=9.6 Hz), 7.43 (5H, s), 8.85 (1H, s), 10.8 (1H, s)

ESI/MS: 378[M+H]⁺, 400[M+Na]⁺

IR (KBr): 3255, 2958, 1725, 1660, 1583, 1484, 1446 cm⁻¹

mp: 148-150° C. (aq-EtOH)

### EXAMPLE 165

N-[5-(1-isopropyl-6-oxo-1,6-dihydro-3-pyridazinyl)-4-phenyl-2-pyrimidinyl]cyclohexanecarboxamide was prepared according to a similar manner to that of Example 162.

¹H NMR (DMSO-d₆, δ): 1.04 (6H, d, J=6.6 Hz), 1.18-1.87 (10H, m), 2.69-2.74 (1H, m), 5.05 (1H, m), 6.90 (1H, d, J=9.6 Hz), 7.31 (1H, d, J=9.6 Hz), 7.43 (5H, s), 8.84 (1H, s), 10.7 (1H, s)

ESI/MS: 418[M+H]⁺, 440[M+Na]⁺

IR (KBr): 3255, 2956, 1732, 1660, 1585, 1484, 1446 cm⁻¹

mp: 195-198° C. (aq-EtOH)

### EXAMPLE 166

N-[5-(1-isopropyl-6-oxo-1,6-dihydro-3-pyridazinyl)-4-phenyl-2-pyrimidinyl]-2-methoxyacetamide was prepared according to a similar manner to that of Example 162.

¹H NMR (DMSO-d₆, δ): 1.04 (6H, d, J=6.6 Hz), 3.36 (3H, s), 4.31 (2H, s), 5.05 (1H, m), 6.90 (1H, d, J=9.6 Hz), 7.34 (1H, d, J=9.6 Hz), 7.43 (5H, s), 8.89 (1H, s), 11.1 (1H, s)

ESI/MS: 380[M+H]⁺, 402[M+Na]⁺

IR (KBr): 3250, 2955, 1730, 1660, 1580, 1484, 1446 cm⁻

mp: 94-97° C. (aq-EtOH)

### EXAMPLE 167

N-[5-(1-isopropyl-6-oxo-1,6-dihydro-3-pyridazinyl)-4-phenyl-2-pyrimidinyl]cyclopropanecarboxamide was prepared according to a similar manner to that of Example 162.

¹H NMR (DMSO-d₆, δ): 0.85 (4H, d, J=5.6 Hz), 1.04 (6H, d, J=6.6 Hz), 2.19 (1H, t, J=5.6 Hz), 5.05 (1H, m), 6.90 (1H, d, J=9.6 Hz), 7.34 (1H, d, J=9.6 Hz), 7.43 (5H, s), 8.89 (1H, s), 11.1 (1H, s)

ESI/MS: 398[M+Na]⁺

IR (KBr): 3255, 2958, 1722, 1663, 1590, 1484, 1446 cm⁻¹

mp: 143-147° C. (aq-EtOH)

### EXAMPLE 168

Methyl 5-(1-isopropyl-6-oxo-1,6-dihydro-3-pyridazinyl)-4-phenyl-2-pyrimidinylcarbamate was prepared according to a similar manner to that of Example 162.

¹H NMR (DMSO-d₆, δ): 1.04 (6H, d, J=6.6 Hz), 3.70 (2H, s), 5.05 (1H, m), 6.90 (1H, d, J=9.6 Hz), 7.34 (1H, d, J=9.6 Hz), 7.43 (5H, s), 8.83 (1H, s), 10.73 (1H, s)

ESI/MS: 366[M+H]⁺, 388[M+Na]⁺

IR (KBr): 3255, 2958, 1722, 1660, 1589, 1484, 1446 cm⁻¹

mp: 197-200° C. (aq-EtOH)

### EXAMPLE 169

N-[5-(1-isopropyl-6-oxo-1,6-dihydro-3-pyridazinyl)-4-phenyl-2-pyrimidinyl]benzamide was prepared according to a similar manner to that of Example 162.

¹H NMR (DMSO-d₆, δ): 1.05 (6H, d, J=6.6 Hz), 5.05 (1H, m), 6.93 (1H, d, J=9.6 Hz), 7.40 (1H, d, J=9.6 Hz), 7.44 (5H, s), 7.44-7.66 (3H, m), 8.00 (2H, m), 8.89 (1H, s), 11.1 (1H, s)

ESI/MS-Neg: 410[M−H]⁻

IR (KBr): 3305, 2976, 1690,1660, 1585, 1481, 1442 cm⁻¹

mp: 116-120° C. (aq-EtOH)

### EXAMPLE 170

4-Fuoro-N-[5-(1-isopropyl-6-oxo-1,6-dihydro-3-pyridazinyl)-4-phenyl-2-pyrimidinyl]benzamide was prepared according to a similar manner to that of Example 162.

¹H NMR (DMSO-d₆, δ): 1.05 (6H, d, J=6.6 Hz), 5.05 (1H, m), 6.93 (1H, d, J=9.6 Hz), 7.32-7.44 (4H, m), 7.40 (1H, d, J=9.6 Hz), 7.44 (5H, s), 8.89 (1H, s), 11.1 (1H, s)

ESI/MS: 430[M+H]⁺, 452[M+Na]⁺

IR (KBr): 3305, 1660, 1585, 1481 cm⁻¹

mp: 106-110° C. (aq-EtOH)

### EXAMPLE 171

4-Chloro-N-[5-(1-isopropyl-6-oxo-1,6-dihydro-3-pyridazinyl)-4-phenyl-2-pyrimidinyl]benzamide was prepared according to a similar manner to that of Example 162.

¹H NMR (DMSO-d₆, δ): 1.05 (6H, d, J=6.6 Hz), 5.06 (1H, m), 6.92 (1H, d, J=9.6 Hz), 7.39 (1H, d, J=9.6 Hz), 7.44 (5H, s), 7.60 (2H, d, J=8.4 Hz), 8.01 (2H, d, J=8.4 Hz), 8.95 (1H, s), 11.4 (1H, s)

ESI/MS: 446[M+H]⁺, 468[M+Na]⁺

IR (KBr): 3300, 1665, 1575, 1470 cm⁻¹

mp: 100° C. (aq-EtOH)

### EXAMPLE 172

N-[5-(1-isopropyl-6-oxo-1,6-dihydro-3-pyridazinyl)-4-phenyl-2-pyrimidinyl]-4-methoxybenzamide was prepared according to a similar manner to that of Example 162.

¹H NMR (DMSO-d₆, δ): 1.05 (6H, d, J=6.6 Hz), 3.85 (3H, s), 5.06 (1H, m), 6.91 (1H, d, J=9.6 Hz), 7.05 (2H, d, J=8.8 Hz), 7.39 (1H, d, J=9.6 Hz), 8.02 (2H, d, J=8.8 Hz), 7.49 (5H, s), 8.93 (1H, s), 11.1 (1H, s)

ESI/MS: 442[M+H]⁺, 464.1[M+Na]⁺

IR (KBr): 3250, 1670, 1580, 1470 cm⁻¹

mp: 171-174° C. (aq-EtOH)

### EXAMPLE 173

N-[5-(1-isopropyl-6-oxo-1,6-dihydro-3-pyridazinyl)-4-phenyl-2-pyrimidinyl]-4-methylbenzamide was prepared according to a similar manner to that of Example 162.

¹H NMR (DMSO-d₆, δ): 1.05 (6H, d, J=6.6 Hz), 2.40 (3H, s), 5.06 (1H, m), 6.92 (1H, d, J=9.6 Hz), 7.33 (2H, d, J=8.0 Hz), 7.39 (1H, d, J=9.6 Hz), 7.49 (5H, s), 7.92 (2H, d, J=8.0 Hz), 8.93 (1H, s), 11.2 (1H, s)

ESI/MS: 426[M+H]⁺, 448[M+Na]⁺

IR (KBr): 3350, 1675, 1560, 1475 cm⁻¹

mp: 116° C. (aq-EtOH)

### EXAMPLE 174

N-[5-(1-isopropyl-6-oxo-1,6-dihydro-3-pyridazinyl)-4-phenyl-2-pyrimidinyl]-4-(trifluoromethyl)benzamide was prepared according to a similar manner to that of Example 162.

¹H NMR (DMSO-d₆, δ): 1.05 (6H, d, J=6.6 Hz), 5.06 (1H, m), 6.93 (1H, d, J=9.6 Hz), 7.40 (1H, d, J=9.6 Hz), 7.42 (5H, s), 7.90 (2H, d, J=8.4 Hz), 8.15 (2H, d, J=8.4 Hz), 8.95 (1H, s), 11.5 (1H, s)

ESI/MS: 480[M+H]⁺, 502[M+Na]⁺

IR (KBr): 3320, 1685, 1555, 1484 cm⁻¹

mp: 160-163° C. (aq-EtOH)

### EXAMPLE 175

6-(2-Amino-4-phenyl-5-pyrimidinyl)-2-isopropyl-3 (2H)-pyridazinone (122.8 mg) was dissolved in pyridine (10 ml). To the solution was added 3-picolyl chloride hydrochloride (142 mg) at room temperature. The solution was stirred for 2 hours, and stood overnight. Concentration of the above reaction mixture gave crystal residue. To the residue was added H₂O (20 ml). Crystals were precipitated and collected by filtration. Recrstallization from 5%-aqeous ethanol gave pure crystals of

N-[5-(1-isopropyl-6-oxo-1,6-dihydro-3-pyridazinyl)-4-phenyl-2-pyrimidinyl)nicotinamide (157 mg).

¹H NMR (DMSO-d₆, δ): 1.05 (6H, d, J=6.6 Hz), 5.05 (1H, m), 6.93 (1H, d, J=9.6 Hz), 7.39 (1H, d, J=9.6 Hz), 7.44 (5H, s), 7.53-7.60 (1H, m), 8.30-8.36 (1H, m), 8.77-8.80 (1H, m), 8.96 (1H, s), 9.11 (1H, d, J=2 Hz), 11.5 (1H, s)

ESI/MS: 413[M+H]⁺, 435[M+Na]⁺

IR (KBr): 3473, 2989, 1695, 1664, 1589, 1479, 1440 cm⁻¹

mp: 163-167° C. (aq-EtOH)

### EXAMPLE 176

6-(2-Amino-4-phenyl-5-pyrimidinyl)-2-isopropyl-3(2H)-pyridazinone (122.8 mg) was dissolved in dichloromethane (10 ml). To the solution were added pivaloyl chloride (96.5 mg) and diisopropyl ethyl amine (207 mg) at 25° C. The solution was stirred for 2 hours, and stood overnight. Concentration of the above reaction mixture gave oily residue. The residue was added to a mixture of water and ethyl acetate. The organic layer was separated, and washed with brine, dried over magnesium sulfate. Evaporation of solvent gave an oily residue. The residue was pulverized with diisoprppyl ether (10 ml) to give N-[5-(1-isopropyl-6-oxo-1,6-dihydro-3-pyridazinyl)-4-phenyl-2-pyrimidinyl]-2,2-dimethylpropanamide (142 mg).

¹H NMR (DMSO-d₆, δ): 1.04 (6H, d, J=6.6 Hz), 1.25 (9H, s), 5.05 (1H, m), 6.90 (1H, d, J=9.6 Hz), 7.36 (1H, d, J=9.6 Hz), 7.44 (5H, s), 8.88 (1H, s), 10.3 (1H, s)

ESI/MS: 392[M+H]⁺, 414[M+Na]⁺

IR (KBr): 3253, 2973, 1706, 1654, 1571, 1482, 1446 cm⁻¹

mp: 138-141° C. (IPE)

### EXAMPLE 177

2,2,2-trichloroethyl 5-(1-isopropyl-6-oxo-1,6-dihydro-3-pyridazinyl)-4-phenyl-2-pyrimidinylcarbamate was prepared according to a similar manner to that of Example 176.

¹H NMR (DMSO-d₆, δ): 1.05 (6H, d, J=6.6 Hz).4.94 (2H, s), 5.05 (1H, m), 6.90 (1H, d, J=9.6 Hz), 7.35 (1H, d, J=9.6 Hz), 7.44 (5H, s), 8.88 (1H, s), 11.2 (1H, s),

ESI/MS: 482[M+H]⁺, 504[M+Na]⁺

IR (KBr): 3253, 2973, 1745, 1654, 1571, 1482, 1446 cm⁻¹

mp: 158-162° C. (IPE)

### EXAMPLE 178

6-(2-Amino-4-phenyl-5-pyrimidinyl)-2-isopropyl-3(2H)-pyridazinone (61.4 mg) was dissolved in dichloromethane (10 ml). To the solution were added benzoyl chloride (56.2 mg) and diisopropyl ethyl amine (103 mg) at 25° C. The solution was stirred for 5 hours. Concentration of the above reaction mixture gave oily residue. The residue was added to a mixture of water and ethyl acetate. The organic layer was separated, and washed with brine, dried over magnesium sulfate. Evaporation of solvent gave an oily residue. The residue was purified with column chromatography on silica gel to give N-benzoyl-N-[5-(1-isopropyl-6-oxo-1,6-dihydro-3-pyridazinyl)-4-phenyl-2-pyrimidinyl]benzamide, which was crystallized from 90%-aqeous ethanol (59.5 mg).

¹H NMR (DMSO-d₆, δ): 1.04 (6H, d, J=6.0 Hz), 5.01 (1H, m), 7.04-7.05 (2H, m), 7.29-7.40 (4H, m), 7.49-7.70 (6H, m), 7.83-7.87 (4H, m), 8.99 (1H, s)

ESI/MS: 516[M+H]⁺, 538[M+Na]⁺

IR (KBr): 3255, 1655, 1570, 1475, 1440 cm⁻¹

mp: 121-125° C. (aq-EtOH)

### EXAMPLE 179

4-Fluoro-N-(4-fluorobenzoyl)-N-[5-(1-isopropyl-6-oxo-1,6-dihydro-3-pyridazinyl)-4-phenyl-2-pyrimidinyl]benzamide was prepared according to a similar manner to that of Example 178.

¹H NMR (DMSO-d₆, δ): 1.00 (6H, d, J=6.6 Hz), 5.02 (1H, m), 6.87 (1H, d, J=9.6 Hz), 7.10-7.15 (2H, m), 7.32-7.42 (8H, m), 7.90-7.98 (4H, m), 9.00 (1H, s)

ESI/MS: 552[M+H]⁺, 574[M+Na]⁺

IR (KBr): 3255, 1665, 1565, 1470, 1445 cm⁻¹

mp: 148-151° C. (aq-EtOH)

### EXAMPLE 180

4-Chloro-N-(4-chlorobenzoyl)-N-[5-(1-isopropyl-6-oxo-1,6-dihydro-3-pyridazinyl)-4-phenyl-2-pyrimidinyl]benzamide was prepared according to a similar manner to that of Example 178.

¹H NMR (DMSO-d₆, δ): 1.00 (6H, d, J=6.6 Hz), 5.01 (1H, m), 6.81 (1H, d, J=9.8 Hz), 7.10-7.15 (2H, m), 7.32-7.42 (3H, m), 7.51 (4H, d, J=8.6 Hz), 7.87 (4H, d, J=8.6 Hz), 9.00 (1H, s)

ESI/MS: 584, 586[M+H]⁺, 606, 608[M+Na]⁺

IR (KBr): 3255, 1670, 1570, 1470, 1440 cm⁻¹

mp: 110° C. (aq-EtOH)

### EXAMPLE 181

N-[5-(1-isopropyl-6-oxo-1,6-dihydro-3-pyridazinyl)-4-phenyl-2-pyrimidinyl]-4-methoxy-N-(4-methoxybenzoyl) benzamide was prepared according to a similar manner to that of Example 178.

¹H NMR (DMSO-d₆, δ): 0.99 (6H, d, J=6.6 Hz), 3.82 (6H, s), 5.02 (1H, m), 6.91 (1H, d, J=9.6 Hz), 7.05 (4H, d, J=9.0 Hz), 7.12-7.16 (2H, m), 7.34-7.41 (4H, m), 7.80 (4H, d, J=9.0 Hz), 8.96 (1H, s),

ESI/MS: 576[M+H]⁺, 598[M+Na]⁺

IR (KBr): 3265, 1672, 1575, 1465, 1440 cm⁻¹

mp: 171-174° C. (EtOH)

### EXAMPLE 182

N-[5-(1-isopropyl-6-oxo-1,6-dihydro-3-pyridazinyl)-4-phenyl-2-pyrimidinyl]-4-methyl-N-(4-methylbenzoyl) benzamide was prepared according to a similar manner to that of Example 178.

¹H NMR (DMSO-d₆, δ): 1.00 (6H, d, J=6.6 Hz), 2.30 (6H, s), 5.02 (1H, m), 6.91 (1H, d, J=9.6 Hz), 7.01-7.39 (10H, m), 7.72-7.88 (4H, m), 8.97 (1H, s)

ESI/MS: 544[M+H]⁺, 566[M+Na]⁺

IR (KBr): 3265, 1675, 1570, 1470, 1445 cm⁻¹

mp: 159° C. (aq-EtOH)

### EXAMPLE 183

N-[5-(1-isopropyl-6-oxo-1,6-dihydro-3-pyridazinyl)-4-phenyl-2-pyrimidinyl]-4-(trifluoromethyl)-N-[4-(trifluoromethyl)benzoyl]benzamide was prepared according to a similar manner to that of Example 178.

¹H NMR (DMSO-d₆, δ): 0.95 (6H, d, J=6.6 Hz), 5.00 (1H, m), 6.93 (1H, d, J=9.6 Hz), 7.01-7.05 (2H, m), 7.30-7.44 (4H, m), 8.09 (4H, d, J=8.4 Hz), 9.03 (1H, s),

ESI/MS: 652[M+H]⁺, 674[M+Na]⁺

IR (KBr): 3265, 1674, 1564, 1475, 1440 cm⁻¹

mp: 159° C. (aq-EtOH)

### EXAMPLE 184

Dimethyl 5-(1-isopropyl-6-oxo-1,6-dihydro-3-pyridazinyl)-4-phenyl-2-pyrimidinylimidodicarbonate was prepared according to a similar manner to that of Example 178.

¹H NMR (DMSO-d₆, δ): 1.01 (6H, d, J=6.6 Hz), 3.80 (6H, s), 5.04 (1H, m), 6.97 (1H, d, J=9.6 Hz), 7.48 (5H, s), 7.52 (1H, d, J=9.6 Hz), 9.12 (1H, s)

ESI/MS: 424[M+H]⁺, 446[M+Na]⁺

IR (KBr): 3265, 1694, 1550, 1460, 1440 cm⁻¹

mp: 216° C. (aq-EtOH)

### EXAMPLE 185

To 1M-borontribromide solution in methylene chloride (70 ml) was added 6-[2-amino-4-(4-methoxyphenyl)-5-pyrimidinyl]-2-isopropyl-3(2H)-pyridazinone (4.72 g) at 5-10° C. The reaction mixture was stirred at room temperature for 2 hours. The mixture was cooled in ice bath. To the cooled solution was added water. Organic layer was separated and concentrated in vacuo to give oily residue. To the residue was added the above separated aqueous layer. PH of the aqueous solution was adjusted to 6-7 with 10%-NaOH aqueous solution. White crystals were precipitated. The suspension was stirred at room temperature for 2 hours, at 0-5° C. for 1 hour, and stood in the refrigerator overnight. The crystals were collected by filtration and dried in vacuo at 35-40° C., to give 6-[2-amino-4-(4-hydroxyphenyl)-5-pyrimidinyl]-2-isopropyl-3(2H)-pyridazinone (4.45 g).

¹H NMR (DMSO-d₆, δ): 1.17 (6H, d, J=6.8 Hz), 5.06-5.12 (1H, m), 6.74 (2H, d, J=8.6 Hz), 6.77 (1H, d, J=9.6 Hz), 6.95 (2H, s), 7.03 (1H, d, J=9.6 Hz), 7.20 (2H, d, J=8.6 Hz), 8.35 (1H, s), 9.80 (1H, brs)

ESI/MS: 342[M+H]⁺, 346[M+Na]⁺

IR (KBr): 3149, 1652, 1583, 1479, 1407 cm⁻¹

mp: 246° C. (H₂O)

### EXAMPLE 186

6-[2-Amino-4-(2-hydroxyphenyl)-5-pyrimidinyl]-2-isopropyl-3(2H)-pyridazinone was prepared according to a similar manner to that of Example 185.

¹H NMR (DMSO-d₆, δ): 1.11 (6H, d, J=6.6 Hz), 5.03-5.10 (1H, m), 6.70-6.80 (4H, m), 6.99 (2H, brs), 7.08-7.20 (2H, m), 8.42 (1H, s), 9.53 (1H, s)

ESI/MS: 342[M+H]⁺, 346[M+Na]⁺

IR (KBr): 3166, 1656, 1573, 1500, 1286 cm⁻¹

mp: 241° C. (H₂O)

### EXAMPLE 187

6-[2-Amino-4-(3-hydroxyphenyl)-5-pyrimidinyl]-2-isopropyl-3(2H)-pyridazinone was prepared according to a similar manner to that of Example 185.

¹H NMR (DMSO-d₆, δ): 0.98 (6H, d, J=6.6 Hz), 4.95-5.01 (1H, m), 6.72-6.89 (3H, m), 6.99 (2H, brs), 7.14-7.30 (3H, m), 8.40 (1H, s), 9.72 (1H, s)

ESI/MS: 342[M+H]⁺, 346[M+Na]⁺

IR (KBr): 3181, 1654, 1581, 1484 cm⁻¹

mp: 204° C. (H₂O)

### EXAMPLE 188

6-[2-Amino-4-(4-hydroxyphenyl)-5-pyrimidinyl]-2-isopropyl-3(2H)-pyridazinone (97 mg) was dissolved in N,N-dimethylformaide (5 ml). To the solution were add ethyl bromide (36 mg) and potassium tert-butoxide (36.9 mg) at 25° C. The reaction mixture was stirred for 15 hours at ambient temperature. The reaction mixture was added to a mixture of water and ethyl acetate. The organic layer was separated and washed with brine. The aqueous layer was combined and extracted with ethyl acetate. The organic layer was combined and passed through the diatomaceous earth column. Evaporation of solvent at reduced pressure gave a residue. The residue was purified with column chromatography on silica gel (methanol:chloroform 5:95-10:90) to give 6-[2-amino-4-(4-ethoxyphenyl)-5-pyrimidinyl]-2-isopropyl-3(2H)-pyridazinone, which was crystallized from 90% aq-ethanol (27.5 mg)

¹H NMR (DMSO-d₆, δ): 1.14 (6H, d, J=6.6 Hz), 1.31 (3H, t, J=7.0 Hz), 4.02 (2H, q, J=7.0 Hz), 5.01-5.14 (1H, m), 6.73 (1H, d, J=9.6 Hz), 6.92 (2H, d, J=8.8 Hz), 7.05 (2H, s), 7.25 (1H, d, J=9.6 Hz), 8.38 (1H, s)

ESI/MS: 352[M+H]⁺, 374[M+Na]⁺

IR (KBr): 3374, 1645, 1585, 1405 cm⁻¹

mp: 204° C. (aq-EtOH)

### EXAMPLE 189

6-[2-Amino-4-(2-ethoxyphenyl)-5-pyrimidinyl]-2-isopropyl-3(2H)-pyridazinone was prepared according to a similar manner to that of Example 188.

¹H NMR (DMSO-d₆, δ): 0.93 (6H, brs), 1.00 (3H, t), 3.7 (2H, brs), 4.92-4.99 (1H, m), 6.80 (1H, d, J=9.6 Hz), 6.97 (2H, brs), 6.88-7.08 (2H, m), 7.25 (1H, d, J=9.6 Hz), 7.29-7.43 (2H, m), 8.43 (1H, s)

ESI/MS: 352[M+H]⁺, 374[M+Na]⁺

IR (KBr): 3193, 1641, 1575, 1482 cm⁻¹

mp: 185° C. (aq-EtOH)

### EXAMPLE 190

6-[2-Amino-4-(3-ethoxyphenyl)-5-pyrimidinyl]-2-isopropyl-3(2H)-pyridazinone was prepared according to a similar manner to that of Example 188.

¹H NMR (DMSO-d₆, δ): 1.08 (6H, d, J=6.6 Hz), 1.26 (3H, t, J=7.0 Hz), 3.93 (2H, q, J=7.0 Hz), 5.02-5.08 (1H, m), 6.80 (1H, d, J=9.6 Hz), 6.85-6.96 (3H, m), 7.06 (2H, s), 7.14 (1H, d, J=9.6 Hz), 7.27 (1H, t, J=8.0 Hz), 8.44 (1H, s)

ESI/MS: 352[M+H]⁺, 374[M+Na]⁺

IR (KBr): 3307, 1620, 1585, 1482 cm⁻¹

mp: 142° C. (aq-EtOH)

### EXAMPLE 191

6-[2-Amino-4-(4-propoxyphenyl)-5-pyrimidinyl]-2-isopropyl-3(2H)-pyridazinone was prepared according to a similar manner to that of Example 188.

¹H NMR (DMSO-d₆, δ): 0.96 (3H, t, J=7.4 Hz), 1.14 (6H, d, J=6.6 Hz), 1.66-1.77 (2H, m), 3.93 (2H, d, J=6.6 Hz), 5.02-5.12 (1H, m), 6.79 (1H, d, J=9.6 Hz), 6.93 (2H, d, J=8.8 Hz), 7.00 (2H, s), 7.08 (1H, d, J=9.6 Hz), 7.30 (2H, d, J=8.8 Hz), 8.34 (1H, s)

ESI/MS: 366[M+H]⁺

IR (KBr): 3376, 1641, 1585, 1482 cm⁻¹

mp: 175° C. (aq-EtOH)

### EXAMPLE 192

6-[2-Amino-4-(2-propoxyphenyl)-5-pyrimidinyl]-2-isopropyl-3(2H)-pyridazinone was prepared according to a similar manner to that of Example 188.

¹H NMR (DMSO-d₆, δ): 0.72 (3H, t), 0.95-1.04 (6H, m), 1.34-1.43 (2H, m), 3.57-3.61 (2H, m), 4.92-4.99 (1H, m), 6.78 (1H, d, J=9.6 Hz), 6.92 (1H, d, J=8.4 Hz), 6.98 (2H, s), 7.03 (2H, m, J=t Hz), 7.22 (1H, d, J=9.6 Hz), 7.31-7.40 (2H, m), 8.46 (1H, s)

ESI/MS: 366[M+H]⁺

IR (KBr): 3421, 1648, 1573, 1452 cm⁻¹

mp: 145° C. (aq-EtOH)

### EXAMPLE 193

6-[2-Amino-4-(3-propoxyphenyl)-5-pyrimidinyl]-2-isopropyl-3(2H)-pyridazinone was prepared according to a similar manner to that of Example 188.

¹H NMR (DMSO-d₆, δ): 0.92 (3H, t, J=7.4 Hz), 1.08 (6H, d, J=6.6 Hz), 1.60-1.67 (2H, m), 3.83 (2H, t, J=7.4 Hz), 5.05 (1H, m), 6.80 (1H, d, J=9.6 Hz), 6.84-6.91 (3H, m), 7.07 (2H, s), 7.14 (1H, d, J=9.6 Hz), 7.24 (1H, t, J=7.6 Hz), 8.44 (1H, s)

ESI/MS: 366[M+H]⁺, 388[M+Na]⁺

IR (KBr): 3194, 1660, 1575, 1484 cm⁻¹

mp: 122° C. (aq-EtOH)

### EXAMPLE 194

6-[2-Amino-4-(4-isopropoxyphenyl)-5-pyrimidinyl]-2-isopropyl-3(2H)-pyridazinone was prepared according to a similar manner to that of Example 188.

¹H NMR (DMSO-d₆, δ): 1.12 (6H, d, J=6.6 Hz), 1.25 (6H, d, J=6.0 Hz), 4.57-4.69 (1H, m), 5.01-5.14 (1H, m), 6.80 (1H, d, J=9.6 Hz), 6.91 (2H, d, J=8.8 Hz), 7.00 (2H, s), 7.12 (1H, d, J=9.6 Hz), 7.28 (2H, d, J=8.8 Hz), 8.38 (1H, s)

APCI/MS: 366[M+H]⁺

IR (KBr): 3180, 1643, 1569, 1481 cm⁻¹

mp: 164° C. (aq-EtOH)

### EXAMPLE 195

6-[2-Amino-4-(2-isopropoxyphenyl)-5-pyrimidinyl]-2-isopropyl-3(2H)-pyridazinonee was prepared according to a similar manner to that of Example 188.

¹H NMR (DMSO-d₆, δ): 0.72 (3H, t), 0.95-1.04 (6H, m), 1.34-1.43 (2H, m), 3.57-3.61 (2H, m), 4.92-4.99 (1H, m), 6.78 (1H, d, J=9.6 Hz), 6.92 (1H, d, J=8.4 Hz), 6.98 (2H, s), 7.03 (2H, m, J=t Hz), 7.22 (1H, d, J=9.6 Hz), 7.31-7.40 (2H, m), 8.46 (1H, s)

APCI/MS: 366[M+H]⁺

IR (KBr): 3421, 1648, 1573, 1452 cm⁻¹

mp: 202° C. (aq-EtOH)

### EXAMPLE 196

6-[2-Amino-4-(3-isopropoxyphenyl)-5-pyrimidinyl]-2-isopropyl-3(2H)-pyridazinone was prepared according to a similar manner to that of Example 188.

¹H NMR (DMSO-d₆, δ): 1.03 (6H, t, J=7.4 Hz), 1.17 (6H, d, J=6.6 Hz), 4.44-4.50 (1H, m), 5.01-5.08 (1H, m), 6.80 (1H, d, J=9.6 Hz), 6.81-6.94 (3H, m), 7.06 (2H, s), 7.16 (1H, d, J=9.6 Hz), 7.24-7.31 (2H, m), 8.44 (1H, s)

APCI/MS: 366[M+H]⁺

IR (KBr): 3191, 1648, 1573, 1492 cm⁻¹

mp: 143° C. (aq-EtOH)

### EXAMPLE 197

6-[2-Amino-4-(4-butoxyphenyl)-5-pyrimidinyl]-2-isopropyl-3(2H)-pyridazinone was prepared according to a similar manner to that of Example 188.

¹H NMR (DMSO-d₆, δ): 0.92 (3H, t, J=7.2 Hz), 1.14 (6H, d, J=6.6 Hz), 1.36-1.47 (2H, m), 1.61-1.71 (2H, m), 3.97 (2H, t, J=6.4 Hz), 5.04-5.11 (1H, m), 6.77 (1H, d, J=9.6 Hz), 6.93 (2H, d, J=8.8 Hz), 7.00 (2H, s), 7.03 (1H, d, J=9.6 Hz), 7.29 (2H, d, J=8.8 Hz), 8.38 (1H, s)

APCI/MS: 380[M+H]⁺

IR (KBr): 3384, 1641, 1583, 1484 cm⁻¹

mp: 143° C. (aq-EtOH)

### EXAMPLE 198

6-[2-Amino-4-(2-butoxyphenyl)-5-pyrimidinyl]-2-isopropyl-3(2H)-pyridazinone was prepared according to a similar manner to that of Example 188.

¹H NMR (DMSO-d₆, δ): 0.77 (3H, t, J=7.6 Hz), 0.94-1.04 (6H, m), 1.11-1.19 (2H, m), 1.32-1.38 (2H, m), 3.66 (2H, m), 4.92-4.99 (1H, m), 6.79 (1H, d, J=9.6 Hz), 6.92 (2H, d, J=8.4 Hz), 6.98 (2H, s), 7.04 (1H, t, J=7.2 Hz), 7.22 (1H, d, J=9.6 Hz), 7.31-7.40 (2H, m), 8.45 (1H, s)

APCI/MS: 380[M+H]⁺

IR (KBr): 3394, 1664, 1581, 1484 cm⁻¹

mp: 149° C. (aq-EtOH)

### EXAMPLE 199

6-[2-Amino-4-(3-butoxyphenyl)-5-pyrimidinyl]-2-isopropyl-3(2H)-pyridazinone was prepared according to a similar manner to that of Example 188.

¹H NMR (DMSO-d₆, δ): 0.89 (3H, t, J=7.2 Hz), 1.04 (6H, t, J=6.6 Hz), 1.28-1.46 (2H, m), 1.56-1.69 (2H, m), 3.86 (2H, t, J=6.4 Hz), 4.99-5.11 (1H, m), 6.80 (1H, d, J=9.6 Hz), 6.84-6.99 (3H, m), 7.07 (2H, s), 7.14 (1H, d, J=9.6 Hz), 7.28 (1H, t, J=8 Hz), 8.43 (1H, s)

APCI/MS: 380[M+H]⁺

IR (KBr): 3313, 1646, 1577, 1471 cm⁻¹

mp: 130° C. (aq-EtOH)

### EXAMPLE 200

6-[2-Amino-4-(4-isobutoxyphenyl)-5-pyrimidinyl]-2-isopropyl-3(2H)-pyridazinone was prepared according to a similar manner to that of Example 188.

¹H NMR (DMSO-d₆, δ): 0.96 (6H, d, J=6.6 Hz), 1.14 (6H, d, J=6.6 Hz), 1.96 (1H, m), 3.75 (2H, t, J=6.4 Hz), 5.04-5.11 (1H, m), 6.78 (1H, d, J=9.6 Hz), 6.93 (2H, d, J=8.8 Hz), 7.00 (2H, sd), 7.07 (1H, d, J=9.6 Hz), 7.30 (2H, d, J=8.8 Hz), 8.38 (1H, s)

APCI/MS: 380[M+H]⁺

IR (KBr): 3386, 1639, 1585, 1484 cm⁻¹

mp: 166° C. (aq-EtOH)

### EXAMPLE 201

6-[2-Amino-4-(2-isobutoxyphenyl)-5-pyrimidinyl]-2-isopropyl-3(2H)-pyridazinone was prepared according to a similar manner to that of Example 188.

¹H NMR (DMSO-d₆, δ): 0.72 (6H, d, J=6.8 Hz), 0.98-1.04 (6H, m), 1.62-1.68 (1H, m), 3.46 (2H, d, J=6.8 Hz), 4.93-4.99 (1H, m), 6.77 (1H, d, J=9.6 Hz), 6.93 (1H, d, J=8.0 Hz), 6.98 (2H, s), 7.03 (1H, t, J=7.2 Hz), 7.20 (1H, d, J=9.6 Hz), 7.31-7.40 (2H, m), 8.49 (1H, s)

APCI/MS: 380[M+H]⁺

IR (KBr): 3322, 1650, 1590, 1492 cm⁻¹

mp: 189° C. (aq-EtOH)

### EXAMPLE 202

6-[2-Amino-4-(3-isobutoxyphenyl)-5-pyrimidinyl]-2-isopropyl-3(2H)-pyridazinone was prepared according to a similar manner to that of Example 188.

¹H NMR (DMSO-d₆, δ): 0.91 (6H, d, J=6.6 Hz), 1.08 (6H, d, J=6.6 Hz), 1.86-1.99 (2H, m), 3.63 (2H, d, J=6.6 Hz), 5.02-5.09 (1H, m), 6.80 (1H, d, J=9.6 Hz), 6.84-6.97 (3H, m), 7.07 (2H,s), 7.13 (1H, d, J=9.6 Hz), 7.28 (1H, t, J=7.8 Hz), 8.44 (1H, s)

APCI/MS: 380[M+H]⁺

IR (KBr): 3315, 1666, 1573, 1486 cm¹

mp: 137° C. (aq-EtOH)

### EXAMPLE 203

6-{2-Amino-4-[4-(isopentyloxy)phenyl3-5-pyrimidinyl}-2-isopropyl-3(2H)-pyridazinone was prepared according to a similar manner to that of Example 188.

¹H NMR (DMSO-d₆, δ): 0.92 (6H, d, J=6.4 Hz), 1.13 (6H, d, J=6.6 Hz), 1.55-1.64 (2H, m), 1.7-1.80 (1H, m), 3.99 (2H, t, J=6.4 Hz), 5.05-5.11 (1H, m), 6.79 (1H, d, J=9.6 Hz), 6.93 (2H, d, J=8.8 Hz), 7.00 (2H, s), 7.08 (1H, d, J=9.6 Hz), 7.29 (2H, d, J=8.8 Hz), 8.38 (1H, s)

ESI/MS: 394[M+H]⁺, 416[M+Na]⁺

IR (KBr): 3383, 1640, 1586, 1480 cm⁻¹

mp: 164° C. (aq-EtOH)

### EXAMPLE 204

6-{2-Amino-4-(2-(isopentyloxy)phenyl]-5-pyrimidinyl}-2-isopropyl-3(2H)-pyridazinone was prepared according to a similar manner to that of Example 188.

¹H NMR (DMSO-d₆, δ): 0.76 (6H, d, J=6.6 Hz), 0.96-1.05 (6H, m), 1.21-1.31 (2H, m), 1.40-1.50 (1H, m), 3.67 (2H, m), 4.92-4.99 (1H, m), 6.79 (1H, d, J=9.6 Hz), 6.92-7.08 (2H, m), 6.96 (2H,s), 7.24 (1H, d, J=9.6 Hz), 7.29-7.41 (2H, m), 8.46 (1H, s)

ESI/MS: 394[M+H]⁺, 416[M+Na]⁺

IR (KBr): 3325, 1655, 1585, 1488 cm⁻¹

mp: 155° C. (aq-EtOH)

### EXAMPLE 205

6-{2-Amino-4-[3-(isopentyloxy)phenyl]-5-pyrimidinyl}-2-isopropyl-3(2H)-pyridazinone was prepared according to a similar manner to that of Example 188.

¹H NMR (DMSO-d₆, δ): 0.89 (6H, d, J=6.4 Hz), 1.08 (6H, d, J=6.6 Hz), 1.49-1.59 (2H, m), 1.65-1.75 (1H, m), 3.88 (2H, d, J=6.4 Hz), 5.01-5.08 (1H, m), 6.79 (1H, d, J=9.6 Hz), 6.83-6.96 (3H, m), 7.09 (2H, s), 7.14 (1H, d, J=9.6 Hz), 7.28 (1H, t, J=8.0 Hz), 8.44 (1H, s)

ESI/MS: 394[M+H]⁺, 416[M+Na]⁺

IR (KBr): 3310, 1656, 1570, 1480 cm⁻¹

mp: 97° C. (aq-EtOH)

### EXAMPLE 206

6-{2-Amino-4-[4-(hexyloxy)phenyl]-5-pyrimidinyl}-2-isopropyl-3(2H)-pyridazinone was prepared according to a similar manner to that of Example 188.

¹H NMR (DMSO-d₆, δ): 0.87 (3H, d, J=7.6 Hz), 1.14 (6H, d, J=6.6 Hz), 1.25-1.63 (6H, m), 3.96 (2H, t, J=6.4 Hz), 5.01-5.15 (1H, m), 6.78 (1H, d, J=9.6 Hz), 6.93 (2H, d, J=8.8 Hz), 7.00 (2H, s), 7.08 (1H, d, J=9.6 Hz), 7.29 (2H, d, J=8.8 Hz), 8.38 (1H, s)

ESI/MS: 408[M+H]⁺, 430[M+Na]⁺

IR (KBr): 3370, 1660, 1570, 1485 cm⁻¹

mp: 184° C. (aq-EtOH)

### EXAMPLE 207

6-{2-Amino-4-[2-(hexyloxy)phenyl]-5-pyrimidinyl}-2-isopropyl-3(2H)-pyridazinone was prepared according to a similar manner to that of Example 188.

¹H NMR (DMSO-d₆, δ): 0.8 (3H, d, J=6.8 Hz), 0.95 (6H, m), 1.14-1.39 (8H, m), 3.65 (2H, m), 4.92-4.98 (1H, m), 6.78 (1H, d, J=9.6 Hz), 6.90-7.07 (4H, m), 6.96 (2H, s), 7.23 (1H, d, J=9.6 Hz), 7.29-7.41 (2H, m), 8.45 (1H, s)

ESI/MS: 408[M+H]⁺, 430[M+Na]⁺

IR (KBr): 3325, 1655, 1585, 1488 cm⁻¹

mp: 92° C. (aq-EtOH)

### EXAMPLE 208

6-{2-Amino-4-[3-(hexyloxy)phenyl]-5-pyrimidinyl}-2-isopropyl-3(2H)-pyridazinone was prepared according to a similar manner to that of Example 188.

¹H NMR (DMSO-d₆, δ): 0.87 (3H, t, J=6.8 Hz), 1.04 (6H, d, J=8.4 Hz), 1.29-1.38 (6H, m), 1.57-1.67 (2H, m), 3.85 (2H, t, J=6.4 Hz), 4.98-5.12 (1H, m), 6.79 (1H, d, J=9.6 Hz), 6.84-6.96 (3H, m), 7.07 (2H, s), 7.14 (1H, d, J=9.6 Hz), 7.24-7.32 (1H, m), 8.44 (1H, s)

ESI/MS: 408[M+H]⁺, 430[M+Na]⁺

IR (KBr): 3330, 1660, 1580, 1485 cm⁻¹

mp: 121° C. (aq-EtOH)

### EXAMPLE 209

6-{2-Amino-4-[4-(2-fluoroethoxy)phenyl]-5-pyrimidinyl}-2-isopropyl-3(2H)-pyridazinone was prepared according to a similar manner to that of Example 188.

¹H NMR (DMSO-d₆, δ): 1.14 (6H, d, J=6.6 Hz), 4.15-4.17 (2H, m), 4.30-4.34 (2H, m), 5.05-5.11 (1H, m), 6.79 (1H, d, J=9.6 Hz), 6.98 (2H, d, J=8.8 Hz), 7.06 (2H, s), 7.08 (1H, d, J=9.6 Hz), 7.32 (2H, d, J=8.8 Hz), 8.39 (1H, s)

ESI/MS: 370[M+H]⁺, 392[M+Na]⁺

IR (KBr): 3178, 1635, 1585, 1482 cm⁻¹

mp: 200° C. (aq-EtOH)

### EXAMPLE 210

6-{2-Amino-4-[2-(2-fluoroethoxy)phenyl]-5-pyrimidinyl}-2-isopropyl-3(2H)-pyridazinone was prepared according to a similar manner to that of Example 188.

¹H NMR (DMSO-d₆, δ): 0.93-1.05 (6H, m), 3.60-4.00 (2H, m), 4.30-4.57 (2H, m), 4.91-4.97 (1H, m), 6.78 (1H, d, J=9.6 Hz), 6.93-7.43 (4H, m), 7.05 (2H, s), 7.22 (1H, d, J=9.6 Hz), 8.45 (1H, s)

ESI/MS: 370[M+H]⁺, 392[M+Na]⁺

IR (KBr): 3189, 1658, 1585, 1479 cm⁻¹

mp: 171° C. (aq-EtOH)

### EXAMPLE 211

6-{2-Amino-4-[3-(2-fluoroethoxy)phenyl]-5-pyrimidinyl}-2-isopropyl-3(2H)-pyridazinone was prepared according to a similar manner to that of Example 188.

¹H NMR (DMSO-d₆, δ): 1.07 (6H, d, J=6.6 Hz), 4.07-4.10 (1H, m), 4.22-4.26 (1H, m), 4.56-4.60 (1H, m), 4.80-4.84 (1H, m), 5.02-5.08 (1H, m), 6.80 (1H, d, J=9.6 Hz), 6.86-7.33 (4H, m), 7.08 (2H, s), 7.17 (1H, d, J=9.6 Hz), 8.45 (1H, s)

ESI/MS: 370[M+H]⁺, 392[M+Na]⁺

IR (KBr): 3183, 1641, 1575, 1487 cm⁻¹

mp: 123° C. (aq-EtOH)

### EXAMPLE 212

6-{2-Amino-4-[4-(2-methoxyethoxy)phenyl]-5-pyrimidinyl}-2-isopropyl-3(2H)-pyridazinone was prepared according to a similar manner to that of Example 188.

¹H NMR (DMSO-d₆, δ): 1.14 (6H, d, J=6.6 Hz), 3.30 (3H, s), 3.62 (2H, m), 4.10 (2H, m), 5.05-5.11 (1H, m), 6.78 (1H, d, J=9.6 Hz), 6.95 (2H, d, J=8.8 Hz), 7.01 (2H, s), 7.07 (1H, d, J=9.6 Hz), 7.30 (2H, d, J=8.8 Hz), 8.39 (1H, s)

ESI/MS: 382[M+H]⁺, 404[M+Na]⁺

IR (KBr): 3328, 1666, 1563, 1475 cm⁻¹

mp: 147° C. (aq-EtOH)

### EXAMPLE 213

6-{2-Amino-4-[3-(2-methoxyethoxy)phenyl]-5-pyrimidinyl}-2-isopropyl-3(2H)-pyridazinone was prepared according to a similar manner to that of Example 188.

¹H NMR (DMSO-d₆, δ): 1.01-1.09 (6H, m), 3.28 (3H, s), 3.57-3.61 (2H, m), 3.98-4.02 (2H, m), 5.01-5.08 (1H, m), 6.80 (1H, d, J=9.6 Hz), 6.86-6.99 (3H, m), 7.08 (2H, s), 7.15 (1H, d, J=9.6 Hz), 7.24-7.32 (1H, m), 8.44 (1H, s)

ESI/MS: 382[M+H]⁺, 404[M+Na]⁺

IR (KBr): 2960, 1671, 1573, 1471 cm⁻¹

### EXAMPLE 214

6-{2-Amino-4-[4-(3-fluoropropoxy)phenyl]-5-pyrimidinyl-2-isopropyl-3(2H)-pyridazinone was prepared according to a similar manner to that of Example 188.

¹H NMR (DMSO-d₆, δ): 1.14 (6H, d, J=6.6 Hz), 2.00-2.19 (2H, m), 4.08 (2H, t, J=6.2 Hz), 4.48 (1H, t, J=6.0 Hz), 4.71 (1H, t, J=6.2 Hz), 5.01-5.15 (1H, m), 6.78 (1H, d, J=9.6 Hz), 6.96 (2H, d, J=8.8 Hz), 7.01 (2H, s), 7.08 (1H, d, J=9.6 Hz), 7.31 (2H, d, J=8.8 Hz), 8.39 (1H, s)

ESI/MS: 384[M+H]⁺, 406[M+Na]⁺

IR (KBr): 3187, 1656, 1587, 1469 cm⁻¹

mp: 179° C. (aq-EtOH)

### EXAMPLE 215

6-{2-Amino-4-[2-(3-fluoropropoxy)phenyl]-5-pyrimidinyl}-2-isopropyl-3(2H)-pyridazinone was prepared according to a similar manner to that of Example 188.

¹H NMR (DMSO-d₆, δ): 0.94 (6H, brs), 1.61-1.88 (2H, m), 3.78 (2H, m), 4.21 (1H, t, J=6.0 Hz), 4.44 (1H, t, J=6.0 Hz), 4.92-4.99 (1H, m), 6.78 (1H, d, J=9.6 Hz), 6.89 (2H,s), 6.94-7.09 (2H, m), 7.24 (1H, d, J=9.6 Hz), 7.31-7.41 (2H, m), 8.46 (1H, s)

ESI/MS: 384[M+H]⁺, 406[M+Na]⁺

IR (KBr): 3156, 1635, 1579, 1486 cm⁻¹

mp: 200° C. (aq-EtOH)

### EXAMPLE 216

6-{2-Amino-4-[3-(3-fluoropropoxy)phenyl]-5-pyrimidinyl}-2-isopropyl-3(2H)-pyridazinone was prepared according to a similar manner to that of Example 188.

¹H NMR (DMSO-d₆, δ): 1.07 (6H, d, J=6.6 Hz), 1.95-2.14 (2H, m), 4.00 (2H, t, J=6.0 Hz), 4.44 (1H, t, J=6.0 Hz), 4.68 (1H, t, J=6.0 Hz), 5.01-5.08 (1H, m), 6.80 (1H, d, J=9.6 Hz), 6.86-6.99 (3H, m), 7.07 (2H, s), 7.15 (1H, d, J=9.6 Hz), 7.29 (1H, t, J=5.8 Hz), 8.44 (1H, s)

ESI/MS: 384[M+H]⁺, 406[M+Na]⁺

IR (KBr): 3191, 1658, 1575, 1485 cm⁻¹

mp: 123° C. (aq-EtOH)

### EXAMPLE 217

6-[2-Amino-4-(4-hydroxyphenyl)-5-pyrimidinyl]-2-isopropyl-3(2H)-pyridazinone (97 mg) was dissolved in N,N-dimethylformaide (5 ml). To the solution were add 2-dimethylaminoethyl chloride hydrochloride (47.5 mg) and potassium tert-butoxide (73.9 mg) at 25° C. The reaction mixture was stirred for 15 hours at ambient temperature. The reaction mixture was added to a mixture of water and ethyl acetate. The organic layer was separated and washed with brine. The aqueous layer was combined and extracted with ethyl acetate. The organic layer was combined and passed through the diatomaceous earth column. Evaporation of solvent under reduced pressure gave a residue. The residue was purified with column chromatography on silicagel (methanol:chloroform 10:90-20:80) to give 6-(2-amino-4-{4-(2-(dimethylamino)ethoxy]phenyl}-5-pyrimidinyl)-2-isopropyl-3(2H)-pyridazinone, which was crystallized from 90% aq-ethanol (36.3 mg).

¹H NMR (DMSO-d₆, δ): 1.13 (6H, d, J=6.6 Hz), 2.20 (6H, s), 2.60 (2H, t, J=6.0 Hz), 4.05 (2H, t, J=6.0 Hz), 5.05-5.11 (1H, m), 6.78 (1H, d, J=9.6 Hz), 6.94 (2H, d, J=8.8 Hz), 7.00 (2H, s), 7.08 (1H, d, J=9.6 Hz), 7.30 (2H, d, J=8.8 Hz), 8.38 (1H, s)

ESI/MS: 395[M+H]⁺, 417[M+Na]⁺

IR (KBr): 3421, 1652, 1587, 1484 cm⁻¹

mp: 162° C. (aq-EtOH)

### EXAMPLE 218

6-(2-Amino-4-{2-[2-(dimethylamino)ethoxy]phenyl}-5-pyrimidinyl)-2-isopropyl-3(2H)-pyridazinone was prepared according to a similar manner to that of Example 217.

¹H NMR (DMSO-d₆, δ): 0.95 (6H, brs), 2.23 (6H, s), 2.28 (2H, t, J=5.6 Hz), 3.73 (2H, brs), 5.05-5.11 (1H, m), 6.80 (1H, d, J=9.6 Hz), 6.96-7.08 (2H, m), 7.00 (2H, s), 7.27 (1H, d, J=9.6 Hz), 7.33-7.41 (2H, d), 8.45 (1H, s)

ESI/MS: 395[M+H]⁺, 417[M+Na]⁺

IR (KBr): 3160, 1664, 1573, 1457 cm⁻¹

mp: 198° C. (aq-EtOH)

### EXAMPLE 219

6-(2-Amino-4-{3-[2-(dimethylamino)ethoxy]phenyl}-5-pyrimidinyl)-2-isopropyl-3(2H)-pyridazinone was prepared according to a similar manner to that of Example 217.

¹H NMR (DMSO-d₆, δ): 1.08 (6H, d, J=6.6 Hz), 2.16 (6H, s), 2.55 (2H, t, J=5.6 Hz), 3.94 (2H, t, J=5.6 Hz), 5.01-5.08 (1H, m), 6.80 (1H, d, J=9.6 Hz), 6.85-6.98 (3H, m), 7.07 (2H, s), 7.14 (1H, d, J=9.6 Hz), 7.24-7.32 (1H, d), 8.44 (1H, s)

ESI/MS: 395[M+H]⁺, 417[M+Na]⁺

IR (KBr): 3421, 1635, 1592, 1484 cm⁻¹

mp: 111° C. (aq-EtOH)

### EXAMPLE 220

6-(2-Amino-4-{4-[2-(4-morpholinyl)ethoxy]phenyl}-5-pyrimidinyl)-2-isopropyl-3(2H)-pyridazinone was prepared according to a similar manner to that of Example 217.

¹H NMR (DMSO-d₆, δ): 1.14 (6H, d, J=6.6 Hz), 2.45 (4H, t, J=4.8 Hz), 2.67 (2H, t, J=5.6 Hz), 3.56 (4H, t, J=4.8 Hz), 4.09 (2H, t, J=5.6 Hz), 5.05-5.11 (1H, m), 6.79 (1H, d, J=9.6 Hz), 6.95 (2H, d, J=8.8 Hz), 7.00 (2H, s), 7.08 (1H, d, J=9.6 Hz), 7.30 (2H, d, J=8.8 Hz), 8.38 (1H, s)

ESI/MS: 437[M+H]⁺, 459[M+Na]⁺

IR (KBr): 3309, 1662, 1589, 1479 cm⁻¹

mp: 161° C. (aq-EtOH)

### EXAMPLE 221

6-(2-Amino-4-{2-[2-(4-morpholinyl)ethoxy]phenyl}-5-pyrimidinyl)-2-isopropyl-3(2H)-pyridazinone was prepared according to a similar manner to that of Example 217.

¹H NMR (DMSO-d₆, δ): 1.02 (6H, brs), 2.26 (4H, t, J=4.4 Hz), 2.36 (2H, t, J=6.0 Hz), 3.45 (4H, t, J=4.4 Hz), 3.75 (2H, ), 5.05-5.11 (1H, m), 6.80 (1H, d, J=9.6 Hz), 6.93-7.08 (2H, m), 6.98 (2H, s), 7.29 (1H, d, J=9.6 Hz), 7.29-7.39 (2H, m), 8.45 (1H, s)

ESI/MS: 437[M+H]⁺, 459[M+Na]⁺

IR (KBr): 3334, 1648, 1575, 1479 cm⁻¹

mp: 115° C. (aq-EtOH)

### EXAMPLE 222

6-(2-Amino-4-{3-[2-(4-morpholinyl)ethoxy]phenyl}-5-pyrimidinyl)-2-isopropyl-3(2H)-pyridazinone was prepared according to a similar manner to that of Example 217.

¹H NMR (DMSO-d₆, δ): 1.02-1.10 (6H, m), 2.42 (4H, t, J=4.6 Hz), 2.62 (2H, t, J=5.8 Hz), 3.55 (4H, t, J=4.6 Hz), 3.98 (2H, t, J=5.8 Hz), 5.05-5.11 (1H, m), 6.80 (1H, d, J=9.6 Hz), 6.85-6.98 (3H, m), 7.07 (2H,s), 7.14 (1H, d, J=9.6 Hz), 7.24-7.32 (1H, m), 8.43 (1H, s)

ESI/MS: 437[M+H]⁺, 459[M+Na]⁺

IR (KBr): 3309, 1650, 1573, 1475 cm⁻¹

mp: 68° C. (aq-EtOH)

### EXAMPLE 223

6-[2-Amino-4-(4-hydroxyphenyl)-5-pyrimidinyl)-2-methyl-3(2H)-pyridazinone was prepared according to a similar manner to that of Example 185.

¹H NMR (DMSO-d₆, δ): 3.68 (3H, s), 6.65-6.88 (4H, m), 6.97 (2H, brs), 7.26 (2H, d, J=8.6 Hz), 8.33 (1H, s), 9.87 (1H, brs)

ESI/MS: 296[M+H]⁺, 318[M+Na]⁺

IR (KBr): 3180, 1641, 1573, 1481, 1411 cm⁻¹

mp: 250° C. (H₂O)

### EXAMPLE 224

6-[2-Amino-4-(2-hydroxyphenyl)-5-pyrimidinyl]-2-methyl-3(2H)-pyridazinone was prepared according to a similar manner to that of Example 185.

¹H NMR (DMSO-d₆, δ): 3.58 (3H, s), 6.71 (1H, d, J=9.6 Hz), 6.76-6.88 (2H, m), 6.95 (1H, d, J=9.6 Hz), 7.05 (2H, brs), 7.21-7.28 (2H, m), 8.41 (1H, s), 10.0 (1H, s)

ESI/MS: 296[M+H]⁺, 318[M+Na]⁺

IR (KBr): 3180, 1646, 1579, 1496, 1440 cm⁻¹

mp: 260° C. (H₂O)

### EXAMPLE 225

6-[2-Amino-4-(3-hydroxyphenyl)-5-pyrimidinyl]-2-methyl-3(2H)-pyridazinone was prepared according to a similar manner to that of Example 185.

¹H NMR (DMSO-d₆, δ): 3.68 (3H, s), 6.69-6.83 (5H, m), 7.06 (2H, brs), 7.18 (1H, t, J=7.8 Hz), 8.39 (1H, s), 9.59 (1H, s)

ESI/MS: 296[M+H]⁺, 318[M+Na]⁺

mp: 285° C. (H₂O)

### EXAMPLE 226

6-[2-Amino-4-(4-bromophenyl)-5-pyrimidinyl]-3(2H)-pyridazinone was prepared according to a similar manner to that of Example 27.

¹H NMR (DMSO-d₆, δ): 6.74 (1H, d, J=9.8 Hz), 6.98 (1H, d, J=9.8 Hz), 7.19 (2H, s), 7.34 (2H, d, J=8.4 Hz), 7.61 (2H, d, J=8.4 Hz), 8.40 (1H, s), 13.1 (1H, s)

ESI/MS: 342, 344[M+H]⁺

IR (KBr): 3316, 1683, 1583, 1473 cm⁻¹

### EXAMPLE 227

6-[2-Amino-4-(3-bromophenyl)-5-pyrimidinyl]-3(2H)-pyridazinone was prepared according to a similar manner to that of Example 27.

¹H NMR (DMSO-d₆, δ): 6.75 (1H, d, J=10 0 Hz), 7.03 (1H, d, J=9.6 Hz), 7.15 (2H, s), 7.27-7.38 (2H, m), 7.61-7.66 (2H, m), 8.42 (1H, s)

ESI/MS: 366, 368[M+Na]⁺

IR (KBr): 3320, 1625, 1583, 1471 cm⁻¹

### EXAMPLE 228

6-[2-Amino-4-(2,6-difluorophenyl)-5-pyrimidinyl]-3(2H)-pyridazinone was prepared according to a similar manner to that of Example 27.

¹H NMR (DMSO-d₆, δ): 6.80 (1H, d, J=9.6 Hz), 7.10-7.24 (3H, m), 7.21 (1H, s), 7.30 (1H, d, J=9.6 Hz), 7.48-7.55 (1H, m), 8.55 (1H, s), 12.95 (1H, s)

ESI/MS: 324[M+Na]⁺

### EXAMPLE 229

6-[2-Amino-4-(3,5-difluorophenyl)-5-pyrimidinyl]-3(2H)-pyridazinone was prepared according to a similar manner to that of Example 27.

¹H NMR (DMSO-d₆, δ): 6.78 (1H, d, J=9.8 Hz), 7.03-7.11 (3H, m), 7.18 (2H, s), 7.30-7.41 (1H, m), 8.45 (1H, s), 13.1 (1H, s)

ESI/MS: 302(M+H]⁺, 324[M+Na]⁺

IR (KBr): 3342, 1623, 1585, 1490 cm⁻¹

### EXAMPLE 230

6-[2-Amino-4-(2,6-dichlorophenyl)-5-pyrimidinyl]-3(2H)-pyridazinone was prepared according to a similar manner to that of Example 27.

¹H NMR (DMSO-d₆, δ): 6.78 (1H, d, J=9.8 Hz), 7.19-7.24 (3H, m), 7.40-7.55 (3H, m), 8.58 (1H, s), 12.96 (1H, s)

ESI/MS: 356, 358[M+Na]⁺

IR (KBr): 3305, 1654, 1583, 1484 cm⁻¹

### EXAMPLE 231

6-[2-Amino-4-(2,6-dimethylphenyl)-5-pyrimidinyl]-3(2H)-pyridazinone was prepared according to a similar manner to that of Example 27.

¹H NMR (DMSO-d₆, δ) 1.96 (6H, s), 6.59 (1H, d, J=9.8 Hz), 6.72 (1H, d, J=9.8 Hz), 7.04-7.07 (4H, m), 7.15-7.22 (1H, m), 8.48 (1H, s), 13.04 (1H, s)

ESI/MS: 294[M+H]⁺, 316[M+Na]⁺

IR (KBr): 3156, 1679, 1575, 1475 cm⁻¹

### EXAMPLE 232

6-[2-Amino-4-(3-furyl)-5-pyrimidinyl]-3(2H)-pyridazinone was prepared according to a similar manner to that of Example 27.

¹H NMR (DMSO-d₆, δ): 6.51-6.52 (1H, m), 6.84 (1H, d, J=9.8 Hz), 6.94 (2H, s), 7.26 (1H, d, J=9.8 Hz), 7.71-7.78 (2H, m), 8.26 (1H, s), 13.1 (1H, s)

ESI/MS: 256[M+H]⁺, 278[M+Na]⁺

IR (KBr): 3187, 1658, 1581, 1490 cm⁻¹

### EXAMPLE 233

6-[2-Amino-4-(3-thienyl)-5-pyrimidinyl]-3(2H)-pyridazinone was prepared according to a similar manner to that of Example 27.

¹H NMR (DMSO-d₆, δ): 6.76 (1H, dd, J=9.6,2.2 Hz), 6.98 (2H, s), 7.03 (1H, d, J=9.6 Hz), 7.13-7.16 (1H, m), 7.55-7.59 (1H, m), 7.62-7.64 (1H, m), 8.31 (1H, s), 13.11 (1H, s)

ESI/MS: 272[M+H]⁺, 294[M+Na]⁺

IR (KBr): 3205, 1673, 1581, 1482 cm⁻¹

### EXAMPLE 234

6-[2-Amino-4-(2,6-dimethoxyphenyl)-5-pyrimidinyl]-3(2H)-pyridazinone was prepared according to a similar manner to that of Example 27.

¹H NMR (DMSO-d₆, δ): 3.60 (6H, s), 6.60 (1H, d, J=10.8 Hz), 6.66 (2H, d, J=8.4 Hz), 6.84 (1H, d, J=9.8 Hz), 6.96 (2H, s), 7.33 (1H, t, J=8.4 Hz), 8.35 (1H, s), 12.95 (1H, s)

ESI/MS: 326[M+H]⁺, 340[M+Na]⁺

IR (KBr): 3133, 1677, 1594, 1469 cm⁻¹

### EXAMPLE 235

6-[2-Amino-4-(4-bromophenyl)-5-pyrimidinyl]-2-isopropyl-3(2H)-pyridazinone was prepared according to a similar manner to that of Example 27.

¹H NMR (DMSO-d₆, δ): 1.01 (6H, d, J=6.6 Hz), 4.96-5.10 (1H, m), 6.85 (1H, d, J=9.6 Hz), 7.12 (2H, s), 7.28 (1H, d, J=9.6 Hz), 7.28 (2H, d, J=8.8 Hz), 7.58 (2H, d, J=8.8 Hz), 8.47 (1H, s),

ESI/MS: 384, 386[M+H]^{+,}

IR (KBr): 3397, 1646, 1581, 1481 cm⁻¹

mp: 204° C. (aq-EtOH)

### EXAMPLE 236

6-[2-Amino-4-(3-bromophenyl)-5-pyrimidinyl]-2-isopropyl-3(2H)-pyridazinone was prepared according to a similar manner to that of Example 27.

¹H NMR (DMSO-d₆, δ): 0.99 (6H, d, J=6.6 Hz), 4.96-5.09 (1H, m), 6.87 (1H, d, J=9.6 Hz), 7.16 (2H, s), 7.25-7.34 (2H, m), 7.35 (2H, d, J=9.6 Hz), 7.55-7.61 (2H, m), 8.49 (1H, s),

ESI/MS: 384, 386[M+H]⁺

IR (KBr): 3183, 1621, 1583, 1484 cm⁻¹

mp: 238-240° C. (aq-EtOH)

### EXAMPLE 237

6-[2-Amino-4-(2,6-difluorophenyl)-5-pyrimidinyl]-2-isopropyl-3(2H)-pyridazinone was prepared according to a similar manner to that of Example 27.

¹H NMR (DMSO-d₆, δ): 0.82 (6H, d, J=6.6 Hz), 4.86-4.99 (1H, m), 6.92 (1H, d, J=9.6 Hz), 7.09-7.19 (2H, m), 7.23 (2H, s), 7.40-7.55 (1H, m), 8.62 (1H, s)

ESI/MS: 366[M+Na]⁺

mp: 244° C. (aq-EtOH)

### EXAMPLE 238

6-[2-Amino-4-(3,5-difluorophenyl)-5-pyrimidinyl]-2-isopropyl-3(2H)-pyridazinone was prepared according to a similar manner to that of Example 27.

¹H NMR (DMSO-d₆, δ): 0.99 (6H, d, J=6.6 Hz), 4.96-5.10 (1H, m), 6.91 (1H, d, J=9.6 Hz), 7.01-7.06 (2H, m), 7.20 (2H, s), 7.24-7.35 (1H, m), 7.43 (1H, d, J=9.6 Hz), 8.53 (1H, s)

ESI/MS: 344[M+H]⁺, 366[M+Na]⁺

IR (KBr): 3421, 1635, 1583, 1490 cm⁻¹

mp: 234° C. (aq-EtOH)

### EXAMPLE 239

6-[2-Amino-4-(2,6-dichlorophenyl)-5-pyrimidinyl]-2-isopropyl-3(2H)-pyridazinone was prepared according to a similar manner to that of Example 27.

¹H NMR (DMSO-d₆, δ): 0.82 (6H, d, J=6.6 Hz), 4.86-5.00 (1H, m), 6.91 (1H, d, J=9.6 Hz), 7.30-7.54 (3H, m), 7.23 (2H, s), 7.66 (1H, d, J=9.6 Hz), 8.67 (1H, S)

ESI/MS: 376, 378[M+H]⁺, 398, 400[M+Na]⁺

IR (KBr): 3303, 1658, 1592, 1486 cm⁻¹

mp: 208° C. (aq-EtOH)

### EXAMPLE 240

6-[2-Amino-4-(2,6-dimethylphenyl)-5-pyrimidinyl]-2-isopropyl-3(2H)-pyridazinone was prepared according to a similar manner to that of Example 27.

¹H NMR (DMSO-d₆, δ): 0.92 (6H, d, J=6.6 Hz), 1.94 (6H, s), 4.97 (1H, m), 6.78 (1H, d, J=9.6 Hz), 7.00-7.17 (5H, m), 7.28 (1H, d, J=9.6 Hz)

ESI/MS: 336[M+H]⁺, 358[M+Na]⁺, 693[2M+Na]⁺

IR (KBr): 3340, 1652, 1583, 1486 cm⁻¹

mp: 212° C. (aq-EtOH)

### EXAMPLE 241

6-[2-Amino-4-(3-furyl)-5-pyrimidinyl]-2-isopropyl-3(2H)-pyridazinone was prepared according to a similar manner to that of Example 27.

¹H NMR (DMSO-d₆, δ): 1.23 (6H, d, J=6.6 Hz), 5.09-5.22 (1H, m), 6.45 (1H, s), 6.89 (1H, d, J=9.6 Hz), 6.96 (2H, s), 7.31 (1H, d, J=9.6 Hz), 7.69-7.74 (2H, m), 8.31 (1H, s)

ESI/MS: 298[M+H]⁺, 320[M+Na]⁺

IR (KBr): 3160, 1650, 1591, 1489 cm⁻¹

mp: 216° C. (aq-EtOH)

### EXAMPLE 242

6-[2-Amino-4-(3-thienyl)-5-pyrimidinyl]-2-isopropyl-3(2H)-pyridazinone was prepared according to a similar manner to that of Example 27.

¹H NMR (DMSO-d₆, δ): 1.18 (6H, d, J=6.6 Hz), 5.04-5.17 (1H, m), 6.84 (1H, d, J=9.6 Hz), 7.00 (2H, s), 7.05-7.08 (1H, m), 7.18 (1H, d, J=9.6 Hz), 7.53-7.61 (2H, m), 8.37 (1H, s)

ESI/MS: 314[M+H]⁺, 336[M+Na]⁺

IR (KBr): 3189, 1675, 1581, 1482 cm⁻¹

mp: 234° C. (aq-EtOH)

### EXAMPLE 243

6-[2-Amino-4-(2,6-dimethoxyphenyl)-5-pyrimidinyl]-2-isopropyl-3(2H)-pyridazinone was prepared according to a similar manner to that of Example 27.

¹H NMR (DMSO-d₆, δ): 0.99 (6H, d, J=6.6 Hz), 3.57 (6H, s), 4.97 (1H, m), 6.63 (2H, d, J=8.4 Hz), 6.73 (1H, d, J=9.6 Hz), 6.91 (2H, s), 7.15 (1H, d, J=9.6 Hz), 7.27 (1H, t, J=8.4 Hz), 8.40 (1H, s)

ESI/MS: 368[M+H]⁺, 390[M+Na]⁺

IR (KBr): 3409, 1648, 1581, 1486 cm⁻¹

mp: 260° C. (aq-EtOH)

### EXAMPLE 244

6-[2-Amino-4-(3-furyl)-5-pyrimidinyl]-2-methyl-3(2H)-pyridazinone was prepared according to a similar manner to that of Example 27.

¹H NMR (DMSO-d₆, δ): 3.69 (3H, s), 6.54 (1H, d, J=9.6 Hz), 6.88 (1H, d, J=9.6 Hz), 6.94 (2H, s), 7.25 (1H, d, J=9.6 Hz), 7.70-7.80 (2H, m), 8.26 (1H, s)

ESI/MS: 292[M+Na]⁺

IR (KBr): 3153, 1664, 1583, 1492 cm⁻¹

mp: 241° C. (aq-EtOH)

### EXAMPLE 245

6-[2-Amino-4-(3-thienyl)-5-pyrimidinyl]-2-methyl-3(2H)-pyridazinone was prepared according to a similar manner to that of Example 27.

¹H NMR (DMSO-d₆, δ): 3.69 (3H, s), 6.80 (1H, d, J=9.6 Hz), 7.04 (2H, s), 7.04 (1H, d, J=9.6 Hz), 7.17-7.20 (1H, m), 7.55-7.66 (2H, m), 8.31 (1H, s)

ESI/MS: 286[M+H]⁺, 308[M+Na]⁺

IR (KBr): 3154, 1664, 1579, 1490 cm⁻¹

mp: 252° C. (aq-EtOH)

### EXAMPLE 246

6-[2-Amino-4-(4-chlorophenyl)-5-pyrimidinyl]-3(2H)-pyridazinone (60 mg) was dissolved in N,N-dimethylformamide (5 ml). To the solution were added 60%-sodium hydride (28 mg) and methyl iodide (101 mg) at 25° C. The reaction mixture was heated at 50° C. and stirred for 20 hours. The reaction mixture was added to a mixture of water and ethyl acetate. The organic layer was separated and washed with brine, and dried over magnesium sulfate. Evaporation of solvent in vacuo gave a residue. The residue was purified with chromatography on silica gel (methanol:chloroform 5:95-10:90), to give 6-[4-(4-chlorophenyl)-2-(dimethylamino)-5-pyrimidinyl]-2-methyl-3(2H)-pyridazinone, which was crystallized from diisopropyl ether (30 mg).

¹H NMR (DMSO-d₆, δ): 3.21 (6H, s), 3.68 (3H, s), 6.76 (1H, d, J=9.6 Hz), 6.91 (1H, d, J=9.6 Hz), 7.40-7.50 (4H, m), 8.45 (1H, s)

ESI/MS: 342, 344[M+H]⁺, 364, 366[M+Na]⁺

IR (KBr): 3354, 1674, 1580, 1480 cm⁻¹

mp: 166° C. (IPE)

### EXAMPLE 247

6-[2-(Dimethylamino)-4-(4-methoxyphenyl)-5-pyrimidinyl]-2-methyl-3(2H)-pyridazinone was prepared according to a similar manner to that of Example 246.

¹H NMR (DMSO-d₆, δ): 3.21 (6H, s), 3.68 (3H, s), 3.78 (3H, s), 6.75 (1H, d, J=9.6 Hz), 6.86 (1H, d, J=9.6 Hz), 6.97 (1H, d, J=8.8 Hz), 7.45 (1H, d, J=8.8 Hz), 8.45 (1H, s),

ESI/MS: 338[M+H]⁺, 360[M+Na]⁺

IR (KBr): 3354, 1653, 1563, 1485 cm⁻¹

mp: 165° C. (IPE)

### EXAMPLE 248

6-[2-(Dimethylamino)-4-(3-fluorophenyl)-5-pyrimidinyl]-2-methyl-3(2H)-pyridazinone was prepared according to a similar manner to that of Example 246.

¹H NMR (DMSO-d₆, δ): 3.21 (6H, s), 3.68 (3H, s), 6.75 (1H, d, J=9.6 Hz), 6.93 (1H, d, J=9.6 Hz), 7.16-7.45 (3H, m), 8.45 (1H, s)

ESI/MS: 326[M+H]⁺, 348[M+Na]⁺

IR (KBr): 3362, 1685, 1562, 1475 cm⁻¹

mp: 184° C. (IPE)

### EXAMPLE 249

6-[2-(Dimethylamino)-4-(3-fluoro-4-methoxyphenyl)-5-pyrimidinyl]-2-methyl-3(2H)-pyridazinone was prepared according to a similar manner to that of Example 246.

¹H NMR (DMSO-d₆, δ): 3.21 (6H, s), 3.68 (3H, s), 3.86 (3H, s), 6.78 (1H, d, J=9.6 Hz), 6.94 (1H, d, J=9.6 Hz), 7.16-7.23 (2H, m), 7.42 (1H, d, J=14 Hz), 8.45 (1H, s)

ESI/MS: 356[M+H]⁺, 378[M+Na]⁺

IR (KBr): 3274, 1685, 1564, 1480 cm⁻¹

mp: 158° C. (IPE)

### EXAMPLE 250

6-(2-Amino-4-phenyl-5-pyrimidinyl)-2-(2-methoxy-1-methylethyl)-3(2H)-pyridazinone was obtained according to a similar manner to that of Example 2.

mp: 180-181° C. (EtOAc)

¹H NMR (DMSO-d₆, δ): 1.00 (3H, d, J=6.8 Hz), 3.16 (3H, s), 3.19-3.44 (2H, m), 5.11-5.21 (1H, m), 6.79 (1H, d, J=9.6 Hz), 7.08 (2H, brs), 7.13 (1H, d, J=9.6 Hz), 7.30-7.42 (5H, m), 8.44 (1H, s)

Elemental Analysis for C₁₈H₁₉N₅O₂

Calcd.: C,64.08; H,5.68; N,20.76

Found: C,64.23; H,5.67; N,20.84

ESI/MS: 360[M+Na]⁺

### EXAMPLE 251

To a stirred solution of 6-(2-amino-4-phenyl-5-pyrimidinyl)-2-(2-methoxy-1-methylethyl)-3(2H)-pyridazinone (195 mg) in dichloromethane (4 ml) was added boron tribromide (0.273 ml) under ice cooling and the mixture was allowed to stir at 0° C.-room temperature for 3.5 h. The mixture was poured into ice-water, neutralized with sat.NaHCO3 and extracted with chloroform (×2). The combined extracts were washed with brine, dried over MgSO₄ and concentrated under reduced pressure to afford 168 mg of crystals. The crude material was purified by silica-gel column chromatography (CHCl3-MeOH, 30:1) to give 129 mg of 6-(2-amino-4-phenyl-5-pyrimidinyl)-2-(2-hydroxy-1-methylethyl)-3(2H)-pyridazinone as colorless crystals.

mp: 222-223° C. (90% EtOH)

¹H NMR (DMSO-d₆, δ): 0.99 (3H, d, J-6.8 Hz), 3.28-3.45 (1H, m), 3.47-3.57 (1H, m), 4.72 (1H, t, J=5.8 Hz), 4.93-5.03 (1H, m), 6.77 (1H, d, J=9.6 Hz), 7.06 (2H, s), 7.05 (1H, d, J=9.6 Hz), 7.34-7.42 (5H, m), 8.45 (1H, s).

Elemental Analysis for C₁₇H₁₇N₅O₂

Calcd.: C,63.15; H,5.30; N,21.66

Found: C,63.23; H,5.31; N,21.69

APCI/MS: 324[M+H]⁺

### EXAMPLE 252

A mixture of 6-(2-amino-6-oxo-4-phenyl-1,6-dihydro-5-pyrimidinyl)-2-isopropyl-3(2H)-pyridazinone (1.00 g), triethylamine hydrochloride (0.51 g) and phosphorous oxychloride (1.44 ml) was heated at 100-105° C. for 3 hours. After cooling, the mixture was poured into ice-water, neutralized with saturated sodium hydrogen carbonate solution, extracted with chloroform, dried over magnesium sulfate and concentrated under reduced pressure to give a residue. The residue was purified by column chromatography on silica gel (ethyl acetate) to give 6-(2-amino-4-chloro-6-phenyl-5-pyrimidinyl)-2-isopropyl-3(2H)-pyridazinone as a solid (365 mg).

mp: 235-237° C. (acetone)

IR (KBr): 3404, 3199, 1645, 1581, 1570 cm⁻¹

ESI/MS: 366 and 364 [M+Na]⁺

¹H NMR (CDCl₃, δ): 1.09 (6H, d, J=6.60 Hz), 5.22 (1H, 7-plet, J=6.60 Hz), 5.48 (2H, br.s), 6.83 (1H, d, J=9.52 Hz), 7.08 (1H, d, J=9.52 Hz), 7.26-7.35 (5H, m)

### EXAMPLE 253

To a mixture of 6-[2-amino-4-(methylsulfonyl)-6-phenyl-5-pyrimidinyl]-2-isopropyl-3(2H)-pyridazinone (386 mg) and [1,3-bis(diphenylphosphino)propane]dichloronickel(II) (56.9 mg) in tetrahydrofuran (10 ml) was dropwise added a 3 M solution of methyl magnesium bromide in tetrahydrofuran (1.7 ml) under an atmosphere of nitrogen. The mixture was stirred at atmosphere temperature for 20 hours and concentrated under reduced pressure to give a residue. The residue was dissolved in a mixture of aqueous ammonium chloride solution and chloroform. An organic layer was dried over magnesium sulfate, concentrated under reduced pressure and purified by column chromatography on silica gel (ethyl acetate) to give 6-(2-amino-4-methyl-6-phenyl-5-pyrimidinyl)-2-isopropyl-3(2H)-pyridazinone as a solid (66 mg).

mp: 213-215° C. (methanol)

IR (KBr): 3433, 3325, 3194, 1639, 1587, 1562 cm⁻¹

ESI/MS: 665 [2M+Na]⁺, 344 [M+Na]⁺, 322 [M+H]⁺

¹H NMR (CDCl₃, δ): 1.28 (6H, d, J=6.62 Hz), 2.36 (3H, s), 5.21 (2H, br.s), 5.33 (1H, 7-plet, J=6.62 Hz), 6.70 (2H, s), 7.30 (5H, s)

1. An aminopyrimidine compound of the following formula (I). 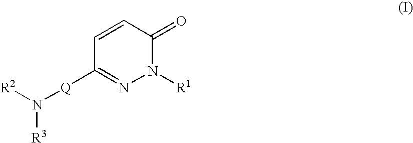
wherein Q is 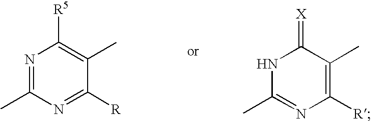
in which
R and R′ are each optionally substituted aryl or heterocyclic group,
R⁵ is hydrogen, halogen, lower alkyl, optionally substituted hydroxy, optionally substituted amino which may form N-containing heterocyclic group, optionally substituted mercapto, lower alkylsulfinyl or lower alkylsulfonyl, and
X is oxygen or sulfur;
R¹ is hydrogen, optionally substituted lower alkyl or cyclo(lower)alkyl which may be interrupted by an oxygen atom;
R² and R³ are each independently hydrogen, lower alkyl, acyl, aryl or heterocyclic (lower)alkyl,
R² and R³ may be combined together with N atom to which they are attached to form N-containing heterocyclic group; or a salt thereof.

2. A compound of claim 1,
wherein
R¹ is hydrogen, lower alkyl, hydroxy(lower)alkyl, lower alkoxy(lower)alkyl or phenyl(lower)alkyl,
R² is hydrogen, lower alkyl, lower alkanoyl or optionally substituted benzoyl,
R³ is hydrogen, lower alkyl, phenyl, pyridinyl(lower)alkyl or —CO—R³¹,
in which R³¹ is lower alkyl, cyclo(lower)alkyl, lower
alkoxy(lower)alkyl, optionally substituted lower alkoxy,
optionally substituted phenyl or pyridinyl,
R² and R³ may be combined together with N atom to which they are attached to form N-containing heterocyclic group;
R and R′ are each 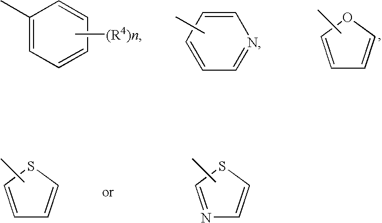
in which R⁴ is hydrogen, halogen, hydroxy, lower alkyl, optionally substituted lower alkoxy, trihalo(lower)alkyl, lower alkylthio, lower alkylsulfinyl or lower alkylsulfonyl, and n is an integer from 1 to 3,
provided R⁴ may be different from each other when n is 2 or 3; and
R⁵ is hydrogen, halogen, lower alkyl, lower alkylthio, lower alkanoylthio, arylthio, lower alkylsulfinyl, lower alkylsulfonyl, —O—R⁵¹,
in which R⁵¹ is hydrogen, optionally substituted lower alkyl, lower alkenyl, lower alkynyl, cyclo(lower)alkyl, aryl or heterocyclic group, or 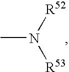
in which R⁵² is hydrogen or lower alkyl;
R⁵³ is hydrogen, optionally substituted lower alkyl, lower alkenyl, cyclo(lower)alkyl, amidino, aryl or heterocyclic group,
R⁵² and R⁵³ may be combined together with N atom to which they are attached to form N-containing heterocyclic group.

3. A compound of claim 2,
wherein
R¹ is hydrogen, methyl, ethyl, propyl, isopropyl, hydroxyisopropyl, methoxyisopropyl or benzyl;
R²is hydrogen, methyl, acetyl, benzoyl, toluoyl, methoxybenzoyl, trifluoromethylbenzoyl, fluorobenzoyl or chlorobenzoyl;
R³ is hydrogen, methyl, phenyl, pyridinylmethyl or —CO—R³¹,
in which R³¹ is methyl, propyl, isopropyl, isobutyl, tert-butyl, cyclopropyl, cyclohexyl, methoxy, methoxymethyl, trichloroethoxy, phenyl, tolyl, methoxyphenyl, trifluoromethylphenyl, fluorophenyl, chlorophenyl or pyridinyl, and
R² and R³ may be combined together with N atom to which they are attached to form morpholino;
R and R′ are each 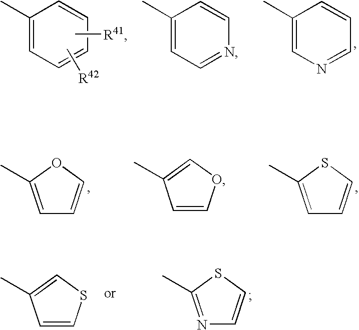
in which R⁴¹ and R⁴² are each independently hydrogen, fluoro, bromo, chloro, hydroxy, methyl, trifluoromethyl, methoxy, ethoxy, propoxy, isopropoxy, butoxy, isobutoxy, isopentyloxy, hexyloxy, methoxyethoxy, fluoroethoxy, fluoropropoxy, dimethylaminoethoxy, morpholinylethoxy, methylthio, methylsulfinyl or methylsulfonyl; and
R⁵ is hydrogen, fluoro, methyl, methylthio, acetylthio, phenylthio, methylsulfinyl, methylsulfonyl, —O—R⁵¹,
in which R⁵¹ is hydrogen, methyl, ethyl, propyl, isopropyl, allyl, propynyl, cyclobutyl, cyclohexyl, hydroxyethyl, methoxyethyl, carboxymethyl, aminoethyl, dimethylaminoethyl, fluoroethyl, carbamoylmethyl, methylcarbamoylmethyl, dimethylcarbamoylmethyl, cyclopropylcarbamoylmethyl, methoxycarbonylmethyl, tert-butoxycarbonylmethyl, acetylmethyl, benzoylmethyl, phenyl, benzyl, pyridinylmethyl, pyridinylethyl, tetrahydro-2H-pyranyl or 1,3(2H)-dioxoisoindolinylethyl, or 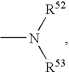
in which R⁵² is hydrogen or methyl,
R⁵³ is hydrogen, methyl, ethyl, propyl, isopropyl, tert-butyl, allyl, cyclopropyl, hydroxyethyl, methoxyethyl, aminoethyl, dimethylaminoethyl, carbamoylmethyl, amidino, phenyl, benzyl, pyridinyl, pyridinylmethyl, furylmethyl or dimethylthiazolyl,
R⁵² and R⁵³ may be combined together with N atom to which they are attached to form pyrrolidinyl, piperidinyl, morpholino, piperazinyl, methylpiperazinyl, imidazolyl, triazolyl or benzimidazolyl.

4. A compound of claim 1,
wherein Q is 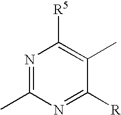
in which R and R⁵ are each as defined in claim, and R¹ is optionally substituted lower alkyl.

5. A compound of claim 4,
wherein
R¹ is lower alkyl or lower alkoxy(lower)alkyl, and
R⁵ is hydrogen.

6. A compound of claim 5,
wherein
R¹ is lower alkyl, and
R² is hydrogen.

7. A process for the preparation of the aminopyrimidine compound of claim 1 or a salt thereof, which comprises,
(1) hydrolyzing a compound of the formula (II): 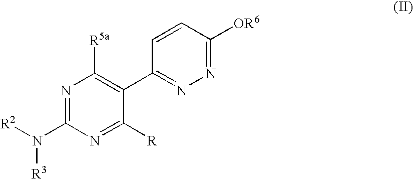
wherein
R, R² and R³ are each as defined above,
R^{5a} is hydrogen, lower alkyl, optionally substituted hydroxy or optionally substituted amino, and
R⁶ is lower alkyl, or a salt thereof, to give a compound of the formula (Ia′): 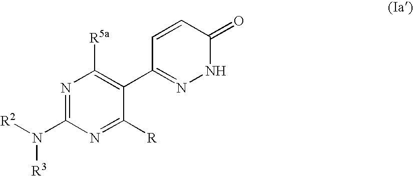
wherein R, R², R³ and R^{5a} are each as defined above or a salt thereof,
(2) reacting a compound of the formula (Ia): 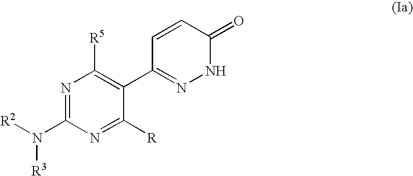
wherein R, R², R³ and R⁵ are each as defined above or a salt thereof, with a compound of the formula (III):
R^{1a}—Y¹ (III)
wherein R^{1a} is lower alkyl, cyclo(lower)alkyl which may be interrupted by an oxygen atom or aryl(lower)alkyl, and
Y¹ is a leaving group,
or a salt thereof,
to give a compound of the formula (Ib): 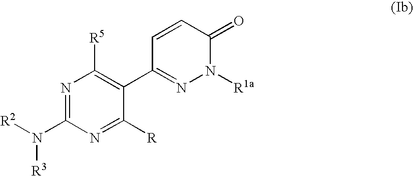
wherein R, R², R³, R⁵ and R^{1a} are each as defined above or a salt thereof,
(3) reacting a compound of the formula (Ic): 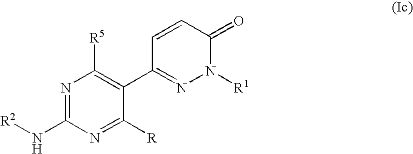
wherein R, R¹, R² and R⁵ are each as defined above or a salt thereof, with a compound of the formula (IV):
R^{3a}—Y² (IV)
wherein R^{3a} is lower alkyl, acyl, aryl and aryl(lower)aryl, and Y² is a leaving group, or a salt thereof to give a compound of the formula (Id): 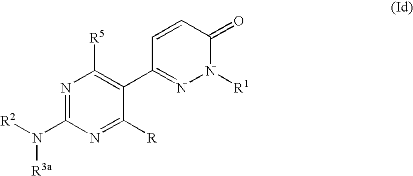
wherein R, R¹, R², R⁵ and R^{3a} are each as defined above or a salt thereof,
(4) reacting a compound of the formula (V): 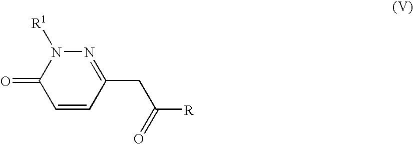
wherein R and R¹ are each as defined above or a salt thereof, with a compound of the formula: 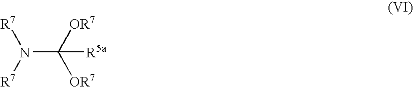
wherein R^{5a} is as defined above,
and R⁷ is lower alkyl or a salt thereof,
and further with a compound of the formula (VIII): 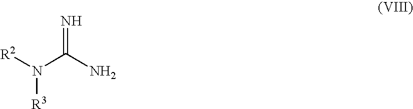
wherein R² and R³ are each as defined above or a salt thereof, to give a compound of the formula (Ie): 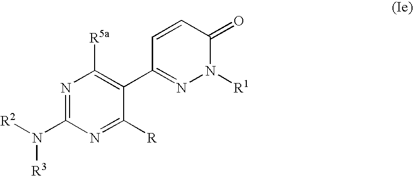
wherein R, R¹, R², R³ and R^{5a} are each as defined above or a salt thereof,
(5) reacting a compound of the formula (XXVI): 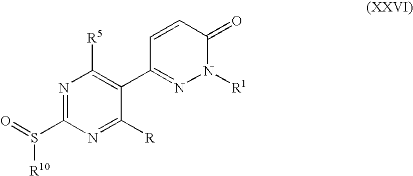
wherein R, R¹ and R⁵ are each as defined above, and
R¹⁰ is lower alkyl, or a salt thereof,
with a compound of the formula (XXVII):
R²—NH—R³ (XXVII)
wherein R² and R³ are each as defined above or a salt thereof, to give a compound of the formula (I): 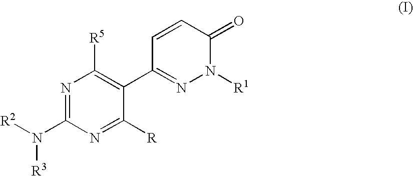
wherein R, R¹, R², R³ and R⁵ are each as defined above or a salt thereof,
(6) reacting a compound of the formula (XXXII): 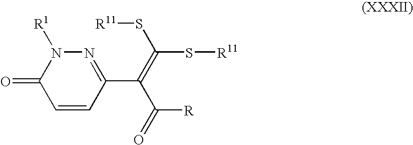
wherein R and R¹ are each as defined above, and
R¹¹ is lower alkyl, or a salt thereof,
with a compound of the formula (VIII): 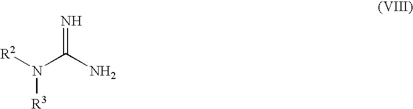
wherein R² and R³ are each as defined above or a salt thereof, to give a compound of the formula (If): 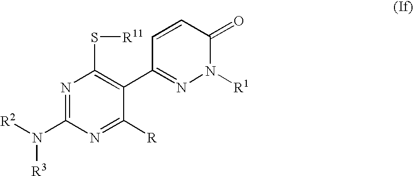
wherein R, R¹, R², R³ and R¹¹ are each as defined above or a salt thereof,
(7) oxidizing a compound of the formula (If): 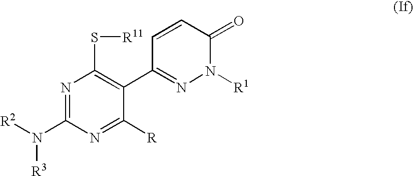
wherein R, R¹, R², R³ and R¹¹ are each as defined above or a salt thereof,
to give compounds of the formula (Ig) and (Ih): 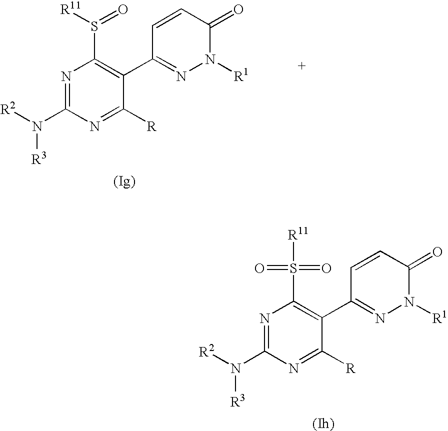
wherein R, R¹, R², R³ and R¹¹ are each as defined above or a salt thereof,
(8) reacting a compound of the formula (Ig): 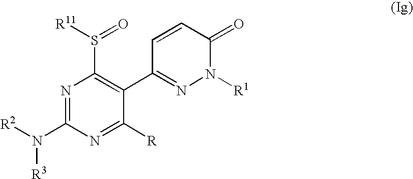
wherein R, R¹, R², R³ and R¹¹ are each as defined above or a salt thereof,
with a compound of the formula (XXXIII):
R^{51a}—OH (XXXIII)
wherein R^{51a} is optionally substituted lower alkyl, lower alkenyl, lower alkynyl, cyclo(lower)alkyl, aryl or heterocyclic group, or a salt thereof,
to give a compound of the formula (Ii): 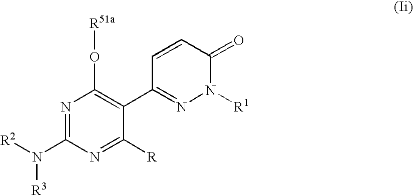
wherein R, R¹, R², R³ and R^{5a} are each as defined above or a salt thereof,
(9) reacting a compound of the formula (Ih): 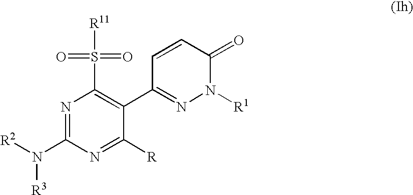
wherein R, R¹, R², R³ and R¹¹ are each as defined above or a salt thereof,
with a compound of the formula (XXXIII):
R^{51a}—OH (XXXIII)
wherein R^{51a} is optionally substituted lower alkyl, lower alkenyl, lower alkynyl, cyclo(lower)alkyl, aryl or heterocyclic group, or a salt thereof,
to give a compound of the formula (Ii): 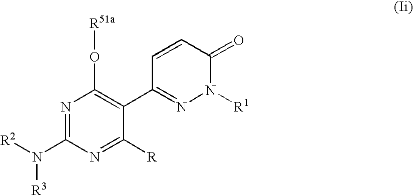
wherein R, R¹, R², R³ and R^{51a} are each as defined above or a salt thereof,
(10) reacting a compound of the formula (Ig): 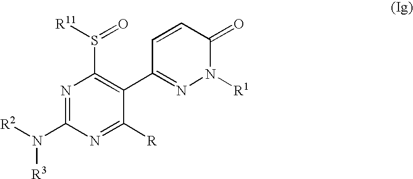
wherein R, R¹, R², R³ and R¹¹ are each as defined above or a salt thereof,
with a compound of the formula (XXXIV): 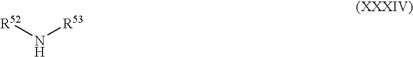
wherein R⁵² is hydrogen or lower alkyl,
R⁵³ is hydrogen, optionally substituted lower alkyl, lower alkenyl, cyclo(lower)alkyl, amidino, aryl or heterocyclic group, and R⁵² and R⁵³ may be combined together with N atom to which they are attached to form N-containing heterocyclic group, or a salt thereof,
to give a compound of the formula (Ij): 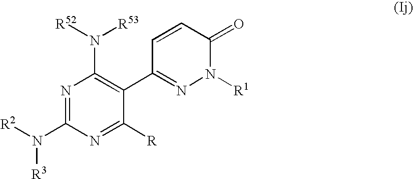
wherein R, R¹, R², R³, R⁵² and R⁵³ are each as defined above or a salt thereof,
(11) reacting a compound of the formula (If): 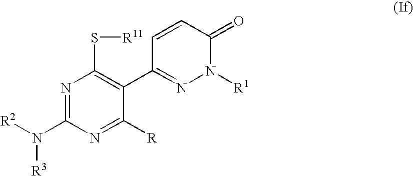
wherein R, R¹, R², R³ and R¹¹ are each as defined above or a salt thereof,
with urea hydrogen peroxide addition compound, to give a compound of the formula (Ik): 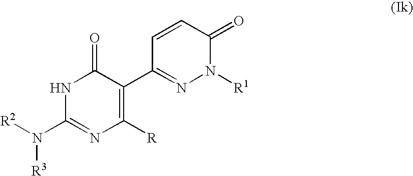
wherein R, R¹, R² and R³ are each as defined above or a salt thereof,
(12) reacting a compound of the formula (Ik): 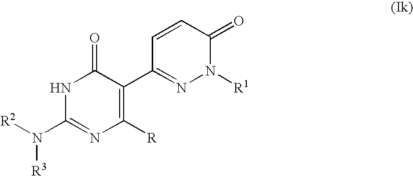
wherein R, R¹, R² and R³ are each as defined above or a salt thereof,
with a compound of the formula (XXXV): 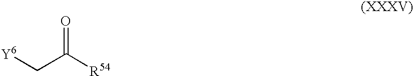
wherein R⁵⁴ is lower alkyl, cyclo(lower)alkyl, lower alkoxy or aryl, and Y⁶ is a leaving group,
or a salt thereof,
to give a compound of the formula (Il): 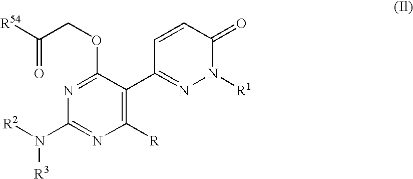
wherein R, R¹, R², R³ and R⁵⁴ are each as defined above or a salt thereof,
(13) reacting a compound of the formula (Im): 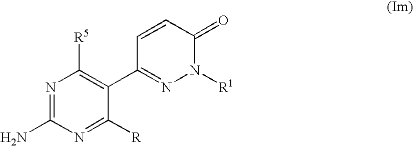
wherein R, R¹and R⁵ are each as defined above or a salt thereof, with a compound of the formula (XXXVI): 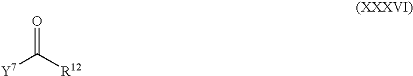
wherein R¹² is optionally substituted aryl or lower alkoxy, and Y⁷ is a leaving group,
or a salt thereof,
to give a compound of the formula (In): 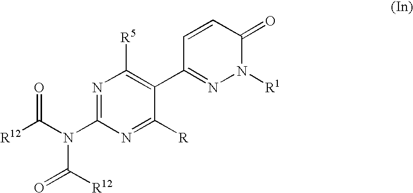
wherein R, R¹, R², R³ and R⁵⁴ are each as defined above or a salt thereof,
(14) reacting a compound of the formula (Io): 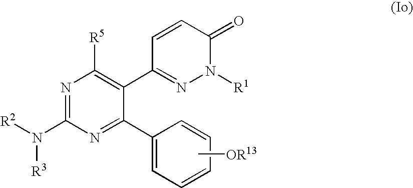
wherein R¹, R², R³ and R⁵ are each as defined above, and R¹³ is lower alkyl, or a salt thereof,
to give a compound of the formula (Ip): 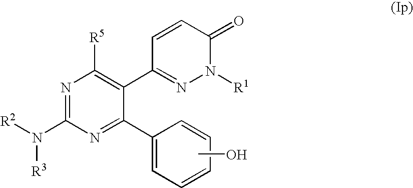
wherein R¹, R², R³ and R⁵ are each as defined above or a salt thereof,
(15) reacting a compound of the formula (Ip): 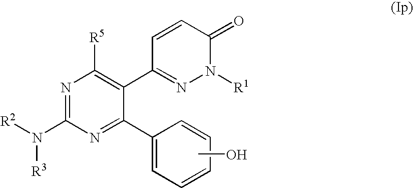
wherein R¹, R², R³ and R⁵ are each as defined above, or a salt thereof,
with a compound of the formula (XXXVII):
Y⁸—R¹⁴ (XXXVII)
wherein R¹⁴ is optionally substituted lower alkyl, and Y⁸ is a leaving group, or a salt thereof, to give a compound of the formula (Iq): 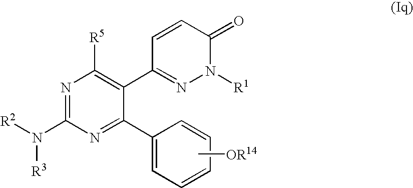
wherein R¹, R², R³, R⁵ and R¹⁴ are each as defined above or a salt thereof.
(16) reacting a compound of the formula (Ih): 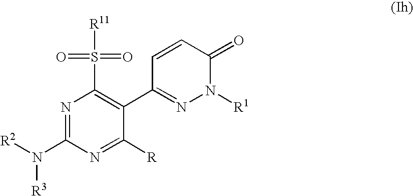
wherein R, R¹, R², R³ and R¹¹ are each as defined above, or a salt thereof,
with a compound of the formula (XXXVIII):
Y⁹—M—R^{5b} (XXXVIII)
wherein R^{5b} is lower alkyl, Y⁹ is a leaving group and M is metal, or a salt thereof,
to give a compound of the formula (Ir): 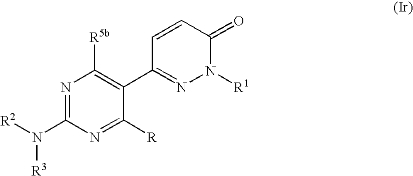
wherein R, R¹, R², R³ and R^{5b} are each as defined above or a salt thereof.

8. A pharmaceutical composition comprising the compound of claim 1 or a pharmaceutically acceptable salt thereof in admixture with a pharmaceutically acceptable carrier.

9. A method for preventing or treating a disease selected from the group consisting of depression, dementia, Parkinson's disease, anxiety, pain, cerebrovascular disease, heart failure, hypertension, circulatory insufficiency, post-resuscitation, asystole, bradyarrhythmia, electromechanical dissociation, hemodynamic collapse, SIRS (systemic inflammatory response syndrome), multiple organ failure, renal failure (renal insufficiency), renal toxicity, nephrosis, nephritis, edema, obesity, bronchial asthma, gout, hyperuricemia, sudden infant death syndrome, immunosuppression, diabetes, ulcer, pancreatitis, Meniere's syndrome, anemia, dialysis-induced hypotension, constipation, ischemic bowel disease, ileus, myocardial infarction, thrombosis, obstruction, arteriosclerosis obliterans, thrombophlebitis, cerebral infarction, transient ischemic attack and angina pectoris, which comprises administering the compound of claim 1 or a pharmaceutically acceptable salt thereof to a human being or an animal.

10. A method for preventing or treating a disease selected from the group consisting of depression, dementia, Parkinson's disease, anxiety, pain, cerebrovascular disease, Meniere's syndrome and cerebral infarction, which comprises administering the compound of claim 1 or a pharmaceutically acceptable salt thereof to a human being or an animal.

11. A compound of claim 1 or a pharmaceutically acceptable salt thereof for use as a medicament.

12. A compound of claim 1 or a pharmaceutically acceptable salt thereof for use as an adenosine antagonist.

13. A compound of claim 1 or a pharmaceutically acceptable salt thereof for use as an A₁ receptor and A₂ receptor dual antagonist.

14. A process for preparing a pharmaceutical composition which comprises admixing the compound of claim 1 or a pharmaceutically acceptable salt thereof with a pharmaceutically acceptable carrier.

15. Use of the compound of claim 1 or a pharmaceutically acceptable salt thereof for the production of a pharmaceutical composition for the therapy of diseases on which an adenosine antagonist is therapeutically effective.

16. A method for evaluation of adenosine antagonism which comprises use of compound of claim 1 or a pharmaceutically acceptable salt thereof.
